SEQUENCE LISTING (35 sequences)

SEQ ID NO: 1 - DNA, 7080 bp, unidentified
  source 1..7080: mol_type = genomic DNA
atggaaaacctgattaaaaaaagaatcaagcaagtcaaaaaaggcgatcaaaatgcgtttgcagaaatcgttgacctttataaagataagatttaccagctttgctaccgcatgctcggcaatgtgcacgaagcggaagatattgcgcaggaagcttttatcagagcgtatgtcaatatcgacagtttcgatatcaacagaaagttttcaacgtggctttacagaattgcgaccaacctgaccattgaccgcatccgcaagaaaaagcctgattactatctggacgcagaagtagcgggagcagaagggctgacgatgtattcgcagatcgccgcggacggggttctccctgaagacgccgtattgtcattggagctgtcggacacaattcagaaaaagattttaaagctgccggacaagtacagaacagttattgtattaaagtatattgacgaactctctttaattgaaatcggggagattctgaatatccctgtcggcaccgtcaagacaaggatacacagaggcagagaggctcttagaaaacaattgagggatctttaagtggggtgatgaaatgagctgtcctgaacagattgtgcagcttatgcataagcatcttgatggagatatccttcctgaagatgaaaacgtattaaaacaacatttgcagtcatgcgggaattgcaaacggcattttcatgaaatggagaaatccgtagcactcgtccgaagcacatcgcatgtcacggcccctgctgattttactgccaaagttatggcgaatcttccaaaagagaaaaaacgcgcttctgtaaaacgatggatcaaaactcatccgatgattgcagccgcagcggtttttgtgattttaatgggcggcggttttttcaacagctggcataatgaccacaatttcagtgtgtccaaacagccgaaccttatcgtgcagaatcacacggtcaccgtccctgaaggcgagaccgtaaagggcgatgtcacagtaaaaaacggcaagctgttcattgaaggaaaagtcgacggcaatgtgaccgtcgtcaacggagaaaaatacatggcctcagccggacaagtcaccgggcagattgaagagattaaccaattgtttgactgggtctggtacgaactgaaatcggcgggccagggcgtcgtaagcgctttggacccgaatgaagatgagtaaagcgtataatcgctttactcttttttttatgcatcgccgtgccggcccatttcacttgagaaatcaaaatgtggtataatgagcacgctatatgatgtacatattgctcatgcagcacaattcataacggaaaaaacgaaatgactgaattcttggaggacgaggaaatggcttttgaggacatcccatttttgcaatacctcggcaatgccgttgatattctccttgtttggtatgtgatatataaattaatcatggtcatacgcggaacgaaggcggttcagcttttaaaaggaatcgtcgtcatcgtattggtccggatggccagttcatatttgggactcagcacgcttcagtggctgatggaccaagcgataacatggggatttttagcgatcatcattatattccagcctgagctgagacgggcgcttgaacagctcggacggggccggttcttttccaggagcggaacccccgtggaggaagcgcagcataagacaattgaagcgattacaaaatcgatcagctacatggcgaaacgccgcatcggcgcactcatcgccattgagcgcgatacgggaatgggcgattatattgagacgggcatcccgcttaatgcgcaggtcagctcggagcttttgattaatatcttcatcccgaatacaccgctgcatgatggagcggtcattatgaaaaataacgagattgcggcggcggcctgttatctgccgctttctgaaagcccgtttatttcaaaggagctggggacgcgtcacagagcggctgtcggcatcagtgaagtgacggacagcttaaccattattgtatctgaagagacgggcgccatcagtgtggccaaaaacggagaccttcacagagaattgacggaagaagcactgaaagaaatgcttgaagccgagtttaagaagaacgccagagatacttcttctaaccgctggtactggaggggcaagaaaaatggataaattcttaaacaaccgctgggctgtaaagatcattgcattgctcttggcgctgctgctttacgttgcggttaacaacaaccaggcgccgactccgaaaaagccgggggaatccttttttccgacatcaacgactgacgaggctacgcttactgatattcctgtaaaagcatactatgatgaagcgaactatgtcgtcaccggtgtgccccagaccgtaaatgtcacgattaaaggctcgacaagcgcggtgaaaaaggcgagacagacgaagaattttgagatatacgccgatatgaataagctgaagaccgggactcataaggtcgaattgaaggcgaaaaacgtatcggacggactgacggtctccattaacccgtcagtcacgacggttacgattcaggaaaaaacggctgagagttttcccgtggaagttgaatactacaataaaagcaaaatgaaaaagggatataccccccagcagccgattatcagcccgaaaaacgtgcagattacagggtcgaaaaatgtgattgataacatttcgcttgtcaaagcatccgtgaaccttgaaaatgcaaatgagaccattgaaaaggaagtgaaggtgaccgtctatgacaaagacggcaacgcacttcctgttgatgttgaaccgtcagtcattaaagtgacggttcccgtagccagtccgagtaaaaagcttcctttcaaaattgaaaggaagggaagtcttccggacggggtgagcgttgcgagcattgactcgaacccgagtgaggtgacggtttacggcccgcaggatgtgctggattcacttgaatttattgacggcgtatctcttgatttaggcaaaattgataaagataccgatatcaatgccgatatcccgcttcctaagggtgtcactaaggtatcaccggaaaaaatcacgcttcatgttaaagtggacagtgaagacaaaaaggattttgacaatgtcgcaatcaagacggtcggactgggaggctcacagcagattgagtttctcgatcctgaatcgcaggcgataaatattacggccaaaggctctcccgccaacattaaggcgctgaaaaagtcggacattgaactgtatgccaatgtctcggacttggatgacggagagcactctgtaaagcttgaagtaaacggcccgcaaaacgtcacatggtccctgtcaaaagacacggttaagatcaagctgacttctaagaaaaacacaagttcaaataaaaatgacggcggctccgctgattccggcaataataacggcacaagcggagacgcagacaaagacaagagcaagaaccgggaagacgacactgacagcgatcagaataaaagtcaaaaccaggatcaaaatcaagaccaggatgaagacggatcaagcgctgattcttcttcaacagaatgaaaaaggagcgatacaaaatgggcaagtattttggaacagatggtgtaagaggtgttgccaacagcgagcttacgccagagctggcctttaaagtggggcgttttggcggttatgtcctgaccaaagataagcagcgcccgaaagtgctgatcggccgtgatacacgtatctccggacatatgctggagggagcccttgtcgccggacttttatcgatcggagctgaagtgatgcgcctcggcgttatttctacgccgggggtatcctatttaacgaaagccatggatgctgaggcgggcgtcatgatctccgcttctcacaaccccgtacaggataacggaattaaatttttcggaggggacggctttaagctttctgatgagcaggaggccgagattgaacgtctcatggaggaaccggaagataagctgccgagaccgacaggcgcggacctagggcttgtgaacgattattttgaaggcgttcaaaagtatctgcaatttttaaagcagacggcggacgaagattttacgggcattcatgtagcgcttgactgcgcccatggtgcgacatcttccattgcgacgcacctttttgccgatctcgatgctgacgtatctacaatggggacgtctccgaacggattaaacattaatgatggcgtcggctctacacacccggaagcgctgagtgctttcgtcaaagaaaaaaacgccgatatcggccttgcgtttgacggagacggcgaccgcctcattgccgttgatgaaaaaggaaacatcgtcgacggcgatcagatcatgtacatatgcgcgaagtatttgaaatcagaaggccgtctgaaggatgataccgtcgtatcgaccgtgatgagcaacctcggtttctacaaagcgcttgaaaaagaagaaatcaaaagcgttcaaacggcagtcggcgaccgctatgtcgtagaagcgatgaaaaaagacggctataatgtcggcggcgagcagtcagggcaccttattttccttgattacaacactaccggcgacggtctcttgtccgcgatcatgcttgtgaatacgctgaaggcgtccggcaaaaccctttcagaacttgcggatgaaatgaaaaaattcccgcagctcttagtgaacgtgaaagtgacggataaatataaggtagaagaaaacgctaaggtgaaggccgtgatttctgaagtggaaaaagaaatgaacggagacggccggattttagtgcgcccgtcaggcaccgaaccgctcgtccgtgtgatggcggaagcgagaacaaaagagctttgtgacgaatatgtcacaaaaatcgtggatgtcgtgcggacagaaatgggagcggagtagcgaagtttataagcagagagcgaacaagacaagtgcgatcagcataaaatgatagtaaacatgatgccggggcggcaaaaaataattgaccgtccttggcacatgctgtaaaatcaagcttgttcagcctttgtttttcacttgcaggaaaggcgggattgtttgcatcttttataattacagcgcccgaactaagcgcccgaaaaacggcttagttgacgaggatggaggttatcgaattttcggcggatgcctcccggctgattcatttagatcacagccgtaaggatttcttcaaaccggaggggcgactctccgaacaaagagaaatcacatgatcttccaaaaaaacatgtgggaggggacgattgaaagtccccttgtaatttgactttcttcgtctccttttacacctttaggaggaagaaaatatgtgtggaattgtaggttacatcggtcagcttgatgcgaaagagattttgttaaaaggattagagaagctggagtaccgcggctatgactctgccggtatcgctgtggcgaatgagcagggcgtgcatgtgtacaaagaaaaaggccgcatcgccgaccttcgtgaagtggtggatcacacggttgaatctcaagcgggaatcggccatacacgctgggcgactcacggcgaaccaagcttcctgaacgctcacccgcatcaaagcgcactcggccgctttacacttgttcataacggtgtgatcgagaactatgttcagctgaagcgcgagtatcttgaaaacgttgaactgaaaagcgacacggacactgaagtagtcgttcaaatgatcgagcaatttgtggcgggaggactcagcacagaagaagcgttccgtaaaacactgactctgttaaaaggctcttacgcaattgcattatttgacggtgaaaacacagacaccatttacgttgcaaaaaacaaaagccctctgttaatcggccttggagatacgtttaacgtcgtggcatctgacgcgatggctatgcttcaagtaacgaatgaatacgttgagcttttggacaaagaaatggtgatcgtgacaaaagacgaagccgtgattaaaaaccttgacggtgaagtcatgacacgtgcgtcttatatcgctgagcttgacgccagtgatatcgaaaaaggcacataccctcactacatgttaaaagaaacggatgagcagccgcttgttatgcgcaaaatcatccaaacgtatcaggacgaaaacggcagactggccgtggccggcgatgtcgctgacgccgtggcggaagcggaccgcatttatatcgtggcttgcggaacgagctaccatgccggtcttgtcgggaaacaatatattgaaatgtgggcaaacgtaccggttgaagtgcatgtagcgagtgaattctcttataacatgccgcttctgtctaagaagccgctctttatcttcctttctcaaagcggagaaacagcggacagccgcgctgtgcttgttcaagtcaaagcgctgggtcacaaagcgctgacgattacaaacgttccgggatcaacgctttcccgtgaagcggattacacattgcttctgcacgcaggccctgagatcgccgtggcatcaacaaaagcgtatacggctcagattgccgtcctcgcgatcgttgcatcagttgcggcagaacgcaacggcgttgatatcggctttgatttagtcaaagaattaggtatcgcggcaaatgccatggaagctctctgcgaccagaaggacgaaatggaaatgatcgcgcgtgagtacctgactgtttcaagaaacgctttcttcatcggccgcggccttgactacttcgtatgtgtcgaaggcgccctgaagctgaaagagatttcttacattcaggcagaaggcttcgccggcggcgagctgaagcacggaacaatcgccctgatcgaagaaggaacaccggtctttgcacttgcgacacaagaacacgtcaacctgagcatccgcggaaacgtgaaggaagtcgcagcccgcggcgccaacacttgcatcatctcgctgaaaggcttagaagacgcagacgacagat

SEQ ID NO: 2 - DNA, 7320 bp, unidentified
  source 1..7320: mol_type = genomic DNA
tcatcctgccggaagtcaaccctgcgcttgctccgctggtatctgttgtgccattgcagctgatcgcttactacgctgcactgcaccgcggctgtgacgttgataaaccgcgcaaccttgcgaagagtgtgactgtggaataggaataaataattcacggtaaacttaaaatatagtcggaaagtttgacccctctggttttagataaaccagaggggtttttagtatgaaaataagaagtgttcgcctgttgataaactgaagaattttatatttataaagaaatccccatatatatattgaaaagaagtaacgcctattttataatataaatatattccagtttaaacccataaatgtaaccattagtaaagaatggtagcgatcaattattggtttttgaataagtatttgtttggtgttttattcccatgtctgattactttttaggacatacttcactcttgaacgggctactcaattagacaataaatctgttatcatgagactacatatatagattatagaatacacattttcttttccagcatgtaagtcacctccagcagttttcatcatgaatatcccattatataaacctaatcaatcgattacacttccatattatgcttagaactttaaaatcccttttgtatttgccgactcctgatccattatgaatacctagactagatatgaaaatcacaacatttttgcaataatccggttcaattcttaaaaaagtcctgacaacaatgataggagtggaatgatgtcaaaccttttagaaatcagaaacatcagcaaatcttttggtcataaagctgttttaaaggatgttacattcaatgtgcctgccggatcaattgtcggttttatcggtgataacggcgcaggcaagtcgacaacatttaaaacgatattagggttaatctctcaagatagaggaaaagtgaaaatctttggtgaggagaatctggataaacatataaagtttaaagaagatattggtgtcgtatttgatgcgatgaatcttcctgcgcaccttacgataaagcagctgaaccatgtatttaagaaactattcgacgcttgggaccaagaccatttttaccggttggttcattctttctctttacctatggacaaaaaagtgaatacattctcgcgcggcatgtcaatgaagttatctgtagctgttgctttgtcgcataatgcaaaattattactcctggatgaagcaacgggagggcttgatccttcttcaagagatgatctgttaaatgaattaaaaagctttgtgagtaaaagcaacggcggtattttactttcatcgcacattatgactgatgtcgagaagatagccagccatatcattattatgaaagatggcgaaattttattaaatgaagagaaagataaggttcttgaaagctataccgttatagatgttgatgaaggccaattggctgagcttgacgaagatatcgttctagcaaaaaggaatcatggatcttattggagtgtgcttgtatcggatcaacagaagttgccgaatggcgtaacggccaacaccatttccatagaagaaatcagcgtctttttaacgaggagtgaaaacattgagaggtctgttactgactaactattacttggtttatcggagtttctttgcatacacggggttggccatattggtatcagcagtcattttttattttggggatgcttcaatgcatcgttttgttgctatgctgattattttgtttgtcgttatgcccgcacttgaggttataaagtatgagagcaagtctggctatgacaggtatgtcctgactttacccgtcagcagaacaaatattgttcaaagtcactatttcttttatcttttggttgttgtgatcggagctgtaatatcttatggtattttccttgtatacagcctgggttctgatgaaccaattgacggtatatttaatattgtctcttttggaacgttcatcgttctctttgccggtgcaatcgtttatcctcttctctacatgataggcccggaaaaatccgatgctatcgtgattggaggggcaatggggggactttttgctacctttggattgcaaagtatcgttgggtatgtagcagaacaagtaccgctgtcatttttgcacataaatccttctttatatgtcccgattatatatctcatgatcggcgttatcttatatatcatttcatttttcatcgccgcagctatatatcataaaaaagaattttagcgcgtgtgtattgattttatgctgatcaagttataacaggataaggtgttgtctttgcatataaagaactttgtgaccttgagggttaatatttagtatataaaaatggggggggcttttatgaaaggcaacatgctatatcatcaatatctgaaggcgaattccgagtatttcgggaaacaagagatgacaatgaatacatatgaagtgaatgacatccctgatacatatactgttatctcagatagcaactcagtatggaaatattatcatgttaagggtgtatcgttaccagagcaggggtggaaaattcatttaacttcattgttagaggacgcacaaagtgtattggatagggttgcccgcttttgtatgggtaagaaaattgaatttaaacacctaaaggatatgcatagttttattaacatgaattcaaaaaatgcaaatcgagcttcttccggcaaatttataacggtgtacccggctaatgaagaaatctttgttgaattgttgcaagacttatctgaaataacaaaggatttcaaaaaaggcccctatatactcagtgataagaggtggaaaaatactaatgtattttacagatatggcggatttaagcttatttttaatgaacatggtgagcactgtattagggatgaaagtggtaatttaattaaggatcaaagaacacccttttatcagacccctgattttgtaaaagactttgatgattatcttaatacgatcaatcattctgacgactcagaacaacagggggaaagccatttagcgaagtatgatatcattactgcactgagctacagtaacgcagggggggtatatcttgcgactcgaaagaaagataacttgaaagttattcttaaagaagccagaccaaatgctggtttagatggaggacccagagatgcgttagaaaggcaaaaaattgaatatgatactttaaaaaagcttaagaacatacatggcgtggtcaatctaattgagtacttccaagaatgggaacattactttttagtagaggaattcattgaaggaagagatttgagacaatggattgcacaagagtatccattctttagagatgaagatggcgatagtatatgtaatcatgctaagaatataaaaaaaatattgttacaactctttgctctgatagatcaaatgcataataacggagttgctatgggtgatttgcagccagcaaatgtgatggtaactgaagatttaaccgttaagataatagattttgagactgcaatgcccgtagagtctgaagaaaaacctagtatggcaacaatcgggtttgtttctcaagaaatgaaggcaggtggttctagagactggtttgggttaaagagattagtaagatatttagcactgccagtgctctcatcagacgatatggatgggtatctgcaagataaccatttatactggataaagatgaattatggagaatcattttatcatttcattttagagttgcaaaaaaaatgtgatgataaaataaaaggttatcacaaatatcttcccagagagattaacctttataacaagaaaagggatatcaacttatattccatgattaataagttgataaagggtctagaaaacaatttaatagatgatgacaggctcgtaaacggtgatattcgtcagtttgaaatgaacggaggaaaattcaactttctcactggggggagtggcgctgcctatattcttaccagatgtaactcgaagtctgagaatgtagataattggattcaaagttttttggtggataacctatcgactggcgatgataatggtttattaactggaaaaacaggaatgctggctctgctttatgagaaggggtacaaggaaactgttttaaatgaattgaatgttctgagaagtaatgtaaatgaaacaaccaacatttctttacgatcaggtctttcaggaataggcctgttcttaatcagtttatattttgagacggaaagtaaagaatacttggagtttgcgaaagagatagaaaaattgattgacctaaataaggttaaagacgagtcattaaaagttaatgactggatggcggtaggtataggtttaattgatgggttatctggcgtatctctgttttattcagcgctttattcagcaaccaataataaaagatatttagaggaagctgagttatttttaaagaaagacttggaaaagacaaaaaaagatgatgtaaccggtgtattacagactttggatgatagaaaccgattattgccatatctctctggagggtctattggagttgctgcgtcaatttggtttttaaaccatgtaagtggacaagatctatttaaagaagaatttagagccattttaaatctatcaaagatacgctgcacaatcagtggaggtttatttgatggcgcaggtagcttccttttaatcccttctatggttgataatagaaaattacggaagggaattcttatggaagtaatggatctgcttaatatctttttaattgagaaaaatgattattatgtttatccggggcaattttcttatagattatctgatgatgtttatacaggcagctcaggtatagtgttagcactaatgggaattgttaaaagcaatccactttattggttaccattggtcaacacagataaatttttagcggaaacaaaaatgaaagatattccagttggaataaattaataatattatccccaatagatgataaatgaatgtctgttaacactcgaaaggaggtgagaaagatgaatgatgttttggaattgcaaaagttagagaaagagacagatggtaaaggtatagctgtggatgcgacaattacaacgacatggacagtaacaacaactagcggttttgtttcttcagtaagtaatcattgttaaaacaatcttgcttcattgaatattaatgaagtataaatgatgaacactgtattggactgtcggagaagcctcaagtttatcatttcttgggtgcttgtacttaaaatcagcacccaagagttcttaattatcattaacaaatcatgaaaaaactatagagggtgacaacaattaaaaagttattagaattggcaaaaaaccctgaaccatttgaagaaggtacacaagaggtatggcttgatccagatagagctgaacttgttttgaaatcacattttgatgagaatatacccggaggcagcaaagggagctcatttgttgatgagactgtggattttattacaaaaatagcaccagtagaatgtaataataaaattattgacttaggatgtggcccggggctctattctcaaaatttagcattaaaaggctataacgtgactggtgtagattataataaaaaatcaatcgattatgctattagtgaagcggataaaatgaatttgcaaatagattataagattgaagatattacgaatctcgaaatcgaaagtgaatttgatgtagctctgctcatttatcaaatctatggtgtttttagtccgaccaacagaagaaaaatacttaacaacatcaatcgcggtttaaaacctggaggattagttctattagatgtcttatcagagaagagttatgaaaaatttgaagagaatttaatgtggttcctatcaaaaaaggacagtctgatatcggataaggaacatttgaccttatacgctgcaattaaatatcctgataatgttactttggcaaaaaatgttttggtttttgatgatgagaaattagtgaattataattattggaaccagcattttagcattgaaaatttagaaaaagaagtgaatgaagcagggtttaaccttgaaaaagtctatgcagatgtcaatggcgaggaatataaaaataacggagagttttttgcagccgtcttaaagaaaaaataaggactaaacgtatctataaatagccgagttgagtacttaaaataaatattccatttaggattcagtgttagccataatagatgaaaggtgtactccttatggctctaacactgagtgttaaaattgataggcctttaagccatattgaaatcgtacattgtgtttgttagcgagaaccagataaagattcagaaaaaagctgacgaatgatgccgtcagcttttctttatgtgttttcttaaaactcctcatactccgccgggtcttggccgtttatgcgtccgtcggggcgggtaagcgagtcgatgatgttcatgtcgttttcatcaagggagaaatcaaaaacagagatgttttcaagctggcgctcgggcgatgctgattttggaatcgggatggcgcccagctggatatgccagcgcaaaatgacttgagaaatggttttgccgtatttgtccgcaatctgtccgagtttttgatcgtttaatacttcgccggcgcgggataacggactccatgattctgtcacgatgccctgttcttcgtgccattggcgctgctgctgctgattaaaagcgggatgcagttcaatttggttgatgcctggctttacgccggtttcacgttcgagccgttcgatatgctcaggcaggaaattgcagacgccgatggatcggatcaggccccattttttcgcatcaatcagtgcctgccatgcttccgtgtagcggtcttgtttcggattcggccaatggacgagatacaggtcatagtagtccagaccggcgcggtataaagattcttggatggtttgtacagctttgtcatagtcatggtagcgcccgggcagcttggacgtcacgtatagctgttctctcggaacagagcttctgcggatggcttctccgaccgtcccttcattttcatagttataagcggtatcaatcagacggtagccgatatcaatggcgctggtaatggcatgaacgccattgtttccattaagagcgtatgtaccgaagccgatcgcgggaatgcttgtcccatccagcagtttcttttgcggaattgtcccagtcatcattaaatccctccttgttttct

SEQ ID NO: 3 - DNA, 7268 bp, unidentified
  source 1..7268: mol_type = genomic DNA
actttaccattattcttacaaatgaaacataataatggaaggaaatgaagtgatccgcacatgaaaaaagctgacggagaaccgccggctttaaatttgaaacatccatgctacaacggggccgaagaccgaccctaacacggcgcagagcgtcatggatacagaactcatggagaccgttaattctccgtactccattgcttttgaggtgccgagtgcatgggaggcgctcccgaatgcaattcctttcccgagcgagctgcggatgcggatccacttgagaagcagcggtcctaagataacgccggaaaaccctgcgatcatgacgaatacgactgtcatggaagggacaccgccgataccggacgcaatctgtatggcgacaggcgtcgtaattgattttgggataattgacaaaatcaaactccggctgatgccgaacagctctgcaaatagcaggccgctgaccattccggagaataaaccggccaacacgccgccgatgatcggaaacatatacttgattaacacccggcgctgcttataaagcgggtatgccaaagccaccacggcggggccgagcagtgaattgatccattgcccgccgatcatatagtctttatacgaaatatgaaaaacaagcagcacggcgataatgaaaatcgtagtcgtcaaaaccggtatgagaaagggatatggaaagcgcacatataatctcaccatagccagataggcggcggccgtgaacaaaattataacgattgcgatacatgcttgctgcacttctttttctcctttctctgattggctttgtgctctaacagctggctggcgtaccctgcggcgatcattgtgacgatggtgctcagcacgatgatcagaaacagagcagccccgctggcggaaaacagtgacgggtaattgatgacgcccgtcatggccggaatgaataagagcggaagaaaagacagcaaaaatccggctccgtcttcaatcattttgacgggaatgatcttaaccgataaacatatgatcagcaggatcaaaccgatgatgcttccgggaatgggcaggtgaaagaactgctgaacggctcccccgataaaggaaaatgcgaataaaataatgatttgcaagatgatgcgtatgactttcacgacaatgctgttctttggagaagagcagcttccccctttcttgatgtttctataagtttctttacttttggcacatatatttgtactatacaaatatatgtatgcaaaatctttctatatgttttctgagtaaatgtgcatatgtttgtattatacaactaatgttgctgttgtcaatgtaaggagtgaggctgtttgtcaaaacaagatccgattgaattaattgaatatgagctcaccacttttatcaggagagctgtttatcttgataactccgaccgtaaaatcggacatctggaaagggctgcgtatttgttattacggcagctggatgaattcgggcctgcgcgtgtgaaggaattggccgaatcatttaagctggatatttccacattgtcacgccaggcagcggccttggagtcgaaagagctgatccagcgtttttccgatccttctgacgggcgggtcagtctgtttgcgattacgaagcttggcaaacagaggttaaaagctgataaaaagatgcgtcttgagcggtatcatcgtatgcttgacgattggtccggcgatgaaaaagaaatgttcggccggcttttgctgcggctgaatgaggcatttatcgattaatgcatgatttttgccgggttaagcaggattatggcgtatacaagggagaatggaagagagtaaggctttacgccattctgcttagcaaaaataatattgaatagggggcggaaaacgtttctcctgggaaataatgtttctcggaggctgcggcatttattatctcaatattcataattcaatgaaaatgaaagcgctttatttttactccattggttcagagtgtaaggtgtttgcctgaaaaaaacaattgtattttccacgaaaatttattatgatggaataaggaaattggagattatataacgataggaagatagaatatttctaatattgcgggaaatgaagattgagttagggtcaacaggtgcaccttctgcttcattatttcacatatatcagagctgttaagaggaggaagcgaagtggcaaacaaagaattaaagagaggcttaggcgcgcgtcatatacagatgattgcgctgggcggcacgatcggtgtcggtttattcatggggtcatccagcacgatcggatggaccgggccgtctgtcctgctggcttatgcgatctcaggtatctttatcttttttatcatgcgtgcgatgggcgaaatgctctatgtagagccgagtacagggtcgtttgcgacattcggccataagtatatacatccgttagccggctatatgacggcgtggagcaactggtttcaatgggtgatcgtcgggatgtctgaaattatcgccgtcggttcgtatatgaaatattggtttcctgaactgccggcatggattcccggcattatcgccatggccattctcggtgcggcgaatttaatttcagtaaaatctttcggagagtttgaattttggtttgccatgatcaaaatcgtcaccattattctgatgattgtggccggaatcgggattattttcttcggtttcggtaacggagggcatgcgatcggattatctaatttatggtcgcacggcggtttctttacaaatggtttttcaggatttttctttgctttgtctttggttgttgcggcctatcaaggggtcgagctgatcggaattacggcaggggaagcgaaggacccgcaaaaaacgctgagaagcgcgattcagagcattatctggcggattttaattttctacatcggtgcgattttcgtaatcgttaccgtatatccgtggaatgagcttcactcactcggcagtccgtttgtttcgacatttgcgaaagtcgggattacggcggcggccggtttgattaactttgttgtgattaccgctgcgatgtccggatgcaacagcgggatattcagtgcgggacgtatgctttatacgttaggcgtgaacggacaggcgccgaaattctttaagaaagtttcttcgagcggtgtgccgatattcggtacgcttgcggtattaatcggacttgtgatcggtgtcgtgctgaactacatcgcgcctccgaaaatttttgtttatgtgtacagcgcgagtgtgctgccgggaatgattccgtggttcgtcattttaatcagccatctcggtttcagaagagcgaagggcgctgagcttgataagcatccgtttaagatgccttttgcgccgtttacaaactatttgacgatcgctttcttactcatggtattgatcggcatgtggtttaacaaagatacgcggatttctttggttgtcggggtgattttcctggcgattgtgacggtcagctattatgcgttcggtatcggcaagcgcatgcctggtgaaaaatcgaaaatagaagacgctgtataattcaaaaagacacctctcttacaggggtgtctttttcggcttgtccgcgagtggtatactggttaaaaaaagcagaaccgggggaaacatcgatgagtcattaccgtacgttattatttgatatagacaatacgctgctggattttaccgcttcagaaaccgatgctcttcaaaaattgttccaaggttcaaagctggagctgacggcagaggtggaagcccgttataaagcgttaaatcaagggctgtggcaggcgtttgaagagggggaaatgacccgcgatgaagtggtgaatacacggtttgcccttttatttaaagaatacggcctcgaagccgacggcgtgctgatgaatcaggcgtaccgccaatacttagaagaagggcaccagatgatagacggagcttatgagctgattgaacggctgagtcctttatttgatttatacatcgtgacgaacggcgtctcaagatcacaatataagcggctgggcgactccggtctgtatccgttttttaaagatgtgtttgtatctgaggacacgggttttcaaaagccgatgaaggaatactttgattacgtgtttgcgagaattcccggtttttctgccgggcacgggcttattatcggcgattcattgagcgcggacataaaaggcggtcagatggcgggaatggatacgtgctggtttaatcccggaaagaaagcaaatgagtccgggatcatcccgacgtatgaaattcattcgcttgatgagctgtatgatgtgctgaacgtggagcgaaaagccgccgctcatctgtaaggagaatacgatgaagattgcacatacgaagttactgatcaggggttttgcattactatcaatctttcatgcgattcttcagcttcctcccggccggggggagctgaaggtttttttgtctgtgctcgtgatgatgctgacggctaatgactgcctgcggccgctatatatgaagagaaggcctgctgtatatcaaggctcttttctcctgagcaacgttatcgcgggagttcttttgtacaatgtctatagtgtggggacacaggccgtaatggtgcttttattggcggaggtgcttattactgatcagcagatttcggctgttttgttaacggtgaatgccgccgcatttgccgcagctttatacgccggaggagcgggtgtcagtgatatcgtgcctccatacttcatcatgtttgcggtgatttatttatatcggaaaaacctgcaggagaagtcgaaggtagaattgctgaaccgagagctgaaaaccgctaatgacacactgaaggaatattcagaccggattgaggaactggccgttacgaaagaaagaacaagaattgcgcaggagctgcatgattcgctcggtcattatctggctgctctgaaaatgaatttagagtttgccgggaagacagccgggcatcagccggacaaagtgaagcagatcattcataaagcgcatgatcttacgaaagaatccattttaaagcttcgccaggcggttacgctgctgcagggctcgttgtccgggcgggcgtttgagcaagagctccgtgacattttcagtcattttcaagaggcgggaggcatacgcttcgagctggagacggacgcggcgatagaagcggaacagccggatgtgaagcattgtctttatcaatccgttcaggaaacgattacgaatgcgataaagcatggacatgcttcatatgtttctgtccggattcaaaaaaaccgaaacatgatattggcgcatattcataataacggctcagcctgcggaacgatcataaaatcaaatggtctgaaaggaattgagaaaagagcggccgcacttggcggagaggctgttttctcgtccgggaaggaaacgggttttgacgtgaaaataaggattccatgtaaggaggaataatcaagtgaatgtcatgattgcagacgaccagtccattgtgagagaggggctgaaaatgattctgagtcttcacgaaggaattcaaatatcgggagaggcttcttgcggcgaagaggttctccggctgctttctcaaaccgaaaccgatgtgattttgatggatatcagaatgcccggtatggacggaatcgaaacgacaaaagcggtaaaagcgcgatatccttccgtaaaagtcatcattctgacaacgtttgaagatgatcactatatttttgccgggctgaaaagcggagccgacggctatctcttgaaggatgctgattcagatgaaattaccgcttcccttcaagccgtttatgaagggaagatgatgctgaatcccaaaatcacggtttccgtcgtaaaagcgctgaacaatatggagatccctgcggtgcaggaagagccgcggtcaaagctgaaggagattctcacgccgagagagcttgatgtggcgaaacacattatggaaggcaaaagcaataaacgcattgccgcagacttatttctgactgaaggcactgtgaaaaactatgtatccagaatcctcgataaacttgaagtgtcaaacagaaccgagcttaccttatatttgcaaaaattgctataaccccgcttcaccctcctctctccctttttatcactaaacatatgacgaaagtaatacccatatcgagtacgaaagtgaatagaaaacaaggatcgccaagagtagaataagtgcagacatgacacaaggagtgctgcacgaatgctcgaattaacggatttgacaaaaaagtacggcgattttaccgctgtcaatcatatatcactgggcgtcgaaaaaggagagatattcggtctgctgggaccaaacggcgccggtaaatcaacaacggtgtcgatgatcagcaccgtgctcccgccgacgggcggaacaatctgcattgacggaatctcattacaggagcagcctgcggccataaaaaaagtgatgggagtcgtcccgcaggaaatcgctttatatcaagcgttaagcgcaaaggataatttggcttttttcggaagtctgtacggactttccggtaaggaactttgtaaacgggttgatgaagtgctggacatcatcgaattacaggaacagcgaaatcaaagaatatctgagttttcgggcggcatgaaaagacgggtgaatatcggtgccgcgctaatgaatgaaccgaaactattcattttagatgaaccgaccgtcggaattgatccgcaatccagacaccacattcttgaaaccgtgaagacgttaaatcaagaaaaagggatgaccgtcatctacacgagccattacatggaagaggtcgaatacctctgtgaaaaagcggcgatcatggatcacggctcaatcattgcctgcgggacgaaagacgaattgaagcagtcagccaaagcctctgataccctgaccgtgaatgcagaggggctgaatgatgatgccgttaaaaaaatccgccgcattcccggtgttgccgaggctgctgtcagccggcagaccattacgatgctcgtctctgcggcagatcgcaatatccttgatattgcggatgatatcaggaaaaccggtatcaccattaagggcattcatttcgaggaagccaatctggaaagcattttcctccggctcaccggcaaaacgctgcgggattcatag

SEQ ID NO: 4 - DNA, 9960 bp, unidentified
  source 1..9960: mol_type = genomic DNA
tcagcttctgagcagtctcataatctcctctttgtttttgaccagatcccttaacgtatatttgtctaaaacggccaggtaagcttgcagggcttcattcagaaggttctttaagttgcaggcgggagatatgatacagagatttttttgggagtcaaagcattctacgatgttaaagtcgtcttctgtatttcgaacgacttctccaatgtttatgtcttccggtgtcattcctaatcgaattccgcctccccggccccggatcgtttcgacatacccgagctggccgagacggtaaatgactttcatgaggtggtttttagagatggaataagtatctgcgatttcttttatgttggaaagttcgtcggaatgtctggaagccaaaaaaatcaaaacgcgtaaagagtaatcggtatagttcgttaatttcatagaatgacctcgaaaaaagagattttgcttctgttacagtctatcatatatgaatctaaaatgaaaaaaaaggaatttgctcccttaacttttaaaaaaaacgtgcgctccttctgagcacacgtttttttcgttaagacatcggcggcttacgatcgtcagccgattgatgtttttcaattttgcgatccaattttgcgagataacgggaggcaaattcttttccgccaagcccgaatgaaagtccgaaggcaagcgcgaaaccgcccagaatcagaataaacgcggcatttacgatcgtgtcggcgacaccgagctgatccagcgccataaagattgagagagtgataatggtatatttggcaagcggcgccaggaatctgaattcctttccgctcagcatgccggcaagcagcctgctgacgagctgtccggccaaaagcccgattccgagaataaatacagcggccagaacattaggcaggtaagcgataataccggttgtgataacaacgagaaattcaagatggacaagctgcagcgcctctgccgtaaagagcaggacgatgatgatctgaacaatcatgccgaccgtctgagaaagcgtcagcttcggggtacccgtctgttcaaagcccattttaccgagaagagagtcgaaaccggcccggtgcaagagcccggatacaatgctgttgacccattttccgatccagatgccggcgatgatcagcacgataccgatgatgatattagggagcattgtgaagaccgtgtgcagcatggctgacgccggttcagaaatacccgccacatcaagccggtcaagcgcagaaatgatcaccggaatgaaaatcagcacgtagacaatcgttccgatgattgccgacaggcttgtatccttcagataaatagaaagccccatacgtgcggcgagcctttccgtgccgatactcgccagaatgtttgtgacgatatcacggacgagacgggcaacaagccagccaattaaaacaatcagcgccgcggcgaataatttcggtataaaggacaaaacgctttggatcatatttgtaaacggccctgatactccgctgatctggagagcggccaagacgccgggcaggaacaagagcaagacgaagtagaaaacgattcgtgaagcggccgtaacggatctgctcatctccgcttcttcagacaccattgtccattttctgaaccggccgttttcagcgagtttcatgcccgcttttttgacgagatagctgaaaagcgaagcggccgcccagcccacgagcagaatcaggccggcttttaaaatgctcggcacagccgccgttatcgctgacagcatgctgacaaaaggcgaagccacggttgtcagatgcagaatgttaaagaatagaataaaaacgatgattaatgcgataaaataaatgactttactgatgactttttctgaggaatagcgcgccggctttttttcggcgaacagcctgtcgtcgatttttgttttttgcaggcctttgtatacggctttttcaatgattttggcaatgccccagccgataagcaggacgagcagtgcgatgatcagattcgggagtttgctgagatacgtcgtaagcatgtctgctgtattcaagtagaatcgctccttttctttttggggtatggtgtgtgttttacccacttacatataaagcaaacggggcggatgtgccgaaagttttcagccggaaaccggtctgttatttcatcttttgagaggaggggtcatacattataaataagtccaagtaggggggattcggttgaacgcggaggagacaaaaggtttacagcgggcaattgaggaaataaccgacattgcgaagggtttcggccttgatttctacccgatgagatatgaaatttgcccggctgaaattatttatacattcggcgcgtacggcatgccgacgagattcagccactggagtttcggcaaacaatttcacaagatgaaactccattatgatctcggtttgagcaaaatttatgaacttgtcattaattccaacccttgctacgcttttttgctggacagcaattcgctgatccaaaataaattaattgtagcccatgttcttgcccattgtgattttttcaaacataactgccgctttcaaaatacgaaccgggacatggttgagagtatggcggctgcggcggagcggattaagcattatgaaagaattcacgggatcaaagaggtggagtcctttttagatgccattctggcggttcaggagcatatcgatccgtcgctcgtcaggccgaagctgctgtggagcgtcgatgacgaagaggaggacgaagaagaaaccgcgtcatcgccttacgatgatttatggggaatggataagccgaagcaggtgaaaaagaaaaaagggaaaaaaccgtttccgcctcggccggaaaaagatattctgctgtttatcgaggaacattcgcgggagcttgaaccgtggcagcgtgacattttaaccatgatgagagaagaaatgctgtatttctggcctcagctggaaacgaaaatcatgaacgaaggctgggcatcctattggcatcagcggattatccgcgagcttgacctgacttcagatgaagccatcgaatttgcgaaactgaacgcaggcgttgtccagccgtcaaaaacgggaatcaacccgtattatttagggctgaaaatttttgaagacatcgaagagcgttatgacaacccgacagatgaaatgaaaaaaacaggggtaaagcccggttccggacgcgagaaaatgtttgaggtcagggaaattgaatcggatatatcatttatccgcaattatttgacgaaagatctcgtgatgcgtgaggatttgtatttattccaaaagcagggcagggactataaagtgatcgacaaagaatggaaggccgtgcgcgaccagctcgtgaacatgcgggtcaacgggggatttccttatctgaccgtcaatgacggggattacctgaagaataatgagctttatattaaacattggtatgagggaattgagcttgatctgaaatatttggagaaagtgctgccgtatctgtaccagctgtggggcaggagcgtgcacatcgaatctgttcttgaagatagagaagtgatgttttcgtatgacggcaaaggagtgcaccggagatatctgtaaaagaagaaaggatgcggggaaacccccgcatccttttttattttccgtgtctcaggatgtgaaagatctttttggcctgatccgtgttgtttaaaagccccgtaaatgactctcttcccggaccgtatgcgtaaacgggaacttcttcgcccgtatgatcgccggttgtccagccgctgttggttttcgcattaaagagcttaataatcgctttgtatgcccccttgtcagcatcctgttttgcggccgtttcgatctgtttgatttccgcttttgaccatttcaggtctgtataacgcgcaagcacggaagatacggatttgcccgctttgatttggtcagccataaatgccggggttttttttgcggcaagaatcggttcaacatgccagtttttatcgtcattcgcaccgatcgtcaatcccccggtggtatggtcagccgtggcaacgacaagcgtatgtttgtcctttttggcaaatgcgatcgctgctttatacgctttttcaaaatccttcatttcgctcattgcgccgactatgtcattgtcatgtcccgcccagtcaatttggctgccttcgaccattaagaaaaacccttttttattcttgttcagcctgtcgatcgccgccgttgtcatgtcttctaatgaagggacggcgtttgatcggtcgattgctttgtcgagtccgccatcggcaaacaggccgagaattttcttattacgatcctttttgagctcctgttttgatgtcacgtagctgtatcccgcttttttgaaatcctttgttacattgcggtctttacggataaagtttgacttgccgcctccaaggagcacatcgactttctgttttccgttgatgcggtcatcaaagtagctgcttgcgatgtcatccatgttctttcgggatttgttatgggctccgaatgccgcaggggtggcgtgcgcaatctccgaagttgcgacaagtccggtggatttccctctttgtttcgcttcttcgaggacggattttacccgctttccttttttatcgactccgatcgccgcgttatacgttttcacacccgtggccattgccgtgcctgccgccgcggaatcggtaatattggaatcaggatcgtcagggtaagtcatcatcataccggtgagattggggtcgaatgccgtcagtttcatgccatccgaaggctttcctttgttaatgaccgaacgataggcgttcatatagggagcgcccattccgtcgccgaccatgaagatgacgtttctgatttcgtttttgtcaggctgtttttttgccgcagcctgttcggagtgctgaagccccgcgcccgcaatcagaccggctgtcagcacggatacggctgccgccggcagaagttttgatcgaacctgtttaaatagactcattctcactcgtcctcctttaatcgtgtctgcatgtctaatggtagggggtgtttgtaaagagcgcctgttcctcctgtaaatttcaggtgaaacattgttagaaatgtaaattccggtgtaacttatgttttcaagaaagagtgaaaaccattcacttttttgaaaaagtcgaggttttttttaacaaggggttatttggaaaataaactgctttgtttttccaaaatacgaggatcacaggaaaaaaacatgacaaattgactcgtttttcgtcacgtttgaaatcaaattgaaatatttaatacccttaaaaacttttttctaaaacgaattagtaagaaatttgtcacgggaagtcaagtctatttctagtggaaattgaaccttataataggaaacagataaaaactttaaaaaagagacacataaaatagatagaatcattcagggaggaaaatatgaaaaagcaacttgttacagcaacaacggcagtggttttggggacgactttgtttgcgggcgcggcatctgcgcaaacgattaaggtaaaaaaaggcgatacattatggggactttcaaatcggtatggaacgtcaattaattccattaaatctgagaacaaattaaaatcggatatcatttacatcggtcagacgctttcgattaacggtaaatcatcctcaggttcaagctcaagcagcaagtcatcatcgtctacatataaagtcgtaaaaggcgacagcctttggaaaattgcgaataaatataaaatgacggtaaaccaattaaaaagtttaaacggtttaaaaacggacctgatccgcatcggacaagttcttaaagtaaagggatcaacttccggttcaacgtcttcaggatcttcgtcatcaaagcagccttcatccagccataatgctgcacagacgagcctgaatgtaaacaagctgatttctgatgcgcaatctttaatcggcacgccgtatgtatggggcggaacaacgacttccggctttgactgcagcggatttatctggtacctgttaaacaaacagacgagtgtcggcagaacgagcactgcgggatactggagctcaatgaagagcgtgtccagcccgtcaagaggagacttcgtcttttttacaacatatcaggccggcccttcacacatgggtgtgtacatcggaaacaacaagttcatccatgccggttctgacggcgtcacaacaagtgatctgaataacagctactggaaacctcgctaccttggcgcgaaaagattctagcagcgatagaaacccgttcattagaacgggttttttatttagacgggacagagctccatttgtttcacctaaaaatgaaaatgtgataaaaaatctaaaaaggttttctttttttcattttcatagagggcgatggcgtacgcgcccgcgctcggcatctgggcgaattcgcacggaatttcaatcatgctgcctgcggcgagttcggcccgtactgctgacagcggcagaaatgagacacccagcccctctttaataaatcgtttcgtaatatgcgtctgtgtcaccttcattgtcctgacgaaagggaactgtattctgacctggcggagcaaatcatcccaatagtcaggatggttatgggtcaaaagcagatattgtccgaacaggtctttcacttcatccgccttccctgttccgggttttcggccgggcggcgcgacgaggacaacggggtcattgtacagggggcggcaagtgagggaagatgattgcacctttaagcagctcagtccgatatcggctgagccgtttctgatttgagaggcgatttcttccgattcgaaaatggtgaccgtcatttcggtttctttattgtcctctgtataacgcttcatgacagacgggagaacggtgtcggcgataagcggcgaaacagcgagtgtcagcgtgtgcgagtagccctggcgcaccctgtgcagttccgccatgctgttttcatagtcttccaaaagcctcatggcaaacggcacgtatgcttttccttcttccgtcaattgtattttccggccgcttcgctgaaacagcttgcagctgatttctttctctaataattttatatgaacggtgacggtcggctgagataaaaaaagcgtttccgccgtttttctgaagttctcatattttgccgcggtgacgaaggtgtgaagccatttaaaatccatagatgcactcctctgattaaaagttttaattaattcccttaaaaatattcattattttttaaatattatatttactataataacagaaagaggataagggggaatgaaattgatttatcgtggattgaaagggattacttgtgcagaaacggctatcagccatattgacggagaaaaggggcatttaatatatcgcggctatgatgcgaaggatatcgcgctgcaatccagttttgaagaagcggcgtatctcattttgaacggggcgcttcctgatgagcggcaattgcttgcgtttcaggaagaacttgcagccgagcgcgggcttccgaaacatttgatcgacttactggaaacgcttccggatgaaatggacgacatggcggtgctgagaacggctgtttcaggtttgggcacagattcatttacatatccgcccaagccggaagaagcggttaaactgattgccgtcacaccgacgattattgcgtacagataccgccgtaaacaagggcaggaagcgttgtcacctgaggtgcgatacggacacgttgaaaactatttgtatatgctgacaggcaggcagccgtccgcagcccaaaagaaagcgcttgaaacgtatatgattctggcgatggagcacgggatgaatgcatcgactttttcagcgagagtcaccgtttcgactgaatcggacttagtgtctgccgtcactgccgcactgggtacgatgaaagggccgctgcacggaggcgcgccttcaggcgtcacgaaaatgcttgaagacatcggggaaaaagaaaatgcagaagcatatttggcagcgaagcttatgaagggagaacgcctgatgggcttcggacatagggtgtacaaaacaaaagacccgcgggcggaagcgctgcgcataaaggctgaagaagtggcgggaaatgatccgtcgcttgacttggcgctttatgtggagaaggaagccatccgcctgttagagcaatataagcccggacggaagctgtatacgaatgtggagttttacgcagcggctgtgatgcaggccattgatgtcgacaatgatttatttacgccgaccttttcagcaagcagaatggcaggctggtgcgcacacgtgcttgaacagtccgctgacaacatgatttacaggccgtctgcgaaatacaccggcgcttttcccgaaacggctcagcagctttaatcaccggcatatttgtccgtttaactccccgcgctgcgtggtatcgtctcagtaataccaattgcaggagtgtgagaatatgaatccgatggacagacagacggaaggacaaaagccgcaggtgcaaaagcgccagcccggcattgagtcagacatggagccgaaaccgctcttggaggacccggatgtaaaaggatcgggcagattaaaaggaaaagtcgccgtcatcacgggcggagacagcggcatcggcgcagcggctgcgattgcttacgcgaaggaaggggcggatcttgccatcctgtacctagacgaacatgccgatgccgagagaacgaaaaagcgtgcggaagaataccaggccgaatgcctgttaattccgggagacgtcggtgatgaggcgcattgtcagaaggcggtccggcaagtcttagatcactacggaaaaatagatattctcgttaataatgcggctgagcagcatccgcaaaacgggattcttgatattacagctgaacagctggagaagacattccgtacaaacatgttttctatgtttcatatgacaaaagccgtgctgccccacctgcaaagagggagcgccatcattaatacaacgtctattaccgcttatcagggagacacatcattgatcgactattcatgcacgaaaggcggcatcgtttcctttacgcgttcaatggcgatgtcattggccgatcaggggatcagagtaaatgcagtggcccctggcccgatctggaccgccctgattccggcaacgtttactgaagaaaaggtggaaaagcatggtctggacactccgatgggaagaccgggacagcctgcggaacacgccggagcttacgtgcttctggcttcagatgaatcttcctatatgacggggcaaaccattcacgtaaatggcggcagatttatttcaacataatacgcaagtcttatcccgcgcttttaaaaagggcgggatttttatttgaacgtacatatgaatacgattttcaaaaaatggaagtctccagccgaatcactaatgttaatatttaacagttgggaatccgctgagttctgttttatgaaaactaaaaataatatatagaattttcaaaaaagttattttcatttttatatttttcgctataatatgacataggagaaaggaatgatgtgctgaaacatttcatgcgaaaaataagaatttagtcattattgctattgttatctgaatattctaacatatattggtttacaaggagtgaagcagatgaaggggagcgtattcagaaagaaaagcattcaggacctgcttgcatcaacgagcggggaaaagtcgcttaaaagagagttaggggcctttgatttgacccttctggggataggggccatcatcggaacggggatttttgtattaaccggaacaggcgct

SEQ ID NO: 5 - DNA, 8040 bp, unidentified
  source 1..8040: mol_type = genomic DNA
gtcacggcggggccggggctgaccgtttcatttgtcatcgccgctcttgcgtgtttatttgcggctctgtcatatgctgaatttgcttcaagcgtcccggtttcgggttccgtctacacgtttacttatgcgacactgggtgaacttctcgcgtttatcatcggatgggatttaatcttggaatatatgctcgccgtcagcgcggtttcggtcggctggtccggctattttcaatcattcttggcgggcttcggcattcatcttccgaccgcattaacggccgcgccgggatccattaaaggcacggtcacattctttaatctccccgcatttgttatcgtgcttgcgattacattcctcctctatttaggcattaaagaatcgaagcgggtcaataatattatggtcattttgaaggttctggttgttctattgtttatcgccgttgcggccgtatatgtcaaacctcacaactggcagccgtttataccgatggggttcggcggtgttttcagtgccgcggcgctcgttttcttctcttttatcggttttgacgccgtctcttccgccgctgaagaaacgaaaaatccggcgaaggatctgccgaaaggaattatcttttcattaatcgtctgcaccgtgctgtatgtgaccgtatcggcgattatgacgggcgtcgttccgtttgcccgctttgaaggcgttgaccatcccgtgtctctcgtcctgcagatggccggacagaattgggtggccgggattattgatatcggcgcagtgttggggatgacaacggtgatgctcgtcatgctgtacggccagacgagggtgatgtttgccatgtcccgtgacggtttggtgccgggagcgctttcaaaagtccatccgaagcataaaacgccgcacgtggtgacgtgggtgttcggcatcctgtcggcattactcggctcgctcgtcccgcttgatgaactggcgaagctggtgaacatcggcacgcttgccgcatttgtcctgatttccgtggcggttatcgtattgagaaagaaacagcctgatctgccaagggcttttaaatgtccgggggttccctttattccgatattgtccattctgttctgcgtgtttttgattattaacctcggcgccaccaccatattccgtttctttatctggctgatcatcggtctggtgatttatttcttatattcgagaaagcattcgaaattaaacggggatttgtaaggagcggcacccgccgctcctttttatttttcggaataatgaaacatattgaaatgggcttaagttatcgcagtgaataagggataatatagggcggaagggaagtggaaagtcgtgtctgaccaaaaaagggttcaatgggcctcaaaaataggatttattatggccgctgccggttcggcgatcggcctgggagccatttggaagttcccttacgtagcgggtacgaacgggggaggcgctttttttcttgtattcgtcctctttaccgtgcttttaggctatccgcttttgctcggtgaatttattttcggaagacggagccagacaaatgcgattgatgcctacaaacaagccgcgccgaagtcggtatggcacctcacgggttatgtgggtgtggcggcctgttttttagttttgtctttttacagtgttgtcggcgggtggattttgctttatgtgttaaaagccgttaccggttctttatccgggctcactcaggagcagtacggcacggtgttcgggacggtcattgaaaatccgatccagactctcattgcgcagcttgtatttatgatcatgacggtggcggtcgtcgcaaaaggcgtgcagaaagggattgaacgtgtcagtgcggtgctgatgccgattttattcgtgctgtttctattgcttgtgctgcgatcagtcactctcgatcatgccgttgaggggattagatttttattggttccgcactttgaagatttgacgccggaatccatcctatatgctttaggacaggcgttttttacattaacgctcggtgtttcggtcatggtgacatacagttcgtatctgccgaaaacccaaaatcttccgggttcggcgggttccattgttatgatgaatgtgctcgttacgctgtttgcgggactggccattttcccggcggttttttcgttcggctttaaaccggatgaagggcctacactgctgtttaccgtgctgccggctgtgtttgaccggctgccgttcggctcactgttttttatcagctttttggcggtctttttcttcgccgcgctgacttcggccttttcgatggtggaaattatcgtggcgactgtggggaaaggtgatgaaacgaaaagaaagaagctgtcgtggacaagcggtctgctcattttcttagtcggaattccgtcctgtttatcatacggtgttctcggtgatgttcatttgttcggaaaaacgttttttgatcttgcggatttcaccgtcagcaatgtgctgattccgctcggcgctttattaatatcaatcttcgtcccgctgaaaataccgaagcaggagctgtttgccgagatgaaaagcggttccgccatcggctatcgtttctttcaggtgtggtatttccttctgaaatatctcgtgccgctggctatcatcgtcgtattgctgcatttaatcggcgttctttcattttaaaaaaacaggcccccgtaaaagggcctgtttttatgtttaaaaaccgaattgcagattcgtatcaaccgcaacgatcgtatatacacagacaatgacgaacagattgagaatgacaccgagcagtcccgggcccgtccgtccttttttaataaacagctcaatgatgccgaacggcacggcggccatcgcaaacggcagcatctgataagaatagaaagtcacattgctgatgattgccacgttcaaaagcagcagcgacaaaatcgcggtaccgactgatatccagccgaaaacattatgctctttaaaaattcttaccaattccataattccacctcaccgatgtttgctgcttttactgttttgtttttcagcagacgttcgatttcttttgtcggccccgtcacgacagagccgtatacgttcattccgtgatgttcaacatagtcgattcggctgcccagattcatgtctttgcgtttcgatattgcatgcgcccattttttatggcgggacataaattcaagggcattgataaattgttcacggtctgtctgtccttgcgtttcgaaagaggacaccatcttggaatctttcccgggaaatcctaacggctcggcataaggactgtctttcagatttttctccgtttcgaccgcgttccataaaaacttcacatcgtattttttaaaagtcgaatatacttgagaagaaggataggccctgtcataagaaagatagacctccgccacgcttccttcaggcaatttttttaatgtctgtccggctttgtcaatcgtgcgttcctttaagccggaaggatgggcgaaataatgctttgcctgtgccgcgtctaaatcatagtagttaacggcaggtgatttgaccgtatctaaaaacatttcaatccgttctccgccgacggatttcgtttttgtgccgatctgcttttgcagcggaaattccgtattcattccaaacagcttcacctggcttttcagtccggaggtatcgacgaggacgtttggcattgtggtcgccaccgtcactgccgcggtctccattaattgattgccggtgctttcttttccccccagaccgtaatagaggtagcttcctaatgtgcaaagcggcaaaatcatcaaagtggagatgacggcgagaactgaaatccgcaaatacgatttgcgttttccgtatgataaaatggccttctgtttttccgggctgatgccgacgtcccattcccggtcgtcctgcatttcgctttcaatcaactcctgatagacttccagcttttccagctcttcttcaaaggcggccatttcccggtcgctcatttctccatttttgtattggtaaaaacgctttttaaattcttcattcatcattaacacctctattataaagtgctttcagccgctgtctggcacgaaataaaacacttttaaaattagcctctgaaatattcataatatgggatgcttctttgtaattcagttcttttaaataatacagtgttaaggcctcccgataattgtcgggaagttcggacatataccctgtcagcacttccttgatttcaatctgatgagccgggctttgaacagccgtctggaaaaggctcccaattaagtcatcggaaatggtcacttcctttttatgttttctgacataatctataaacgcatttctcgccacttgaaaaagccatggctttactttgctgtggtcgtaggagtgaatgtgtatgtatgccctcataaaggtttcctgcagcaaatcttcagcgagatgcttgtcttttgtcatggagagcaggaaccggtaaacatcattcatatacatttggtaaatttcatcgatcgtcactgccttttctcccctctatgttatacacgcataagaaaggtttaaagttgcacgttccatgcaaacggcttaaattcattttcacctcatcaaacaccatttctctatagtacccaaaatccgtcataaaagatagagatcggcggaggaaaaatataattattagtaccaggtaataaaaaatattgttataatagagagagagaacacttttgatcattacaacaaaggaagtgcgttatgtataagttttgtgcaaatgccatcaaatgcattctatccctgcgcggaggagtgaaagtgtacaataaagagaatcttccgtctgataaaggattcgttatcgcgtgcacgcatgcgggctgggtggacgtcctcacgctcggtgtcgggatattgccccatcagattcattatatggcgaaaaaagagctgtttcagaaaaagtggatcggacgttttttgaagaagattcacgcgttcccggttgaccgtgaaaatccgggcccgagcagcattaaaacgccgattaagcttctgaaggaaggaaagattgtcgggatttttccgagcggtacgagaacatcagaagacgtcccgctgaaaagaggggccgtgaccatcgcccaaatgggaaaagcgccgctcgttccggccgcatataaagggccgtctacgggaaaagcgctgtttaaaaagggaaaaatgcggcttatcatcggcgagccgatttttttggaggacttttccgatcttgcgccgaaggagaggcttgcggccatgacagaagagctgaatgccagaattaaagagcttgagaaaaaacttgaacaactgtaaatgaaaaaaagcttgccgtctgtatcagacgcgcaagcttttttgttatgcttctttttgttcggcggtttccagcagttcatcaaacgccagttttttcacttcgatatatgcgacgtggtggccgtcaatttcattaatgataaattcacagccttcttcgataatcgagtcgtattgttcgacgtcgtatttctgggtaaggaaccagccgccgatggtgtcgacctcgtcattttccaaatggatgccgaggagttcgttcacttgatcaatcaggaccttgccgtcaagcatgtaatggccgtcgccgagtttgcggatttcgttgatttcatcgatatcaaattcatcacgaatttccccgacgatttcttcaataatgtcttcaacggtgacaagccccgcggttccgccgtattcatccgacagaatcgccatatgaacccgttccttctgcatttttacaagcagatcgtgaatcggtgccgactcaatcacatggatgatcggattaacaaaatcggtaatcggcgagtcgactgatacttcgccgctgatgcaggcggtcagcacttctttaatgttaataacgccgataatattatctttgtcaccctcttcgacagggtaccgggtatatttttcaatctgaatgatgtccatcatttcagaaattttgatatcatgcggcagactgacgatttccgtacgcggaatcatgatttcttttgcaagtctgtcgtcaaattcaaagattttgttcacatatttaaattcagactggttgatttcaccgcttttatagctttccgataaaatgatgcgcagctcttcttcagagtgggcaagctcattttcagacaccgtctccagaccgaacgccttcgttaaaagacgggcggagttgttcagaagccagataaacgggaacatgatgcggtaaaaccaaatgagcggtttcgaaaacagcattgaaacggcttccgctttctggatggcgactgttttcggcgccaactctcccacgaccacgtgtaagaatgtaatgatcgcataggcgagaataaatgaaataatatgcgataatgagcttggtacgtttatctttgtgaaaagcggatgaagcaagcgttcaatcgttgattcgcctaagacaccaagaccgatggaagtcaatgttatgccgagctggcacgccgataaatactcatctaaatgcgtggtcacttttttgacaccgattgcggctttatttccttctgctataagctggtcaacccgtgagcctctgattctgattatcgcaaactcagatgctacgaaaaatgccgtcagcgcgattaaaaccgcgactaaaatcaaattcacaatgtccaattagaaccttcactccttttgggaagatgaaggtaacacctcctgttagtaagataatggtttaacttaacagcagcataaatgattgaagcactgcaagcttttcaggagccagcctttttttcagctgggcgcgctcctctggttcaagcttttcaaaccgattcaagatcacggagatgttttgattcatgtgatcaatttccgatgccagatttccaatatcgtcaggcagggctgaagagggaaactggttctccagtcgatcttttatatcagacagggccaatcgctgctttttacaagcgactataaactttaaccgattaagtgcaagtgtatcgtagtaacgatagttcgaacaagacctgtcaggctttaaaagcccgaggtttgtgtagtaatccaccgtgcgtttcgttacaccggcaagctctgctaattcactgatgcgataagtctcagccccatctaaatccctccccaaccatcacgttatagttatgagaatagtttaatatatgaaggggagtttgcacaagtttattgttttccgtctttctcctgaaacggcgcggcgggctgtctgaaaaagcgttgactgcgccatgtaaatctttcataatcattgtaaatagaaacggaagaaaaagacagaagggacagatgacagtggccggtttatcacgggaagtgacacagaatctggagaaaggttcatggatacgcaaactatttgatgaaggcgctcggctgaaaaaagagttcggcgccgataaggtgtttgacttttcaataggaaatccggttgcggagccgcctgaaacatttaaaaaggcgctttgcgaagcggctgaaacagggcggcacggatacatacaaaaccaggggcttccggaggcgagagaaaaggttgcgcgctttttgtcagagcggtttcagacagaaatttccgccaggcatacagtcatgacggtcggagccggcggggcgctgaatgtcgcgctgaaaagcattttaaaccccgaggaagaagtcatcctacttgcgccttattttgcggaatataaagcgtatgttgaaaattaccgcggcagacccgtcatctgcccgctgactgactcatttgagattgatattgaagcggttaaaggtgcgatcaccccccgtacgaaaggactgatcgttaatacgccgcataatccgacggggactgtgctcagccagaaaaacctt

SEQ ID NO: 6 - DNA, 9600 bp, unidentified
  source 1..9600: mol_type = genomic DNA
aacgaattaggcgccttattaaaagaagccgaagagcggaacggccaaagcatatttattttatttgatgaaccgtacagccagctgatctacggagaagagcaggcgaatccgtttcaggcgtatcaccgcgtgattttagtcagttcgttcagtaaagatctcggcattgcgggggaaaggctcgggtatatcgcggcaggcgcccaaatgcctgagggcgatcttctgacggatgcgttcgttttctgcaaccggacgctcggctttgtgaacgctccggtcatgatgcagcgcgccgtggcgaaaatggatgatctttcgattgactctgaaccttacagaaagcggagagaccttatggtggacatcctcacagaagccggcttcgaatttgaaatgccgaaaggaggctttttcgtctttccgaaatcaccgattgaagacgaggtggcattctgcatgcatgccgcacagaaatacaaactattgatcgtacccggcagcggatttggaaaaagcggctatttccgattatctttcagcgtgccttcagaacagatcagacagtcgcgcgatattttcaaagcactgtatcaagatttttcttcatagcttccaatacataaagatgggagaacggacaaatgtcgaggagtaataatggcacgtcagacatcagcattacaggaatcggagtcttaaccgcggtcggacaaggcaaggaggcgtttctcgatgcgcttatggaggggcggcacgcctttgatgtgatgaaacggcccggccggcagaaaggcagttcgtttatcggcgcggagcttagcgaattaacgccgccggacgcgctgtcgaagcgcttgtaccgaagggccgcattatcaggaaagacggcgcttatcgctttatatgaagcgtggcatgacgctcaattagacggcgctgatcacagccgcaccggtctgatcatcggcggctcgaatatgacgcagcgcgaattgctgcaggcctttgagaaaaacggtgaaacgccgcaatttgtaaatccgacatacgggctttcttttatggatagtgatttgtcggggctttgtacggagcaattcgggataaaagggccggcctttacggtcggcggcgcgtcggcaagcggccaggcggcggttattcaggctgctcaaggaatcaaaggcggcctgtttgaccggtgcatcgtgatcggggcgctggccgatctgtcgtacatggagtgtcatgctttccgttctttgggagcgatgggcactgatacatacgctgaacggcctgaagccgcctgccgcccgtttgaccggcatacagacgggtttatattcggagaggcgtgcggggcgattgtgctggaacgcgctgaatctgccgaacagaggggcgcacgatcgtgggctggcttaaagggctggggaatggcttccgatgcgaaccgcaacccgaatccttcagcagagggcgaaatgacggtgatccgccaggcgctggagcgggccggtctttcagcaaaagagatcgattacatcaacccgcacggcaccggttcgccgatcggtgataagactgaatgccgggcgctttctgaaagcggcctgtcgcacgcgcggattaatgcgacaaaatcgattaccggacacggtctgagtgcggccggaacggtggaaatcattgctgctctcttgcaaatgcgggaaaacaagcttcatccgatccgaaacttaacggagccgattgacgattcatttcagtgggtgaccgacgggccgagcgcccataccgtacaccatgcgttaagcttaagcatgggattcggggggattaatacggcgctttgtttagaaaatcatttccgaagcagataacggcagccttctttttgaaggctgttttttatatgataaaaatctgaaaatgatgaaagaatggagttgtgatacaataaactatacaaatgacaatcatgctttgacttaggaggtggcaggatggggccctctgtgtagtcaatcacccatttcgttcctctcgctttaaaaaaacgttgaaatcaggaggtgacgtccttatccttcaattcgggataaggtgaaatggacgatattgacagtttgattttaatcggtgtgctcattgcgttaacggcatttttcgtggcgagtgaatttgcgatcgtcagagtgagaagatccagaatcgatcagctcatttcagaagggaacagacgggcggtgctcgcaaagcgtatcattacggatcttgatgagtatttatccgcctcgcagcttggcattacactcacatccatcgggctcggcgtactgggagagcctgcatttgagcggatgcttcatcctgtgtttcagggcatggggctgccggattctgctgagcatgtgattgcttttgctgcggcgtacgggctgattacgtttctgcatgtcgtcgtcggtgagcttgcgcctaaaacggttgcgattcaaaaagcggaaaagctgacgttatggattgccggaccgcttcggttattttacgtgttcatgttcccgtttatttgggtgctgaacggctccgcgagaattctgaccggtttggccggattaaagccggcatctgagcatgaaggctcccactctgaagaagagctgcggatcctgctgtcagaaagtttaaaaaacggagaaatcaatccgtctgaatatacatttgtgaataaaatttttgattttgataatcggatcgccaaggaaatcatgatcccgagaaaagaaatcgcggctgtttccactgatatggacgtgccggaaatgcttgatatgatgctgaaagacaaatatacgcgctggcccgtaacggacggggacaaagacagtgtcatcggcatgatcaatacgaagcatctcttttcagatctgctgtttatgacagaagatgaaaaagcggaagtatccatcacgtcttacgtccgcccggtgattgaagtcatcgagacgatcccgatacacgacctgctgataaaaatgcagagagaacggattcatttggcgatattgtctgatgaatacggcggaacgtccggtcttgtgacgaccgaagacatccttgaagaaatcgtcggcgatatcagagacgaatttgatgaggacgaacagccgcagattcaaaagctgtccgaacgccattatgtgctggacggaaaggttcggattgatcaggtgaatcatcttctcggcgcagggattgaagaggaagtcgacacgatcggcggtcttgtgctgaaacggaatattgacatctgtgcgggtgagtcgatacaaatcgggggatacactgccctcatattagaaatggacggccggtttattaaacagcttgaactcaaagaagaacagggccgcccgggcggtcagggaacaataaaccgccagctgcccgtgcctgaaacggtcatgcttcataacgcaaccttgagcgaaaaataagctcaaggtttttttaaatggacatgataatgaagccgcaccagaaccccaaaaggcttccggctgctgaaacagcgatgtagagcaggctttcctttatgcgattgttcatcatcagctgcacggcttcaatactgaacgtggaaaaggtcgtaaacgcaccgaaaaagcccgtcgcgatgatgatgccggcagaaggattatccaggcccaggccggtgaacatgccgaggccgaaggagccgagcaggttgaccgcagccattccaagcggcatcggaaaaccttttgttctttctgaaattgttttacccagcagagaccgtaataccgctccgatacccgctgcgataagcatcaggacaatcgtcatggcgtatcatgcccccttcccgctgcgtgaagggcaatccgccgtccccagaaaatgccggcacatgcaagggtaatgccagagactaatgtcgcggcaaaataacaaaaagccgctgcgggagacgattgcagcagaaaaatggtttcttttgcgaaggttgacatcgtcgtgaaacctccgcaaaaaccggttcctaaaaaggcggtcaaaagagggcgtttagcccgaatgatgaaataacccgtaataagaccgagtaaaaggcttcccgccgcattttcgaggaaagtggccgccgggaaagaactgctgtgaatgtgcgcgctcatttcaaagcggagcaatgcgccaagcgcgcctccgataaaaattgcgatgtattgtttcatatcagccatccttagaaataaaatccggaaaatcagttacaatgccgtcaactgccatcatttgcagtttttcggccgtcttttgatttctgaccgtccaggcaaatactttcaggccgtgtctgtgagcggtggaaatcaagaaccgattcaggcgggtatatttaatattgacataatttacggctgatttgtaggcggccagctggcttttggaaaacatcccgaagctgggtcttgcaattattgcagtcggaacggacggatacatgtcgtgaaaggtctgaattgacttgaggctgaatgattgaacgatagggcggatggaaaaggaaaacctgcttaacagctggccgactgctttttcaagaccgacttgagaaacgtgccccttcagttcaattaaaaatccgatttcagaatgataacgcttcagaacctcttcgagcaggggaatccgctcccctttaaactcagccccataccagctgccggcatccagagactgcagcttcttcagcgtaaaatcttttacaaaaccggagccgtccgtcgtccggtcgactttgtcatcatggatgacgacaatctggccgtcttttgtgagctggacgtcaagctcaatgaaatcagcgttcatttcggcagcgatatcaaatgccgcaaatgtattttcgggcgcgtaaccggatgcgccgcgatgagcgatggtatacaagcttgtcagctcccctcatgggcatttctgttattatatcagattcgtatatgcacagcgattcaaactggtgaatgtgcgaaacagtatttttttggaataaagatagtgcttttttcggtttacaatccgcagtgatgatttataatggcaaaaaggaggcgagatcatgggaaaagggagattgaaagtggaagaaagaattaaggccgaaaccgatgctgaaatccaaaaagcgactctcctgaaacagacacaatcaaagaagaaatagtgcctgaccctgttgtatctggctgaaagatgaacgaatgtgtgaaaaaccccggctgtgctctgatgccggggtttttcaggctgcctaaagacattaaaagaagctgatgtccgaatgttttagtataatggattttcgacaagacaaaggagcatatgccatgatgacacaatttcagagcttttttacgcttgaccatattttgcagatcagtatcagcatcggaattttagcggcttttctcgttttgagaaaactgtttgtcaaatacttttttcagctcatttacaaattggcgaacaagccgaaaaccgacatattcaaacagattgccgtagcgtttgacaagccggccagatggtttttcgtggcgctgggtctatttttggcgattgagtattcgccgtttttagacgggcagatggacattgtcaaaaaattgtaccgctctctgatcgttgcgctgatcgcctgggggctttgcaatctgacatcggcgtcttcgtttttatttcataaagtcaatcagcgctttgagcttgatatggatgacattttggcgccgtttttatccaaggtgctgcgctttgtcattctcgcgctcagcgtcagtgtgatcgcgcaggaattcaattatgatgtaaacgggttcgtggccggactcggacttggcggattggccttcgcgcttgcggctaaagatacgatcagcaattttttcggcggcattatcatcattacggaaaagccgtttacgatcggcgactgggtcgagacgtcatctgtgacggggggcgttgaagatatcacattccgaagcacaaggttccgcacggcgcagggggcgcttgttaccgtacccaattcaacgctgtccatggaagccatcacaaactggacgaggatgacgaagcgccaggtcacgttttccgttcgtgtttcttatgcgacgccgattgatcgtttagagcggtgcatcacaaggctcagagaaatgcttcgtgaacatgaaggcgtgcatgatgatacgattatggttaactttgacgtgtttgcggacagctattataatctgtttttcaacttttacacaaagaccatcgtctgggcggaaagcctggatgtgcggcaggatatcaaccgccggattattgatatttttgctgaggaaggcgtggaattcgcctatccgggccagaatgtattcgtaaaacagaaaagccacgatgatccggttcatgtgaaggatcattccggagaaaaagaacgggcataacaggtgccgttcttttttcatgatatgataaaaagaaaaggcggtggaagagtggaagcgtttatgacattgatcaatcaggcggaacgtatcgtggtgctgacgggagcgggaatgagcacggaatccggcattcctgattttcgttcggcgggcggtatatggaccgaagacacatccagaatggaagcgatgagcagggaatactttgaacgccgtccgcgtgaattctggcctaaattcaaacagctctttcagatgaaaatgagcggaagctatgagccgaatgccggccatacgtatttggcggatcttgaaaagcggggaaagcaggtagacattttcacacagaatattgacgggctgcataagaaagcgggaagcagcagtgtctatgagcttcacggttcaattcagaccgccgcatgcccccgctgcggagcgcgctacgggctttctcatctgctgaaggacgaagtgccggaatgtacggcggaaatcggtgatcatgcggtatgcggcaccgtattaaaaacggatgtcgtgctgttcggcgatctcgttcagcattttgaaaccctttatgaaaagcttgataaagctgatcttcttcttgtgatcggcacttcgcttgaagtggcgccagcaaggtttgtccctgaggatgcgggccggattcccggtttgaagcaggtgatcattaaccttgagccgacatattgcgaccatttatttgatcttgtcatccatgagaagatcggtgaatttgcaaaacggatcagcgccgcgacataagaaaagccggatcatatacagatccggcttttcttattcatggacgggaaacatttgatccgtcagaacatcaaaggagtagccctgggctttcagtttttggatcaggctcggcaaagcgtgcaccgtcgcaggcagatcgtgcattaaaatgacatgcggcctgcttgtatgaacgctttccaaatatgcgagctgattcaatacttccggcacgtaccgtgtgtttttgtacttccagtcttcactgtctatcgtccagtcccagtacacgaatccgcctttttcgagattcttaatctgggcatccgtcaatgacggtttactgccgtaaggcgtgcggacaagatccgtttttactccggtgattgcctcgagtgtatcgcgtccctgctgcatttcgttcagcggtgatgtcggtgtttgataaaacaggtgattatcgtgcgtcacgccgtgcaggccgagcgcgattccttcctgcttcgctctttgcacggaatgcttataggctttcatccgcggttcaagcataaaaaaagtggcctttgcgccatttgccttcaatacattcaacagctgttcagtatatggagacggcccgtcgtcgaaggttaaatataccgtttttttccctgggtcacgctctctctgcaaatgtttcattaagatgtcgggagaatcatttttcagccgcaggctttctgcttcatcccgaagctgcccgtttgcggtcacggtgacctgtttgttttttgcggcggaagcggaatatcccattatgacgattaccagaagcagtccgcacaaaactgctaaactcgtaagtttaaaagccaatgaaaacagagaaggggaagcaccttcttttgtgttgcgcataacaatctctccagtagcataagtttctgcataatgccgataacagcggaggcttgcgaagtgccataagttaacagcatatgtatgtcgaatcaagtatacaggaacaaaactggaaaagacatagaaaagatgtcgaataataagattttaagaaaaaaataaactccgcagccggctcgttcagtatgatagaatagttgacaataacagctgagaatgcacatcacggcagataacgatgaggagggtttacatgaagacattagtaaacggccagttggttgatcgtgaagaagcggcagttgatattgaggaccgcggatatcaattcggtgacggtgtgtatgaggtcatcagagtgtataacggcgccttattcggtcttcgtgagcacatcgaccgtcttttcagaagcgccgcagaaatcggcatcaccctgccgttctccgcagaggacatagaatgggatctgcaaaagctcgtacaggaaaacaaactcattgacggcggggtctatatacagacgacgagagggaaggcgccgcgcaagcatcagtatgacgaaggattggaaccgcagacgacggcatacaccttttcagtaaaaaaaccggaaaatgaacaaaaagccggagcgcaggccataacggcggaagacaaacgctggctgaggtgtgacattaagagtctgaatttactgtataatgtgatgattaaacaaaaagcctatgaagcaggcgcttatgaagcggtgctgataagggacggagcggtcacggaaggaacctcttccaatgtatacgccgtcataaacggcactgtccggacgcatcctgcaaatgagctgattttaaacggcattacgcggatgaaactgttagagctgatgaaagaaaacgggattgaggtgcgggagaaggctcttaccgaagacgaactcagggatgcggatgaaattttcatctcgtcaacgacggctgaattaatccctatcgtaacccttgacgggaaaccggtcggaagcggagcgccgggacctgtagccaaaacgattcaggaggcttttcagtcgcacattcagcaggagctcggcgttcagtcataagctgaaaaaaggccggaacacatccggcctttttgcttcagtcagataaaaagagattcgatatgctgcacgagcacacgggagattttctcttttgaaagacgctcaggttcaagcatggcatgcagcgccaatccgtcggcgatggcgtacattttctccgcttcgatctctttatcaagattcggtttcagaagcttattttcatccagccagtcaatcagaccgcggatatatgccaagatcccgtcctgctggggttgaaactct

SEQ ID NO: 7 - DNA, 8754 bp, unidentified
  source 1..8754: mol_type = genomic DNA
tctcccttatgtctggcataagctgtaaaagcgaaccatacctccatttcagccatcgtttcttcatctgtcgggatgatttcaagcatcagacgcaccgctttttctttcggcggcagatctttcatggcaacttcgttaattctggcggccgctttttcttttacgagtgtcatcgcataccctaaaagctcatcctgggtggtgaaatagtgcctcagcgcgcctaaagaaagacccgcttcctgggcgatgtttctcactgtcgctccttccattccccgctccaaaatcgtccgccaagtggcttcggctatttgttttcttcttttttcatgatcgattattttaggcatggttccagtgtaacaggaaatattttttcttgcaattttgtaatacagttgtattaaataaaaagcagtgatacaatgcattttagaaaacgataagggggaaccggattgaacgcgattgcttggttgattgtcatttgtgaggctgctttttgggcggctgtcatcttcggattggcggcgcgctatatattcaggcggcaaaagctcggaatgttttttttcgctctcattcctttattggatgccgtactgtttctggcggccgcagctgatttatacagaggggcggcggcgactatggcgcatgcggtggcggccatttatatcggagtgtctgtcgccttcgggaaaagcatgattcaatgggcggatgagcggttccgctattatgtggtcaaggaaggggcaaaaccggtcaaacggtacgggctggattatgccaaacattatgcaaaaggatggggaagacatgtattagcatttgtcatcggcggcgttcttttagccggaacggtatttctcattcaggacgcttcccgcaccgaagcgctcagcaagctgttaaagggctggtctgtcgtattgtgcatcgattttttgatcatggcgagtttttttgtgatgccgaaaaaaagaagaaacagccgggagctgtaaaaaaagcagcttttgattaagctgctttacagaaagaattattttacgatcagcacgggaatatcggatttcgatgacagtttttcactgacgcttccgaataaaagttttttcagcctgttctgatcccggctccccatgacgatcaaatccgccgatatacggtcggcatgctcgataatggcatcagcgggatcaccttctaaaatgtcgatatccccgtcaatctggctgtcgttcatcagcattctcgcttccgcaacgatttcttccgtacggtcttcataaatcatcggatcaggggagattctttccgcctggagcgggtcaggaacgcttgcaatcccgccgccgatatacgttgccccggctcccggcctcgggggatcaacaatcgtccgggtatcttttgcattgtgggaatgtacgaccgttaaatccgcgtgaaatgtttttgaaaggtctatggctttttggagcgcttttttactatcatcacttccgtcaaaagctgctatcaaccggtccgcgtgaaacatcggcctcatcctttcagtaaaaaaggtttgttttacaatacccgaatggcagaaatgtaaaacctgcattgacaaaaaaaggatttgtttatatgatgaaggaacaatatagggaaaaaggaggtatggatcatgtcaaaactaaagcttgtcaactcatcttcagctgaataccggcgtgactcccatacagcttccgttttctgaacatttgcttaaaaacggggcagtcgcgcataaacggcgggtctgtaccttatttataataataagaggcatggagctgaggcttcatgtctttttttatacaaaaaaatgaaagaaagacaggtgtgatgcatgtggcggtattagcgaaaacaatgaaaaacaatccggaaaagcttgagggcggctgattgatgaaaggggatgacagtatttgtttactgtattaaaaaagctcgcgtggttttttaaagcctattggctgcgctatacgacagcgattgttctgttattgattgtgaatgtcattgaaatgtttccgcctaaacttctcggtaacgcgatagacgatatgaaggcgggcgtgtttacaaaaagcgggatgctgttttacatatgtgtatttctcgcattaacggcagccgtctatttactgtcttatttttggatgtatcagcttttcggcggggcgaatgtcatggagaagattctccgttcaaagctgatgggaaggcttcttcaaatgtcaccgccgttttacgagaaatacagaacgggcgatttaatggcgcgggggacaaatgacttgcaggccgtcagtatgacgacgggcttcggggtcttaacgctcgttgattcaacgatgtatatgctgacgatttttttgacaatggggcttctcatcagctggaagctgacatttgccgcgattattccgctgccgataatggcggctgcggtcagcctgtacggaaaaaagatccatgagcggtttacggaagcgcaggatgctttcggatcattaaatgacaaggtgctggagtcggtatccggcgtccgggtcatccgggctttcgtacaggaaaaaaatgatgtcagccggttcggcgatatgaccgcggatgtatatgagaaaaacatgaaggtggcgtttatcgattcactttttgaaccgactgtgaaacttctcgtcggcctcagctatctcatcggtctcggctacggcgcttatctcgtttttcaaaaccggctgacgctgggagagctggtatcgtttaacgtgtatctcggaatgatgatctggccgatgtttgcgatcggagaattaatcaatatcatgcagcgcgggaatgcctccttggatcgtgtcaatgagacgctttcctatgagccagacgtgacaggacccaagcaccccgccggcatcaaggagcccgggcgtcttgtttttgaacaagtgactttcacgtatccgagcggagcgggaccggcggtttctgatgtatctttttccgtagaaaaagggcagacactcggcatcgtcgggaagacgggaagcggaaagacgacgatcattaagcagctgttgcgtgaatatccgcccggcgcgggagcaatcacgtttacaggcgttccgattcaggagattccgcttgaggagctccgcgggtggatcggctatgtgccgcaggatcatattttattttcgaagactgtaaaagagaatctgctttacggacggcaggatgcaaacgaagaggaaatcggcagcgcgctgaaagatgcgcattttgaaaacgatatcggcatgctttccgacgggcttgagacgcttgtcggcgagaagggcgtagcgctctctggcggacaaaagcagcgcatctcaattgcgagagcgcttttgaccgatcctgacattctcatattagatgattcgctttctgccgtagacgcgaaaacggaagccgccatcattgacagcatccgtaaaaaacggcggcataaaacaacgattatcaccacccatcggctctcggcagttgaacacgcagatctcattctcgtcatggatgaaggccgggtgtctgaaagaggcacgcaccgggagctgttggcgcagaacggctggtatcgaaaacaatatgagaggcagcagctgtcctctcttgaagaaggggggacgggcgcatgaagaccgggagaagattatggaattacgcgcttcaatataagaagctgctgatatgggcggtcgtcctgctcattattgcggtcggcactgagctgaccggcccgtttatcggaaaaaaaatgattgacgaccacattctcggcattgaaaaaacatgggttaaagcctctgcggacgatccgggcgccgtcaggttccgggggcatgattatgtcagagaagaccggctcaaaccgggggcggacaaaaccggagaggcgcacatttatcaagtcggtgcgggctactattttgttgaccgtcccgtcagttttgacggaaaccgttcggtgtctgacggccggctgacaatatcaaacggtaaagaaagcaggtcatatccggccattacgttatcaagacaggagatttttcaattttaccgtcccgaaataaaaggattgctgacgttgaccgccatttacgcgggactgctcatgttttccgtatgttttcattacggccagcattatctgctgcagatgggagcgaaccggatcatacagcgcatgaggcaggatgtattttcacacattcagaaaatgccgatccgctactttgataatctgcctgccggaaaagtcgtggcccgcattacgaatgacacagaagcggtcagggatttatatgtaacggtgctgtcgacatttatcacaagcggcgtttatatgctcggcatttttacggcgctgtttatgcttgacgtcaggctggccgccgtttgcctgctgattattccgattttaatgatctggtcggttgtgtaccgcaaatacgcatccgtcttcaaccaaaaaatccgttccattaacagtaacatcaatgcgaaaatgaacgaatccattcaaggaatgccgattattcaggcgttccgccgcgaaaaggaaacgatgaaggaatttgaagaattgaatgaagctcattttcgctacaaaaacaaaatgctgtccttaaattccctcatgtcccacagtcttgtcaatgtgttgcgcaatctggcgtttgtcgggctgatctggtatttcggaggcgcgagtctgagtgcggccggaattgtttcaatcggtgttttatatgcatttgttgattatttaaaccgtttgttccagccggttacggggattgtcaaccaattttcaaagcttgaattggcccgtgtctctgccgccagagtattcgagcttttacaggaggaggaaacagaagaagaaggcgctcccgcagacagaaaggcggaagggacagtggaattccgcgatgtttcttttgggtatcgaaagggagacgaggtgctgaaacagatcagctttacagctgaaaaaggggagacggtcgccctcgtcggccatacggggtccggcaaaagctccatcctgaatcttctgttccgtttttatgatgctcaaaaaggggatattctgctggatggtcagagcatatacggcatgtcgaggcaggagctgaggtcgcatatgggcattgtgctgcaagacccgtatcttttctcgggcacgatcggctccaatgtgacactcgatgatcagcgtatttctttggagcaggcggaagaagcgcttcgccaagtcggcgccgcaagtcttctgcaatccctgccgaagggcatcagagagcccgtcaccgaaaaaggcggcacactgtcctcaggccagcgtcagctgatttcttttgcaagagcgcttgtgttcgatccggccattttaattttggatgaggcgacggcgcacattgacatcgaaacagaagcagtcattcaaaaagcgctgcaagtcgttcaagaagggcgcacgacatttattatcgcccatcggctttccacgatcagacacgcagaccagattctcgtgctggaaaagggccgcattattgaaaagggaagccatgacgaattaatggcgcaaaaggggcagtaccaccaaatgtatgaattgcagaaaggaacggcgaagtccgcggtgtaatcggaaagggtgaatcaaagattcgccctttttttcattcagtaagccagtcccacccgttttctgacaaactgcgggctgatcagctcacggtacagaaagtgcgccgtgtctccgacggtgatttcagcgccgtcttcaggaaggcgatcctgttcgaaccggtaaactccggcagcatctccgtcaggaaggtaggtgggacagatgatcgtccagtcaagatcctctgccagcagcatctcgtatacgcgggcgtgttcctttgcggctcttgttgattttcgttttgattctgccgattcaaaacggtattgatcaggcataagccgtgattgaaggatacccgcggtgccgatcgtaatcagacgctgaacatgatgccgtttcatgctgtctattatatgggtcatagcggctgaaagggtatcgtcaccgtctgtgccgagacagctcacgactgtatccgctttggaaatcaatcggcttacatcttcccgccgcctcgcattccctgttatgatttgtttttcaggaatgggaaggactgttccggctgatctgacaagagcgaatatctccatgtgtccatgtttctgcgcttcttttataaaagcctgtccgactcttcccgttgctccgagtaaagcgatgttcatgtaaaaaacctcctcgccgctcattatatcattcaaagcagcggggaaaagaatacctgtgaataaacgggaaatggtgggcaacatattcattgaccaaagaaacacttacgaactgaaacagctcccaattgactgaggcaatgaatgacatgttttccctccttttttcctagggtatgtggaataggaaaaaagggtgacggcataaaagaagacgcggtatcccgcgccttctgtaagccatttattgagttattgtacgccttggccgcccatgttctgctgtgcgaaagatactaaacgtttagtgatttctcctccgacagaaccgtttgcacgagaagttgtttctccgccaaggtttacgccgaactcagaagcaatttcgtatttcatttggtcgattgcttgagcagcgccgggtactactagttggtttgaagagttgttgtttgccatttgtatgatctcctttataaagtagtatggttgggtttgctggaattgcaccagccgatgattttcggaacgaaaaccattgccagctcggcttcacccaatgctgtttgccgctttgtactacgtattatggtgaatcaaacggcatctcattcggaaaacggtgtggtaaatagcggaaggaaaaagaaaagcccagcctggtgcggctgagcttttagctttcagtcatatatgtgataaagcattaattcatgcagctcttttacgagagccggctcttcttttcccggatgtgtgtgtccccattcaatgtctccggacttgtggtaaatcccttcgtaatgcgtaccctgccagtaaaatgaaatccgccatccgggcagtttctcttttttgaataacggtttccagctgaagtgcgaaatcatgacgttcacgctccttatgggaaatgttgatcttgttcattgtagctcatcgttcaatgcctgtcccgggtaaacctaaaaaaagacagcctcaccgccaaagtttttccggggcggtgatggaccgttctgataaatacgacgcgtattcaaaatttctccgttttgtcaggtaatcgaccgatctcagcttcggatgggaaaaaatgctgcgcccggggcgggaattagcttcaaacatccaaactttccctgaatgatcgacgccgaggtcaaaaccgatttcgccgataaaaccttcaatgtgctcatttaacacgcggctgaccgccaaggcggcttttgaaagtttccgcatcatgccgactctctcgcgcggatcgccgtataattcagctaacgtcctgacttctccgccggttgccagatgcgtcgtgatgctgttttcaccggaaatttttgccgcaatggccgtcactgtccatttcccctcggcgtttttattcgtgtgcaccctgaaatcgacggggcgcccatccagccggatcaggtcaatgccttgctgaattaaaaatgcggacgggtcattgttttctttaaatgatgtcatgaaggatgccgcatctgaatactctgccgtcaccaccgttttttcctcattcgtgtgtttgacagataagccggcatctgttttttccaccttgtaaatccccgtgccgagtgcgccgtctgcaggtttgatatagacggtgtcaaacgtgctgcataacgtttctaacgcttctgcattcgggtgcaggacagattttggcagaagtgaagcggtgagactgtttttttccagaagcttatgaatgtcccatttattaaagaattgcggattaaaccacggaatctgatagtcattcatcagcctttgttttgtctgctgtaaaagcggaacttcttccgtttttcggctcgggagcctgtcatatacgacattcggaagcgggacggccgtccgtttccagctgttttctttataaatcagcgcttggacggtgccttcttcccagttgatctgatgggccccgaaaacgaaacaatatccgttcgcctgttcatgaagagagagcatcttggaaaagaaggctgtacggtctcttaacgggcgggacggcgatgatgtgaacccggctgtgaaaacgccgatcagcggtccgatgtgcaccgtctggccgtgtatgagaaagtcagcccggcttttatgcggtatcaacagctcctcaaacaaatcttcagaaaggagcgcgcttttttcaagtgattgagaaaaggctacacggcatttgacaaccttggtcccgaaggcagcggataaaagcccttcccgtttcatggaggaggggagaaacagcgtatttttgctttttttatcgatgccgacaatcatccctttatgattcac

SEQ ID NO: 8 - DNA, 9900 bp, unidentified
  source 1..9900: mol_type = genomic DNA
atgatttacactgtaaccctcaatccgtctgttgactatatcgttcacgttgaagattttgccgtaggcggtttaaaccgttccagctatgacacgaagtatcccggcggaaaaggcatcaacgtttcgagattactgaaaagacaccatgtgccgtcaaaagcattggggtttgtcggcggatttaccggaaattatattacgtcatttttgcatcaggaaggtctggaaacggctttttctgaggtagatgaagacacgcgtataaatgtaaaactgaaaacgggcgatgaaacggaaattaacggacagggcccgacgatttcagacgaagtgttcaatgcctttttagaacagttctccgctctggaagagggagacatcgtcgttttagcgggaagcattccttcttccctgccgcataacacgtatgaaaaaattgctgaggcatgcagaaagcagaatgcccgggtagtcgtggacatctccggtgaagcgctgatgaaagcgacagaaatgaaaccgtttttgatgaagccgaatcatcatgagcttggcgaaatgttcaatacggtcattgattcagccgaagctgccgttccttacggtaaaaaattgattgaacaaggcgctgaacatgcgattgtttcgatggctggcgacggtgcgcttttatttacgaaagaggcggttttcctcgccaatgttcctaaaggaaagcttgtcaattcggtaggcgcgggtgattccgtcgtcgcgggattccttgcgggaatttcaaaacagctgcctttggaagatgcgttccgtttaggagttgcttccggaagcgcgacggcattctctgaagaactcggaactgaagagtttgtacagcagcttcttcctgaagttaaagtcacacgtctatagaggaggaaagaaaagtgaagataactgagctgttaacaagagatacaattcatctcaatatagaaagcaaagagaaagaaaacgttatcgatgaaatggtgacagtgcttgacaaagccggcagattaaatgacagagaggcttacaaagcagccattttaaatcgtgaaagccaaagctcaacgggaatcggcgaaggcattgccattcctcatgcgaagacggcgagcgtcattaacccggccatcgcattcggccgttcaaaagatggtgtggattatgaatcattagacggtcagccggcacacctggtctttatgattgcggcaaccgaaggagcgaataatacgcaccttgaggcattatcaagactttcaacgcttctcatgcgtgaggaaatccgcaaacagcttcttgaagctgaatcagaagatgcgatcattgacatcatcaatcagcatgacaaagatgacgatgaggaagaagaagagcaggaagaacaggcagcgccgtcaggaaagggcaaaatccttgccgttacggcatgtccgacaggtatcgcccatacgttcatggcagctgacgcactgaaggaaaaagcgaaagagctcggcgttgacattaaagtcgagacaaacggctcaagcggtatcaaacataagctgacggctcaagaaattgaagacgcgcctgcgattatcgtggcggctgataaacaggttgaaatggaacgctttaaaggcaaacatgttcttcaagtgcctgtaacggccggtatcagacgtcctcaggaactgattgaaaaagccgttaaccaagatgcgccgatttacgagggcagcggcggaggatcttccgcttcagatgaagacggcccgaaacagaaatcaggcacaagcatcggaagtactttctataaacacttaatgagcggtgtcagcaacatgctgccatttgtcgtcggcggcggaattctcgttgcgatttcattcttctggggaattcattcagctgatccgaaagacccgacatacaacacgtttgccgcagcgctgaactttatcggcggcgataatgcgttaaagctgattgttgcagttcttgccggattcatcgcaatgagtattgcggaccgtccgggatttgcgccgggtatggtcggcggatttatggctacgcaggcgaatgccggattcttgggcggactgattgccggattcttagcaggttatgtcgtagtccttttgaaaaagctgtttgtctttattcctcagactcttgacggattaaaacctgtattgatttatccgctgctcggtatttttatcaccggtgtcctgatgcagtttgtcataaacacacctgtcgcagcgtttatgaatttcttaacaaattggcttgaaagccttggaactggaaaccttgtcttaatgggtattattttaggcggtatgatggctatcgacatgggcggcccgctcaataaagcggcgttcactttcggtatcgcgatgatcggggccggaaactatgcgccgcatgcggccatcatggcgggcggaatggctccgccgcttggaatcgcactggcgacaacattctttaaacacaaattctctaaacgtgaccgtgaagccggtattacgtgctacttcatgggagcagctttcgttacagaaggcgccatcccgtttgcagcggctgatccgcttcgcgtcatccctgcagccgttgtcggttctgcggtagcgggcggactgacagagtttttccgggtaacgcttcctgcaccgcacggaggaatattcgttgcattcattacgaaccatccgctgctttacttaatgagcatcgtgatcggagcgattgtaacggcggttatcttgggaatcgtcaaaaaaccggttgcagagaaataagcaaaaagcgtccattacgggcgcttttttcaatcatgtgcgcatagaagcgttatgtattttatgatgagttatggtaaagtaaagatggtacatgaaccgaaacagtgatcgtctaggaggaaccacgttgactgaagaacaaaaacctacttctgaaaaatcggtaaaaagaaaatccaacacatattgggaatggggaaaggccatcattatagcggttgcacttgcgctgttaatccgccatttcctgtttgaaccgtacttagtggaaggatcatcaatgtacccgacattacatgacggtgaaaggctgtttgtcaacaaaagcgtcaattatatcggagaaatcgagcggggggacatcgtcattattaacggcgatacgtcgaaagtccattatgtaaagcggctcatcggaaaaccgggagaaacggtggaaatgaaaaatgacacgctttatataaacggcaaaaaaatcgcagaaccgtacctcgcgagcaataaaaaagaagctaaaaaactgggagtcaacctcacgggagatttcggccccgtaaaggttccgaaaggcaaatacttcgtcatgggcgacaatcggctgaactcaatggacagccggaacggactcggtttaattgccgaaaaccggatcgtcggcacctcgaagtttgtgttcttcccgtttcatgatatgcgtcagacaaaataaaaacgccgcgctgcccgatttggacagcaggcgttttttatttggcaaacgaggcggcgacggcgatgaacattaccgtggccagcactgatccgacaatggccagaacgctgaaaaaaagattcacctgctgcttctctttgccgcgtttttcaagcatccgttttgaaaagagatgcgaaaacgtgtaaatgagggcgattccgacgtacaagccgatgtcacgtgcggtaaagcctgtgacggccagaaaaaccccggtgaagaaaatcataagacagtattctgtgagtgttaaaagcttcaacttgttcctccgtgatgaatatgtatatatatctattgtatcacagcgctccgggtttgacgcctgcatcttttcaaacccgtatattcgcagggaaacatttctcttcccctttattcatggtatacttttagtgatgatagacacgatggaggattctaaatgattgctgtaaataatgtgagcttgcgttttgcggatcgcaagttatttgaagatgtcaatattaaatttacgccgggcaactgctacggattaataggcgcaaacggtgccggaaaatcaacatttttgaaaattttatccggtgagctggagccgcagaccggcgatgtccatatgagcccgggcgaacgtcttgccattctgaagcagaaccactttgaatatgaagacttcgaagtgctgaaagtcgttattatgggacataaacgtttatacgaagtcatgcaggaaaaagacgcgatttatatgaagcctgatttttcagatgaagacggcatccgcgccgctgagcttgaaggagagtttgcggaattaaacggatgggaagctgaaagtgaagccgctattttgttaaagggcctgggcatctctgaagatctgcatacgaagaaaatggctgacttaggcggttcagaaaaggtgaaagtgctcttggcacaggcgctgttcggcaagccggacgttctgctgcttgatgaaccgaccaaccacttggatctgaaagcgattcagtggctggaagagtttttaatcaactttgagaacacagtcatcgtcgtatcccatgaccgacactttttaaataaagtgtgcacgcacatcgccgatcttgattttaataaaattcaagtgtacgtcggcaactatgatttctggtatgaatcaagccagcttgctttaaagctttctcaggaagccaacaagaaaaaagaagagcagattaaacagcttcaggagttcgtggccagattcagcgccaacgcctctaaatcaaagcaggcgacatcaagaaagaaactccttgataaaatcacgcttgatgatatcaaaccgtcatcacgccgttatccttatgtcaatttcacgccggaacgtgagatcggaaatgacgtgctgcgggttgaaggcttaacaaaaacgattgacggggttaaggtgcttgataacgtcagctttattatgaacagagaagataaaatcgcttttacgggacgcaacgaacttgccgttacgacattgtttaaaatcatttccggggaaatggaagcagacagcggcacatttaaatggggcgtgaccacttcccaggcgtacttccctaaagacaacagcgaatattttgacggtagcgacgcgaaccttgttgactggctccgccaatactcgccgcatgaccaaagcgaaagctttctgcgcggtttcttaggacgcatgctgttctcaggagaagaagtcaacaaaaaatcaaacgtcctttccggaggagaaaaagtgcgctgcatgctttcaaaagcgatgctttccggcgcgaacattttaattctggatgaaccgaccaaccacttagatttggaatccattacggcgcttaacaacggcttaatcagctttaaaggcgctatgctgttttcatctcatgaccatcagtttgtacagacaattgccaaccgcatcattgaattaacgcctaaaggcattgttgacaaacaaatgtcttatgacgaatttcttgaaaacgcggatgtacagaaaaagcttgaagagctatacgcgtaataaagaagcaggggagactctgcttttttatttatgaaaaaagttttcaatcttctgacttcccgttacaataaatgaatacataacttgaaaagagacggaggaatgcatcatgaagtttttagttgtcggagctggaggagtgggcggatatatcggcgcgcgtcttgcagaaaaggggaatgacgtgacgtttctcgtccgcaagaaacgggcggaacaattacaagagacgggattgaccgtcaaaagtgcaaaaggtgatgtttcaatcaagccgaaattaatgatggcgggcgaagaaggagcatttgacgcggttatcatcgcttcaaaagcgtattctttgcaggacgtcattcgggatgtcaaaccgtttgtcaaaccttcgtcggtcattattccttttttaaacggatacaggcactataaccagctgtttgaagctttttcaaaacggcaggtgctgggcggcctctgctttattgagagcgcccttaacgacaaaggggacattcttcatacgagcgctgcccaccgctttgttttcggtgaatggagcggggaacgcacggaacgtatgaatgagctggaaaacgcatttgcaggtgtgaaagcggatgtcatcatcagccgggatattgaaaaggacatttggaaaaaatatatgtttattgccgcgcaggcgggagtgaccactttgtttcaacagccggtcggaccgattttagaaacagaaggcggccgcaacacggcaaagcgcctctttgctgaaatcggagccattttcaaggctgccggcatcccgaaagatcctggtctggaagaagagagttatcgcacattgacaagcatgtcttaccatatgaaatcgtcgatgctgagagatatggaaaagggcagggtgacggaaggtgatcatttacacggatttttgcttgagaaagcgaaaggcttgtcagttgatacacctgttttggaaaccgtctgcgccaacttgcaaatgtatgaagcgaaataaaaaaaggaggcctgatcgcctcctttttttattaaaatgcccagtttccgtttctgaagatcggttctcttttgccgtccgcagtaatgccgtcgatattcatatctttggaaccgaccataaaatctacgtgtgtaatgctttcatttaagccctctttgacaagctcttcccgtgtcatttgtttgccgccctcaatattaaaggcatacgcactgccgatcgccaagtggttagaggcgttttcatcgtacagtgtgttgtaaaataaaatatttgattgtgaaatcggtgaatcgtacggcactaaggccacttcaccgagatagtgtgagccttcatcggtttcaacgatgccttttaaaatctcctcgcctttttcagctttcacatcgacaatgcggccgttttcaaaggtaagagtgaagttatcaataatgttgccgccgtagctcagcggtttcgtgctggaaaccgtgccgttcaccccgtcacgctgcgggagggtgaaaacctcttctgtcggcatgttggccatgaattcgtgcccgctctcatttacgcttcccgcgcctgcccatacatgttttttcggcagttcaatggttaagtctgttccttcagcctgataatgcagggcgctgtaatgttttttgttcaggtgttccactttttcatgaaggttttgatcatgttttttccatgcttccacagggttttcttcatttacgcgggtcgttttgaaaatttcttcccataaaagctgaacggctgtttcttccggctgatccgggaacacttttttcgcccagcctgttgaagccgctccgatcaccgtccagctgaccttatctgattgaatatattgtctgtatgtatgcaatgcttttccggcggctttttggaacgctgcaatccgtttgccgtcagtgccttttaacaggtcggggtcggatgagacgacagaaataaacgcagccccttttttcgcgagctcttcttttccttttgcttcccattcaggatactcttcaaacgcttcaaaaggtgccatctcgtattttaatctggacagttcatcgtcctgccaatttacggtgacgttttttgcgcctttttcataagaatgtttggcaagcagtctgacaagttcccggacatcagttgaggcattgattacgacatgctgtcctttttggacattgacgccgacttctaccgccagctgagcatatgtatgtaacttttcagtaaacgaactcatcgttcattcctcctcatcggtttttctcatcatatcaaatatcgcctcataaaggaaagaaaggagcacaaacgaaaaagacacagcttttgctgcgtctttacgggtgattcagttttacggcgccgccggccggagaatggggcatattggccagtgatatgctcataccccccaaaataattccgcttaaaatgacggctactgctacgcgtcttcccacatctctaagcatatgcttcctccttaatgtttctccagttctcacaatatgagccggccggagaaaaaagaagcacaatcttattatttaatcgctttttgcagcttgtcgatgacgtctaatgaacgtcccgtgccgatagccactgattcaagcggatttgccgccaaatgaacgggaacgacgatttcatctgtcagccattctttcatcccgtttaaaagggagcctccgccggttaacacgacgccgcggtcaacgatatcaccgctgagctccggcggacaatcttcaagtgtcgctctgatcgcttcaagaatatgcaggagcgactcacgcatcgcgtgctgaatttcattagattgcagtctgattgtcttcggcagaccggttacaaggtcacgcccgcggatttccatcgtttccggcacatgttcgatcaatgcaaaaccgatttccattttcacttgttctgccgtacgttccccgatcagcaggttgtatttttttctgacgaacgaggcgatatcttcatcaagctggtcgccgccgattctgatcgaatggcaggatacgacgcccccgaatgagatgatggcgacttcagtcgtaccgccgccgatatctacgacgacgtttgcgaccggctcgtcaacagggaggtcggccccgattgcagcggctacgggttcttcaatcaagtgaacgtttttcgctccgcagtttttgacagcgtcactgatggcgcgtcgttcaacggctgttgagcctgaaggcgtgcagacgacgacattcggtttgcggaaagtcattccgattttttttccggcttttttcataatgtgttttaataagtcagttgtcatatcataatcagcaatgacaccgtctttcatcggccgaacggcgacgattttccccggcgtctttcctatcatgcttttggcatccgttccgatggcaagcaccgctttcgttgttgtatctacagcgacaacggaaggctcgtttaaaataattcctttatttttactgtaaacaagtatattagcggttcctaagtcgattccgatctcagttgattgaaacatgttttcacccaatccctatcctgatttaattgattctacatttaaagtttctgagaaaaaccacgatttttgggggtttccgccgggaatgaaatgtttttgtaaaaaaggagggaaagtctgtatcccatatttcaaaaggattttcttttgtaaaaaaacattcagttccggatgctgagaaacttttgctccgttcctttcgtctacgtttatgaaggattttccttggacataaagaagagacattaaaggaagtaggggatttctatgaaatcgattggtgtagtaagaaaagtagacgaactcgggcgtattgtcatgccgattgaattgagaagagcattggatatcgctattaaagatagtattgaatttttcgttgacggagacaaaattgtcttgaaaaaatacaagccgcacggcatttgcttaatgacgggggaaattactcccgaaaatcaggagtacggcaacggcaaaattattttaagtcctgaaggagcgaaaatgctgcttgaggaaatcgaggaagcgttaaaagatagatcataatcagcaaaaagggcggagcatcattccgtccttttattatgaacttgtgcaaaaactcacgggaaaagtaagcgaacctgctcaattaattgggaatagttatcattttttagt

SEQ ID NO: 9 - DNA, 9960 bp, unidentified
  source 1..9960: mol_type = genomic DNA
caataatgcagtcaacaatgttacaatatatgtagaaatgctagcggcaggtgggactatgggaaaatatcaagcgcgtatcatttccttaatcttggcaatgatttttattatgttttgggattatttattttatatgattgaacggaatccgattaactggcccgttgacatcacctttaccgccgtcacattaatcagcgtctggctgctggcttattatgtggataataaaaagcagctgctggcggagctgaaggacagcgagcagaaagcaaaggcgctttcagatgagaaaaaccggatcatggacaatctgcaggaaatcgtttttcagacggatgcacaagggaatattacatatttgaatcaggcctggacggcgattacgggccactccatcagcgaatgtatgggcacgatgtataatgattattttataaaagaaaaacatatcattgattatattatctcccaaatcaagcagcagtccccgtcaggaatgtttacggccaaatatattaaaagagacggcacgatcttttggggagaagtccattataagctgtattatgaccgcagacagaaatttgtcggaagcctggggacgatgtctgatatcagtgaacggaaaaaagcggtcgacgagcttctggaaacgaacgaacgccttgccatgcaatctcaaaagctggccattgccggggagcttgcggcaggaatcgcccatgaagtaagaaatcctttaacgtctgtcagcggctttctccagctgatgaaagcccagtatccggaaagaacggattattttgatatcattttttcggagattaaaaggattgatctcgtgctgggggagctgcttctgctcgccaagccgcagaccgtgacgtttaccgctcatcatctcaaccatattttacggcaggtggcaacgcttcttgacacgaacgcggtgctgtcgaacactgtaattgagaaggatttcaggcagactgagtcctgtatggtcaacggtgacgaaaaccagctgaaacaggtatttatcaatattattaaaaatgcgattgaagcgatgcctgacggcgggaatattcatatttatacaaaacgggatgaagaatacgcggtgatcagcatacaggatgaaggaaacggcatgtcgaaagaaaagcttgaaaacatcgggaaaccgtttttctcaacaaaagatcagggaacgggtctcggtcttccgatttgtttacgaattctgaaggagcataatggtaaacttaacattaagagcaaaaatggagaaggcagtacgtttcaggtgattctgccgttagaagcggctgctgtccaaaaggaggaaaaataacttgcttctttttgtatacggtttgttacggaagcgtgaagaaaaccatgaactgctgaaaggcgctccgtgtgtgtgcgagcaggctgcgataaacggcgtgctgtacgaaacggataaaggtcagcctgcggcgtctttagcagaaacagcttttgtgtacggggaattgtatgaaacagatcatcagatgatccgtaagctggatgcgtattatgctgattttgatcgaaaagaggttgccgtgacgacggatgccgggaataaaaccgccttcgcatatgtcgtaaagcccgaccagtgtgcttcattcagccgtatcaaaagcggagactggaaagaataccgttttatgaaacgggaggatgacagtccggtttattatttcgcttacggctcctgtatggacaatgcccggtttaaacaggcgggggtcgaccacttttttgccgagccggtcggaggggcggttgttgaagggtattcgacgagattcacattaaggcgtcctgacggttccagagccgatcttgttgaagacgggggacgaactgaaggcgtgctgtacaagctcccgtttgaagcggcaacctatctgtacaaaagagaaggtgttgataaccacacataccgtcccgcgttcatagaagtgtgtgccggcggccgtgtatacagaggcgtcctgacgtttctcgttcttgataaagctgaggaaattgcgcctccgggtcactatcaggaagaaatcgaacgcggagcggacctgtatttatcgcccgccttttcagaaaagcttaaacgctatatgaactcattgccgaaaatgtgatactattcctattagagatattgtttaaaggagtactgttatgagaaaagagtttgccgttatcggtctcggacgcttcggcggaagtatttgcaaagcattaagtgaagaaggccttgaggtcatggcgatggatattgatgaagataaagtaaatgaatacgccaaaatcgcatcacacgcggtcatcggcgattcaaccgatgaatcggtgctgaaaaacttagggctgcgcaactttgaccacgtcattgtggcaatcggcgaaaatatacaggcgagcattttaacgacgctgatcttaaaggaactcggcgtgaacacgatcacggttaaagcgcagaacgattatcatgagaaagtgcttgcgaaaatcggggccgaccacatcgtccaccctgaacgtgacatggcaaaacgcatcgcccacaacatcgtatcaaataatgtgcttgattatttggagctgtccgacgaacacagcattgtggagatcgtggccaacagcaggcttgcgggcaacacactgcttgatctggacatccgggcgaagtacggcattaatatcgtggccatcaagagagaaaaagaagtgatcgtatctccgctggcgactgaaatgatccaaaaagaagatattctcatcgttatcggctctcttacggacatttcccgatttgaaaaaagagtgcttcacacaaagtgaaaagccgtccgtaaaaggacggtcttttattatgtcagaatagaaagacatttatcgtgtaagcggtattttctgcaatttttattgactatgatcaggcgatccgatagcatattgtaccatataaagaaaaagaggaggcctcattttgaacaaagaaatactggctgaacggctgaatgcatcagccggcagacaaaaagccgataccgtcattaaaaacggaaaaatcatggatgtatttaaccaggaatggatttctgcagatatcgccattacgggcggagtgatcgtcgggctgggcgaatatgaaggggaagaagtcattgatgcagaaggtcaaatgatcgttccgggctttattgacggacatgttcatatcgagtcctctatggttactccgattgaatttgctaaagccgtcctcccacacggagtgactactgtcattactgatcctcacgaaattgcgaacgtttccggagcaaaaggcatttcttttatgatagagcaggcgaaaaaggcgccgctgaacatccgtttcatgcttccttcctgtgttcccgccgctagctttgaacgctcaggagccgtattaaaagccgaggatttaaagcctttttatcaggagaaagaggtgctcgggcttgcggaagtaatggattatgtgtctgttgcggaaggtgaagaggatatgcttcagaaactgcttgatgccaagcgtcacggaaaacggatcgacgggcatttagccggactgtcttcagatatcatcaatatatacagaaccgcaatggtgtcaaatgatcatgaagtgacgacaaaagaagaagcgctggacagaatcagacggggaatgtatgtcatgctgcgcgaaggatcggtggcaaaaaatacgctcaatgtgctgccggcggtgaatgaaaaaaacgcgcggcgctttttcttttgcacggatgataagcatgtagatgatctgctttcagaaggaagtgtgaaccaccaagtcaaaatggcgataaaggcgggacttgatccattccttgcgtatcagctcgccagtttaaatgcggctgagtgctacggtcttgaaacaaaaggggcggtagcgccgggttttgacgcagatctgctgttcatttcagatgtacgggaggctgccgtcacgaagaccatggtcgccggccggaccgttgccgagaacggacgcacggtttatgaacagtctgccggttcctattcgccggaccaggcgctgttggatacagtgcggctgaaagcgccgctgactgaatcggattttcatataccgattcaagagggtaagaaaatgaatgtaatcgaaatgataccgaatcatttggagacgagaaaaaaagaagtgcctgcaccgtctgctgacgcgtttcgtcctgatacagtaaatgatttattaaaaatcgccgtcgcagagcggcacagcggcgaaaaaatgataggactcgggattgtacaagggttcgggttaaaagaaggcgccatcgccacaaccatttctcacgattcccataatatcatcgccgtcggtacaaacgatgccgatttggcaaaagcaatcaacaggctccgtgacaccggaggaggattaacagccgtgaaaaacggagaattgcttcactccgttccgcttccgatcgccgggcttttgtctgataagtcggcggaatgggtcaatgacagcttgggcgtgcttcatgaaaagcttcctctgctcggattcaacggtgatttcaatccgtttctcacattatcgtttttagcgcttccggtgattccggatattaaaatgacagcagccggtctgtttgatgtcaaagcctttcagcatattccgctgcaataaaaaaactgcactcattacgagtgcagttttttttagacctccataatgatcggaaggatcatcgggcggcgttttgttttttcgtataggaacggagcaagtgtatcggtaatttcgtttttaatttcggaccactgcgttgttttccgatccattactttctccagatggttcgaaatcagctgctgcgcgtcattgatcaaatcaccggattctctcatgtatacaaaacctcttgagattaaatcaggccccgccgaaaccttaaagtcgtccatattgatgctgacaacaacgataacaaggccttcttctgaaagaatgcggcggtcgcggagcacgatattgccgatatcaccgattccgcttccgtcaatgtaaacggagccggacggaattttgcctgccacagcggcttcttcgcctttgagggccagcacttcgccattatccatgataaagcagttttcttcaggtatgccgcaatcagtcgcgagtttgacgtgcattttctgcattctgtactcaccgtgaatcggcataaagaacttcggtttgatcaggcggagcattaatttctgctcttcctgtccgccgtgtccggatgtatgaatgtcattcaatgaaccgtgaatgacttctgcgcccgcacggtacagctggttgatggtgcggcttacgctgattgtgtttccgggaatcggagaggaagaaaagacgaccgtatctccgggattaatggaaatctgccggtgcgtcccgttagcgatgcgtgataacgccgccatcggttcaccttggcttcccgtacataaaatcgttaccttattggcaggcaaacgattgatttcgttatgctcaataaacgtatttttaggacaggtaatgtaaccgagattttgtccgatttcaatagccgattccatactgcgtccgaatacggcaacttttctgccgtttaaaacggctgcttcaatcacctgctgaagacggtgtatattcgaagcgaatgtcgcaaatataattctgccgtccacttttcggaaaatatcgtgaatgctttcgcctacgcggcgttctgacatggtgaaatcaggaacctcactgtttgtactgtcagaaagcaggcacagaacgccttctttcccgatttcagccatctttgtcaagtttgccggttcgcctaccggtgtgaaatcgaacttaaagtccccggtatgtacgatattgcccggcggtgttttgacgacgattccgtatgaatccggaatgctgtgtgtcgttctgaagaaggatacagacgtcttgcggaacttcacgatgtcgtcttcaccgatgatgtttaatttggtctgtcttaacagcccgtgctcttcaagtttattccgaagtaagccgatcgcgagtttaccgccgtaaacgggaatatttacttgtctgagcaggtacggaataccgccgatatggtcttcgtgaccgtgggtgataaacagacctttaattttatcttcattttttactaaatacgtgtaatcggggattacgtaatcaatgccgagaagttcatcttccgggaacttgattcccgcatcaatcagtacaatttcatcctgaaactgcaccccgtacgtatttttcccgatctcgcccaatccgccgagggcgaaaacggcagtctggtcattttttacaaatttcattccgctcataactccaatactttaaaattttcgctctgcttctcatattctaaaaaagcaccgtccacaggctgaacaaattcaatgttgtatttctgatctttcagtttcgttctgacgtcccgttctgaaacgccctcaatgtaaagtgaatctgttttttcccgaacaggtacttcgtcagccttctcttgataaaataccttataaatcatccaaatctctccttaaatgccgtatgttagactcgcttattctattatattcgaaaacggattataaggaaagtctcgagcaaaaaagggactttcaaaaaccgaaagatcccttctacgcgatgactttttttcggagcatcccgccccactgtcttctgagtttcttccgtaatcggcgcagcatcgtatacctcctttaaagccctttatagcagtagtatgcggaaaaaatgaatttgttttcgtgggtaaaaatggaatttcatcaaggcggttttccggaagaaaaactttctgtatgatgatattgaaaaagacacgactgataaacgggaaaaaggagaaattgacatgaaaccaaaattgatcttttttgatattgacggcacgatctatgaccatgataaacatattcccgatagcgcaagaaacgccgtgaaggcgcttaaagaggctggccatcacgtttttatcgccagcggaaggtccccgtttatggtaaaaccgattctggaagagcttgagattgaatcatttatttcatacaacggccagtatgtcgtatttgaaggtgaggtcatttataaaaatccgcttcctgaagatgcggttgcgcgtttattaaagcaggcggcagaaggggagcaccccgtcgtatttatggcggaagatacgatgaaaacgaccgtccccggacatccgcacgtctcggaaggaatcggcagtctgaaagcgccttatcccgaagttgatgaaacattttataaaggaaaagaaatttatcagctgcttttattctgccgcggacatgaagaaaaagcatatgccgcttttccggagtttgattacgtccgctggcatgaattgtcaacggatgtgctcccgaaaggcggttcaaaagcggaaggcattaaaagagtgcttgagcggcttccgtacggcgtaagtgacacttatgcattcggcgacggactaaatgatcttgagatgatccgttttgtcggcaccggggtggcgatgggaaatgcggttccggaattaaaagctgccgctgatttcgtcacaaagccggtggatgaggaaggaatcagctgggccgtcaaagaactgggccttttgaaataaagaaaaccggcactttgggaagtgccggttatttttaacggtcaatggcaatggcgttttcaggttctttgaacggattttctttatcaatatggtcgtaaaacatcacgccgttcagatgatcgatttcgtgctgaaacacgatggccggaaaacctttgaggcggatgtcaatctgttctccttcaagcgtcaccgcctttacgcggatgcgggcgtagcgcggcacatagccgggaatgggttcgtctaccgacaggcagccttctccgctcgtcaagtagcttttttccaccgagtggctgacgatcttagggttaaaaagagcatagctgtataaagtgcctttcgcatcttctgcatggacggcgatcatccgtttttttacgtctgtttggggagcggcaagaccgacgcccggacgaagcttgtatttctcggctaattcaggattttggctgtttttcacaaactcaatcatatcagcaagatgctgtttttcttcgtcagaagcgggaagctgaacctcttcagcgatctctctgagcgcgggatgtccgtcgcgtacaatattttccatagtaatcaagaaacttcactcctaaagcttaatatgtaaattatagcgaatttctttatcgcggcgcaacgaaacaattggccggccatagcaggccggccgatgttcatggtgtataaggtcagcagccgcagcctaaataagcggcaccgacgatgattaataagataaacaatacgacgattaatgcgaatgtgcggccgtatccgccgccgcagccgtaaccgtaaccgccgccataaccatatccgccgccgcaacaaccgccgccaaatccgccgccaaatccatatccgtacataaaaaaacctcctcttttaattacgctgaccctctgtcagcagggccggcgtctactataaactatgtaaaagccaaaaagatgtatggtcatccgccctgttttctcaattttttacgcaagtattctttgaaagcaccgtaagcgtctttacacggtcatgccaattacagctcagctattgtcaaaaagcgttcgggcttctatagtggaaattgtcaagattgggggattttaagtgaatatgattcaaaaaaaggtattagcaggaatcggagcgctcggcgccgcaagtatgcttcttgccggctgcgggggaaaagatccggttgaatcgcttcatgactcgatggaaaaagccgttcaagcggaaaaaccgtttcaaaaagagcagaaaacattagaaaaattggagaaaaaagagcataagctatacgacagcgcggttaaactgaatatggatgattaccagaaaattgtcacactgtctgatcaggctctgagcaatgcaaatcagcgaaaaaaacatctgaaagcagaaaaagacagtattgatgattcaaaaaaagcgtttgaatccgcaaaaaaaacttctcaagaaataaaggataagaaagtaaaagaaaaagccgggcatgctgtcgctttaatggagaaaaggtatgcatcttatgatcttttgtacaaaaagtacgaaaaagccatttcacttgataaggacctttataaactgattaaagataaaaagctcacattagcgcagctggaagagcaaatcagcaaagtaaactcagtttacgaaaaggtgcataaacaagcagatgagttcaatcaatttactaaagactacaacaaagagaaagaactattatttcgcaaatagtatagtgaaggcatatcggcatgggctgatgaactgataacaaccgagttcagccgcctgtaaaaaggcggctgaagaattggtatatacgtgtgtttcccattgacaacaagtaaatcatggtatagtatttatttagggaaatattcacataaaaccaatcgttgtttcggcacgttggaatgtttttattatattgatatatataaaacagaatgaaaatgtgattttaattatcagaaaattctatctatttatatttacggcgatatgatatttgtacatgctttttgcttttaaacctttatccggcataaaggtttttttattttgcttagtaatgcttattaagtaagggtgctgagagtaaacg

SEQ ID NO: 10 - DNA, 9960 bp, unidentified
  source 1..9960: mol_type = genomic DNA
acaattttaaaattgaagtgaaaaacgttaatcaaaatcggaacgaaaagaaacggcagaaatacggaacaggaggaaaacaaatgcttgcttacgtctttcccggacagggagcgcagtttaaaggaatgggcagagacttattcgatgaatttagtgagctcattaaaaaggcagatcatattttaggatactccataaaggatttatgcctttcagatgaaaaagagaggctgactgatacgaagtatacacaacctgcaatttatacagtgagcgcactgcaatatttaaagggtttaaaagaaggtcatactcgccctgatgctgttgcaggacatagtctgggtgaatatactgcgctttttgcggccggcgtttttaattttgaaacaggtctccggctggttgctaaaagaggcgagttaatgagtgaggcttccgagggcggaatggctgccgtgatcggactggatgagcatcacataaaaaaaatacttcaaaagtatgaattcggccaaatagatatcgccaattacaatacatcttcacaaattgtgatagcaggggctgcagaagagataaagcgggcagcctctttttttgaaaaagaaggggcgaaagcttatatcgtattgcaagtcggcggagcttttcattctcgtttcatggaaagtgcacagcgtgaattcgcggaatttatcgaggagtttcatttttctgaattgaatttcccagtcatctctaactatacggcaagaccttataaacaagaagatatcaaacggaatttaattgaacaaataacaaactcggtgaaatggactgaaagcatcaggtacttaatgggacaaggcgtcactgtgtttgaagagatcggtcccggaaacattctgactaaattaattcaaaccatacaaagagatgaggaaccgcttaatattagtgcgccggaagacagtctcttgttttctgaagggaacaaggacaattcggaaagcgagaaagagaagggcctcttggccaaaaatatagaaagcagtacggcttcaccggtgtcccgattgggtgatcaatctttcaaagaagattacggtttgacatacgcgtatttggcaggtgcgatgcatagagggatctcttccgctgaaatgctgatccgcctcgggaagaaaggaatgctggggttctttggcacagcgggtttaactataagggaaattgaacaggcccttatttctattaagaagacactcaaaaatggccagccctacggaatgaatctgcgatttgagccggatcatcaagatagagaaaaggaattgatagatttatatattaagcatgacgtacgggtaatagaagcgtcttcgtatctttccgtcagcgcgccattaatttattacaaagcgcatcagttgaagatggacaaaagcggcagggtcatacctcataatcggatcattgccaaagtttcacggccggaagttgcggccgcatttctgagtccgcctcctcaaagtatcgtaaagagaatgtgtcagcagggtatgctgacggaagagcaggcggagtggctcagccgtgttccggcagcagacgatatcattattgaagcagattgcggcagtaataccgaacagggctcattgacggcgatgctgccgttttttgttaacctgcgggatgaaatgatggaaacacacaaatacgcaaggaagatcagagtcggggccgcgggaggaatcggaactcccgagtctgcggcgtctgcttttttaatgggcgctgattttattatgacgggatccattaatcaatgtacagttgaagccgaaacaagcggttttgttaaagaaatgctgagtgagacaggaatatgcgacactgcttatgcgccttcggagactctgtttgagttcggaacaaaggtgcaggtgctgaaaaagggcacgctgtttccggtcagggcaaataaattatttcaaatctaccaacaatatgagtctttgagtgagatagatgaaaagacaaaaattcagctcgaaaatgtttattttaacaacacttttgatgaaatctacgaaagcataatagataaacagccgaacctggcccaaaaagctgagcgcaatcaaaaatataagatgctgttactatttaagtggtatttgcagagaggatgtctgcttgctttggaagggcaggaagaacaaaaggtgaatttccaagttcattgcggcccgtctttaggcgcttttaatcattgggtaaagggcacggatttggaatcatggagaaacagacatgtcgatgacattggtgaaaaaatgatgaatgagaccgaaagtctgctaaggcgcagattggatacgttatttctacactaaagaaggggcgggtggaggccaatggttcatttgaataaacagagtgcggatatcgcgataatcggggttgcttgcagattccccgatgcagatcattatcatgaattctgggaaaacataaatagcggcaaagattccatccgggaagtgccgattgaacggtgggatcccgatctttattattcagctgatatcagtataccgaacaaaacaatcagcaagtggggaggttttattcataaagctgaccatttcgaccataagttttttaatatctctccccgggaagcgaaaaacatggatcctgagcaaagaatccttttagaagaaacttggcggtgcatcgaagattccggggtctctttgcgggatctccagcgtaaaaccacttctgtttttgcgggtattatgaattctgactaccggcagacaggtcatgctgaatcggaaatcgacagttacgcctgtttaggaagtttcgaagctatgctctccaaccggctttcatacacattcggtttgaccggaaaaagcatgtctgtaaatgccgcttgtgcatcgtcgcttgttgctttacatgaagcggttaagtcattgcggacaaaagaaagcgattatgcaatcgcatcgggagtaaacttgaatttccaacccggaaaatatatttctttttctaaagccagaatgctgagtcctgacggcaaatgtaaaacctttgacgcagcggcgaacgggtacgttcccggagaaggggcaggcgttgtattgctgcagcgccttggtgatgcagtaaaagccggtcaccatatatacgggatcataaaagggacagctgttaatcatggcggtaaatcccgttccataaccgctcccagagcagaagcgcagtgccgggtcatctcagaagctttacacaacgcttctgtcgatgctgcgacggtttcttatgtagaagcacatggcacgggtacatctctgggcgatccgatagaaatagaagcattgacaaaagcgtatcggcaatatactgaggatactcaatattgcaggatcggaagtgtgaaaacaaatatcggtcatacagaagcagcggcgggaatggccggattgataaaagttctaatgatgatgaaacatcggagggtgccgaaaagcctgaatgtacatacggtaaatccaattatacagtttgaaaaaacaccgtttaggccggcgcttgattcaagtgattggaaggcggcagacggggctccgctcagggcgggaatcagctccttcggttttggaggtgttaactctcacgtcatcatcgaagaatttatacatacaaaagacgaatctgacagaaagggaagggttcccttccttttttccgctaaaagtgaagaaagcatgaaacaaatgatacataattggagaaccttttctgacgattctgaggaattttgcaaaatatcaaatgaagtgatcagcagcacccttttaacaaggacatcgtttgattacagggctggtttattggcaggcagcaaggaggaatttaaaaatcagtttaaagagggattgagttttcggaggaaacaagattctgaatgggcgcttgccatcggtgacttcaaatgggaaagttataaacaatttgccgttttgaaacgggtgttccctgatttagaaaagaacgtaaggccggtgctgaagggtataccaacattgcacagttcatttaaaagaaaatggacacctgagaaaatccgcttgtattctttagttatcaatacggaaatcataaaaagtatcctggcatccggttttaagccaagcctcattacaggcgccggagacggatattacgtcgctcttgcagcagcggatatcatcaaaattgaaaaggccatttctgtaatcacaggcgctgaaaaaccttcgcgcatatcctgtaagccgccgtccattccatttgtcaatccgtacacttcaaaaacgtacccgccggttcgattaaatgaagattatgtaaggatgctgctgaacggtctgagcttcccggctgattttgcagaccgcctgtacgggaaaacacggctgcttcttgagcaccaatatacgtttaaagggtttttagaagagtgggataaagcgggaaagactcaggaaatcgagattttatcttatattgtgaatcgcacggcggataaaagaaacgcattgccggctgatgaggaagcggcgatcatggctatccttgcctctatgagaagactgtataagagatgggatttaacagattatgatggattctttaataatgattatgttctggaaatattaaatttactggatgaaggaatcttgtctaaagaactttttctgagcatgttattgaaaaagcaagacggaattaaacaggctgttgcttgtattaatgagcagccggcaggtttttatgataaaactcgttttccgttgatgacagaatacaatcaaatggatgccgtgtctcaggagatagctgaccatgtgccgcttcacacggaagaaaagggaacggagccggatgtgaatatttctgatttttcagtttttcaattcggatcgctccctccatcagcaaaactaaacaaagcggctgcttttgaggcggacaaaccagccgatgaggctctatatcattcatttatgcaaatgtggctttcaggagaagagattgaatgggcgaaactaggtgatgatcataaaagaaaagcaccgcttccgccttatccgtttgagcgctctcaattttctttaaaccctcagccggtaaaatcagacgcggctttttcagaaaagaagaatcaggcactttatacgccggtatggaaagaaaccgcacttgagaatgaagcgggcagccgacaagcctgtactattttgttgttctccgggaataaagactattttgaagagctgaaaaaaagagagaatacgtccgcatatcttattatgcccggtgagcgtttagagcagataagcgacagtgttttcacgatcaacaggcagcgtcccgaagattatcaaggtgtcattcgtctgttaaaggaaaaaggcgcagcgcccgatgcaattttgcatacgtggtctgaaaaacagcctgaactggaagaaacactcgaatcaggaatttattccgtgttttatctgactaaggctttaatcagcgagaaccttataaacaaagtaacgttcatttatacattcagcgcagatcaaaaagaaaaattgccgtattttgaaggtgtgaacggttttttaaaatcggtaaaacaagaatacccgcagcttcattgtaagacggtttgctttgaacatgattctgcatccgtttcattttcagagaaggctgacattctttataaagaaataaacacagcgcctgatcggcatattcatatcacttacagaggctgcgtaagatatgcacggatgttagtgaagcagagagaaacaaacagaaagaccattcaataccctgaaaaagataaagtgtatttaattacaggaggattcggggggattggcagtaaagtcgctgaatattgcgcgaaaactgaaagatccaccattatcctatcggggcgatctgttttaaacggagataagcagaaacatttagaatacctgaacagctttaaagcaaaggtttattatatacaggcgaatatatccgataaagaagcggtaaaaaaaatgattgacggtattaccgatcagcatgcaggtcttcatggcgtcattcattgtgcaggcgttatcagagactcgctgctttttcgaaaaacagagcaggaactttcagaggtgatggctccgaaagttcatggtacattgaatcttgatcatgcaacgaagtcgctcgaattggattttttcgccgttttttcttcagtcgtctctattgcagggaacgaaggccaatgtgattatgcatatgcaaacggctttttagatcattttgccgaatatcgggaacagaaacggcaaaattctctgcgcagcggccgcacgctgacaatcaattggcctttatggcaagacatcggaatgggggcgaacctgtcaggtcaagggcggacgtctctttttcatggttccgggctcgaagaacttcctgctgaagacggaacgcaatggttttcgcggattgtaacttcgggtcagctgcataatattgcagtctgcggtgatcatgagcgctttgaacaatttatgcggcagagatttttgacggacgaaacagagacaggcagtgccattcctttggctcaaggcggtattttggatcagacagttttgtttttaaccaatatcatctcctctgagatcggtgcagaacgggaagagatcgatcaagatgtgcatctggctgaatatggaatggattcggttatgattcatcaatgcaacacaaagctggagcagtcactgggagcaatttcgaaaacgcttttatttgaatttcatacaatcagagatttagcagatcatcttgtaaaggaatatccggatcggctggggcgtttattttcacgggaggaaaaaggaaaacagattcagagcattacccccgccagaccggaacaaaacatcactgcacctgcggcggaggaaaaagaacatcttgacgggatagcggtaatcggtatcagcggccgttatcccggggctgaaaatatcgaagaattgtgggagcttctatcaaaagagagaaccgcaatcacagaaattccgaaagaacgatggaattatgagacctactatgatccagaccccgctaaagcgcatgaggggaaaatttattgcaaatggggcggatttttaaagaatgctgaatcatttgattctttgtttttccaaatatcgccgagagaagcagaagcgatggatcctcaggaacgaatatttctcgaaaccgtatggaaagcatttgaagattcgggatataccattgaagggttgagaaagcagaccgaaaaacatgtcggtgtatttgcgggagtgacgaccaattcttacaagctgctcggcgaaagaataccgacggacaaaaacgatgtacttccaaattcgttcccatggtctctcgcgaacagagtgtcttacacatttgactttacaggtcccagtgtccctgtagatacggcttgttcttcatccttaacggcaattcacatggcttgccaaagtttaacaaagggtgagtgctcggttgcggttgcgggaggagtaaatttgtatcttcatccttctgactatttataccgatgtcagatgggaatgctttcaaaatcgggaaaatgcagaagttttggagaaggcggcgacggtttcgtgcccggggaaggtgtaggggccgttattcttaaaccgcttaaagatgccatacgtgataatgatactgtgtacgccgtcattaaaggaacggcaattaatcatggcggaaagacaaatggctatacggtgcctaatcctgccgcacaggcggatttaattcaaggagctcttaaagcggccggttggagcgccagaagtgtcagctttattgaagcccacggcacgggaacgccgctgggtgacccgattgaagtccgaggcctgaccaaggcgtttagagcttataccgaagatgtttcatattgctctctcggatcgattaaatctaacatcggccacctggaagcggccgcgggtattgccggcctgacaaagattatcttacaaatgaagtataaaaaaatagccgcaacactgcatgctgaacgggtgcatccggatctttatctggaagagacacctttttgcctccagcaggaaaccgccgactggataaaacccgcgaatgaaaaacattctgacagcaaatatccgcggcgtgcgggattaagttctttcggagcgggcggggcaaatgctcatatccttgtggaagagtatgagtctgctgctgactgtcatacgaggcgtgaggaaagtgaggaacttatcgtactctcagccaagcattcactgagtttacgggaatcagcaaaacagctggaggaatatcttcaaaaggcccgtcataagcagggcgagtttttacagcaagcgggaacaaacaagccgcttcacctttctgatattgcgtatacccttcttgaagggcgtgaagccatgccggaaaggcttgctgtcatttcaaataatatcgatgatctgatgttgaaattaaatcacttttataatggaattgatgatcacgaaatatttagagggaaaaaaccgaatcataaacgcagacagcagcaatcggagcttccgctcaaatccggcagcaccacattagccgaaatggctgaattctgggtatcaggaggcaatttaagctgggatgacttccgcaccggcagaaaaccggcaaaaattgccctgccgacttaccctttcttaaaaaacagactgtgggtgtcaaataaggcacaaacaacatcagcagctgataaagctcctttatttgatttttcagcaggtgagtgccgattttataaaacgctttctgattctgaatattatgtaaaagaccatgttgtacatgggaaaacggttctgcccggagcggcaatcatcgaagcggccagagaagcgggagagaggctgtacggaaaatcagtcataaaactgagtcaggttgtatgggcggttccgataaccgttgaaaaagaaaagacgataatgatagatctgctgccgaaccaaaaagacggcagatttgtggtgaaaaccgatatgcaacctgaagcggcagttcactgccaaggaaacattatcacatgcggagatcaagatgcctctcatacagagcattttcaattggacagctggttaaccggacagaaaagattaatcagcggcgctgagtgttacagggatttccggcagagcggtttggactatggaaccagttttcaggtaataaaacagctgtttatccatgaaaatgctgttcttgcacgaatatcacttcctgagagcattcataaaaaatccggcacgctgctggaaccgtgtttattggatggcatttttcaatccacaacagggtttacgttcggtcagcaagacgatcacaccgcatatttgccttttgaattaggtgagctggagattcttcaggcaccttcggcaaacggctatgcttacattacagctgccgatgaaagacaacatgatgatgtcaaaaggtttcatatactatttatggatgaaaacggatctgtaagtcttaaaataaaaaacttagcgttgaaggcattaagggctgcaccgcagactcaggatgagcggcattcagttaattattacactgatatgtggaagcgggcccctctgcttcatcagcctgaagccagcggtttaaacagctgccttgtgatttcgctccgtcgggatatatcagaaccgctgatccgccaggcatataaagatatcggcctgaacatgatctttgtccgccatggcggcagttttcaagaaaaagaagctgattcctttgaat

SEQ ID NO: 11 - DNA, 9960 bp, unidentified
  source 1..9960: mol_type = genomic DNA
tgaacagcgtcaaacttgacggctatcagaaattgctggcgattcttaaagaaagacaagtgacaattgaccgcgtcattttaatcgggatgagccctgaaacgggaattgagcagaacgttcgtgaaaagctggaagaggctgttttgccggttttcaatgtaacgaaagctctgttagcaacaatcccaagacataaaatcctgatatcacatgtatttttcacaaggaaaaatcaaatcttaccttatcaaatggcaatcagcggcttttttaagtcgcttcatttagaacatcctatgttgtccggtaaaaatattgaaatacacagcgacgaaattcagttacagctgaacaccggtctgcctgaattaaaaaaccttgtgaaagaaacaatcatcagcgatgatacttcagaaatcagatacagagatggaatcaggcaggtaaagcactctgcagaaattgaaagtgccgagccccccgaaaagatggaagtaaaagaagggggagtttacctcattacaggaggaacaggcggtctgggactgattttcgccagacatcttgtgcgaaaagggagaataaaacttgtccttatcggcaggtcagagctttctgaggaagccgtaagcaatgtaaaagagctcgaaaaaaacggatcagaggttttttatgtaagaaccgatatttctgataaggaaaatgtcaataacctgataaaactcgtcaaaaacaaattaggtcctgttaaaggaattattcacagcgccggtgtgataaaagattcattactcatcaataagcgtgaagaggatctcgattcagtcttggccagcaaggtttacggaacgatatggcttgaccggtatacagacagtgaacctctggacttttttgttatgttttcatctatcagcgcaataatcgggaatgccggacaaactgactatgcttatgcaaaccggtttatggattattttgcggaaatgagagcggcggaaaataaacctggaaaaacagtctcgattaattggcctttatggaaagaaggcggtatgcaaattgatattcattcagagaagttcctttatgaccgtttcggaatgctgcctctgggaaaacgggaaggccttgcagcatttgatcaagcaatgacagggtgtgaacatcaaataatggtatttagcggtaatgaattaaagctggaacgaatgataacgattgacagcggaaaaagtgatccggcaatcgccgacgaaaatcacgaaccgaaagagctgtcaataactgaatctttaaaaagcactttgacgcgcatcatcaccggcgtattaaaaattgaagaacgggaatttgattttgaactgccgataagtgaatacggatttgattcaattatgtttacaatgctgggcaacgaattaaacgaaactttaggtatatcaatgatgccgtcagattttttcggtcttactgattttaatgaacttcttgagtttttaaagagtgagtattccgaggagcttcatctctgcggttctttttcttcagatgaatctgcggcgaaagaccggagagaaaatctgattgagggcatcgcaaagatgatgtcaagcattttgaagattgacagaaaggaaatagaagaagagacttcattcggcgagtacggatttgattcgattacgtttaccagactcggaaatgaaatcaatcaacaatatggtacggaattattgccgtccattttctttgaatgcaattcactggctgaaatgactgattatgtaatcgagcaatacggagaaatcctttttgctgagacagaatctttttctgcagctgacgaaaaaacattgtacagcacgcctccgccggaatcttatgaacattcccctgagcctgaaaacaaaacttggcaagaggaaagacatatgaatgagccggttgcaatagtgggaataagcggccgtttcccccaaacagatacgctggaagacttttgggaaggaattgaaaaagggcgggatatgattacagacattcctgaagaccgttggaactggaaagagtttgacggcgatccgatcagggacgtcaatagaacaaaaataaaaaaaggcggatttatcaaaggcgcggacagttttgatccgttgtttttcgggatttctcccgccgaagcagaattaatggatccccagcaaagaatttttatggagactgtatggaaaacaatagaagatgcgggctataagccatctgatttggcagggacgaaaaccgcggtgtttgcaggggtcgccacaacggattatgctgaacgtctgaatgaaaatggcgtctctgttcaggcgcagaccgcaaccggtttaaatcattgtatattggcaaatcgtatttcttaccagctgaacctgacgggtccgagcgaaccgattgacactgcctgttcaagttcgttagttgcactccatcgtgcggctgaggggattcaaagaggcgattttgaaatggcgattgcaggcggagtgcatattatgacaagccctactctgtttatagcttttgataaagcaggtatgctttccaaagacggaaagtgcaaaacgttcgataaaagcgctgacggctatgtaaggagtgaaggggcggctgcagtattgcttaagccgctcagcaaagcacaggcagacggtgatcatatatatggtttgattaaagggtcagctgtaaatcacggcggccgcgccaattctttgacagctccgaatccgaacgcacaggcggacttattaaccgaagcttggcggaaggcgggggtcagccctgccactgcgggatatatagaagcgcatggcacagggacaagtcttggcgatccgattgaagtaaacagcatcataaaggcatttaaaacgttatatcaagaatgggacattccttttgcgaaacagaaatgctgcggaatcgggtctgtgaaaacaaacatcggccatctcgaaaccgctgccggaatagccggagtgatgaaagtgttactggcatttaaatatcgcaaacttccgggactcgcccatttttcagaactcaatccatacatcaaactggaaaatacaccattttatatcacagaaaccgctcaaaagtggaatccgataaaagatgaagagaacaacgatctgccgaggagagcgggtgtcagctcattcggattcggcggtgtgaacgcgcatattgttttagaggaatatagggaacctcaaaaagaagtgccggaaaacaacggtaacacacatatttttgttttatctgcgaaaaccaaagatcaattacttgagtatatccggcttatgcttcagtacttggatcgcgtgattgccggtgacatgaccgttaatgccagtgacatggaatttacattgcaaaacggccgggaagcaatgaacgaaagaattgccgtatgtgcagcgagtctgactgagctgagagataaatacgtcagctttcttgatgacagcaaggtgcaaaaaggcgtatttataagcgaaaacactgaagagcccacagctgtttctgaatatatgattgaggaggctcttcaaagcggaaaagaggagctgctcgcacaatggtgggccaatggagccgagtttgaatggaagacaatcggcaataagaaattgaatagagtgccgctgccaacttatccgtttgcaaaagagagttattggttcaccccgaaaaagaacaaaagtgaggttaaggttaatagcaaaagtcttcacccattaatcagcgagaatgtctcaacattaaagaaacaagcatttaaaactgttatttccggaactgaatttttcgtcactgatcatcttgtttcaggagactatgtgctgccgggagccgcttcgcttgaaatggctcttgtttgtgcggaaataagcggagaaagtaaagtatataaaataaaagacgtgatgtggaacagacctatattatataaaggaaaaacgctgcacatttataccgacttggaaatgaaacaagataagttgaacgtaacgatatgtaaagagggtccggatggcgtgcagcaaatctgcacacaaagtgaaacagtattcggagacataaacagaaatgctgagtatgtcgatattgataagttcaaacgcagcagtaaggctgtcataagccgggaagagtgttatcaccgcttcagtcgtatgggcctgacatatcatagcagttttcaaacgttgaaagccgtatatcgcggtgatatggagttaatggcagaattggagcttccggctcatttagcgaacgggttttcacattttacactccatccttcattaattgacggcggttttcaagcggcggcatttttgttaagagataatgagaaagacggcttatttgttccatattcacttagagagcttcacattaagagggcgcttactgcccgatgccatgtctcggtgagagaaaccggtttttatacgtataacattgattttattaacgagagcggtgaaatagccgtgagcatgaaagacttttctgtcagaaaagtgaccgggggagaattaaccgcaggacgtaaaaatgaagatcaggaaattctcttcttattagaacaattgaaaaacggtgaactttctgctgaccaagtttatcagctgctggagggaaaatagtgcagatcaaacaaatactttccttaattgaagaacagcaaatgtctcctgatgcaggactggagctaattcgcacttacagaaaagaacaaatggaaaaaaaggcggagccggaatcacaagctccgtccccaaccattttttttaagaacgattgggtaaggacaaataacattgatatgccgaatcagcaggcactggggaatatgctgatttttgatcaggataaccgtctgtttcagcatgccgagatgatgaatggagatcaaaaccgtgtcattctcgtaacgcccaattctgattatagaattgaacggaatagatgcacaatcaatccttctgatgaaaatcatgtcgaaatgctgctgtctgatttggctgaaaacggatttacacctgatgtcgtgctttatttatggaacggagaaatcagagaattcactgaagaatccgttaacgaaactcttcgcagagggataaactcgctctttttattatcaaaagctttaatgcggagaaacgaacctgttacattattgcatatttattggacaagcggtctgaatcagccgtttcatgaagctgtcggagggttcatgaaaacggtaaacatggaaagctccaagcttaaagcgaaaaccatacagatacgccatgaggattctgacaaaaatcctccggtttccgagatgtgggatgctgcaatcagtgaaatacagcgctttcgaacggatgaatgtgaggttttaatagactcacatacccgttatgtgaaagggctgcagcatattgataatccggaacacgataacggatctgtaacgtatcaaaaaggcggtatatatatcattaccggaggagcgggaaaactcgggctgctgtttgcggaacacatcgctaaagaaaaccaagccgtgattatacttacaggccgttcttttttaaccgctgatcaaaaagaacggatcagtaagcttaatgagagcggctctattgttgaatatcagcggacggacatcacagatcgaacagaggcagagcgattcattttcggcgttaaacagcggtacggcagcataaacgggatcattcatgctgccggcatccttcgtgactcatttcttgtaaataaaagtctcagtgattttacggacgtactgaaaccgaaagtttacgggacaatctggctggatgagctgacaaaagacgaagatcttgatttttttgttctcttttcttctttttccgcttacggaaatgccggccaaagtgattatgcgtatgcaaatcattttatggacagttttgcagtgcttcgatctcagctctcccgaaacggaaaaaccctttcaatcaattggccgttattgaaagatggcgggatgagtctcgacagcaatggaatcaaccaaataaaagaaaaatacggaatgactccgatgcgaacttcctctgttttggaagcgtttctgaaaatgctgcggtcttcatttaccagcttaatgcctgcagaaggaaagcggggtaaaatgataaaagcgctttcgattcaaagaaatacagctgcagcagatcaagatacactcacggcggaacacgatgaagaagaggaatatgctgcgaaagaaaaagtcaatgactatttaatcaagatcatcagcggtgtcttgaaaatccaagctcataaaataaatgcgaaggactcttttgagaaattcggaatcaactcacttatgatcatgtcacttatttcaaagcttgaagatactttcggggatcttccaaaaacaatcttttttgaatatcagaacattgaagagctttcaaacttcttcctaacatcttttaaagaaaaagtgcttaaggccgcaggggcagctccggtaacaagagtgcaaaagaaaagagaaaatctgagtaaaaaacagtaccggcagcagaaggatacatctgcaccgagcatggttcagccgggtgaggatattgccattatcggtgtgagcggaagatatccgaattccgatactcttgaggagttctgggaaaacattgccggagggaaaaactgtattattgaaatccccgaggaacgttgggatttcagatcgaattacagtcctgacagaatggagaagggaaaaatcaacagtaagtggggcggttttatcagcggtgttgatcaattcgatccgctgtttttccacatttctcataaagaagctgagctgatggaccctcaggagcgaatttttctcgaaacctcctggcatacctttgaagattcaggatatacaaaagagaagcttaacggtatgaaagccggtgtgtttgtcggagccatgtacggacaatatcaattattcggcgtagaagaaactgcgcggggtaatccgattgcgctcagttccttcttttcatctattgcgaacagggtttcatacttttttaatctctcaggcccgagcattgcactggatacgatgtgttcatcatcgctgacggccattcacttggcatgtgagagcattaaaagaggggaaagtgaaattgcacttgcgggcggcgttaatgtgtccatacatcccagcaaatacctttggctgagtcaaggtaattttgtttccaccgaaggattgtgcagaagtttcggagagggaggagacggctatgtcccgggagaaggttcgggagccgtcctgttgaaacctttggaaaaagcaattgctgatcaggatcatatatacggagtaattaaaggaacatctgtaaatcatggcggaaaaaccaatggatttacggttccgaatccgaatgcacaagctgagcttattgctgaaaatttcaagaaatcgggcatcagtccgcgttccgtcagctacttggaagcacacggaacgggtacctcgctcggagatccgattgagatcgcaggattgacaaaagcgtttgagaaatttacggacgataaacaattttgtgccataggttcagtgaagtctaatatcggacatctggagtctgccgcaggtatagcagcccttacaaaagttcttttacaatttaagcatcagaaattggcgccatccattcactcggaacagctgaaccaaaatatccgatttgaaaagacgcctttttacgttcagcagaaagaagaagagtggcggaaagaatcaggcgcctccaacaggagagcagcaatcagttcgttcggagcgggcggagcaaatgcccatgtcattgtggaagaatatcaaaaccatgcggacacacaaacaataagcggtccgtttgccgcagttctctctgccaaaaatgaagacaggctgaaagaatacgctcggcgccttctgcaattcacagagcgggagacaaaggcctcggcagctgatatcgcttttacattgcagacatgcagggaaccgatggaagacagacttgctgttgtagcttccgatttgccggaacttaaggagaagctgagaactttctgtactgaaggaacaattggagagggcgtatacaaagggaatgttacgaaagattttgataaagtctttatctttcatgatcaaaagatgaaagataaattcctcgaggtgatgatggagacaaaggacttgaatcttcttgcaaaattatggtctttcggagctgatattcattggagagagttatatgaagaatataccccgcaaaaggtcaagcttcctttatatccatttgctgaggagtcttactgggtgcctcgaaaccaaagtgcaacatttccggaaatattgccgcggcagtcttcactgcacccgcttctggattcgaatgaatctactctggaacagcagttttttaaaacgacattaagagaaaaagagatttggttaaaagaccacatcgtaaaaaacaagagtattctcccggcagctgcttttcttgaaatggcatgcgcagcgggaacgcggtcatatcgaaagaaaaaggtcactcgtttaaaaaacgtgatatttgccagaccggccatgcttttgcaagagagtctgaccatttacactcacatttccgtaaaagacggcagcatattatttaacatgtcacctgacgaggatgctgctgtaaggactggatttgtgacgggggaattagattatgatcatcattacagtgatgatgatatagagaacatcgacagacaagagatgataaacagaagtgcttttgtccgtgaacaagatgaatgttacgagtattttcgggaaaacgggttcctgtatgggcgcgcttttcagccgattcaacgtttatggggcggaaaaacggaagcggttgcacagttattgctgccaaaagaagccgcatatgctcttgaacattatacattgcatccgtctttaattgatggggctctgcaatctttgttcggaattacggacggaagagaagaggcatctgtttatgttcctttttcaataggagagattacgattcggaaaaaagtttcagcagaatgctgggcttatgcgcgtaaaacaggccaaaaaggtcaatccgagcttatttacgatattcagctgcttaatgaacaaggagaaagacttgcaggtttgaaggatgtaaccattcgtaaaaaagaaacgttttccgcggaggcacagactcgtgcaacagcggattccaatggaaagaacgagagagtaaaagagctcctcagacaattaaaagcgggcgaactgactgcggaggaagcagattatgcgctggagcgtatgcaggattagtttatcattttgcggcagtgccatttgtaaaggggatgaagatttttatgaaagaagcccgtcatattagaaaaaaagtactttctgacatagaaagcggcatcatttctttggaagaaggagtaaagcggatacagcagcttggttctcattccgcaggggataacggacaggcgatatattatcagacggtctggcagcggacagatctcagcattcatgtaaaaggcggccgacgtgagggcagccttttattatttgataaaaatgaagatgtgagtaaagaacttgctgcattgcatgatacaagggttattttggttaagccggggaaagagtttcgaaaaaccggggacggtgtatttgaaatcgacccccgctctgaaagagattacataaacctgttaggatggcttatagaagaagagaagtcagatatacgggaagcggtgcatttgtggtcagacatgccgttt

SEQ ID NO: 12 - DNA, 9960 bp, unidentified
  source 1..9960: mol_type = genomic DNA
gaaaagacagagagagcgattcgtgaacaatgcgacagcggagtctattctttgtttttattacaacaggcgctcttcaaaaataactgccgcaaaccgatcaaactgttatatggatatttttcacatctttctggagaacagcctctttacgcggcggcaagcggatttttgcggacattgcagctggaaaacccgttaatctcagcaaaaagcattgagatggagcagtcaatagcagctgttcacgcagctgatatcatacgcaatgagctgagtgatttccagccttcagatgtagagattcgttataaagacatgcagcgttatgtcaaacagctcaaaacgtatgaacccgaaaaaaattcccgaagcggagatgtgccgctgagagaaaatggagtatatattatcgcaggaggcgcgggcggattaggatttacctttgcagaatatttggcttttcaaacaaaatgctgccttgttctaacaggtagatctccgctttcagatcatataagaaaacagctgagacgattagaggatgccggttcgtcagctctttatgttcaggctgatattaccaataaggaagaagccgaatatgtagtcaaaacagccaaaacttcttatggaaaagtaaacggggtcattcagggcgcgggaatcattcgtgactctttccttcttaacaagtcacgagaggaattcgatcaggtaatcagaccgaaagtattgggtacgatgtggctgaatgatgccgttgagaaagaaaatcctgactttttcatttgcttctcctctacttccgcagtgttaggaaatgtcgggcaatctgattacgcgttcggaaacagttatatggatcatttcatgaatatgcgttctgcccgggattctgatacgatttctctctctctgaactggccgctgtggaaagaagtcggcatgcaagtggatgagcgcacggtagagacaatgaaaaaggcgggtttttatcctttagaaaaagaggaaggtaaagaagcgtttgcagtcgcattgtcctccggtttttcgcaatttactgtattttacggagatgagcaaattgacaatcatgttcagcaattatatgaacgaaaagacaatattcaggcagatgaaggaacagggagaaatatcgaatcatttgattcagagaaactgaaagctgaaacatctgatttcatcataaaagtgattgctgcagaattcaggatgcctgcggacaaaatagatgcagaagaggcattggaaaaatacggacttgattccgttatgattatcaatatgacgaatgagcttgaaaaacatttcggcgcgctctcaaaaacgctgttattcgaatatcagactgcggcagaattgtcacagtatttcgttgaggagcacagggatactcttcttcataaactccggttaggagaaaacgggagcagcaccccatcacagaagaaaaaagatcatgaacagacagccgatgttaacgggaaacagaaaatccagatgaaaaatcaggtgcagcaggagaagtatcatgacaatgatatcgcgattatcggtatcagcggacgttacccgatggcaaatgatcttgatactctgtgggaaaacttattgaacggtaaagactgtataactgagatacctgaggatcggtgggattcggggctgcactatcagcccgcagcgggcgacgaagtaaaaaaccaaagcaaatgggggggatttattgacgatgtgtatcattttgatccgttgttctttaatatttctcctgccgaagctgaattaattgatccgcaggagagactgtttttagagacggtttggcacaccatagaagatgcggggtacgcaaagaaaacgcttgaacgaaaaaacgtcggtgtttttgcaggggttatgtacggccaatatcagctgtacagtgttggtcacgaagcggccgtttcttcctcatatgcatcaatcgccaaccgggtatcgtacttctttaatttcagcgggccaagtattgctttggacacgatgtgttcctcatcattaacggctgttcattttgcctgtgaaagtttaatcaggaatgaatgtgaaattgcaattgcaggaggcgtcaatttatccattcatcctcataaatatgaactcctgactcaaggcggatttttatccagtgatggccggtgcagaagcttcggttccggaggagacggctatgtgccgggagaagggacaggtgccgtgctgctgaaacctgtaaaaaaggctattgcggacggtgatcagatttattcagttattaaggcaacagccatcaaccacggtgctaagacgaatggatatactgtgccgaatcccaaagctcaaagtgaggtcattacgagagccttaaaaaaaggatttattgatccaaacagcatcagctatatagaagctcacggaacaggcaccgcgttaggagatccgattgaaatcaccggtctgaccagagcattttcaggtgccgaacgcgataagaaaacatgcgcgatcgggtcgattaaatcaaatatcggacatcttgaggctgctgcaggcatagcgggcattacgaaagttattcttcaaatgaaacataaaacgattgttccgtcactgactcattctgctgaattgaatgaaaatataccgtttcaaaaaacacctttttatgtaccggacagtgccgaaaaatgggagtcatccgttcccaggcgtgccggaatcagttctttcggggcaggaggagctaatgcccatgttattttagaagaatttgcggatgaagctatctctcctctcgcggctgaagaacctgaacagcttttatttgttttttctgcaaaaaatgccgacaggctggcagaatatgcaaaacgtgcaaaagaacatatagcagccagagattatccattagaaacacttagaaatacagcatttactttacaaaccggcagagaagaaatgccgcacagattggctgttatcgcctccacaagagatgaattgcttaaaggactgacagactatactctcggaaacgcgacggatcatatttttacaagcggcaaagacacaacagaagctcaaaggatgaataaagctgagattgaagaagcagccgcaaacagggatcttgtgaagattgctgaatactgggttggcggtaacgggatagattggccgtcactttataaagatgtcactttgagacgggtatcagcacccggatatccatttgccaaagagctatgcagaatagatgcgctaaatatgggaagccgccggatgaacgttaataacgatctgcatccttacatttcatgcaatatatctgattttaaagcacagaagtttcttatcaatgtaagcgataccggtttgtatataaaaacaaagtatcaagacaaggaaatgtttccgtttctcagccagattgaaatggcgagagctgccggagcgatggcttcagggaatccgattattaaattaacagagctctcctttagagaaccgatgttacaaagcggaagcaatgagcagatccggattgtattaaccccggataatcagggtgcatcgtattctatagaaaaacagagcgactcatccatttattcctcagggcggctcgaattagaaggcggggcgtacgagaacggaaatattcatcttgagccgtttctgtctcagcgcgcagaccgcataccgtatgaagcgttttatcagcggcttgccgaattcggatacagctgcagtgacagtctgaaagcggcagagtactgcgtcagcaggaacggacaggtacttttaaaaataaaagcaaaggccgggccgaaagggtgcataatcaaaccggaagttattgagagcgtatatcaagccgtcatataccttgcgggggaaaatgccggagagctcccggaaaacattaaagaatgtacgatttttgatcatgagactgaacccgtttatgtatatgcagaacaaactggagaaagcgattcggcttatgatgtgtatgtattagacaatgccggcgggattttgatggaactgagcgggctggtttttggcgaaaagcctaataaaaatgtgcgtttttatgagcataagtgggatcaaaaaaaacctctgattgaaatcaaagatagggagccaaaaacaattctggtctttgcaaaagatgaagcactggctggagctctaaaaaatcaatcagacagccgtgtcattctcgtcacgccggatcagcggcagaccgatcaaaatctgctgacattctgtgtaaacccggaagtgacatcagattacattcgtttattcgaagaattggataaaaaagggttttctcctgatgtcatcttgcatacgggagaaacattttacgcgggtgaccgccttctcagcggcctggaatcaattttttatttattaaaagccctgagcgcccgcagtgacgcctctctttatcttcattctctgcttcttgttcactcttttaaagaaggcgtgaatgatccgtatacaggcgctctttcaggctgtttaaaaacgacgaatatgttggtgccatccgcagagaatgtgaagtcaatcggtttttcagaacgtatatcaattgaaaaaatggctgaattttgtcttaacgaaatggtttcaggcatggaacggcaggcggaagaagttaaatatataaatgacaagaggtttgttaagacattaaaggagtatgtattcaatcatacagagcactctcatcagctgaaacaggaagggatttacctcataacgggcggtttgggcggtctgggcctgattttcgcaaaatatttggcctctcaatatcaagcaaaattagtattgacgggaaggtcaccgttaactgctgataaacgccataacattgaccggctgcaggcgcttggaggagatgcggtatattatcaagctgatgccgcagatcaaaagacgatgggacgggtcatagaagaaacgaaacggacttggggctctattcaaggcgttatccattcagctggtcatgccgatgatcggttatttacagagatgaatctggctgatttttcagatggcatgactgctaaaattgaaggcacggtatgtttagatgaattaacgaaagatgagccgcttgatttctttatcgtattttcttctatttcttcggtcttcggagattttggacaggttaactatgcgttgggcaattattttcaagatcaatatataaattggagatccgaacagagccgccggcaagagagacaaggcaagagcatttcgattaactggccgttgtggcgtaaaggcggaatgcatttaaacaaagaagctgaagaagcatatctttccgcatccgggtttgattatttggaaacggaagaagggatagcggcttttgagacgattttggcaagtgactgcccgcaaatttcggttgttaccggggatgccgaaacgacaccagcaagaaaagatctgccgcatcaaaaagtaaatacgcttccggttcgtcaagaggaaatttcttatacagcaccggcttatacagacaatgttctggaagaactgaagcaaatggtatcggaattattgaaaattaacattgaacagctggatacggcagagaatttcggcaatctcggctttgattctatcagtttgaaaacattagctgtccgactgaataaaaaatacaagcttaagctgacacctgccgttttctttacttacagccatattaagtctctttcagaatttctgaacagtgaactgcgtactgttgcaaaagggaatacagatgcagaaaaaaacatttctgaaaaaaggaaagtagtgcacgctgggaaaaccaccgggaaaaccgtgaagccgccgcgaagttttccgtttgaaaaaaagacaacaagacaggctgaccatcatccagagaagcagccagttgccattatcgggatgagttgtgtttttccgggatcaaaaaatgcagatgagttttggatcaatttaatcaatgaaaaagatttgattcaggaaattcccccggatcgctgggattggaaaaaatacgccgaaggcaaaggaagcagcgaagtacattcgaaatggggcggctttatcacagatgccgataaattcgatgaggcgtttttcaatattaatccgcgggaagcagaactcatggatcctcagcacagaatttatttgcaggaaacctggaaagcgatagaagatgcgggttacaaagcttcaaagctgtctggcaaaaatatcggggtgtttacgggcattcaatttaatgattaccgccagctgctgatgaggcattcggacggggcgcatgcgtttgcgggaaccggaaacagccaggcgctcatcagcaaccgtgtatcccactattttaatttcagaggtccgagtgagtccattgatacagcctgctcaagcagtcttgtagcaatccacagagctgtgcgctcgattcaaaatggtgaaagtgagctggcggtagcaggcggggtttcgctgctcttagatccagtcacgcatatgtatactgataaactcggtattttgtctagggacggccggtgtaagacgtttgataaacttgccgacggttatgtccgcggtgaaggtgcagctgttctgttattgaaaccattgcaagcggcaatgaatgacagagacaatatttacggggtcctgagaggaaccggagaaaatcacggaggaagatctgcttcattgaccgctccgaacccggaagctcaggcagaactgttaacgtctgtttacaaaaatgccgatattcctgttgatacgatatcttatattgaagcacacggaaccgggacagaactgggagatccgattgagattgaaggcttgaaaaaagcgtttggatcttttgagccgcattcaggaaagaatcatgatcaatattgcggcatcggttcagtaaaaacgaatatcggtcatttagagcccgcctctggaatgccggggctgattaaagtattactatccatgaagtataaaacactgcctgccacacttcatgtcaaggaactgaacccttacattcaattaagcggaacgccgtttcatattgtcaataaaacaatgaagtgggccagattaatagatcgaaaaggaaatgaaattccgcgccgtgccggagtcagttcctttggattcggcggggcaaatgctcatgtcatcgtggaagaatttgaatacggacaagataaagcagtaactggcgagcgggaacaaatcattgttctatcggccaaaacaaaggaaagactgatggttaaagccaaagagttaacaacgtttttacatgaagaactatcagttcaattcagtcagcaagaggtgcaggaacgtgatttgcaagaggcggttcgggaagcggcagcgggtatcctgcgtttagataccgcagatatagatgcaaagacacctctttcggaattcggatttgatccgttggcttattccgaattaatctcttttataaaccaaatgtgcggcacagaaatatcacttgacatatttcaggaattaaagacactgagcgagctgtgccgctatatcagcagcatccggaatcgatcagctgataaaccgcataacggcaattttagagaaaagtccggattcggtcttgaagaaatagcatatacgctgcaaaccggaagggaaccatttgaatgccgccttgcgatcacttgctcgaatacaggagatcttcttgaaaaattaaacggttacatccaggggagcagactgcaaggtgtttatgaaggaaatgtaacggattttgatagtctgtacgctcctttattcggcggccccgaaggtcatgcatatgtacaatcactgattcagcaaaacaagccggccaaactggctcagttgtggacgatcggttgtgaaatcagctgggaagacttgtatgaggaggaactcaagccggttaaactgtctcttcctctctatcctttcagcaagaacagacattggattcatttaaataaagatgaagcgcagaaggctgaggctttctcgaacactgagcttcacaactcaaataaagatacagcagatggagaaatcatgtcgcttcttgaaaaagcacaaacaggtgagatgaatacgaaagagacgtcagagctgatagaggagttgcttttccatgaatgaaaaaacgatgcaaattgagcaaatcgtgaaaaaggtgaaagaatgcagcctgacgcctgaagaagggctggaattaattaagagcttggggaaaacccatctatatgaaatggtatgggatagacatgcatttaaaggcagtaaaaaattcccgcatacgaaagagccaattttatttttttgcgaagacgacagcatgtataccgtaatgaagaggcagcttgaaggttatgaggcgcctttcatttacgtgacttccggcgagcgtttcgaagattgccggaacggtcgttttacaatgaactttgcaaaagatgaggattatgatgcgttatgcggtgttctgcgttctcaaaatataagaccccgccatattattcattttttggctgccgggctcttcaaaaatacggaagatgcaatgagaaagcagctcaataaaagtctgtattccttgtttcaaatgtttcaggccttcatggcgaacaaactttgtccgaaagcagaaattttgtatctgtatgaaaatgccgagggcgaggttcagccgatttataacgcggttgaaagttttcttaagaccgtacaggccgaaaatccgaatttcacatgtaaggctgcagagctgaaatcaatgtttgacgagccgtttacaaagcagcatattgcagatgtcatctcttttgaatggaacaaccaatgtaaaacggaccactttacctgctatgagccgaggcattattataaaaggcagcttcaaagagtaaaaaaagaagatggggagaaacatagtttcagtgtaaaaaagaacggtgtgtatttaattacgggaggagccggcggcctcgggtatctgtttgcggaatatctggcgaaacaggcagaagtcaagctgatattaaccgggcgttccccggcctcccgtgaaacggcgcaaaaattgagcgctcttgaaaatctcggcgctgaagccttatatgtcccggcagatatttcaaaggaaaaagagacggacgctctgattaagcacattaagcagacattcggagaattaaacggaattttgcacagtgccggacttgttaaggatgcatttatcataaaaaaaacgaaagaaagcatagaagaagttatagcgcctaaagtattcggaaccgtttggctggataaagcagctgaagaagagcctctagatttcttcgttatgttttcatccctttcggccgttctgcctaatgcgggtcaaagcgactacgccttcgctaacggatgtatggacggattcacccagtatcgaagcattaagggacgtcccgggaaaacactttctattaattggccgttatgggatgcgggcaatatgacagtcgggccgggagagctgcaggctttaagacatgcagggctggaacttttatccgcccaagcagggcttgccgcttttcaggacagtatgagccgctctgcttctcagctggctgttatcagcggagacaaagatagaataagtgaactactctcaactgatcataaagaaatagagactgtgcctgaaaacaacagtaacctgccgcaattacatgaaaaagatctgaaaaagcaaacggtccgtcttttgacagagatcgtcggagcggaaacgggaagaccggcggacagtattttacccgaggatacgtt

SEQ ID NO: 13 - DNA, 9960 bp, unidentified
  source 1..9960: mol_type = genomic DNA
tgaccgttacggaattgattctctaacgattttaggtcttaactcccgtctggaaaaatattttggaagagtgtctaagactcttttctttgaatacagcagtgttgaagagctgtcagagtattttcttgaacactatgagcatgagttgcagaaggcgtttttaccggcagccgaacagcgaattgatcattcggctccggcgaaacgggagaagctttccgtttcccgcataccatccgcaccttctgaggaagatattgcaattatcggtatagccggccgctacccgatggcgaaagatgtggaacagttttgggaaaatatttcttccggaaaagattgtataacggaaataccgaaagagcgctgggattttcggaaatattatcacacaggtgacaatgcccgtcatgacagcaaatggggaggttttataaaagacgctgataaattcgatccgctgttcttctcaatttcgcctcgtgatgccgagttaattgatcctcaggaacggttatttttagagacggcatggcacacgtttgaagatgcgggttacacaaagaaaaagttgaaacggcataagacaggtgtatttgtcggtgtcacctacagccactatcagttatttgcagccggtgcttcagatgatgaggaacgtcaggcgctcttgtcttcttattcgtctatcgcaaaccgggtgtcatatttctttgacctgcacggacccagccttgcaattgatacaatgtgttcttcttcactttctgctgttcattttgcatgtgaaagtttaaaaaggggagaatccaccctcgccatagccggaggcgtgaatttatctttacacccggataaatatactttactttcccaaaatcattttgtttcaagcgatgggcgctgcagaagctttggagcgggaggagacggatatgtgccgggagaaggagtcggggccgttcttttgaaatcgctttctgccgcgaaggctgacggagacaggatttacggtgtgattaagggaacatctgtaaatcacggcggaaaaacaaacggatacactgtgccaaatccgggcgagcaggcaaacgtcatcagtgaaactcttgataaggcggggattgatgcagacagcataagttatattgaagcgcatggcactgggacatctttgggagacccgattgaaatttcaggtttaaccaaagctttgcacagctcgaaaagcgattatcgttgtgcaattggttcggttaagtcaaatatcggacacctagaggctgcggcgggaattgcttctattacaaaagtgctgatgcaaatgaagtataaacagcttgctccttcccttcattccgataacttaaatcctaatattgattttgaggaaacaccgttttacgtgcagtctgaactgactgattgggagacgccgcaaaatcaagccagaaggtcaggcatcagttctttcggcgctggaggatctaatgcccacattatcatcgaagagtataagaatgaagaaaagatggaaacggtacagagcaacaagacgcaaattattctgctttctgccaaaaatgaagaacggctcagactcttgattgatgcatatataaatgaactggggccaaaaacgtcactagctgatgctgcgtacacctcgcagaccgggagaaacgctatggaagagcggctcgctgttgaggttagcagcattgacgaccttcgcaataagctgacgatgtatttgcggtcagatcataagttaaatggtttatataggggaaaggcccgccgagacgctggtcaaaaggatgccctgtcagccgcccactttaaaagtgcaagagaaatggccattcactgggtgaacggaggagacgttcaatgggaagagatgtacactgcggaattaccgaagcttatctctcttcccgcatatccttttgaacggaacagatattgggtgcctgccgcaaaagcagcacagaaacagaaaaaaacaggtgtaccgcaaagtttgccacacgcaccgactgacttatcatttgatgtatttaaaaaatcgtggaaaaaacaaaaggctgcgccctccgcttttatgaaaggacaggtgatgatcctggctaatagtgaaagtgagcggcttgcattcgaattgtttgattcagatcctctgattgaatccgctgtcatttgcgcagataactgggatcattcggatggaaaagccaaaaaaacggctctagcggtcatagaagaaaatccggaaatcatcggcatgattgacctgtcagatttgcggagtgatcaaaacggggcacgcaccggcacaatcgggaagctgacactgctgcaggaaatcataaagacacgtaaacatcttttcgttttacatgtgaccagagggctgcagacatttttgaatgacaagccgtcgttagccggagccgaaacagccggactatataaaatgctgagtgcggagtatcaaaagataaaatcaaaaacaatagatgtagacggtcatgtcgggactgcggcggaattgagacaggtaatcacagatgaagcttgcgctgacgggctgtatagtgaaatctgctaccggaacggtgaacgttatattccatacgtcgagaaattacatgcagcggaaataaaccggaaactatccggtctgccagtatgccgtacggatcaaaccctggttattacagggggaacaagagggatcggagcagaacttgccagacattatgccggcaagggagtcaaaaaacttgtgttgatgggagtaaccgaccttccggaatctgataaaatagccggcatgttaaaagacaccgatgaaaagtcacctctttattcgaaattacaactgctgcatgagttggatcaaaagggagttcaggtggaagtgtattccgggccgctcacggaaaaagaaacattacaacgcttcttttcagaggtgagactgaagttcggcaaaatcggcggggtgattcattgcgctggtgcagccaatcattcgaatccggcttttattcataaatctgatcaagaaataagagaagtatttgagccaaaggtgcaggggatgcaggtacttcatgacatttttattgatgacgaacttgatttcttcattttgttttcatctgttgcgagcgcttttcctttattaggcgccggaacaagtgactatgccgcggcgaacacttttatggattactttgcggcgtatcagcatgcagccggcagaacatattatcaatcaattaactggccgagctggaaggacattgggatgggcgaggtgacaagtccatcatatcaggcacttggtttactttcacactcaacagaaacaggtattgccatgctggatgaaatgatgacgataaacggatacccatgccttataccggcaaagacacatccggagaaattcatgctgcagcagctgctccagacaagaattcaagctgctaaaaataacccggatgctgaggaaaaagtgatttctggtcagggagacaatgaagctataatcaggaagttaatggagattttttctgcggagctgaaaataccttacgggaatttagatcaggatacgtcttttgcggattacggagttgattcaatcttactggttcagcttgtcaaaaaagcggaagatgcatttcaaataaaaattgaaccatcagcttttctggagtatccgagtttttcacaactgggcgtctatttaaacgaattatttgcaagtgaggggatcagtcctcaaattgattcagaaaacattgaggttcggaaaaaagagcaacatataaatcaaaatgcttccgaagtacgggacagcggatctcaaatacaacaaaccagtctgaaccaaatcgctattatcggaatgggctgtgagtttccggatgctccggacacagaaacgttctggacaaacttgaagaaaggacaaaatagtgtccgcgaagtcccggcgtcgcgatgggatatttcttcatactactctgacacctatgaaaagggaaaaagtatcagtaagtggggcggatttcttcaagatattgagtctttcgatcccgcgttttttaatatgaacaaaagtgaggcatcagagcttgatccgttaatcaaaaaatgccttgaggtcagtgtccagacattgagagacggcggctatacaaaagaagaagtatcaaacaaagaagtcggcgtcttcatcggttcgcgaaccgggacgtttatgtcgaaaataggttccccttcaaaaaatacgatcgtgggtataggacaaaatttcataggcgcgcatatctcacacttttttaatctgaaaggaccgaatctggttgtagacacggcttgttcttcttcattggtcagcgtccatctcgcttgtcaaagcctgatgtcaaaagagtcagatatggcgattgcgggcggagttgacattttgctggatcaaaagccttacgtcatgctgagtgaggctcgggcgctttcacctgaaggaaaatgtcatacatttgatgagaaggcagatggctttgtaccgggagagggatgcggcacagtccttttgaaaagacttgatgacgcccttgccgaaggtgacagaatttacgcggtaattgactcatccgcggttaataatgacggacagacaatgggaatgacaacacctaatccgaaagcgcaaagcgcagtgataaaaaaagccctgaaaaaaggcggaattgatgcagatactctcagttatgtcgagacgcatggcaccggtacgatgataggagatccgattgaattaaaagcgctgacagaagtgttccgggaggttacggaagaaaggcaattttgcggagtaggcagcgtaaaaacgaatttcggacatttaatgagcgcagcggggatagcgagtattataaaggttgccttatccataaaaaatcagatgctcccgccgacattacattgtaaaacgccaaatccgcgttttaaatttgatcagtcgccattctttccgaacacgtctttaaaagaatggcgtcccagaaaaggggttttgagagccggaatcagttcttttggattcgggggcacgaacgcacatatgatattgagcgacagccatctcacattaggatatggagagacaaaaagacacccgctttctccggttatattcaataaggaacaatgctggatcgacaatggagaaactccagaaaaggaagagtcagaattgctgattcctttacttgatttcaaagaattataaagaaaagaaaaggaatgagacaggtggccagtaatcaaaactatcagttcacattaacaattaaacatacggactatattgtcagggatcaccgggtgcatggcacccggattatacccggtgtcacatttttagatatgatataccgtatggcgctgagcaaaggtttgcctattgaagaactcgagtgccagaatattttgtatttggaacctgtagcaacatcggatacctttgacagagaaatccgcatccgttttcagggattggaacagggatgggaaattcaggcgaagagcagaaaggtaaaagatgaccgcatcctggatgaaagctgggatgagaatatgacctgcaggctcatgcagaaattcccggaaagcatgaggtcaattcatatcgatgaaatcaaacatgcctccgtccgtgaagccgatatggagatatgttatgactatttaaaaaaggttgatatagagcattttgaatttatgaagagtctggggaaggtttatgtgtctgatcattctgtacttgcagagttgtcgctcagccaaattgccaatcaatataccggctattttcaattgcatcccacttttcttgatgcgtctaccatcatatcgatcctttatcaggcatgtcccgaatattttacacaaggatctccggcaagccggataaagacgtatatccccatccatattgaatctttccgtatggcgaagcgcctggaagacaggtgttatgtgtacatagagaagcaagaccaaaatcatgattcaggggatgtcatccaagtaagttacggtttgtacgatgcatacggggaggtgatcgcttattttacgcggctgtctgtgaaacatattcgctcaaaagatttaattgaaaacctgaagcagactcgtttggaaactgaagcggcttctcagccaaaagacagcctgatcagaaaagattcaatcataaatcgaagcgaaagcatcgagacacaccttaaagaattaatatccggggtttcaaatatagatctgcgtgacataaggacagacattggtttttatgaacagggcctggattcagcttctttgcttgccattgtcaaagaacttgaaaacaaaacaggggaacagctgtatcctacactcctgtttgaacagaaaacgatccgggaactcgccgcgtatattaaagaaaatcaaagaatgtccagttatttcttaacgcgaaatcaaccggctgaaaagcctgacatgaaagaaagtaagaacgaaaatctttccgctcttataacagaaggagacattcagcagctgattgcagatatcaaagggactgcccctgaaacactctctgtggtaaaaggtttttatgaacaaggcctcgattccgtaacacttatgcaagccgtaaaggcattggaagagaaaacaggcagggaactgtaccctactttattgtttgagtatccgacgattcaaaaacttgcaaactattttgcagagagcggcgaaaaaacttccgttgaaaaagacctccctcttcatcatggagagaatcatacggctttgacctatttaacgccgcattggagacaaaaggctgcggcttgggacccgaattgcggaaataaaaagaaaagaacatgcctgctgtttcaatttgacagagagctttttcattttatgcgggaaaatgctgaaaatggattcgaaaatacacagatcattctcgttttatcagggaatcgtttctcctatcacggagacggtgtttatgagatagatcacagggaggaagctcacttttacaggcttgcagctgatctgaaaaaaagggggcaaatgcccgacaccgtggtttacgggtggccggaagggtcatttgccggcccggaaaaaataaatgaacagctttctaagggggcttacccgctattctttttcagccgggcattgatgcgcagcaagacggaaaaaaaggttgtatcgcttttcctttatccggcggatcataaaaatgatcagccgctttacgcttcagtccatggcgcagggcagtctattgtgcttgagaacccgaaactggcccttaagacaatcgggatatcagtttcagacaagtcatcgttgacggaacttacagctgaaatcatcagagtcgaatctacggaagcgcatccgggagaaaccgatctttattatgaaggaaatgtgcgctgggtccgtgccttacgggaagcagaaccggctccaaactcagatttctcctttaaagaaaacggagtatacatgattacaggaggggccggcggcttaggccttatatttgcagaacatatcgcagcccaaacgaaagccaatattattttagcgggccgttctgaacttacggaagacaaaaagaacaaaatcagccgaatgaaagcaggcggttcttcggttcaatatataagaggtgatttttcagatagccgcgatgcagagcgaatcataaaaaccattaaaaggcaattcggagaaatcaacggtgtgatacacagtgccggtgtgacgcacgacgctttattgattcaaaaatcaaaagatgaaattcaggaagtgatcgctccgaaaataaatgggacggtcttattggacagcttgctgcaaaccgaacctttggacttctttgtgatgttttcatcaagcacctcgatgtacgggaatatcggacagacggattacgcattggccaaccggttcatcgatcttttcgctgagtggcgtgaaggaatgaggcggaaaaaagaacgctccggcgtctcagtagccatcggctggccgctatgggaagacggcggcatgaaagtggataaacaaactgaaaaatggatgaaacaaaaaatcggtatggttccccttagtaaaaccaatgggatcaaagcttttcaagattcactctctctgaacgcatcacagctgatcgtgtttgaaggtgacaaagaaaaattggaatctgcacttcaagataccttcatagaggatgaaagccgtgagatccaaataaatgaaacagctgatacaaggcatgaggacattgccattatcggattaagcgggaagtatccgatggctgaagacttacctgaattttgggagaatttaaagagcggaaaagattgtattacagaaatcccgcctgaccgctggaatatggatgatttcttcagggaagacagagatatgaaaggaaagtcttacagcaagtggggaggttttatcagcggagccgatcaatttgatccgctttactttcatatttccccaagggaagcagaacttatggatccccaggaaaggctctttttagaaacggccaaacattgttttgaagactcagggtacccgagaaacatgctggaaaagatgaaagtcggtgtatttgtcggtgcaatgtggggccaatatcaactatttgaaggagaagaaaatggagtggctttctcgccggcgtccgtctattcttcaattgccaatcgggtgtcttattattttcatttaaaaggtccgagtttggctctggatacaatgtgctcttcatcactgacctctattcattacgcatgccaaagcatacattgcggagagagcgatatggcattagccggcggagtgaatttgtcaatacatccgaataaatataagttcctcagtatgggacagtttctatcaagtgacggcagatgccggagttttggaaaagatggaagcggttatgttcccggtgagggagtcggggccgtattgttaaaaccattgaaaaaagcaatagaagatcaagatcaaatatacggtgtgattaagggaagcgccgttaaccatggaggaaagacgagcgggtattccataccgaatccggatgcacaggcggaggtcatttcagaagcgcttcgtaaagctgacattgatccgaataccgtcaattatattgaagcgcatggaacgggtacgtcattaggcgatccgattgaaattcaaggactgaaagattcctatcaattgaaccatccttgcgccatcggttcggtgaaatcaaatatcggacatttagaatctgcggcaggaatcgctgcggtaacgaaagtgcttctccaaatgaagaataagcagcttgttccttctctgcattccgatattgttaatccgtttatagattttgatcaggttccttttcaagtgcagcggaaagcggctgaatggaatattccgcaaaacagcaatgctgttcatcctttgagagccggggtaagcgcttttggggcaggaggaacaaacgcgcatcttatattggaagcgtatgagccgccaaaagcttttgatcatcaaagcggccgagtgatctttgttttatcggctcaagacaagcaacggctgataaaatatgcaaaggttatggctgattttctatcagaaaatgaatcgaaggataacaatcataaactgcgtcttcaggatattgcctatacacttcagacgggacgcgatcatatggaagaacggctggcgatagtggcagattccgggcgggtcattgctgaagagctttcagcttatatcaggggtgaaaaaagcaacttcagtcataccggtaatacattc

SEQ ID NO: 14 - DNA, 9960 bp, unidentified
  source 1..9960: mol_type = genomic DNA
aggatttctattccgggacaaccgctgatcaaacagcggtattggatagcggaaaaaggggaagcacttgtttcagcggatatgagtaagccgggtcaccctttcttggggagtaacacgtccgattttaatcagttcagtttcacgaaaaaaatcacccgtaatcatccgttcgcactaagtctgtctgaaggaaacgtgtcctttttcccttatgcatctttggctgaaatggctatcgtcaatgcagcggcggcatttggacaaccagttcaaacattgactgatttattgtggaccgaaagcatgaacggaataactgatatacgggaactgataattacgctgacgcctgagcgcgactttgcgcggtttgacatatatgcaaacacaaaaaatgagaataaagtacgtcttgccaggggaaatgcgcatattgctaagcctgaaagcggtatgtttcctgaagcgcttgatattgctgaagtcaaatcaaaaatgactgaacatcctgctgaagcgccttttgtcattgaatcagcgaaaaacagtgacggaatgctgtcgcttcattcattgccggatgagctttatgcaaaaagagagaattttacgatctctcctgaagcgcttgagtccgcgattcaagccagtgcgttttttgtcagcggagaaaccttcccggaattgatcggaatgggagcggcggatgtctatcaccctctcgctcaaaatttttatgtctatactcaaaaagagaaccggcagaacagccgtgatgtaacgtgtcatatgacgtttacagataaaaacggaagcattctggcagagtgcagagatgtgagattcagatatggtcatgaaaaagacactgttcagacggacgacgaactcattcgtctcctgcgtcaattggaaaccaatgatattgatcagaaggctgtattgaaacaattaatgggggaagaatgatgaacggaaaacgtgaacaactcagcgatattcttgatctgattcaaacaggacaaatgtcggctgacgaaggctatgcccgtgtgacatccattcagaatcaaaaatcacgggcatcgaaccatccgcttttatattacaagccggaatggacagaatcgttcattccgagtaagaagagcgtacccttgacgggattgctcctgatttttgcagaaaatgaacggcaaatacaagaattgagcggtttgtttaaggactgtactctcttgtttgttatccgcggaaaagaatttgctgttttacatgatgatacgatttgtatgaaccacgtgaaaaaagacgattatctaaagctgttccgatacattcatgatcattatcagctgcccgaaaagattcttcacttgtggtctgattctcaggctgacatgagtaaggaaggtatacagaaacaattggaaaacggattgttttctcttctgtatattacacaggcgctgattgaactaaaatggaaaaagataataaagctgacgcatatttttcaaacagagaataaagaacaactgccgctgcatcatgctctcggcggattggtgaaaaccgttcaccaggaacaccccaattttcactatcagattatcaatattacgcaatcagagggtgcgggagccgctctttcagcaatagcggatatatatcagagagagtgtgacgaaaaacgggatatccattttgaagtgcgctatgaaaacggcagacgttttacgaaaagctataataaagccggctcattcgatttaacccagccgctgaaacatcatgggaattatctgattacaggaggcctgggcggtctcggttatatattggcaaaacatctgtcggaacaatggaaagcgaatctcatattgacagggcgctctgatttaactgatgagcaggagaaaaagatcgaatatttaagaagttcaggttcaacagttgaatatatccgatctgatgtttcaaaacaaagtgatgctgaacggatgtttgcttttgcaaaagagcagttcggcaccttacatggagtatttcacagtgccggcgtactccgtgactcttttatcttaagaaagacaaaagaagaaattgatcaagtcacaggttcgaaagtttatggcacgctgtggctggcggaagaaatcaaaaaaatgaaagttgaattacttgtcttgttttcatccacatcggcagtcttcggctcagccggccaatgtgactatgctttcgccaatgcctgtttggatcagtatgcagcagttatgagcgcaaaagagtctgccgaaaggatagtgtcagtcaattggccgctatggacttccggcgggatgaatattgacgatgaaggaatatcctacatgaaacaggaactcggtcttgacccgttaggtgacggtgacggaatacaggctctggagaacagcttgtgtcaatcatctgaacagctgattgccctgaaaggccgtaaagaaaagattgaggatgtgctgaagcgtgcaggcacaatcagcttacattcagaccagcaggaagatgccgatttgcaaaacgatgaattaagacaaaatatcatcagttatctgaaaaatataatgtcagaggaattgaaattaccggcctcactcatagatgaaaaacaatatctggagaaatacggcattgattcagtgatgatcatgcgtctgaccaaaaagctggagcaaagtatcggcagattgtcgaaaacattattttttgaatatcattcaatatcgcagttagctgaatatttcctgagtcatcacagcgaacgaatgatgaaactgctgaaaccggccggaacatctgaacttgtctcatatcaaaaaaagccaaaaagaaaaccagtggaaacaaaagcgccggtacgctctgaaggagaagattcaaaagagcacatggcggccgatcccctttctgctgatatagccattatcggtgtcagcggcaggtatccgggtgcggaaaacatcagggagttccagaatgtgctgatgaatggacgggattgcgtaagcgaaattcctgatgaccggaaagaaatgctggagggaagctgctataagtggggaggttttctgaaaaatccagaccggtttgatcctttatttttccggatatcaccgaaagaagcggcttttcttgatcctcaggaacgtctctttttagagacagtgtacaataccatagaggatgcgggttataaaccttcagatctgacccgcaaaaaggtcggagtatttgcaggcgtcacatatggaagctatcaatttttcggcgttgaagaatcacttaaaggaaatgaactggccgtcgggtctccattttctgcaatagcaaacagaatatcatatttctttaactttaaggggccgagtatggcggttgatacaatgtgttcatcctctcttaacgccattcacttagcctgtgaaagtatacgaagaggagaaagcacacttgctgttgccggcggggtcaatcttacgcttcatccgaataaatatacattattaaatcaaagccgcttctttgcaagtgatggccgatgccggacattcggcgagggcggtgacggttatgtaccgggagaaggagtgggatccgtacttttaaagccggcggcggaagcggtaagggacggagatcatatttatgcgctgattaaagcgacagcggtcaatcacggagggagaacaaacggttttacggttcctaatccaaatggtcagtctgaactgatacaggacgcgcttcagaaagcttgtattgatcctcgtacaattagttatattgaagctcacggaacgggaacttctcttggagacccgattgagatcaccgggttaacaaaagcgtttcgggaatttacggagcacactcaatattgttccatcggttctgtcaaatcaaatatcggccatcttgaggccgcagcgggaattgccggtttgacaaaggttcttttgcaaatgaaactgaaaaaactgtttccttccatacatgcggagacgttgaatccaaatattgattttaaagcctctcccttttatgtacagcggtcattgcaagattggaagaaaccgaaactgcaggaaaacggaaaaacagtaactgttccgagaagagcggggatcagctcattcggagcgggaggaacgaatgttcatatcattatagaagaatatgaagaaacaaacgtctcaacagaacatgttgagaacgaaccgcatattgtcctgttatctgccaaagacaatgaacgtcttcttgagtatgccgaaaacatgagagattttatccagcgttcactaagtcctgaagtaaaggataagcagcctgatatgagagaaaaggtgtatgaagtactcaaaccgtttgtttcggaattgcttcatatccatccggaagaaattaatattgctgatcacctgagtgaatttggcgtagggcctttggaattaagtcggatggcggattttattaacgaaatgtttcatattgaactcacgatgtatgaattaactgtttgcccctccattcaagcggttactgaatacgtattgaaggaatatgccggagcgataagaaacctgttcccgaatgaagtgatggaaggcagccaatcggaaagcgtcgttttacgggatctggcatacactacacaatcgggcagagaagcgatggaggccagagcggcgattaccggtacctctcttaaagaaatcaaagataaattatttgagttttgccgcggtgacaagcaagttccaggcgtatatgttcatcagcaatcagcagatgcatttgctgaaaaaatgaaggcgttaacggaaggcacagcaggtgaattgtttgtaaagaccattattgagaacggtgaagcggaaaagctggctcaattatgggtttggggggcggatatcgactggaaatccgttcatcaacatttacagcagccgggtaaacggatctcaattccgacttatccgtttgcaaaagaacgttattgggttccgtccaggactgagaagacagagatccctcagcattcttttcaacatcacttgcaaaaaaaatggagggagaatccgtttgtaaattcatccgaaacaaaagagtacggactgacagtagcatttgtaaatcaagacacagaatcaattgcccgcaccatttgttcggatatagatcaggagaatctgattgtcatcaaacatcactcttcatttataaaacactctgatcaaatgtttgagtgttctttattagatgaaaaccacgcatacgacgttatccgtgagatcttctcgggcggtgagcggctggtcgaaaatatatttgatttctcagatatcggaaaccagagtgacaatcagaacggcatcccatattgtaaattcttatttatacaagaaataattaagcggagtaaaaaggaaacaccattatccattttacaaatcaccaatggcttgcaggtatttaaagcggaaaaaccaaagctgacgggggcagaatcagcggcttttctgaaagccctttctgcggaatacggctttgttcatgtgaaaacaatagatatagacagcatgaaagaaacgcatgagctgaaacaaatctttgagaatgaaagggcgcaatgtaccggagagaatgaagtatgctaccgcagcggcgtcagatatgttccgtattttgaagaagaaagaatccataccggcgggcctgtttcatttcctgcggataaagccatcgtgattaccggcgggacagggggcattggcagagcaatagctgaagatttggtgaaacggggagtgaaaaaactcgtgctgacgggtacgcgtccattacctttgcgaagtgaatgggatcatttgctgaaagagggcagacaggacgaaaagacagtcagcaatataaaactgtttcaaagttttgaagagaaaggtgtcaactatctgtattacagcggttctctcacaaatgaagagaagctgcgttcattctttcaccaagtaagccttgaatttaaagatataagcggtgtcatacactgtgccggactgcacagcggcggaaatccggcctttattcataaaagaatgagtgaatttaagacggtgtacgggccgaaagtcactggacttcagaatttggagcggatttttaataataggccgctggatttctttattctattttcatcaattgcagcacagtcacccaaactgtcgaaaggaatgactgattacgcgtctgcaaacgcatatatggactatttcgcggcttatcattttcaaaaggggagaacgtatttcacgtccatcaattggccgagctggaaagaagtcggaatgggtactgtgacgagtcctttatatgatgaattgggattggactctctttccacacgggaaggactggcgattttgtatcatgcactggagagaaaatcggccaatatcattcctatcgtgaaaaaaggaacagtgtttcatgcagacagtctcctgaacatcaataaaaccggcggagcttcggttccagtccgaagacaggagactcccgacacttttgatcattcagaagggcttgtcagtgaactttctgccgtatttgccgaggaattgagcattccccttgaaaagcttaacccaaatacggattttcaggaatacggtgtggattctatactacttgtcagcattatacatcgaatagaagcaatgattcagaaaaaaattgaccctactgtgcttcttgaacacaacacgattcatggtcttgcggaatatatcggtaagacggcgccgccgttgaagaaacaaatcgaacagactgattctcaaagcagcaatcagcaagatatgcaatctgtgacgagccgaaatacagaaccgcagacggcttcctctgcaaagatagctgttatcggaatcggatgtaagttccctggagcggctgataaagaggaattttgggacaatctgagaaaagggaagaatcatattaaagaagtacctgattcccggtggaatatagaagaaaattactctccggtgcagcaaaagggaaaaagcatcagcaaatggggaggatttatcaatggaatcggcagcttcgatccggagtatttccgcatggaagaggagaaagccatccaaacggacccgctgataagacatttcttagaaacgagtgtacagactgtacgggatgccggttatgaagaaacagaattatcgggaaaaaaagtcggcgtatttgtcggttccagaatgggcggttacggtcacaaactgccgaaagaaagcagcagccacatcgtcgggcttagtcaaaactttattgcttcacatgtgtcgcatttctttaattttcacggtccaagcatggtttctgataccgcttgttcttcttcattagtcagcattcatttagcctgccaaagcataaaaaacggtgattcagagatggcgctggcaggaggggcggatctgcttttggatgaaattccttacctgatgttaacagaaggaggagctctttcgccggacggtcagtgttttacttttgatgaaagggcaaacggctttgtacccggagagggttttggcgctgttctgttaaaaccgctggagaaagcgaaagctgacggagatcggatatacggtatcattgaagcatcagctattaataacgacggtgcgacaatgggaattacgactccgaatcctaagcttcaagagcaggttattcaagatgcatttgaaaaagccgatatcactcccggatcagtcagctacgtcgaaactcacggcacaggcacgatgattggagatccgattgagcttcaggctctgacaaaagtattccgaagagacacaaacaaaacgcaattttgcggagtaggaagtgtgaaaaccaatatcggacacttattaagcgctgccggagcggcaagtttcattaaagtgattttatcaatatggcataaggaaattccgcctacattgagaatcagaagttcacaatgaaatgcctcacgtttgttcagattacaccgaagcagcatccaaatgggtaaatggaagaaagatagattgggagacttttgaggtgaatcggaatgctcgcattgtgaaacgccaaatccgcgctttgaatttgaaaagtcgcctttttatccgaatacagagctgagaagctggaaggatggaaaacggagggcgggcatcagcgctttcggttttggcggaacaaacgcccaccttttggtaagcggcctggatgattcatatcattccgtcagaactccgctgccgcctgctgagtttgtcagaaaagagtactggccggattcaaatcggaaaaaacaaatcaaacagaataaaaaacggcttcttgaaattattgatgaaacagagtagcacctgctctgtttctttactttgtaaaagaagaaaggttgtcgagttgcatgaggttcagcgtattagtcaaacatgacgattatattgtttcagaccatgtccttcatcaagtgcgtacgatacccggcgtaacgtatctggatttaataacacgatttttagctgagaaagggatatcaccccgaagttttgaaatcaaacatgttttattccgtgaacctgttgcgacgtcagtccattttgataaaaaagtcaccttccagtttacggaacaagaaaacggctggcttgttgaaacaacaagcaggaaatgggtcgctgaaaaggcggaaggtgaagcgtcagtcaacttgcaggcggaaatcataagaaaaaatgcgataaacgaaaagacaattttaatcgaagaattaaagaaaggggccgtccggacttcggatgctgaatccgcctatcgtaatttgcgcagaggcggcctgattcataaagattttatgaaattggaaggtaaggtatatgaaggtgaaaattatattctgggagaggtcagtttaagcggactttcaaaagaatatacagatgacttttacatgcatcccgcatttttggatgggtctctcagcttgttgggaatccttacatcagacttgccggatgaaggaacaaatgcgtttatccctatatatattgagacattccgggcactgcataaaacaactgggtcttgttttgtgtatattaataaaaccagtataacgcagccgccttctaaagacattttatatgcggacatagaagtatacaggcctgatggagagcttgctttttattatgagagattcggtgtgaaaagagtcagaaatgaaaaatcaatacaaaggctggagcattcccaaaaagctaaagaggtcggcagcgcaacgaacaataaagaaatgatcagaaataatttgatccaagagatagccggcatgattgaaaaacctgctgaaacagtgaagaatgatatttccttttacgagatgggcttggattctaaacaattattagacttatccaaaaaattagaactgatgttaggttcaaagctgtacccgactttattatttgattactccacaatcggtgaattgacggaatatctgcaggagaaatacggagccgacattacagactctatctctgatacagttcagcaccaagacagtcataaaacattaacagatgagctgtcatccattattggagaggctacaaacacaccgccaaataaagcggacaaacaccaatctttttacgaactgggtcttgactcaaaacagctgctgcatatatcgaaaatgctcgaggaaagattggatacacaattgtatccgacattgctttttgattataacaatattcatgatttagcggcttatttagaagaaaaatatgatgtgcgtaatacgccggccgcagcttttaaagaaaaaacgcaaac

SEQ ID NO: 15 - DNA, 9960 bp, unidentified
  source 1..9960: mol_type = genomic DNA
gaaagagccgtctgaaaagccggtgcttgattccttgtatttacaaaaagaatggatagaatctccattaaagtccggagaagtacctaaggaaaatacattgattttcgatttcggcagttcattgagtgatgaaatccgaaagtacatcggcagtgaaaatgttatttatgcggaagccgccgctgaatatgaaatggtaaatgaaaagcattataaatttaatccggagcagtctgctcattacaccagactgttagaagatcttaaaaagtccggaacgtttccggcgtcaatcattcattacctgtcaaaaaaagcttatactgaggcagcgttagatctgactcacagcttttattcaatcctttatttaagcaaagcattgatccgtcaaaaaacaaaagaaaacatcaacattttgtacgtacatgacaagggtcaatatacaaatccggtttacgccgcgattggaggcttttttaaaacgctgaatgcggagcagccgaatataaagtgtaaaacggttgagatttcttcaagacaaaccattgaaaaagcacctttgcttctcaatgaattaagtgccggacatcatcaggctgtgaaatatgacagcgatcaaaaaaggcgggtgacagcatataaagacacttcgccgcaaacaggacaaaaggccgcaaagtttgcaaaaaacggggtgtatctcatcatcggaggattaagcggcatcggctggattatttcagcgtttctttatgcaaatcacggagccagcgttatcattgccgggcgatcgaaaatgacagcagacatggaagaaaaattagacagcatccgatatgacggggcaagcatttcttatatccaggctgacatgtcagataagcagcaggtcatcaagttagcggaaaaaattcatgctgattattccgaaatcaacggggtcatttacagtgcaggcgtgttaaaagactcactcattatgaataaaacagacgcggaagctcaagcggtgctgcaatcaaaggtcaatggtgtgattcatttggatgaagcgctcagaaatatgcctttagacttttttgttttgttttcttccatctcctcagaaatcggaagtctgggtcaagccgattacgcctatgccaatcgttttctggagcattttgccgagtggagggaatgtatgagaaaggcggggggaagatcaggaaaaaccatcgctgtaaattggcctttctggaaagacggaggcatgtcagttgatcccggcattcaagacattatgaaaacggagtacggacttcacccattagaaaatactgccggaatcaaagcttttgaacactcgcttctcggccgaggccaccagctgatcgtgttatacggagaaaaagaaaaaatacaaaacgctttcaagccgttatccgcccgttcagcaccgcctctggtaagagattcagcgattgcagccgattttgatgcttcgtcagtcgttacggaagaagccgagagtcctttccgaatactcggcacggcttccaatcgtcatctcgacgattatttgcattattggagcagactgaagaaaggagcccttcagcacaatcaaagcgatgccgcaaaaaaactcatcgctttttaccagacagaagaaacggaaaccgttcattttctggcaaacacagaaaatgcccggcatatggaaatattgatgagcggtaagggtgaaacaattttactgatttgcggttttgctaccactgcttacatgtggaaagaacaaatgaaggagtgggcagatcaatttaatgtaattgtcatacacacaccgggttacgggctgagtgaggggagcgccgattttacactgagcgggctggctcagacttttatcgaagtgctgaacgagataggggttgcttggccgatccacattgcggcagcttcttggggaggcatgatagggcagaggatagcctatgaataccctgaaaaagtaaaaacgctcattctttccggaagttacaccgcatacgaagaagaccctgaaagcagtgtcgaggaaaaactgaaaaatgattttatagctataggaaagtcgcatgaatatgaaagtttatacagtaatcaattcttaaatgcttctattgaacgttttatgaagatacatcaacaagaaggctattctacgatcggctttttaaaagacatttatacaagaacgcttatccttacgggtgagaaggaaagcgtagttgatcagaaagaaacaaaaaggctgcagaaactgctgagcaatgcagaattaaagaaaattaagggagcggggcatcttccgaacatcacacatccggaagaatttaataaagcggtgtcagaattcatcaagaaatcagtaagtaagggatgaaaacgatacatacagtacagggggagaaaacaatgaaacaaacattaagcaatccggcatttgatatgaaacaaattaacgctcttaacggccattatcagacgatgatagacaatggagatctgcaatgtgcgagttatatgatgtcaagaggaggtgaggtttttgcagctgagtcgttaggggaatttaccggcggccaaaaagaaaagcaaacatttcagcttgatacaatcagagaaatcggttctttgacaaaagtgtttaccgctgtagccgtcatgcagctcgtcgaaaaaggtttgctcgatctgaaaatgccggtcaagctcattttgccagcgtttgataaaccgggtttcggagaaattaaaattttgcaccttttgactcatacggcgggattaagttttgagctggatattcaaaaggctgaaggcattgacttaacgaatgaggaagaatggataaactatctggtcagtacgcctttggagtacggagtggatgaagcatggaactattccagaaccggctttgttatacttggcatcattatttcaaaagtaacaggcgtatcctacgaacagtatgtaacaaagcatattattgaagcgctcggattagaaagaacgtatttttatgtgcctgatgctttaaaagaagaagtttgtgtgatcaatgagcacgagtgcgtacagctggaaaaaagtcatcacccgtattttccgaataaagcgacaagcggtctgtactcttcgttgcgagatatttggaagttagctgaaatgtttagaaataaaggcagattgaaagataagaagctgcttggaagaaaaacagtcgaggcgatgctgagaaatcaaataaagcccggtcttcctttttacttcttcggagcgcccagagaggaaggcggctttggtttaggcattaatttgtggccggccggtgaccattatttcatgacagaaggcaccttctcacatcttggaatggggtggtgcggcatgttttctgatccagccgaagattttacgtatgtatttttcactccgatttccgagtttcatcctcatgccgtgctgacgccgctgaatatcgtgtgggcgggtattgaataattagacaaaagcatcgtcccgtctgttcggcaggcgggacgattgctaactgcccgtttttatcccgcggcatcttttcaaagcctgacccgaagttaaataacttaatttactctttgtaattttttttaattatagtacagttgagcatttttataatacagtttaaagtcttttatagtacagtttggcgcaatatgatcttatgttttataagcggtttcaaaatgacgttataaataattgacggctttgtttaattactgtaaactatggttgttgatcatattgtactaataaaaaatctttttttattagtacagtttaaacgtcattatctttgcttaagcgctatgtgaatttctgaatgattaattttagaattgcagagcatgataagactgtttttaatacggatggctaaacggtatcctaaccctgttaaatcaaaagaagaggtggtttaaaaaatggctgcaaagacaaaaacttcaattgttgatagcaaaaaacaatttgatgctattcataagcagtttgaaacgttccaaattttaaatgctgaaggagaagtcgtaaataaagacgcaatgcctgatctttcagatgatcagttaaaggaattaatgagaagaatggtgtatatccgcattttagaccagcgttcaatttctctttcccgtcaaggacgtttaggtttctatgcaccgacagcgggccaggaagcttctcaaattgcatcacacttcgcgcttgaacaggacgactttatccttccgggttatcgtgacgttcctcagctgatttggcacggtcttcctttacatcaggcattcctgttctcaagaggccattttaaaggtaatcagatgcctgagggagtaaatgctttatcacctcaaattattataggtgctcaaattatccaaaccgcaggtgtagctttaggtcttaaaaagcgcggtaaaaaagcggtagctattacttacactggtgacggcggagcttctcaaggtgacttctatgaaggaatgaactttgccggagcatttaaagcgccagcgatcttcgttgttcagaataaccgctatgcgatttcaactcctgtagaaaagcaatcatccgcacagacaattgcccaaaaagcagttgccgtcggtattacaggtgttcaagtagacggaatggatgcacttgccgtttacgctgcaaccgcagaagcacgccagcgcgcaattaacggtgaaggtccgactctgattgaaactcttacattccgttacggacctcatacaatgtccggtgacgatccgactaaatatcgtacgaaagaaatagaaaatgaatgggagcaaaaagacccgctcgtacgtttccgcaagttcttggaaaacaaaggactatggtctgaagaagaagagaacaaagtgattgaacaggccaaagaagaaatcaaacaagctatcaaaaaggctgacggtgaatcaaaaccgaaagtaacagagctgattgagaatatgtttgaagagccgacgttcaacttgaaagaacaacttgaaatttacaaagcgaaggagtcgaagtaagccatggcgcaaatgacaatgattcaagcgatcactgatgcgttacgcacagaactgaaaaacaatgaaaacgttctgcttttcggggaagatgtaggcgttgccggaggcgtattccgtgcaactgaaggccttcaaaaagaattcggtgaggaccgcgtatttgacacgccgcttgctgaatccggtatcggcggtctcgctctcggtttaggcctgcaagattaccgtcctgttatggaaatccaattcttcggattcgtatatgaagtaatggattcaatctcaggacaaatggctcgtctccgctaccgttccggcggacgctggacttctccggtgacaatccgttctccattcggcggaggggttcatacacctgaacttcacgctgacagtttggaaggccttatcgctcagcagccgggtattaaagttgttatcccttccactccttacgatgcaaaaggactgctgattgcctctatcagagacaatgatccggttgttttcttagagcatatgaaactttaccgttctttccgtcaggaagttcctgaggaggaatacacaatcgagcttggcaaagctgatgtgaaacgcgaaggtactgacctttctatcatcacatacggcgcgatggttcatgagtcgcttaaggctgctgaagagcttgaaaaagaaggcgtttctgctgaagttatcgaccttcgcactgtaagtccgcttgatattgatacgattattgcttctgttgaaaaaacaggccgcgcaattgtcgtgcaagaagctcaaaaacaagccggcatcgctgccaacgttgtagctgaaattaatgaccgcgcgattctgagcctggaagctccggttctgcgtgttgcggcgcctgatacagtatttcctttctcgcttgctgagagcgtatggcttccaaaccataaagacgttcttgaaactgcgaaaaaagttcttgaattttaatcaggcatcataaaagacggtaaagagagtccttttctcttatatgaaggagtctgtttactaggttcaatgcttgcgaatgcataactaaaatttgcttaatcaactaggaggtcgaaaactgtggcatttgaatttaaacttccggatatcggggaaggtatccacgaaggcgaaatcgtaaaatggtttgttaagccgaacgacgaggtagaagaagacgatgttctggctgaagtgcaaaatgataaagcagtggtagaaattccttcacctgttaaaggaaaagtattagaattaaaagttgaagagggaacagttgcaactgttggacaaacgattattacgtttgacgcacctggttacgaagatcttcaatttaaaggaagccatgattcaggagatgcgaaaactgaagctcaagttcagtcgtctgctgaagcaggacaggatatctctaaagaagagactcctaaagagcctgcaaaagaaaccggtgcaggacagcaggaccaagtggaagctgatccgaacaaacgcgtgatcgccatgccttccgtacgcaaatatgcccgtgaaaaaggcgtggatatccgtaaagtaacaggttcaggcaataacggacgtgttgtaaaagaagatatcgacagctttgcaaatggaggagcggctcaagaagcggcaccgcaagaaacagctgcacctcaggaaacagctaaacctgcagcggctcaggctcctgaaggcgaattccctgaaacacgcgaaaaaatgagcggtatccgtaaagcgatcgcgaaagcaatggtgaactctaagcatactgcaccgcacgtaactcttatggatgaagttgacgtaacaaatcttgttgctcaccgcaaacagttcaaacaagttgctgctgatcaaggcatcaagctgacttatctgccttacgtcgtcaaagcattaacatccgctttgaaaaaattccctgttttaaacacatctattgatgataaaacagatgaagtgatccaaaaacattacttcaacatcggaattgcagcggataccgaaaaaggcctgcttgtgccggtagtgaaaaatgccgaccgtaaagccgttttcgaaatctctgatgaaatcaacagccttgctacaaaagcgcgtgaaggcaagcttgcacctgcggaaatgaaaggcgcttcttgcacgatcacaaatatcggttctgccggaggccaatggttcacaccggttatcaaccatccagaggttgcgattctcggtatcggccgtattgctgaaaaagcaatcgtccgtgacggtgaaattgtagctgctcctgtcttagctctttctctcagctttgaccaccgtatgattgacggagcaactgcacaaaatgcgttaaaccacatcaagcgtttactgaacgatccacaacttattttaatggaggcgtaatgttatggtagtaggagatttccctattgaaacagatactcttgtgattggtgcgggacccggcggctatgtagctgccatccgcgctgctcagcttggacaaaaagtaacagtcgttgaaaaagcaactcttggaggcgtttgtctgaacgtcggctgtattccttcaaaagcgctcatcaatgcaggccatcgttacgagaatgcaaaacattcagatgacatggggatcactgctgagaacgtaacggttgactttacaaaagtccaagaatggaaagcttctgttgtaaataagcttaccggcggtgttgcaggtcttttaaaaggaaataaagtggacgttataaaaggggaagcttatttcgtagacagcaactctgtccgcgtaatggatgagaactctgcgcaaacatacacatttaaaaacgcaatcattgctaccggttctcgccctatcgaattgccaaacttcaaatatagcgaacgcgttctgaactctacaggcgcacttgcgcttaaagaaattccgaaaaagctcgttgttatcggcggcggatatatcggtactgagcttggaacagcgtatgcgaacttcggaactgaattggttattttagaaggcggagatgaaattcttcctggcttcgaaaaacaaatgagttctctcgttacccgcagactgaagaaaaaaggcaacgttgaaatccatacaagcgccatggctaaaggcgttgaggaaagacctgacggcgtaacagttacattcgaagtaaaaggcgaagaaaaaacggttgatgctgattacgtattaatcactgtcggacgccgtccgaacactgatgagctcggccttgagcaagtcggtatcgaaatgactgaccgcggtgtcattaaaacagacaagcagtgccgcacaaacgtaccgaacatttacgcaatcggtgacatcattgacggacctccattggctcacaaagcgtcttacgaaggtaaaatcgctgccgaagctatttcaggagagccggctgaaatcgattacctcggtattccggcggttgtattctctgagcctgaactggcttctgtcggttacactgaagcacaggcgaaagaagaaggtattgaagtcgttgcagctaagttcccgtttgcagcaaacggacgtgcgctgtctcttaacgaaactgacggcttcatgaagctgatcactcgtaaagaagacggtcttgtaatcggtgcgcaaattgccggcgcaagcgcttctgacatgatttctgaattgagcctggcgatcgaaggcggaatgactgctgaagatatcgcaatgacaattcacgctcacccgacattgggcgaaatcactatggaagctgccgaagtagcaatcggaagcccgatccatatcgtaaaataataatgtaacaaagaaacagcccccgtttgtaaaagcgggggctgtttttattgtgcaagcacatgctgcataggtttgatgatatcctcttttttctttacggatccctcaatcttcatgaccacatgccgatgattgatcaccatcaacgcagggaacgtgttgatttcctcttcccatccctttctgttgtcataaaccttcatttgatcgacatgatggggatatttcttttctaaatcaagaatcgcatcgtaataaggcacttctttatctatatgattttcatcggaaaaaaacaagatttgagtgtgatgaatgctgtcgatccggttttttgtgaatggactgacagtagaaaccgaacagcttgacagaacgaacagcatcaaaaaaaagggaatcaccgtacggtagcgcatctatcttactcctctgttttatataatatatgggttcatccatttataaggactatatcaagtgtatcacgagaggtgaaaaacctaaggcttgttactgaattgttatataattgaaaccgtttttaatgaagaaataatacgtgtgacaacttgcgcagccgatgtgtaaaaaaggcacattgtttttcgtgagagcgctgttttttcgttatactgggagcaaaaccatatcaggagggattgtcatgtataaagccgctgcagctgtcatatgtatgtgcttactcgccggctgccaatcaaatgaaaagatgcaaacaaacaaaccccattctcaagccgcggccaatcatgccaaacgggaaaaccaccatacaaaagaggaaaaacagaaaaaacagaaggcggagccccgttactatatcaatccgaaaagcttttctgtggaaaaaatggatgaacagaaagagaaaaaaccgatcgcattggtaacgatcgatgacgcgcctgatcagcatgccgctgagattgcggacaaactgcacgatctcaatgcgtc

SEQ ID NO: 16 - DNA, 8212 bp, unidentified
  source 1..8212: mol_type = genomic DNA
agcgatcttttttgtaaacggacattttttgaaaaaagatgaaaataagaaagcgcttaagcatattcatgagttaggttttatgatcggtaatcatacaatgactcatcaaaacttaaaagatctttccgaacaaaaacaatgggaagaaatcattggcgtcaataaagcggttgaaaaggtaataggagagaaacccgcatttttcagggcgcctttcggaagcaatacggactacagtgacagacttataaaaaaagaaggtatggtgaaaatgaactggaccttcgggtatgattgggatcagaaatatcaatccaaaaaagcgcttaccgatatcacactgaacagtccttatctgcgggacggggccaacatattgatgcatgaccggaaatggacggcggaagccatgccggacattgtaaaaggattgcgtcataaaggatatactgtgattgaacctcgagctataaaaagaccgtaaaaaataaatgtgttatacaatttatcagttgacatattaaatgtgacataataatataataacaacaagtgaaaattaagcgttttcaaattctgagaaatggggaatgacagatgggtacaattgtttgccaagactgtaacgaagcgattcattactttgaggatgagaaggtcacgacgttatacggaacatgctgcggacaatgccagtgtcatcttgaagaagagtaaaaagcgtaaaaaggcccgaaacgatttcgtttcgggcttctttatgtttattggatggctttttgctctttgatgactctgatcatttcaagcgttgcatcttcgggaccctgcacaggaaggccggcgtccaagtttttaaaaatgtagctgatattttcttcagtaatgatctcgcccggaataaagatcggaattcccggcgggtacaccatgacaaactctgcgatgatccgtcccgcggattcttttaacggaatgacttccgtatttgcgtaaaacgcgtctcttggcgtcatcgctaacagaggaatttcaggaagcagaacttcggtttgctgatgagcatagtcattgtcggaagcctgctttgaaatgtctgtaagcgcgcttacaagacggtcggcatctcccgctctgtcgccgggggtgaaaatacataaaatattatacaaatcggacagctcgacttcgatattatatgattcgcggagccatttttcaacgtcatgacccgtcaggccgaggtttttgaccgaaatgattaatttagtcgcatcataactgaatgcagctttagaccctgtgatttcttcgccgacacagcggatgccttcaatttgattgaggcgtttccgtgtctgatccgccagcagaatcgtatcttcaagaaggtcgcggccttctgtagcgaggcgttttcttgcgacatccagtgaagcgagaagcagataggatgttgacgttgtcgtcagcatgcttaaaatcgactgtactctttctttggaaacaagaccttctctcacatttaaaatggagctctgcgtcagggagcctcccaatttgtgcacgcttgtggctgccatatcagcgcccgcctgcatggcggacagaggaagcttatcatgaaaatgaatatgcacgccgtgcgcttcatcgacaagcacaggcacatcgaaagaatgcgcgagctttacaatgctgctcaagtcagctgccacaccgaagtacgtcggatttatgacaagcaggccttttgcgtcagggtgctccgtcaaagcccgctcggcggactcaagcgtaatgccgtgagaaattccgagctcatcgtcaatttccgggtgaataaaaatcggcaccgctccggaaaatacaatggccgacataatggatttatgaacgtttctcgggatgatgattttatctccgggtccgcatacggccatgaccatcgtcataatggcgccgctcgtcccttgtacggaaaagaaggtatggtctgcgccgaatgctcgagcggccagatcctgggcttctttaatgatgccctttggcgcatgtaaatcatccagaggttcgatattaattaaatctatgcttaacgcgttttctccgataaattgtctgaattccggatccattcccgcgccttttttatggcccggaatgtggaattggacaggctgtctgtccgcgtgctttttcagtccgctgtacaggggtgtttcatgttgcggcaattgttttcccacctttatcataatacctacggattagtttgtattacattcagttttattacccgttttttaaacgtaaaacaaatgaattatattatgaacttgaaagaaagtaaagaacaatttatgatctatggaagatgcttattttgagcatgattataaaggagtgaagacatttggaatatcaatatccgatggatgaggactggacgacagaggaggccgtggatgtcattgctttttttcagcaggtggaaaacgcctatgaaaaaggagctgaccgagacagacttctgaatgcgtaccgccgctttaaagaaatcgttcccgggaaagcggaagaaaagaaaatctgcagccaatttgaagctgacagcacctattccccgtatcggactgtgaaacaggcgaaacaatcagatcaagcgattatcaaaatgtagacaaagcgagccggcaatcggcccgctttttatgttatgagactttctgagtcagtgtgtataaaaaggaaagcttcctaaaagcatcttcaatggtttgcaggaattcattgttgttcatgttgatcacgtcttctttttgcagctgaatgccgcacaaaagctctgcttttttcactgtctgcaggcgttcaaataatgttttcagctgctcctgatccatgtcggactgcttgtacgcacctggcttcgtatgatccggcgaccatacaaaatgaccgggaatctgttttgtgatgtcctcctgcttcgcttccagcaatctgccgtattcctcttttatcggagattcataaatgatggcaaaccatacaaacatatgggtgtcccataaaccgatctgaaagtgagggagtttcttatacccgcgtttgctgttggcaaaggcaacccagctgtcagccggcgggttgacagatcttctcgcgtgcttggcgacatgcgggaacatttcatctcctgtcaatgctgacaatgtcggtgcgaaatgctctcctaacaaggtcagcttcggacggacttgttcttttaacacctccatgcgcggatctaaaccttctattgtaaatgtatcgaagtcttcttttgtaaaacgcatctgagtcatctttttcctccttatcgtaagtattcatattttaccacagccgccgccggccgcaaaaacattgagaacgtttgagccgagaggattggtggtaaaaaacgaataggtaaacacacaattttaagaatgttgttacagccgaatataagtttccatacgatataagaagtatcattaataaattggaaaaatgggatgcttttttaacaaatgatctctggaaggggcgaaagacaatgaggcaagtggtgaaggaaggattcagggaggaaaaaaataaccgtgtagctgtctggagactagaggttgattatgaattggcaaccctgtatgaggccatgcaaaaagagaatgaagatcaaatcgatctcagcaaaagaaaactggaacgtctcagaaaagaatggattcggcttaacggataaagaatagtaaaaacgaaccgcaggaaatggttcgtttttacgtgagaaggcatttttttcattttccttgctttttcatcgcctttatcagtatgctatagaataagctatacatagaaaagaggtacacggtatgacaaattggacggaaattgatgatatcgcaaaacaatgggtaaaagaagccggaacgagaatcagacaatccatgcaggaaagcttgacgattgaaacaaaatcaaatccgaatgatttagtcacaaatattgataaagagacggagcgctttttcattgaccgtatacaaggaacttttcccggacaccggattctcggagaagaagggcagggggataaactccgctcccttgacggaatcgtctggattattgatccgattgacggaacgatgaattttgtccatcaaaagcggcatttcgcgatctccatcggcatctttgaagacggaaaaggcaaaatcggcttaatttatgatgtgatgcaggacgaactgtatcacgcattcagcggaaaaggggcttatctgaacgacacgccgcttgctcctttaaaagaagcttccattgaagaagccattattgcaatcaatgccacatgggtgacggaaaacagaaggattgacccgagtattttggcgccgctcgtaaaacgggtcagaggaacgcgttcctacggttcggcggcacttgagctggccagtgttgcggccggccgcattgacgcatacattacgatgaggctcgcgccgtgggattacgctgccggctgtgtgctgctgaacgaagtcgggggcatttacacgaccacagacggcgaaccgtttacgtttttagacaaccacagcgtgcttgcgggcaatccggcaatacataaaattatctttgacgactatttacatgctgacaggtagagattttgaagaagaagatctcgggtttctggagcagctggcggcatcacacccggtgtggaaaaacgaagagttcggaacaaaaaatgcggcggaatttatgcttgcatactccatgtacaatggaacatggctcgtatgggagattgacggcgctcctgccgccgtcagttttcatttggaatgggcgccttcaaacggaaaaccgtggctcggaaccgtggccgtcgctccggaggcaaaagagaaaggaatcggacgcgcggtcattgaagagattgccggccggctgagaggggagcataaagcgatgttttcagctgttcccctggacaggcaggaatgggttttgtttttaagccaatgcggctttgaacagtttaagcttgaaaaagacgatcgtgaaaagacatatatgatcatggtgaagccattgtgaaatgtttcagatataaaaaagagaccggatttctccggtctcttttcttgtttacaagccgaccgcattccaggccgcttctacgcttgcagcgtcttgagagccgtaaaggtcccgcgctgattgaatcaaagctgcttttgcatctttaaagtttgatgacggagtgagatataccgttagtgcgcgatagtaaatctgctccgcttttttcacgccgatttttgtaatcgtgttgtaagcggctttgttcggaattccgctgtttgtatgcacgccgccgtagtcgccggcatcagtgttcggaaggttttgataatttttgtaatggtcgggctgtccgtattttgtcggagtggataaactgcggagagccggctggctgaccgtaatatcttcaccgatatcccagtcctcagtatcgttgaagtatccgaatacatcagagaaggattcatttaaagcgcccggctgattttcatagttcaggttggctgtttcctgtgtaacaccgtgtgtcatttcatgggccgttacgtccattgaaccggaaagaggcgagaagaatgagccgtcaccgtcaccgtaaatcatttggtcgccgatccaggccgcgttattgtatttgctgccgtaatgaacggaagatacgattttgccgcctttattgtcgtagctattgcgtttaaacgtctgatagaaataatcgtacactttgccgagattgtaatgcgcatcaacggcagcgcgctgagaagaagttgtgaactggttggtagtgcttgatacgagtgtgcccggcaggttatattgtcggttttgcagatcgtacgtaataatttgcgttccggtaggtttagaaagatcacgcattacatatttgccgctttcagaagaaatatttaatgagaccgtttttcctttaagagtcgtacctgttccggttgcagcggcatgctccactttgttttgctttttcaggactttccctgtttccgcatcaacggttacttcccagttagctggttccggttcgatgtagcggatggttacatcataggcgaggcggtatttaccgtcttttgtggccgctgctttcagctcggctttgtttttgtttgtaacgttgccgttagagacggcttcaggtgatttgccgattgctttaaaagcatgatccagcgcctgatttgccgataattttttgctgttagccgttttggcagaagcatcgttgtttaattcaccgttaatcgcatagacattgttggatttatcgacgtgaataatgacttgcgagtctttcacaggcacaccgttaacgacaggcacataacggaagtgcttgtagccgagatcatcggtcgtgtggtcaatcagcttcagtctctcagaagggttgcctttgaagactttgccgttttgtttcaagtagtgcttgattgctttgtcactgactgaaggcaactcagactgcaccaaagaatgcttcggcacaaagttcgtcaggttttctttaagctgaggattctcagcggcctgaacacccggaagactgatagttaaactcataaaggaagcggcgacagcaacagacaatttcttacctaaacccacaataaatccccctttttgaaaataatgaaaacttatattgttataataaaccagcattctctcattctgcaatataaaactagactccgctgatgaattgatagaatcgtcctggaaaggcttccggcggccagcatcgggggataagccgagaggcacgtgttgaatcagacatgagtcgaatgatgcgatttattctgaaaaaaatgacaaatgaagtcgggaaaaatgataggggaaaaatgttccgatctgtaaaactttttttatagccgtttgtctaaatgagtgaggccttaataaacgaaaaggattgtttcagatgtcagagattttcagtcattttattcagcatttcgacagatatttcaagagctgtcttacgaagagattgctgatgttttggactggagtctttcgaaggtgaagagcaccctgtacagggcgagaatgcagctgaaacagcacttttgggaaaaaaggaaggacggacagatatgacttgctgtttaatgagagatttactccctttatatattgaaggcgattgtgaaacagaaacggagcggtttatttcccgccatcttgaatcatgcggtaagtgcgggagtttatatcatatgatgaaggagccgcttgacctcggcagccctgagatgaaggcgccagcctgttatgcagaagaagaaaggcggtttaaggaacggtattacggaaagctgctgataaaagcagcctgtttgttcggcgctgtatttcttatcatgctggtccttaaactgctgatttaaaaaaacgcccggctgaatgaccgggcgttctaatccgtttactcagccggatgaaaggctgtataaatgccttcaagcaattcctggggcagtgaattaaagaatccggcacacagctttgcttctttttttcctgctcctgctttttcagcaagttcctgatcaaatgtatacacaccgtcttcttcaatcagcgcagattgaaggatatgaagactttgggaaaaagcggtgaacagctgatcaacgataagggcctctttttccgtaaagccgagctccaccgctttttctttattaaatgtatattcgccgtccgttttctcaatacattgggaaagtacggttaaaaattcggttgaactgtcccacagcttttgaatgatctgggcttgttcttgatctaatcccaattcaaccgctttttcgccgtcaaaataatagacaccgtctttttcgtaaatcgattggctgatgatgtcaatgagttttgccgcgtcttccggtgttttctctgcggaagcctgcaggacatgcggttcatttgcctgaatcgtttttgcgccggcagaatcaagcggactgaatgcaagcaaggcggctgaggcggttgcggcggcagttacagcggttttcttcaaaaaagatgttttcat

SEQ ID NO: 17 - DNA, 9960 bp, unidentified
  source 1..9960: mol_type = genomic DNA
atggaaatagcaaaaaaagcagaaatcattgcagtcggctcggagcttctgctcggccagatcgccaatacgaacgcgcagtttatcagtaaagagctggcggaaatcggtgtaaacgtgttttatcataccgctgtcggtgacaatccggagcggcttaagcaagtcatccgcattgccgaagagcgctcagacctgattattttttcaggcggcctcggaccgaccaaggacgatttgacgaaagaaaccattgcaaacacactggggcgcccgctcgttctgaatgacgaagcgttccggtcgatcgaagaatatttcgcgaaaacaaaacggaccatgtcgccgaataaccgcaaacaggcgctcgtcattgagggctcagacgttttggcgaatcatttcggcatggcgccgggaatgcttgcagagcacggctcaaggctttatatgctgcttcccgggccgccgagcgagctgcgtccgatgtttgaaaatgaagcgaagccgctgcttcttaaaaagctcggttctaatgagaaaatcgtttcaaccgtgctgcggtttttcggaatcggtgaatcccagcttgaagctgatttagaagatatcattgatgcgcagacgaacccgacgattgcgccgcttgccgctgacggagaggtcacgctgcgtttaacggcgaaacatgccgacgaaaaagaaacagaacggcttttaaaagaaacggaagccgccattttagaacgtgtcggtgaatttttctacggatatgatgatacgtctctggtgaaggagctttcaaaagcatgcagacaaaacggaattacgatttcctctgccgaaagctttacaggcggcctgttttcagagtgggtgactgatttaagcggtgcgtctcaactatttgcagggggcgttgtctgttattccgacagcgtgaaacagcatgtgctcggcgtgaaagctgagacgctcgcagaaagcggagcggtcagcaaagagtgtgcgaaagagcttgcagccggtgtgagaaagctgaccggaagcgatatcggcatcagctttaccggggttgcgggtcccgacccgcaggaagggcatgcgccgggccgcgtctttatcggcatttcggcggaaggaaaagaagaggtgcacgaatttaactttgccggctcaagaaccggaatcagaaagcgcgccgctaaatacggctaccatctcattttgaaaatgctggatcaaaaataaagttttaaagaggcgttttctctgatttttaagttttacaagggaaattatgctcttttgcagaaaagcgaacgcggaaaaaatccgaatatgcgttcgcttttttcttggcaaatgccagtaaacaaggtatagtatatgtagtggcaagataaaggaggaaaaaataaatgagtgatcgtcaggcagccttagatatggctcttaagcaaatagaaaaacaattcggcaaaggttccatcatgaagctcggagaaaaaacggatacaagaatttcaacggtgccaagcggttcccttgcacttgataccgctctcggaataggcggatacccgcgcggacggattattgaagtatacggacctgaaagctcaggtaaaacgactgtagcgcttcacgcaatcgctgaggttcaggaaaaaggcggacaggcagcatttattgatgctgagcatgctcttgatcctgtttacgcgcaaaagctcggtgtcaatatcgaagagcttctgctttctcagccggatacgggagagcaggcgctagagattgctgaagcgctggtgcgaagcggagctgttgatatcgtagtcgttgactctgttgcggcgcttgttccaaaagctgaaattgaaggtgacatgggtgattcacacgtcggtttacaggcgcgtcttatgtctcaggcgctccgtaagctttccggcgccatcaataaatctaaaacaatcgcaatctttattaaccagattcgtgaaaaagtcggcgttatgttcggaaatccggagacgacaccgggcggccgcgcgctgaaattctattcttccgtgcgccttgaagtgcgccgtgccgagcaattaaagcagggcaacgacgtcatggggaataaaacgagaattaaagtcgtaaaaaacaaagtcgctcctccgttccgtacggctgaagtggacattatgtacggtgaaggaatctccaaagaaggggaaatcatcgaccttggaactgaactggatatcgtgcaaaaaagcggttcgtggtattcttatgaagaagaacgcctcggtcagggccgtgaaaacgccaagcagttcttaaaagaaaataaagacatcatgctgatgattcaggaacaaatccgtgaatattacggtttagacaataacggtgtaacggacaaagcggaagaagttcaggaagagatggaattagaagaataaaaacacgatcaaaagagcggaataagcaatttccgctctttttttgtgtgtttcaaaaaaacactggctgtttcggaaaattttatctggaatgttacaacttttttcacccttcactcgtctaacatcatggcgaaagttgaaattatatctgaggaggaaaaaagatgacgagcccccgccgcacaagaataaataaaaaaagaaagaagaagctgatgaaaacgggagggctgtttctttttctgctgcttgttactgtttttattatgatctggaccgtcacagcgcatcatcgggagaaggaatccgttaaaaacgctcccgccgctcaatctgatacttcaaaagaccagactcaacagaaaaaagaaaagacgcaaaaagagaaaacacctaaatcaatcgatcacaataaacagatcagcaattacttgcaacagatcgggttcagcggcacagcaatgatcgtaaaagatggaaaagttgtgctgaaaaaaggctttggctatgcggaccgaaaacaaaaaattaaaaacaatccggagacatcatattatatcggttcttcacaaaaagcgttaatcgcgacggctgtcctgcagcttgaagaaaaagggctgctgcagacgagtgaccccgtcagtacatacttgccgaattttcctaacggcacgcgcattacattgaaaaatctgctcaatcatacgtccggcataaacgggcatattgaggggcagggaccgatttctccaattgatttaatcaaggatatagaataccgcggaattaaacggcagcccggggtttgggattacagggattccaactattcagtgcttgcttatatcgtatctcaagtcagcggggagtcttatgaacaatatataaaagaacacgtgttcaaaccggccggaatgaagcatgccggattttacaaaacatttgccaaagccaaatatcctgccgtcggctacaaagtcgatgagagcggcagtcttgtaacgccgcaccttctggacttatcacagctgtacggggcgggcgacatgtatatgagcgctgaagatctctataaatttgataaagcgatcgtaaacggcacgctcctttcaaaagaaagcttcacaaaaatgtttacagcaggaagcagtgccgcttacggcatggggttttacattgatccgggaagctattccaatcacggtgttatgcccggttacaacattttgaacagcttcagccattcggcaagcaaatatgtcatattactgtcaaacattcagaataacgccaaattaggacaggtgaacaatagaatttaccaattgcttaaccagccggactaacagagccgtcattcacgaaaagcggatctaggtatggtatgatatatacatgggcgaagatccgagctgaaggcgaatcgttaagctggcaagcttgacaagtatttccgacacaattacaatgaagttgtaacatttgtttttttaacatgattgttaaaccatttaaacttgacaagggcatgagagactcattccctgtgaaggttctgtatgttgagaaaatcagacaatgtacatgccggcacttatttaagcaaaaaccaagttcatagcaagaggaggtgaaagtatgaccccaatgacggttctcatctccattttgctgactctgctcggtttagttgttggctactatgttcgtaaaaccattgccgaagcaaagattgcgggcgcacgcggtgctgccgagcaaatccttgaagatgcaaagcgtgatgctgaagcactgaaaaaagaagctctgcttgaagcaaaggacgaaatccatacacttcgaatagatgctgaacaggaagttcgtgaaagacgaaatgagcttcaaaaacaagaaaaccgtttacttcaaaaagaagaaaatcttgaccgtaaacatgaaggaattgataaacgggaagcgatgttggagaagaaagatcattctctgaatgaacgacaacaacatattgaagagatggaaagcaaagtggatgagatgattcgtatgcagcagtcagaactcgaacgcatttcgagcctgacacgggatgaagcgaaacaaatcattcttgaccgggttgaaaatgagctttcacatgacatcgccatcatgacaaaagaaaccgaaaaccgtgcgaaagaagaggcagataaaaaggcgaaaaacattctgtcgcttgctttgcagcgctgtgcagcagatcatgttgccgaaacgacggtatcggtcgtcaatcttccgaatgatgagatgaaaggacgtatcatcggccgtgaagggcgtaacattcgtacgcttgaaacgcttacaggtattgacctgattattgatgacacacctgaagcagtcattctttccggatttgatccgatcagacgggagacggcaagaatcgctcttgataaactcgttcaggatggccgtattcacccggcgcgcattgaagaaatggttgaaaaatcccgccgtgaggtagatgactatatccgggaaatgggtgagcagacgacgtttgaagtcggcgttcacggactccacccggatctgatcaagatcctcggccggttgaaattccgtacaagctacgggcagaacgtactgaaacattctatggaagttgcgtttttagccggtttaatggcttccgagcttggagaagatgcaaagcttgccaagcgggccggtcttctgcatgacatcggaaaagcaattgaccatgaagtggaaggaagccacgtggaaatcggagtggagcttgcgaccaaatataaagagcatccagtcgtcattaacagtattgcgtcacaccacggagacgaagagccgacttccattatcgccgttctggttgctgccgctgatgcactgtcagccgcaagaccgggcgcgagaagtgaaacgctcgaaaactatatccgcagacttgaaaagcttgaagaaatctccgagtcttacgaaggtgttgaaaaatcatttgcgattcaggccggccgtgaagttcgcatcatggtaaagccggactccattaatgatttagaagcccatcgtttggcacgtgatatccgtaaacgaattgaggacgagcttgattatccgggccacattaaagtgaccgtaatcagagaaactcgagccgtagagtatgcaaaataaagtgatgcttagcgcatcactttatttttttgagtccctgaaaaggaaggaatggaatgtatgagaattttatttatcggagatgttgtcggttccccgggccgcgacatggtaaaagaatatgtcccgaaactgaaagcgaaacataaaccgcattttacaattattaacggagaaaatgcagcacacggaaaaggactgacagaaaagatctaccacagcctgatggaagcgggagcagatgccattacaatgggaaaccatacgtgggataaaagagagatcttcgactttattgatgatgtgccgcatcttgtccggccggcgaatttcccggaaggtacgccgggcaaaggcattacctatattaaatcgaacggaaaagagcttgccgtcattaatttacagggacggacgtttcttccgccgcttgatgatccgtttgctaaagcggatgaattaatagaagaagcgaaaaagcgcacgccgtacatttttattgattttcacgccgaagcgacaagcgaaaaaattgcgatcggctggtacacagacggaagagcctctgccgtggtaggcacccatacacacgtgcaaacggcagataaccgcattttgccgaaaggaacggcatatattactgatgtaggcatgacgggtccgtacgacggaattctcggtatggacagagaaacggtcatcaagcggtttaaaaccaatcttcccgtccggtttacggtggcggaaggtaaaactacgctgagcggagtgctcattgacatcgacgatcaaacgaaaaaggccgtaaatatcgaacggatattaatcaatgatgaccatatgttctttgaataggtgatcatgtgtttattttataaaaatattaaaaggaaaagcaggaatatagcaacttcttagtgaatatagtagaaatggaaggtagcccgctatttttgaaaagcattgtggggatggctttttggatagcaacatctaataatgattgttctaatgaacactcacttatccattgtaaaactaagggggagcagaaatggaaatcttaaaagtttcagcaaaatcaagtccaaattcagtagcaggcgcgcttgccggtgtattgagggaacgcggagccgccgaaattcaagcaattggggcgggtgccttaaatcaggctgttaaagcggttgcgattgccagaggctttgtcgcgccaagcggcgtagatttgatttgtattccggcttttacagatatacagattgacggagaagaaagaacggcgattaagttaatcgtcgaaccccgttaaaaacaaaaacatgacattctcaacctgcttgcgtaaggcgagcgggttgtttttattgttgtcatattttctcacaatattatattgatagtacgaagagatgataaatggctttattcatataatattcaaaaaaaatcccgcccatttttccggacggaagcagttgttttctcggagtggacactggtgtttacctcctttttatatcatgttacaatccagttattacagtgttttttcgaggaggagtgtacatgttgggaggaaagatgaaagctatcgtcaaaaatgaaaacgctttcggagcaactctgaaagaaattcccataccttccattaatgagaatgaagttctcattaaagtacaggccgcatctatatgcggtacagatgtgcatatttataattgggatcaatgggcgcagaaacgaatcaaaacacctcaagtattcggacatgaattcagcggtatcgtagcggaaacgggcaagcacgtcacaaatgtcaaaacaggtgactacgtttcggctgaaacccacttcgtctgcggtacttgtgtcccttgtttaacaggaaaacatcatgtgtgcacccatacaaagatcctcggcgtagatacagccggttccttcgctgaatatgtcaaagtgccggctcgaaatgtttggaaaaatcccgctgatatggaccccgccgtcgcttctgttcaagagccgcttggcaacgcggtgcatacggtgctggaaagcagcctgcttgcgggcggttccgctgcaattatcggctgcggaccgatcgggctgatggcggtggcagtggctaaagcggccggtgcatcccgtattgcggccattgataaaaatgattacaggctggctcttgcgaagaaattgggagccacgatcaccatttcagctgataaggaagatccgcttgaggtgatcgacaccttaacaggcggagaagggatcgaccttgtctgcgaaatgtcaggccacccggcagctatagcacaaggattaaagatggcggccaacgggggaaggttccatatgctgagtctgccggaacgtccggtgacggttgacctgacaaatgacgtggtgtttaaaggacttaccgtccaaggcatcacaggcagaaaaatgtttgaaacatggcggcaggtatcgcacctgcttgaatccggccttgttgatctgacgccggtgatcacgcatcagtttccgcttgaagactttgaaaaagggtttgacctgatgagaaagggccagtgcggcaaagtcattttgattccataaggggagggttctataatgaaagaatttgaatttctaaaagcagagcttgataaaatgaaagaaaataaaacgtggcagcaaatgaaacaaattgaaacgaagcagggtccgtcggtggaagtcaaaggggaaaacgtgatacagctttcatcgaacaattatctcggtttgacgtcgcatccaaggcttgtcgaagcggccaaacgcgccgcggaagaattcggagcgggtacggggtccgtccggacaatcgccggaacattcacaatgcacaatgagctggagaaaaaattggcaaactttaaaaaaacagaagcggcacttgtttttcaatcggggttcacaacgaatcagggcgtcttatcaagcattcttacaaaagaagatatcgtgatctctgacgaactgaaccatgcttccattattgacgggatcaggctgacgaaagcagataaaaaggtgtaccgccatgttgatatggacgatttggaaagggtgctgaaaaaatcaatgaattaccgcatgcgcctgatcgtcaccgacggcgtattctcaatggacggcaacatcgctcctttgcctgacatcgtcaaacttgcggaggcgtacgatgcctttgtgatggtcgacgacgcgcatgcttccggcgtactcggaaaaaacggaaggggaacggttaatcacttcgggcttgacggaagagtccatatacaggtgggcacattaagcaaagccatcggagtgcttggcggttacgccgcagggtcgcaggttctgatcgattacctgcgccataaagggcgccctttcttattcagcacttctcatccgcctgcggtaacggccgcatgcatagaagccattgacgtcctgctggaagagcctgagcatatggaaaagctgtgggagaacacggcgtatttcaaagataaactggttcaaatggggctgaccctcacaaaaagcgaaacaccgatcgtgccgatattaataggggaggaagaaaaagcccaggcattatcagaccttttattgacccgcggcgtctttgcacaaagcatcgtgtatccgacggtcgcccaaggaaaagcgagaatccgcaccattattacggcggagcatacaaaagaggagcttgaccgggccctggaagtaatccgttcagccgcaaaagagtttcagcttgtgtaagggggaaatgattccctcttttttatttcttattgattcttattctcaataaggtgtacaataataggctggcggttgtgccaaacgcttttaaccagtatacttgagggtggaagtttcggttgaaaggagacattcgaacatgaatgaaaagcaaaagcaggaaagcggacaagtgaatccagcggacaaaaaatccgaaaaggattacagcaagtattttgaagccgtatatattccgccttctttaaaagatgcgaaaaaacggggcaaagaatccgttcaatatcataacgattttaaaatctcagaagaatttaaaggtctcggtgaaggcaggaaattttatatccgcacgtacggatgccaaatgaacgagcatgatacggaagtcatggcgggtatttttatggcgctcggttatgaagcgacaaattcggttgatgacgcgaacgttattttattaaacacttgcgctatccgg

SEQ ID NO: 18 - DNA, 9960 bp, unidentified
  source 1..9960: mol_type = genomic DNA
gaaaatgctgaaaacaaagtgttcggagagctcggccatttaaaggcgctcaaaaaaaataacccggacttgattctcggcgtctgcggctgcatgtcacaggaagaatcagtcgtaaaccgcatccttaagaagcacccgtttgtcgatatgattttcggcacgcacaacattcaccgtctgccggaactgttatcagaggcatatttatcaaaagagatggtcattgaagtgtggtcgaaggaaggggatgtcattgaaaaccttccgaaagtccgcaacggaaaaattaaaggctgggtcaacattatgtacggctgtgataagttctgcacatactgcatcgtaccgtatacccgcggaaaagaacgaagccgccgtcctgaggaaattatccaggaagtcagaagacttgcaagcgaaggttataaagaaatcacgctgctcggtcaaaacgtgaatgcgtacggaaaagattttgaggatatgacgtacggactcggtgatttaatggacgaactgcgcaaaattgatattccgagaatccgattcacgacgagtcatccgcgtgactttgatgatcatctgatcgaagtgctcgccaaaggcggcaaccttcttgaccatatccatcttccggttcaatcaggaagctcagcgatgttaaaaatgatggcgcgcaaatatgacagagaacgctatctggagcttgtcggaaaaattaaagcggcgatgccgaacgcttcactgacgactgatattattgtcggttttccgaatgaaaccgatgaacaatttgaagaaacgctttcgttatatcgcgaagtggaatttgacagcgcatatacgtttatttactctcctcgtgaaggcacgccggcagcgaagatgaaagacaatgttccgatgagggtcaagaaagaacgtcttcagcgcctcaatgatttggtgaaagaaatttcagccaaaaaaatgaaggaatatgaaggcaggaccgtcgaagtattagttgagggagaaagcaaaaataaccctgacattcttgccggctatacggaaaaaagcaagctcgtcaacttcaaagggccgaaagacgccatcggcaagattgtacgcgtcaaaatcgaacaggcgaaaacatggtcgcttgacggtgtgatggcaggagaagcaatcgaggtgaaataataatgacgctttactcaaagaaagacattgtacagcaggctaggaaccttgcgaaaatgatttctgaaacagaagaagttgattttttcaaacgcgccgaggcgcagattaacgaaaatgaaaaagtctctgcgattattaatcagattaaaaccctgcaaaagcaagccgttaacctgaagcactacgaaaagcttgaagcgctgaaacaagtggaaagtaaaattgacgccctgcaggaagagcttgaagggattccgatcattcaggaattcagagattcccagattgaggtcaatgaccttcttcagcttgtcgcacatacgatctcaaatcaggtgacaaacgaaatcattacatctaccggaggcaacctgctgacgggagaaaccggttcaaaagtaaaacattcgaataacagctgttctatttaaacacggggccttcatcggcccgtgttttttttgcttttgagaactaattgtaatatgtactaaattttcattaactcaccgttttgtcccattttactggaaagttttatcatacttaaaagtaaagtttctgggcagggctgcatacaatgaaacagaaactttgtatttttatatttttattataaaaatgcacatgagacaaatgcccagcataagataacacgaagaagaacaaggaggcctgccggaatgtctgaatacagggaaattattacgaaagcggtagttgcgaaaggaagaaaattcacccaatgcaccaacacgatctcacctggagaaaaaccgtcaagtatattgggcggctggattatcaaccacaagtacgatgccgaaaagatcgggaagactgtagaaattgaagggtattatgatataaacgtctggtactcttacgccgataatacaaaaacggaagttgtaaccgagagagtaaagtatgtcgatgtgattaaactcagatacagagacacaaattacttagatgacgaacacgaagtcattgccaaagtgctgcagcagccgaattgtcttgaagtcaccatttctcctaacggaaataaaatcgtggttcaggcagagagagaatttttagcagaagtggtcggtgaaacgaaagtcgtcgtcgaagtcaatcctgactgggaagaagacgatgatgatgattgggaagacgatcttgatgaagaattagaggacattaaccctgaatttttagtcggagatcctgaagaataagttgaggctaggggagtttaacaccccaagtctcttttttatatataaagatatcactcttttgtttatgttataatgggattcggataagagaagatacatagtgagggattaaatgatggctagttacacgcctatgatacagcaatacttgaagataaaggcagagcaccaagatgcctttttattttttcggcttggtgacttttatgaaatgttttttgaagatgcaaaaaaagcgtcacaagagcttgaaattacactgacaagcagagacggaggttcggctgataaaattccgatgtgcggcgtgccgtatcattccgcggcggcttatattgaacagctgattaaaaagggctataaagtggcgatctgtgagcagacggaagatccgaaggcggctaaaggcgttgtaaaaagagaagtcgtccagctgatcacaccgggaaccgtgatggatggaaaaggcattcatgaatcggaaaacaactttatcgcttccgtttccgaattccgtgacggatacggtctcgctttatcggacttaacgactggagaaaacctggccgtatttattgagcggattgaagatgtcatgtcagaaatttactcagtcggcgcaaaggaaattgtcgtatcaagcaagtttaacgaacatacagctgctcagctgaaagaacggtgcggcgccacgatttccatcgaagacggggaaataacagaacgcattgaaatcgcaaatcacttgccgggcgaagagctgacagagacgtttatgcgtttatatacgtatttgcagaaaacgcaaaaacggagcctggatcatctgcagccggttcaagtatatgaattggaagaagcgatgaaaattgacctgtattcaaaacggaatcttgagctgacggaaacgatccgttcgaaaagcaaaaaagggtcattgctttggcttttagacgagacgaaaacagcgatgggcggccggcttctcaaacaatggattgaccgtccgctgatcagggcatcccaaatcgaagaacgccaggaaatggtcgagacgctgatcaatcatctgtttgagcgtgaagatctgcgcgagcgcctgaaggaagtatacgatttagaacgtctggcgggccgggtcgcattcggaaacgtcaacgcaagagatttaatccagctgaaagaatctttaaaacaagtgccgagcattaaagagcttgtagggtctcttaaccataaaaaagcaaaagaacgcgccggcttgattgatccgtgcggagatttgcttgacctgttagaagaagcccttcatgaaaacccgccgttatcgttaaaagaaggcaacttaattaaagacggctatcatcagaagctcgatgagtaccgtgacgccagcaaaaacggtaaagactggattgccagactggagcagcaggaacgcgcttatacgggcatccgttcattaaaggtcggtttcaataaagtgttcggctattatattgaagtcacaaaagcaaaccttcatctgcttgaggacgggcggtatgagcggaagcagaccctcacaaacgccgaacgttacattacgccggaattgaaggaaaaagaagcgcttattttagaagcggaaaacaacatctgcgagctggaatatgagctttttgccggactgcgtgaaaaggtgaaacaatttattccgcgtctgcagcgtcttgccaaacaaatgagtgagctggatgcgctgcaatgctttgctacgatcagtgaaaacaggcattacacaaagcccgtcttttctgataatgaagtaaaagtcattgaagggagacaccccgtcgttgaaaaggtgatggacagtcaagaatacgtgccgaacaactgcctgatgggcgattcgcgggaaatgctcctgattacgggtccgaatatgtcaggtaaaagcacgtacatgagacagatcgcacttctatcgatcatggctcagatcggctgttttgtgcccgcaaaagaagcggttctgccgatttttgatcaaatcttcacgagaatcggggcggcggatgatttgatttccggacaaagcacctttatggtggaaatgctggaagcgaaaaacgcgattgtgaacgccacaaaagacagcctgatcctgtttgatgaaatcggccgcgggacatcaacgtacgacggaatggcgctggcacaggcgattattgaatatgttcatgatcatatcggcgctaaaacactgttcagcacgcattatcatgagctgacggtgctagaagataagcttcctcagctgaaaaacgttcatgtgcgcgcagaggaatataacggaacggtcgtgtttctccatcaaatcaaagaaggagccgcagataaaagttacggcattcatgtcgcccagcttgcggagctcccggacgatctgatcagccgcgcgcaggagattttgaaacagcttgagcagacgggagataaaccggagcttccggcggcacctgagaaaaagcccgccgtcaaagaagaaccggcacagctgtccttttttgctgacggtgaaaaagaacagaagacaccggctgtttctaataaagaaaaacaagtgcttgaagcttttaagtcaatcaatattctcgatatgacgccgcttgaggcgatgaatgaaatgtacaagctgcaaaaaaaattaaaatagaaagaggtgacggacttggcaaaagtcatccagctgtcagatgaactctcaaataaaatagcggccggcgaggtggtcgaacggcctgcctcagtcgttaaagaattggtggaaaacgccattgacgcaaacagcacagtcattgaaatcgacatagaggaagcggggctttcgtcgattcgggtgctcgataacggcgaagggatggaaaccgatgattgcaagcgggctttccgccgccacgcgaccagcaaaatcaaagatgaaaacgacttgttccgggtgagaacactcggtttccgcggagaagctttgccaagtatcgcgtctgtttctcatctggaaatcaaaacgagcatcggcgagggagcgggaacccatctcattcttcagggcgggaatatcatttctgaacagaaatcctccagcagaaaaggaacggagattatcgttaccaacctgtttttcaatacacccgccagattgaaatatatgaaaaccgtgcatacggagctcggcaatattacggatgtcgtcaaccggattgctcttgcgcatccggaagtatcgatccgtctccgccatcaggggaaaaatctgctgcagacaaacggtaacggggacgtgcgccatgtgctggccgcaatttacgggacggcggttgcgaaaaaaatgataccgctccatgtcagctcgcttgattttgaagtcaagggctacattgcgcttccggaaatcacgcgtgcatccagaaactacatgtcatctgtcattaacggcagatacattaagaatttcccgcttgtaaaagcggtgcatgagggctatcacacacttctcccgatcggccgccatccgattacgtttatcgaaattacgatggacccgattttggtcgatgtcaatgtccatccgtcgaagcttgaagtccgcctcagcaaagagacggagctgcatgagctgatccgtgacgggataaaggaagtgtttcaaaagcagcggcttattccgagcgcccagctaccgaaaaaatcagcgccggcaccggccaaaaacgaacagcaatttatgacattcgaggaaagcgcgcctgagcggaaacttcctgaaaaaacgcctgagccgtcttactcaccgatgaagctgagttcggtcgtaaaagagcctgagccggtcatagaagaagagtccctcccggatgaagcaggtcacggagtgccgccgaagatcggcatgccggtaacggaagcgcctgtttccgatattccggagcctgaagtggaggctgactctgaagaaatacgggaagaaagagttccggtgatgtatccgatcggccaaatgcacgggacatatattttagcccaaaacgaaaacggcttgtatatcattgaccagcatgcggcgcaagaacggattaaatacgaatatttccgggaaaaagtcgggcaggttgagcctgaagtgcaggatatgatcgtgccgctcacctttcattatgcggcaaatgaagcgctgatcattgaccagcatcagcatgagcttgaaagcgtcggagtatttttagaagcattcggcacgaacagctacatcgtccgctgccatccggcctggtttccaaaaggggaagaagctgaactgattgaagaaatcattcagcaggtgcttgacgccaaacagatcgatattaagaaactgcgggaggaagcggccattatgatgagctgcaaaggctcaatcaaagcgaaccgccatttgcgcaacgacgaaatcaaagcgctcttggacgacctgcgccgaacgacagacccgttcacatgcccgcacggccggccgatcattatccatcacagtacgtatgagatggagaaaatgtttaagcgtgtgatgtaataacaacgtttatgtgagactaaaccgttatttacccacaagcagccttactagtttgaatgacccagcctttcagagataacttttgagttttttaatttctggcataattgttgcatggtataccgtaaatcatgaagataatttgtttaacatccagctgtttttctgtcatataaaaattgtttatgggtagtaattggggttggcgatgtagtatcctgtgtttggtgtaaccgtctatatcgtcaattgactgcattaatagtgaaataatggagaaaacgattcctgaaaaatctcatgctatttgtgaagaatttaaataaaattggaatttggcgggttatactatccgcaagttagtcaagggggactagtaattagcccccttatatttagttcttcctgcatttttttctatttttgattaaactgaaaaccactaagagaaaatacaccgacagcaacaatataaaatgaaatttccgctgcggcggtgttaatattcttaaggacaaggataagccctattaaaattataggaatgatgtaagttagaattcttatagcttttgggctcggttcagcattacttttatcttccatagttacctccgatcttaaacactttagaaaataatcactttaaagcttaacacaggataaatttgttaaacaccaacaatagccccgtttaaaggaaaagcataagagttttagtaatccacaaagttgctgccgggttgccagttacttttagagcacccgcatagcacgcatcttcaactgcaccagtaaaattgccaatgatattaagaattatttcaaaaccgccgattccacccatagcagctttaactttagtaggtattgcgtcccgagctgctctcagtgctttatagctcaggacaaatcaccttgagcactaaactccgacccatatcacatagtctatttgtctaaatcttaatatttactttttacacttaatgattttatgttagttttgtttttgcacagtaaaaagataactttatttagccctaacaaaaacatctgagctccttgtcaataatcaaatataaatcgtcttcaactgacgtcttttttcatgcgcgacgctttcgtgaattgttgcagctagacttgtttttgaacggttttctcgcaaaagaaaagagtgtaaattaagccgtacttttctcagttcaattatgttaatgtcaacataacgttgaattcggtgtaagaactaaggtattctgatatatttcgagagcccgtgtggtttagtttatctgaagaaaaggattgaaataataatcttaattgcgggtcatttctaaaaactgattatctcctccggccgcttctgtaattgcttctcaaaacaaaagctcaatttgttttctttttatccgaaactcatattcatccttcattttctgaataaaattccctaaagaattcatctgaattaatattctgatttccttatatatcagcatagcaatttgtaaaaaaggcccggactggtgcttttggcgggagcagaaagttttttaaactttaccggaaaatccatggaaaaacgtggtgccatctggtaaagtggttacagtcggctgggcggcggcagcagtctgcccggatcttttaaatgggaatggtgacaatggatcatacatatgaagtgcatcaaatcaaaacttatcaccagatgtggtccaactattgttatattatcgcagaccgctcaaaaaaatctgcgatagcggtggacccgtcctgggaaatcgataaaataaccgataaactgcatgaattggatgttgatttatcagctattttattaacacattcgcattatgaccatgtaaacttggcggagccgcttcagcaaatctatcattctgatatatacatgtcgtcggcggaaatcgatttctatcaattccggtgcagaaacttaatagcgcttgaagacggacaaacctttgcggccggagggtttatcatcagaagtatattgaccccgggacatacggccgggggaatgtgctatttgctatctgatcacctttttaccggtgacaccgtgtttacagaagggtgcggtatatgtgaggacagggggagctcagctgaagatatgtttcacagtatacagaggatcaaagcctctattccgccgcatgtgcgtgtgtatccagggcactccttcggggagaagccgggtcaaaaaatggagagtcttttgaaaaacaatatttactttcagattgaaaaaaaagaacatttcgtgaatttccgcaacagaaaaaatcaaaaagggctgtttcattttaagtagcgtataatttggttctatctaactagtttttcttttaggccacggaacttctgaattgaaatgatagtcattgctcgcttttgggtgatgaatggctgagtaacggtagtttgcggataaaagcgccgggcaggccgaatgggacattctgcttatgtctgttccaaaagccctgagtccgccttccgtcagggaaaaataggcattattcaggattccgcataggataaattaacttgacatggtatttttgtgaagttactatcaacttgaacatgcttcttaactgaatccgagaaaggatggctgagaaaatgatcacatacatattccccggacaaggttctcaaaagcagggaatgggaagctcattgtttgatgaatttaaagacttaacggagcaggctgatgaaacactcggatattccatgaaacggctttgtcttgagaatccgtattccaaccttcacaagacgcagttcacccagcctgctttatatgttgtaaacgcgttaagctatttgaaaaaaatccaagacaatgacataaagccggattatttggccggccacagtttgggagaatat

SEQ ID NO: 19 - DNA, 9960 bp, unidentified
  source 1..9960: mol_type = genomic DNA
aatgcgctgtttgcagcaggtgcttttgattttattaccggcctgcaacttgtccgtaaaagaggcgaattaatgagtatggccactgacggaaaaatggccgccgttatggggttaaccgcagctcaagtgtcagacgcccttcaaacgaacggattacatacgattgatattgccaatatgaattctcctcaccaagttgtcatatccggacgcaaagaagacattgaaagagccaaaagcgtattcgaaggattaaaagacgttacgatgtttcatccgttaaacgtcagcggcgcttttcactcgagatatatgagcgaagcaaaacaagagttcgaaaaatttttacagtcatttcatttttctgctatatcaattcctgtcatttctaatgtgcatgccagaccttatgagcaagacggaatccactccgttctggccgatcagatcgaccattcagtgcgatggaacgacagcatccgctatctgctggacaaagggcggatggaatttgaagaagtcggcccaggccatgtgctgacaggacttattcaccgcatcaaaaaagaaactgaagcttcacccgccatgtagcggaatgcaagtcaaaattcttatctcacacaatagtgccagccatacaacatcacgaccacttcacctgcacacgacatcacgaaaaactcacaaatcaatattttgaaaaaataaagagcaaaaaggagaacaaatgaaccagccaattgttttcatgttttctggacagggttcccaatactatcaaatggggaaagagctgtttgcacataacgccgcttttcggcagaaaatgcttgacttggacgattttgcagtcagccgtttcggttactctgtccttaaagagatgtatcatacaggcaatcgtctgtctgatccatttgaccgccttcttttcagccatcctgccatttttatggctgagtacgcacttgcatatgctttggagcaaagaggcatacgtcccgattatgtgatcggagcaagcctcggagagtatgcagcggccgctgtgtcgggggtgctgagcgccgaggatgctttagattgcgtgctggaacaggcccgaatcgttacggaaacttgccgtaacggcagcatgcttgcgatactgggtgatccggctttatatcaagatgatccgctgctcggcgaacatagtgaattggcatctgtaaactatcattctcactttgtcatctcgggagagcgggaacatatcaaaaagatcatggacgatctgagagaaaagcagattcctcatcagctattgccggtatcttacgggtttcactcagcgctggttgatcaggcggaacagccgtacaagcgctttttagctcagaaatcaatccggactccgtttataccgtacatctccagtgcgaccggaggggctgagaccgatatacaagcggactttttctgggatatcgtaagaaagccgattcgattccgcgaagcgcttcaattcgcagacagccgccaaaaaggattgtatattgatgccgggccttccggaacattggccgcatttgccaaacagattcttcctgcaggatcggctgaacgcatccgtgccattatgacgccgtttcataaagaacaaacacatttgcagcaaatagaagacagcattttatcaccgcccggaaggagactgtaagatgatcagctttgtttttccaggccaaggctcgcaaagaatagggatgggggaggacctgttcggccgctatcctgaattaaccgcaaaggccgatcacattctcggatattccattcaggagctgtgccgggacggagaacgtctcaatcagactcaattcactcagccggcgctctatgtcgtcaatgccctcagttatttgaaaaaaacagaagaaaccggcttgaaaccggattttaccgccggacacagtttgggcgaatataacgcgttatacgcttcgggcgcctttgattttgaagaaggcttacagttggtcaaaaaaaggggcgaactgatgagccacgccaaaggaggcggcatggcagcggtgatcggcctgactcatgagcaggtgacggatgttttaagagaaagtcacttggatatgattgatatcgccaacatgaacacaccgcagcaaattgtcatttccggatataaagaagatattgaaaaagcagcatccgtttttgaagccgtaaacggcgtaaaaatggttcaccgcttaaatgtcagcggcgcctttcactccagatacatgctggaagcgaaggaagagttttcacggtttattgaatctttccattttaagccgctgtccatcccggtcatttcaaatgtgacggcaagaccctatgagcagagagagctgaaagagacactcgccggacaaataacgggatcggtgaactggacagacagcatccgttacttaatgggaagaaagaacatgagctttgaggaaatcggtcccggaaaagtgctgacggggctgatacaaagaattacggctgaagcggagccgataacagatgaaatcaaagtcccggctgaggctggaaaaagctcaattacggcagcgtctttaggaaatgaagagtttaagcgcgattatcaattgaaatacgcctacttggccggcggcatgtaccgcggaatttcttcgaaggaaatggttgtcaaactggctgaaaaaggaatgatgggctttttcggaacgggcggtttgaacatcgcgcacgttgaggacgccattttgtcaattcagcatgagcttcgtgacggcggttccttcggcataaatgttgtacataatatgaagcatacagattcagaagaaaaaatgattgatcttttactgaagcatgggattcagaacttggaagcatcagcgtttttaacggttactcccgctttagtaaggtttcgcgctaaggggttaaaacgcggtgccggcggacaaataatagcgcgtcagcggattatcgccaagctgtcccgtccggaagtagcggaagcgtttctcagcccggcgcctgatcacattctgcagaaacttgcagccgaaaacaaaattaccgctgaagaagcaagtctgatgagagaaatacccgttgcgcatgacatctgcgtagaggctgattcaggcggccacactgacggcggagttgcttacagtctcatgccggcgatcatccgtttacgagatgacatgatgaaaaaataccgatacggcaaaaccgtccgcatcggagccgcagggggaatcggaacgcccgaggctgccatggctgcatttatgctcggcgctgattttattgtgaccggatctattaaccaatgcaccgttgaggcagcgacgagcggcctcgtaaaggacctcctgcagcagatgaatgtgcaggacacggcctacgcgccggccggcgatatgtttgaatcaggcagcaaggtgcaggtattaaagaaagggctgttctttccgacccgcgcctctaagctgcatgaactgtatcagcgtcaccgctcaattgaagaaattgatgaaaagacactcagacaaatagaagagaagtattttaaggcaagtgtaagcagtatttatgacaaagtgaaggctcattattcgaatgaagacatttcaaaagccgaacggaatccgaaacagaaaatggcgctcatattcaaatggtatttcaggcaaagttctgcttcggcgattaaaggagacccggatgccaaggtagacttccagattcattgcggcccggcattaggcgcctttaatcaatgggtgaaaggtacggagctggagtcctggaaaaaccgccatgccgatggtatcggcatgcggcttatggaagagacggccacgcttttgaatcagaagttaggttcatttttgcaaacgtgttgacaagacgaaatgatagattgaaaacatggaatattcataaaagatgaaacagggaggaactgacaatgagtaaagaaagaatctttgaattgctgcagaacacaatctacgaggttttacctgatttagaaggcgaacagctttcacaggaagaccggttagttgatctgggggcggattcagtggaccgcgccgaaattatcacgatgatgcttgaagaattatcactgcagatcccgcgtgttgagctgacgggagtgtcaaacatcggcgaattggcggaaaagctttatgaaaaggtacaggctgtctgataaaacggtggaaaaatcggaaactcaaaattcatattggaggcgaagttcatggcggctgccggaatagaagcgataaatgtgtttggcgggacagcataccttgatgtcatgcagctggcggagtacagaaacttagaccctgccagatttgagaatttactgatgaaagaaaaagcggtggcgcttccgtacgaagaccctgttacattcggcgttaacgcggcaaagccgattatcgaccgattgacagaggctgaaaaagacagaatcgagctgctgattacctgctcggagtccggcattgacttcggaaaatcactcagtacatatattcatgattacttaggcctcaaccggaactgccgtttatttgaactgaaacaagcctgttattcaggaacggcgggtttgcagatggcgcttaactttattttgtcgcagacatcccccggagcgaaggcgcttgtcgtcgcgacggatatttcccggtttttaattgccgaaggcggcgatgcgttaagcgaggattggtcttacgcagaaccgagcgccggagcgggtgccgtcgccttattgatcggtgaaaatccaattgtatttcaggcggatgcgggcgctaacggctactacggctatgaggtgatggatacatgccgtccgatcccggacagtgaagccggtgacgccgatttgtcattgatgtcttacctcgattgctgtgaacagacatttcgcgagtacaaaaaccgtgtgccgggcgctgactacaaagagacgtttcactatcttgccttccatacaccgtttggcggcatggtcaaaggggcccaccggacgatgatgcgcaagcttgcaaaagcgaaaaacgctgaaatcgaacaggattttcaaaccagagtagagccggggctccgctattgccaaagagtcggaaatattatgggagcagcctctttactggctcttgcaagcacgattgaccaagggggattcgacacttctaagcggatcggctgtttttcctacggctccggatgctgttcagaattttacagcggcgttgtgacgcctgaagggcaagcgcggcagcattctttcaatatagaagagcatctgaacaacagatacaggctgtccatggaagagtatgaagaactgtttaaaggcagcggtctggtgaagttcggaacccggaatgtcaaattggatattaatatgattccgaacattttagaccatcatgccgggagccctcgtttatatttggaagaaatcacggaatttcatcggaaatacaggtggatatcgtgacctatgaaacgataaacgtccgttttcagcaaaatgtctgcttcatcacctttgatcggcctgaagccaacaatacgattaatgagcgtttagtggaggaatgcctgcatattctttctgaatgtgaagactccgtgacctccgtagtgcttgaagggcagccggaggtattttgtttcggggcggattttgatgagatgtaccggaaaatgaggcagggaatgcagcatgcggaatcgcaggagccgctttaccgtttgtggctggcattaaaatccgggccgtacatcacaatcgctcgcgtccgcggacaagtaaatgcgggaggtgtcggttttgccgccgcgtgcgatatcgtcattgcagaggagaatgcttcgttcagtttatctgaactgttattcggactttatccggcctgcgttctgccgtttttaattcaaagaatcggatttcaaaaggcgcattatatgacgattatgacaaagcccgtgccggcacgccaggcgcttgaatggggactttgtgacgtatgcgtcccggactcagcaacagagctgagaaagcatttgctgcgtcttcgccctctgtcgaaaaaagcagtcatccgctataaacaatacatggattccttaaacgaccttgttcatcgttcgaaatccattgctctagccgaaaatcaagcgatgttttcagatccggataaccaatcagccatcttccgttatgcagaaacggggcggcttccttgggaaacggagaacagttaggaggatatcatgccgaacgccgcagttgaactgctcgaaattgaaccggatattatccaagtgacaatgcaggataaagcccataaaaacgccttctccaaagaactgacggatggattgatggaagcattcacccgtataaaaaaatcgtcagtctataaagctgtgattttaacaggatacggccattactttgcttccggaggcacacaggaaggcctgctcaggattcagcagggcatcacgaagtttacggaagaccgcatgtactctttggcgctggaatgtgatattcccgtcatttccgccatgcagggccacggaatcggaggcgggtttgtcatggggcttttcagtgattttgtcattttaagcaaagaaagcgtgtataccgcaaactttatgaaatacggtttcacccccggaatgggcgctacctttatcgttccggagaagcttggggcaagcctcggccatgaaatgctgttaaacggcggccactaccgcggcggtgacctggagaaaagaggggtgccttttccggtgcttcctcggcaggaagtactagactatgcgcaggaattggcccgtgagcttgcggaaaaaccgagaaactctttaattacattgaaaaggcacttggtgacgccgtttcgcgagcagcttccggccgtcattgagaaggagttaatcatgcacgatgagacgtttcatgatgatgaagtgaaaaaacgaatcacgacgatgtacggagtataagacatacatcttacatacagatgaaatggaggcagctctatgaaaagtgatatgaaggcaagcgggttactcaagccatctgtcagctatggaaaatcattgcggctttcagcggatcagccgatgacgattcctgaagtcctgcacaaaaccgccgcggcagcgggagatcaaaagggcatcacatatatacagcctgacggcaccaaagtgtatcagacttacagcggcctgaaaaagatcgccctttcgattgtgaaagggctccgccaatcaggggtgaaggctcaggatgaagtcatattacagctcagtgacaattctcagctgattcctgctttttggggctgtgtttttttaggagcgatcccggttccgcttgcggccgctccggcttatacagaaatgaacagcgggacgcaaaaattgaaagacgcctggacgcttttgaatcagccgtatgtcatcacaagccgtgacgtgctgccggagatgactgaatgggcggaggagcaggagctttccgggttttgcgctttggtggcagaagatctcagtgcgcatgaaatggatacggactgtcatcatccccgtccggaagatttggcgatgctgcttttgacctcgggaagcacgggcacgccgaaagccgtgatgctcagccatgaaaacatcgtctgcatggtcaaaggaaatattcagatgcagggctatacaagtgaggatgtcacgtttaactggatgccttttgaccatgtcggcggcatcggaatgcttcatctgcgtgatgtttatctcggatgtgaggaaatcaacataccgagcgaatccattttgatggatcctttaaaatggctcgacctgattgatcattaccgcgcttcggtcacgtgggcccccaattttgcctttgggctgcttgccgatttcgcagaagagattcaaacacggaaatgggacttatcttccatgcgttacatgctgaacggcggggaggccgcggttgcgaaagtcggacgcagaattatggagcttttagagccgcacggtctgccggctaatgccatccgtcccgcatggggaatgtccgaaacgtcttcgggcgttattttctcagatgagtttacactggaaaacacgagtgacgacgatcgtttcgtcgagatcggtctgccgatcccggggtttaatatgagaattacggatgaccgcaatcaagtggtggaagaaggagaaatcggccgcttccaagtgtccggtctgactgtaacgagcggatattatgagcgcccggacttaaatgagagtgtctttacagaagacggctggtttgaaacgggagatctcggatttctgcgtgaaggccgcctgacgataacggggcgcacgaaagatgccatcattatcaacggtgtgaattattacagccacgccattgaatcagcggttgaggaattgccggaaattgaaacgtcatatacagcggcatgcgccgtccggccgaatcaaagcacgacagatgagctggcgattttctttgtgacatccattccgttggatgagaaccgcatgacgaagcttcttcatcacattcaccagcacgtgacacagagaatcggcgttacccctgattatctgcttccggtcgcaaaagaagacattcctaagacggcgatcgggaaaatccagcgtactcagctgaaacattcattcgaacaagggcaatttgattcattacataataacagacaagaaggcggcaattcagacacggcttctgcagagaaaaaaattgaacgggattttatccgttttctgaaagaggaactttccatcgcggatgagcttgttgatccaaatacaccgtttcaaagtcttggcgtcaactccatcaagatgatgaaactcgccagatcaattgaaaaaacgtatcacatccggctgacggcaagagagctgcacaaaaacccgacgattggcgctttagcggcttatactgctgaaaaagcggcaaacatgtcagcggagcatcattcccaaaaagccgaaccgccggcagaacgggagaaacggcaaacggcgccggcgctttcagaagtccaaaaagggctgtggacattgcagaaaatgtcgcctgaaacgactgcataccatgtcccgctctgtttccgattcacaaccggaatcgacaaagagaaactgaaacaggcgtttcagctcgtattaaagcagcatccgatgctcaaaagcgccgtgaatgaaacagacggcgggttttatttcagagaatcggcagatccttttgtattctcggaggaagatatttctgacgtcaatgaatcacaaatctccgctctgatacggaaaaaagtgaaagagccgtttgtcaaagaaggcgggcctttgctgcgcgtgcagctgctttcaaaatccgccgcggaacattatcttttgacggtgattcaccacatcgtgtttgacggtatttcatccattacatttattcacagtctgctggatcattatcaggcgctgctggagggaaaagaagagtttgcggccgccgttcccggcattcatcccgatttcgcggcttgggaaaagcggtatttagcgggtgaagaaagcaaagctgcccgcgcttactggatgaaacagctcgggggcgatctgcctgaccttcagcttccgtcgatgcgcacggacagcagcgttccggagtttacagaagatacgctgacacgccgtctttccgaacggctgctgaataatctaacggcgtttgccgcatcccgttcgattcatttgtctaccgttatgctgagctgctatatggtgcttttaagcaaatat

SEQ ID NO: 20 - DNA, 9960 bp, unidentified
  source 1..9960: mol_type = genomic DNA
acggataaggaagatattgtgacaggcatgcctgcaatggtcagacctgaagaacgctttgacggagtggtcggccatttcctcaacatgcttcctatacggagcagagccggacgaaaagaaacatttgctgactttgttcaaaaggtgcaggatgccgttctggacggactggatcactccttttatccgtttccgaaaatggtgcgtgatgtgcatgcgaaacccctgcagaacggttcgcctgttttcagcaatgcctttttctatcagaactttctgcagcattcaagctatcaatcgatgctggatgaatacaaggaattctcctgtgagtttgtaaaagacattcatcaggagggggaatatgatcttgtatttgaaatgtgggaagaagcatcaggcatggatcttacgattaaatacagcaccgaattattcgacgaagcaggggcggccagacttttcagccaatttgtacaggccgctgaagctttgacggcgaatccggatatgccgcttgagaacgtttctctcatgacaaaaagagaggagcacatgattcttaagacttggaatgagacaagccgtgaatatccggatgcctgcttccatgaattatttgaacgtcaggcggcagaaacgccggacgcttgtgcggtcgtgtatgaacagcaaaagctgacgtaccgtgagcttgatgaaaaaagcacgaagctggcgctctatttgcaggcgcacggtgcaggccctgatgatctcatcggcatttatacagaccgttcattgcatatggctgtcggccttctcggcattttaaaagccgggggagcgtatgtgccgcttgatccttcttatcccgctgacagattggaatatatgatcgccgacagcaggatttcaatgtgcctgacgacggctgatttagagcactctttgaactggggaggcgtgcagacaactgccattgaccgcgattggtctcatattgaaagtacagccgcggaaagaacgtcgctgaagcggttagtgacacctgacgatctggcgtatgtcatttacacgtcgggcagcacgggcaaaccgaaaggcgtgatgattccgcaccgggcgctgaccaatttcttaatctcgatggcgaatgaaccggggctttcgtctaaagacaaactgcttgccgtcacgacatattgctttgatatcgcggcactggagctgtaccttcctttaatcaaaggagcggagtgcaacatctgtaagacagaagtcacaaaagacgcgcggaagctgaaagagctgattcaagagtacaaaccgacgatcatgcaggcgacgccgtttacgtggaaaatgctctttcattccggatggtcaaatgaagaaaaagtgaaaatcctttgcggcggagaggcgcttagcgaacaattgaagcagcagtttctcgatacgaaaagtgaggcttggaatatgttcgggccgacagagacgacaatctggtccgcggtgcagcgtataacggaaaatgagagcgcgcttacgatcgggcgcccgatcgccaatacccgcgtgtatattatggattccggtctcaaccctgttttagaaggggtgccgggagaactttgtattgcgggagacggattggcaagagggtatttcaacaagcctgagctgacagataaagctttcgtcagccaccggctcgaactgggatcaaagctttataaaacaggcgatatggcgcgttttcttcccggcggccgcattgaatacatgggacggatggacactcaagtcaaaatccgcggctaccggattgaacccggagatattgagagccggctgaatgcctatccggccgtacaggaaagcgtagtggttgtgaacaatcactccggaaatgaaaagctttgcgcattttatatcctgaaaaacggagagccgcttccatccgcgaaggaattgcgcaatcatttgaaacaagcgctgccggcctatatgacgccagcttcgtttatccgtcttgaagagctgccgctcacaccgaacggaaaagttgaccgcaagcttcttgctgcgcgcgatctgacggaaaagcagccggacacacaaacattttcatcatctcatatccagcaggcggtgctggcgatttggcaggatgtattgaaaagcaaagatatagagcttgatgaccgcttttttgatgcggggggagattcgcttcttgccgtgactgtcactgaccggatgacgcaggaactggattgtgaattcagtgtaacggagctgtttgaatacgcgacggtaaaagacatcagccggtacatcacagaacaaaaaccgaaagaaaccgttttggctcctgtgccgcaaaagaaagtgaatacagaagaccgggaacacccagcgggagaaattcccgattattatgaggacagtgtcgcggttatcggtatatcctgtgagtttccgggagcgaaagatcattatgaattttggaataacatcaaagaaggcaaagaaagcatcaccttcttttcaaaagaggagctccgccgatccggtgtttctgaagaactggctgatcatcccggcttcgttccggctaaatcagttttagaggggaaagaaatgtttgatcccggtttcttcggcttttcaccgaaagatgcggaatatatggaccctcagctgagaatgcttttgctccattcatggaaggcaatcgaggatgcgggatatatctcaaaagaaattccggaaacaagcgtgtatatgtctgccagcacgaactcttaccgcagcctgctgccggaggagacgacagcacagctggaaacgccggatggctacgtatcatgggtgcttgcccaaagcggcacgattccgacgatgatttctcataaactcgggttgaaagggccgagctattttgtccatgccaactgctcatcttcgttaatcggcctgcattcggcttttcaaagcctgcagtccggcgaagccaagtatgcccttgtcggcggtgcgacccttcatacggaatcaagtgccggatatgttcatcagccggggctcaatttttcaagcgacgggcatattaaagcgtttgacgcggctgctgacggaatgatcggcggtgaaggagccggcgccgtgctcttgaaaaaagcgtcggatgccgttaaggacggcgaccatatttatgcgctgctgcgcggaatcggcgtcaataacgacggagcggataaggtcggattttatgcgccgagcgtcaaaggacaggctgaggtcattcagaaggtcattgatcagaccggcatccatcctgaaacgatcgcgtacgtcgaagctcacggcacaggaacaaaactcggcgacccgatcgaactgtccgcacttcaatcggtgtacggccgatatacagataaaaaacaatactgcggcatcggttcagtcaaaacaaatctcgggcatttggatacagcggccggaatggcgggctgcatcaaggttgtcatgagcttatatcatcaggaaatcgcgccgagcatcaattacaaagagccgaacccgaatcttcatttagaagactctccgttttacgtagcgggagaaaagaaagagttaacccgtgagaacagagcgcaccgcatggctttaagctctttcggtcttggcggaacaaacactcacgcgatttttgaacaatatccggatgcatccgaagcagcagacgctgccggtccgttcatcattccgttgtctgccagaaaaatagatcgtctgaaggagtatgcaaaacagctgctggcttttctggaaagaaaaacggacgccggcctcgctgatcttgcctacacgttccaagtcggaagggaagcgatggaagagcgcgcggcctttatcacaagcggcactgcggagctcaagcggcagcttgccgattttatcaatgataagccggctgtaacaggctgcttcagaggagaaaaacagcaggcgaaagatattgcctggctttctgatgatgatgacagcgctgaactcattgagagatggcttgcaaaaggaaaaggaacgaagctctgtgaaatgtggagtaaaggtgtcgccattaattggcacaagctttacaaagacaagcatccgaaacggatcagcgtgccggtctacccgtttgcaaaggagccgtactggccgaaaaaagcagaaaagaaaacatctgccgcacacaccggagtctcagttttacacccgctcgtgcagcaaaatacgtcagatttggccgttcagcggttcagctcacggtttacaggctcagaatttttcctgaaggatcatgtggtcagggaaaaagcggtgcttccgggagcggcatatttggaaatgtcttatgaagcggtaaaacgggcgcttggcggcttattaaaagaccaagacagaataacgctccatcataccgtctggatgaaaccgatcgtcgttcacgaacaagagcgccaggtccatattgcgctgtttcctgaagaggacggcatcatctcatatgacatgtacagcgtaaacgctgacgatgaagaagtgctgcacagccaaggacgggctgaaatcataaaggctgaacgagagcctgaggccgacctttctgccatacaaaacagatgcacagcggacattcttgatccggcggccttttatcgggaaggacggagcagggggatgttccacggaaaagccttccaaggcattaagtccgcctttatcggcgagaaagaagtgctttcggacatacagcttccggattctgtttctcatacgaacggacaatttacgcttcatccgagcatcattgattcggctattcagacggcgacgatctgcattatgcaggagttttccggacaaaagctgattctgccgtttgcgctggaataccttgaagtcgtcaaaccatgcacaccgactatgcgggcgcatgcacgcttcagcgaaggatatcaaaccggtgattccgtgcagaaagctgacattgacctgttcgacgaaacaggagcgctttgcgtgaaggtgagaggattctctacgagagtgcttgaaggcgatgcagaatcggctcagcggccatcaggctcggaaaaggtcttgtttgcaccggtttggaaagaagccgccgcatccgagaaagcttctgtccatgctgatgagcatttgatcatactttgtgaagatgcctgccgtataaaagataaagcggcatccggcctgaaagacgccgaactgctgattgcagaaggtgagggtgaaacagccgctgaacgcttccaaagctttgcgcagacaatcgctgaacacatccggcggatcatcagcgaaaaaaggcagggccgcattctggttcagaccgttatttcagcagacggcagccagcaattgtttgcggggttaacaggtttgttaaaaacagcagagctggaatacggaaaacttgactgtcagctcatcgagatggaatcaatcggcgaaatgccggctgtcgctcaaaaactgaaaaacgacagtcatcggccgcatgataaacatatccgttatacaggagaaaaacggtatgtgaaggcttgggaagaaatgcatcctgcaggtggcggcgccgtctggaaggacgaaggcgtatatctgattaccggaggcgcaggaagcctggggttgatgttcgcaaaagaaatcgcaggtcgcgtaaaacacccgaaaatcattctgaccggccgctcaaatctgacagaagccaaacagcgtgagcttgctatgcttcaggactcaggcgcaaatgtcgtctacaaaaagctgaatgcggcagaccggcgggaaaatgaagcgctgataaaagagattacggcggaattcggggggcttaacggaattattcacgcagcgggagtgatacaagaccgtttcctgctgcaaaaaacgaaggaagagttcagggaagtgcttgatccgaaagtgatcggcaccgcttatttagatgaagccagcaaagaatgcaatcttgactttttcgtattgttctccagtgtttccggcgttctcggcaatgtcgggcaggctgattatgccgccggcaacgcgtttatggatgcatacgccgcttacaggcaatcccttacggagaatggcctgcggcacggaaaaacggtcgcctttaactggacgctgtggaaagatggcggtatgcaggcgggagacgagacggaaagaatgatgaaaaaagctttcggcatggttccgcttcaaaaggcgtcaggcctggctgcattttatgaaggaatggctcaggaaaagccgcagcttttagcagctgaaggagacgcagcaaaattaaaacagtcgttcctgtccgtttctgaaactgaaaagccgcagatggagaaaatagagcctgtatccgccgtttcgggagacaaatggcacggtgcgcttaccagattggtcagttcaattttaaaagtcggccaagacgagattgatattgatacggaactgagtgaatacggttttgattccgtatcctttacggtctttacgaatcaattaaatgaagcgtatcagctggagctggcgccgacgatcttcttcgagcacgggacgatccgcagtttagcagagtatttgacggatgaaacagaagcaggtcttccttttcagccggaagaaaaacacgatgcagaagaatctcttcaagcgcttcacaccgctttgacagccatggtaagcggcattttaaaagtcggccaagatgagattgatattgatacagaactgagtgaatacggttttgattccgtatcctttacggtctttacgaatcaattaaatgaagcgtatcagctggagctggcgccgacgattttctttgagtacggaacgatcagcaatctcgccggatatttggcaaaagagcatccgggccgtttcggcgaaaagaagaaggagtctccgaaaaatgagcagccaaaagcccaaaaaccgaaaacgcatcggaaaaagcgctttgcaacggtaatgaacgctagggcagcgacacaagagccgcgccgcttggacccggttgccattgtcggaatcagcggccgattcccgggcgcgaaggatatagaggaattttggcgcaatctgaaagaaggaaaagattctatcacaaccattccgaaagaacgctgggattggcaagcgtttgatggcgacccgaaccttgaaaggaacaaaacgaatatcaagtggggcggctttatcgacggaatagctgaatttgatccgctgttctttgggatttcaccgcgggaagcccaatatttagatccgcagcagcggctgttattgacttatgcatggcgggcgatagaagatgcgggctgtaagccggaaagcctttccggcacaaatacgggtgtctttatcggcaccggaaataccggttataaagatttgttcacaagggcaggacttgcgccggaaggacatgcggcgacaggcagcatgattccttccatcggtccgaaccgtttgagctatttgttaaatcttcacgggccgagcgagccgattgaaacggcatgttcaagctcgcttgttgcgattcaccgggcggtatccgctatagaaaacggagaatgcgacatggcgattgccgggggaatcaacacgattctgacggaagaagcgcatatcagctacagcaaagcaggcatgctcagcaaagacggaaaatgcaaaaccttctccaaagatgccaacgggtatgtcagaggagaaggcgcgggaattctgatgctgaagaaactgacagatgcagaacgtgacggcaatccgatttacggcgtgatccgcggtacggctgaaaaccacggcggccgggcgaatacgttaacctcgccgaatccgaaactgcaggctgatttattggtgaaagcatatcggaaagcgggcgtggacccgagcaccgtcacgtatattgaagcgcacggtacaggaacggaattgggcgacccgattgaaataaacggtttgaaagcggcgtttcacgagctggcgaaaacaaatcaagagcccgaagtctccgatcaccgctgcggcatcagttcggtcaagagcaatatcggccatttagagcttgccgcgggagtatcaggcgtcatgaaagtgcttttgcaaatgaagcacaaaacgctggtgaaaagcctgcattgcgagacgatcaatccgtacattcagctcgatgacagtcctttctacatcgtgcgggagactcaggaatggacggcagcgaaagatagaaacggaaatgcgattccgcgccgggccggggtcagctcatttggcatcggcggggtcaacgcccatatcgtcattgaagagtatgttccgaaagcagccgcgcagcctgagcacagcccggaaaatccggcaatcatccttttgtccgccaaatcaaaagaaaaactgtttgaacaggcgcagcagctgcaaaaagcgatccgtcaaacgccgtatacagatcaagacttggcgggcgtcgcttatacgctgcaaaccggaagggacgaaatggaagagcgcctggccattcttgcagcgacgatggcggaattggaaacaaagctggaagcatttatggaaaatgagaaaaacatcgccgggctgtataccggccagtccaaccgcaataaagatacgtttgccttgttcacggcagatgaagatatggacatcgtcattgatgcctggatcagaaaaggcaaattcgccaagctggcagaggtatgggtgaaaggcggcgtgttgaattggaaccggctgtatgaaaaaggtacgccgcgtttactcagtcttccgtcttatccgttcgcaaaagacacgtattgggtgcctgagaatcctgacaaaaataaggtccatgcagaggaacggatgaaatgtattctgacgaaacaatgggaactgagcgcgctcgaactgcgtgaaccgtgcccacgttccgttgcgattttgacaggcggggacacgatcaatctcgcagaagagattgcagcgcatatgccgaatcaccgtatcatcacatcatcaggccttaccggtgaatatgactggcaggcgtttgacggagttatcgacctcctcggcgcagcgaaagaagaagcttctggagatatgcccggtatcaaatggcttcagcaattgattgagcatgggcataaagaagggatgacactgctttgtgtcacgaaagaccttgagtcattaaataaagaggccaatcaaacaacaggagcaaaacgggccgggctgtacagaatgctggcatgcgagtacggtcatctgtcttcaaggcatctcgaccttgaaggagcgctttctgatgacaaaatggcgaagcagattgccgatgagtttcatgcccgtactgatgacgcggaagtttgccggagggacggcttgcgctatcgcgctgtattgagcggatcagaaaatatgaatatgcaaggcgagagaattgattttccggacaaccatgtgcttctgatcaccggggggacgagggggatcggtcttttatgcgcacgtcattttgccgaacattacggagtgaaaaaactggtgcttacaggccgtgagacccttccgccgcgaagtgagtggacgggcggactgaatggggtgacggcttctgtaaaagcgaaaattgaagctgttctcgacttggaatccaaaggtgtacaagtgaaggtattgtcagtcccgttagctgataaagccgggctgagacaggaactgagtcagatcaaacagacgctcggatctatcggcggcgtgattcactgcgcgggtgtgaccgataaagagacgctcgcattcatcagaaaaaccgatgaagacatacagcgcgtgctcgagccgaaa

SEQ ID NO: 21 - DNA, 9960 bp, unidentified
  source 1..9960: mol_type = genomic DNA
gtagacgggcttcaggcgttatacagtgtgctcagcgaagagccgctgaagtttttcgtcttgttctcttccgtgtcagctgcgataccggcgctttcggcaggacaggccgactacgccatggccaatgcgtatatggactattttgcaaactcatacaaagagcagctgccgatcgtaagcattcagtggccgaactggaaagaaaccggaatgggcgaagtcacgaacaaagcttataaagagagcgggctatacagcatcacgaatgcagaaggattacagctccttgacggcatacttcttgaatcagcaagaggcccggtgctgccggccgttctgaatcctaagctgtggaagccggagcgattcctgcggcgaagcaggcaggatcagccaatgagccctgctatggtaaaaccggcggaaaaaaccgctgatactgccgcagacaacgctcaattaacaaggaaaacagaagaatggctgaaagaattattttctgaagagctgcggattgaccaggatcagcttgagacagacgtattattccaagattacggagtggactccattatccttgctcagcttctgcagcggatcaatcggaatctctcggcatcgcttgacccgtccattctgtatgaatatccgacgattcaatctttcgcaaattggcttttcgaaggatatacagaagttttgtcagaacggtttgatttggcggcggctgagcctgttatgaagaagccggctgagccggtaattgtaacacaagccgttaaggtgcaagaagagccggccgaaaaacagattcaccgggaagatatcgcggtcgtcggcatgtcctgccggtttccgggggcggcttccttagaggcatactggtcgcttttggctgaaggccgttcggctatcggtcctgttccgcctgagcgatggggtttgaaaacgccttattacgcgggaatgcttgatggaattgatcaatttgatccggacttctttttactggctgaagaagatgtaaaggcaatggaccctcaagctttggccgctctggaagaatgtcttaacctgtggtatcacgcgggctatacgccggatgagataaagggagaagcaataggtgtgtatctcggcggacgcagccgtcacagaccgggtgaagagaaactgctggatgcgaaaaacccgatcgtcgcgctgggccagaattatttggccgcaaatctctctcaatattttgatatgcgcggcccgagtgttgtcctcgatacggcttgttcttctgctttagtcggaatgaatatggccgtgcaggctcttgtaacaggagaaataaaagcggccgtcgtcggcggtgtgagcctgtttgaatcagaagaaacccataaattatttgaacagcgcggcatattaagtaaagcgcagtcgttccatgtatttgacgaacgcgccgatggagtcgtgctgggcgagggtgtcggaatggttcttttaaaaaccgtgtctcaggccattgaagacggagattctatttatgccgtagtgaaggcggcatctgtcaataatgacggaagaacggcaggcccggctacgccgagtctggaagcgcaaaaatccgtcatgaagacggctcttgaaaaaagcggaaaacagccggaagacattactcatattgaagcgaacggttcaggcacggtcgtgacggatctgttagaactgaaggccattcaatccgtctaccgttcaaaagacgccggcccgctcggaatcggatctgtgaaaccgaacatcgggcacccgctttgcgcggaaggaattgcaagtttcataaaggttgttctcatgctgaaagaaaagagctttatcccgtttttatcaggtgagcatgaaaacacacattttgatagagaaaaggccaatattcagttcagccgcactctggcagactggccgtcaccaatgccggcggcaggaattaactgctttgccgacggcggaacaaatgcccacgtcattgtggaagcgtgggaggaagacgaagggcgccgaatcaagcgccaccctttaacaccgcctgcattaaataagcggctgatttcgccggacagcaaagaagaaacgaacgaaaaagctgcggcaaacatctgggatacctatgaagtggaggtttaacgtgaagaacttcaataagcaaatcactttatcgctgaaaaatccgtttattttacatcatgacgtttacggacaaaaagtccttccgggattggcttatattgatatgttttatcagattttcaaagagcgcggttattcatacgacgggcttcagctgcgccatttatccatatatcagccgcttatcgcgaaagaagacagcgccgtcaagcttacggttgaatgtacgaaggtaaaagaagggcgctggaaacttaccgcaaaaggggctgaggtcagcggcggacgagtccagccggaagaacgcatttacgtcaaagcggaaatgcacgagacggcacccgcagttttcagtgacacgcttgatcttgactctgtcaaacgcgaggcggaacgcaccagcgatttaaatgagctgtatgcacaatgcagaagccaggaattggttcacagcggatacatgaaagcggaaggagaagtgtttgaggagcgggacggcgttacctttgatgtatcgctcggaaaagacgcgatgggacaggccggagggtttttgttccatccgactcttcttgacggaagcggcgtcggcgcaaacctattgatctcatcgctgttaaatggggaaaagaggctgtttctgcctcttttctatgaatcttttacagcttccgaacctttgcaggccggctgcatcacgagaatcaaacgttcgtcgcttcggcaggaaaaagagctcatttacattacgctggagttttttaacgcatccggccggaaagtagccgagctgaaagatttcacaagcaagctcgtgcgggatgccgagctgatcaatacagaccgtaagcctgctgagaagtctcctgaaaaagccggtttaccggaagttgcggcatcgcttgaaggtggacacccgcatgaggaagcagaacggtatgtcatgaatctgatggctgaaaaaatcggcaagccgcttgaacagattgattctcaagtcggctactatgaaatggggctgacatcgtccggactgcttgacgtcgtcgaaacgatcagcgaaaaaatcggcgaaaccctttctccgactttgctgtttgaatatacgacggcagcagagctggcggctttcctgacagagcagtacggaagccatttcagcgcgtctcctgcaaaaatagagaagcaggttgagaagcaaacagaaagaagacggaaaagcagtgttccgcaagatgaagatatcgcgattatcggcatggccggccgctatccgcaggccgaaaatattcatgaattctgggaaaacctgaaagaaggaaaaaactgtgtcaccgaaattccggagtctcgctgggactggcgcaaatttgaaaatgtaaagtcgccttccggaaagagcatttcaaaatggggcggatttattgatgatccggattgctttgatccccaatttttccggattacgccgcgggaagcagaaacaatggaccctcaggaacggctgtttttacaaacatgctgggaaacgattgaggatgccggatatacgccgaagtcgctcgcagaggcgcgcggacggaataagagacagcgcgtcggcgtcttcgcaggggtgatgcataaggattatacgttggtcggggctgaagcttcttctgaagatcatgtattcccgctttctcttaattatgcgcaaatcgcaaacagagtttcatatttttgcaattttcacgggccgagcatggcggtcgatacggtctgttcatcctcgctgactgcggttcaccttgctgcggaaagcattcggcgcggggaatgtgaatcggcggttgccggaggcgtcaacttgtcgctgcatccgaataaatatctgacatacggtttatgggacatgttttcaacagacggggtttgtcatacattcgggaaagacggagacggctatgtaccggctgaaggaatcggtgccgttttattaaaaccgctgcgagaggcggtaaaagacggagaccggatttatgcggtgataaagggcagtgccgtcaaccacgtggggacagtgagcggaatttccgtgccgagtcctgtcgcgcaggccgatttgattgaagaatgcctggacaaagcgggtatcgatccgcgcacggtcagttatgttgaggcgcacgggacgggaacttctctcggcgacccgatcgaggttcaaggccttgtcaaggcgttccaccagtacacacaggacaaacagttctgttcgatcggatcagtgaagtccaatatcggccatgcggaatcggcggcgggcatcagcggcttaagcaaggttgccctgcagcttcaccataaaattctggtgccgtctcttcattccgaggagctgaatccgtatttagattttgaacaatcaccgttttacgtccagcatcataccgaagcatggcagcagccttcagtgaaggaaaacggaaacgaaaccgtatatccccggcgtgcgggcatcagttctttcggggcgacaggttcaaacgtgcatctcattctggaggaatttatccctgaagaatcacacggaacagtggctgaaccggcagaagaacagacattcattttcccggtttccgcccggaatgaagaacggctaaaagagtatgttgcgaagctgtctgcatttattcatcccggcctgccgcttgaggatgcggcgtatacacttcagatcggccgtgaacagatggaggagcgggttgcgttcaccgctcagaatatgacacagctcaaagagcggctggaaggctttctggcgggtaaaacggtcagcggatgctggcggaaccgagccgataaaagcaaacaggcagagcttgctccggacttgatacataattggctttcttccgggagtctggagatgattgccgaaaagtgggccggcggaggaacgatggattggaatctcttgtacagtgaaaacaaaccgaatcggatcagcctgccgacatatccatttgaaaaaatccggtattgggtgccgaaggcggagaaaagagaatcgttgccgcaaaaaagggcaggcatgctgcatccgctccttcatgaaaatgtatcaaacatgtcaggtgtccgtttccggtcctcttttagcggcggagaatttttcctgaaagatcacgtcgtcaaaaacgagcggacgttaccgggagcggcttatctggaaatggtccgggcggccgttgaacacgcggcagaaacgaaggaagaaggaacagtccgtctgaaacatgtcgcatggatacgtccgtttacagcggcggatgagtccgcagaactgtatcttcgtctttatgataaagaaaatggggagatgggattcacggtttacagtgaatcggcttcctcggaacaagagcccgttctctatgcgcaaggaagcgcggcgtttgtccgagcgggcgaaccggccaaaatcgagattgacaaattaagagagtcacgccgtctgatgatcacacccgacgcttgctatcaggcatacaaacagatggggattgaatacggagatgctcacaaagcggtcacggaaatctatgcaaaagacagcaaagtgctcgcgaagctctcactcccgccatctatttctcatacaaaagatcagtttgtcctccatccgagccttttggatgcggctttccacgcgacaatcgggttaatgcttgacagagacgggactgacataaggccgatgctgccgtttgctctcgaacagattgatgtgttgaaaagctgtaaaagcgatatgtgggtttccattgcgtacagcggcgaaagcaagcctgaagatgccgtgcagaagctggatattgatgtatgtgacgaacaaggcaatgtatgtgtgaaaatgactggttattccgcacgggaaattgaaacggataaaacagatacactattttttgaacacgtatgggaagagaagaccgctgtacatgaaaaagcgccgactcatttcaaagcccgtcacgtgatgatatcagaccggtttacggatcaaatgaacagcatacagacagacttcagctgtcatgtgctccgtgatcagcataagagtgaagacgaacggtttgaagcttgtgcgcttcaggcgttcgaagaagtgaagaaaatcctcgcttcaaattccggcgggcgcgagctcattcagattgtcgtgcctcaagaagcggatacgcagctcctttgcggcttaacggggcttttgaaaacagcggcgcttgaaaacccgaacctgaccggccagctgattgaattggagccggacgtcaaagcagacacgctgtcccaatatgtaaaagaatgcagcgctcatcctgatgacaagcatattcgctatgaaaacggcaagcgctatacggcacaatggcaggaaacagggcttgaacagttcggcgccgaaaagccgtggaaggataaaggtgtttatctgatcacaggcggtgcgggcggactcggatttttattcgccaaagaaatcgcccgacatgcaaaagacgccgttcttattttaacggggcgttctctgttggataaccgtaaagaagtccagcttcaagaactgtccgatctcggcgcgcaagccgtgtacaaacaaacggacatgacggaaaaacaagaagttcaatcgctcacagcggacatctgccggaagtacggcggattaaacggtatcatacacagcgcgggaacgataaaagacaactatattctgaaaaagcacaaagatgaaatccgtgatgtactggcgccgaaagtctccgggctggttcatcttgatgaagcggcaaaggacattgagcttgattttctgattcttttctcttccggagccggaagcgccggaagcgcgggccaggctgattatgcgatggccaatgcgtttatgaacgccttcagtgaaaaaagaaaccgtaaaaaagcccgaaatgaacgctatggacacaccctttcaatctgctggccgctttggaaagacggcggcatgacggttgatgctgaaacggaagcgatgctgacaaaagaaagcggcatggtccctatgaaaacagaaaacggcatccatgctttgtaccgcgggtgggcggcaaacacatcccaaatcatggtgacggaaggctacgccgcaaaaatccgggcgtttctgaaaaaatcagcggatgatcagccggataagggcagcgaaacggaagaaccggcaggtcttacagcaggcggagagaacatgcttgcagaaaaaacagagcatttcttgaaacatgtgctttcggaagtaaccaaacttcctgtcggcaaaattgacgccaaggcgcctatggaagattacggaattgattcgattatgattatgcatttgacgggccggctggaaaagagcttcggttcattgtcaaaaacattgttcttcgagtaccagaatatcgaatcgctggcgcagtattttatgaaagcacaccgggacacgctgctggaagtgaccgggatgaaaacagaagaaacccccgcagccaaaccggcgctgtctgagaaagcgaaagtaaagcccgttttcaaacggaatgaaggcaaaaaaggctttacgcaagtccgcacagacacgcggaaaaattcagtgacggagcgtgaagatatcgcgattatcggtatatcaggacgatatccgcaggccgaaaatcttcaggaattttggaagaacttgagtgaaggcactgactgcataaccgaaattccaaacgatcgctgggatcacagtctttattacgacgctgataaagataaagaaggcaagacgtacggaaaatggggaggatttttgaaagacgtggacaaatttgatccgcaattcttctctatttcgccccgggatgcaaaattgatggacccgcaggagcggttattcctgcaatgtgtctacgaaacgatggaagatgccggttacacgcgcaaaaagctcacggaaaaatcgggtgatttgcttggcgccaatgtcggtgtttacgtcggcgtcatgtacgaggaatatcagctttacggagccgaagaacaggcgcgcggaaaatcgcttgcgctgaccggaaatccgtcatcaattgcgaacagggcttcctatgtattcggttttaacggcccgagcatggctctggatacgatgtgctcttcttctttaacagccattcatctggcatgccaaagcttgcggaacggggaatgcgaggcagcatttgccggtggtgtcaatgtatccgttcatccgaataaatatttgatgctcggccagaacagatttttatcaagcaaaggcagatgtgaaagtttcggagaaggcggggacggttatgtgcccggtgaaggagtgggcgccgtactccttaagccgctttctaaagcaaaagcggacggtgatcatatttacggcctgatcaaaggaacggccgtgaaccatgatggaaaaacaaacgggtattccgtaccgaatccgaacgcccaagccgcggtgatcaaacaggcgttaaaagacgcgggaaccgatccgagggcggtcagctacatcgaagcccacggcaccgggacttcactcggcgacccgattgaaatcacgggtctgacaaaagccttttcagaacaaacacaagataaacagttttgtgccatcggctcagcgaaatccaatatcgggcactgtgaaagcgcggcgggcatcgcgggtctgacgaaggtgctcctgcaaatgaaacataagcagctcgcaccgtcactgcattcccggacgcttaacccgaatattgattttctcgctacgccgtttaaagtgcagcagacgcttgaagagtggaaaagaccggtcataaatgaaaacggagtaaataaagaattgccgagaaccgcggggctttcctcattcggagccggcggcgtcaacgctcatatcgtcatcgaagaatattccgctgatgaggataaggagacggcttttgccgcacctcatccgtcgatgatcgtcctttccgcgaaaaatgagcaacggcttcaaaaacgggcgaaacgtcttcttgatgcactgcggagcgggcgttaccgggaagcggatctgagccggatcgcatatacgcttcaagtcggacgtgaaccgatggaagagcgtctcggaatgattgtcagcaatttgcgcgagcttgaagagaagcttgacgaatttacgggcggcaaggagtcaattgaccagctctaccgcggacaagtcaaacagaataaagacacgatggcgctgtttacggctgatgaggacatggaaaaaaccattgaggcgtggcttgaaaaaggcaaagccgcaaaggtattagagctttgggtgaagggccttccgctgaattgggataagctgtatcaaatgggcagaccgcaaaaaatcagtcttccggcctacccgtttgcaaaagaccgttattggatcgacacatcagctgatgcagctgtcaaaagaccggccgaaacacagtctgcgccgatcgcaccggcggccgccccggaaccggtcattgaagtaacaactcttcacgaggagactgtcaaagaaaaaccgtcaaacattacactgcagactttgagcggcgcgaaaccgaaaagaaccgtacagcccgcttcccgtccggcggtttccgctgaaaaagtaagtgccgtattgaacgaaagtttggcagacgttttatatatggatgccgacgatat

SEQ ID NO: 22 - DNA, 9960 bp, unidentified
  source 1..9960: mol_type = genomic DNA
cgacgcagacgacacatttattgatatcggcatggattcaattaccggtcttgaatggataaagtctgttaataaagcatacggcacgtcccttacggtgacgaaagtatatgattatccgacaatacgccagtttgcggctttcttgcaaaaagagctcgggacagaaccgatccttgaggaagcaaccgaaccgcagaaaacagaggccgtgaaaaaaccgtcaaacatcatgctgcagcctttaaacgctcataagcagcatgtgacacaagcagaagcggatattctggaggcgatgtctgaaacggcagcttccgccgaaccggaagaaccgattgaagcggtgcatgacgagttatcaaaagcactggctgatgtattgtacatggagcggcatgaagtagatattgatgaggcctttattgacttgggtatggattcgattaccggtcttgaatggataaaggccgtcaataaacggtacggcacggactgtaacgtaacgaaagtgtatgattatccgacaatccgtcaatttgccgattttctgagtacgcagccgtccgtccgcggcagaaaaaaacaggcggttcccgtacgtccaaagccgcttacgcagcaacatgccccgcaggagaaaacacctgccgagccggagagattaacggttcaaaaggaacctgaaccggtgcaggccaaacgggagcctaaagacgagatagcggacgcgatagccatcgtcggcatgtcgggcaagtacccggatgcgccggacctaaagacatattgggacaacttagcccgtgcgaaaaacgccattcgcgacatcccgctttcccgctgggatgtaaacaaatactatgatccggccttaaacaagaagggcaaagtatactgcagatcaatcggtatgcttgacggcattgaagagtttgatccgctgttctttaatatatctccgtctgaagcggaacttatggacccgcagcaccggatatttttacaggaaggttttaaagcctttgaagatgctggttacagttcaaaagaattaaacgggaaaaattgcggcgtttatttggggattatgaacaatgaatacggcatgatgttaaacaaacatcagacaggaggaagcgcgacgggcaacagtttcagcatcgcggcggcccgcctgccttattatctcaatttgaaagggccggccattccgatcgatacggcatgctcttcatctctcgtcgggactcatttggcgcgtcaggcgctgttgaatcatgaaattgacatggcgcttgtcggcggtgtcaccttgtatttaacgcctgaatcatacatcagcatgtgtgaagcaggaatgctgtctcctgacggccaatgtaaagcatttgataacagtgcgaacggatttgtgcctggcgaaggcgcgggggcgctcgttttgaagaggctgaaagatgcggaagccgatcaagaccatatttacggagtcattatcggatctggaattaaccaggacggaaaaaccaacggcattaccgcaccaagcgcgaaaagccaaatggatttagagcgccaagtgtatgaagcgcacaacatccaccctgagagcatcacctatgcggaaatgcacggcaccggcacaaagcagggggatccgattgaattagaagccctgtcttctgtatttaaggaaaaaacggatcgcaaacaattttgcgccatcggttcggttaaatcgaatattgggcatacgtcggcggcggcaggtgtagcaagcgtccagaaggttctgctgtcgatgaagcatcagcagcttgtgccgactcttcatttcagcacgccgaatgaacattttgatttcgatgactcgcctttatttgtcaataccgaactcaaaccttgggaaacaggcggagcaccgcggcgggcctgtgtcagctcgttcggctacagcggcacaaacgctcatctcgttatcgaagagtatcagccgaaagaccggggaagccgaaagcctgatgagacaaacatttttgtgctatcggcgaaaaaagcgcctcaggtaaaaacctatgccgagtctgtgatcgaatttgcaaaagccaatcagaatatcaatctttcagatatggcgtacacgcttcaaaccgggcgtgatgcgatggattaccgtcttgcggttacagccggttcgagagaagaactgatcagccgtttaaccgattttgtaaacggaaatacgggtgaagggatatatgaagcccatatcaaaacggctaaaaaagaaatcaaagcatttgaaacggacgatgacctgaagcagcttgtgaggtcatggttcgataagaaaaaatacgccaaaatagcggaattgtgggccaaagggcttcagatggactgggacagactctatcaaggcaaaaaaccccgcagaatcagtctgccggcttacccatttgcgaaggagccgtactggctgcctgagccgaaagccgtaccgcaggaaacagaagaccggcgcgaggctgtgcttttagaaaagcaatgggttccgtgtgagatcgattcggccggtgagaaaagcggcaagacggtcatgattctggctgatgcaagaacagcggagctagccgaagagactgcaaaacatttcgctgactcgttcattctcccggtcgattccgctggcactgcagctagcgattggaatcacatagatggcgcaatcgatctgacgggatgcggagacgaagacccttcactggaagggatcagccggctccagactctgattgatacaagccgccggcaagcgcggccgctgacgctgctatatgtaacaaaaggcttggagccatttcagcagaaaacgattcatcttgcaggcgcggcaaaaagcggcctgtacagaatgcttcaaaatgagtatcaaaacgtgacgtccagacacatggatgccgatacagatacggatcagcagacgcttgccaaacagattgcagatgaattttcggcggactgcacagaaccggaaatctgccgccgaaacggacagagataccgtgctttattcagggagattccaagcgggaagcgagcagaacataagcccgcaccgtttcctgaagaccatgtgcttttcattaccggaggcacgaggggactgggtctcttatgcgcccgccatttcacagaaaaacacggcgtgaaaaaagtagtgctgacaggcagggagccgattcctccccgtcataaacggtcagccgttaatacgaggctgaaagagaagatacgggcgattgaagagcttgaagcaaaaggagtgcaagtggcggtgatcagtctgaaattgtctgatcatgatgcggcggcagcggaaattgaaacgatccggcgcacgatgggaccgatcggcggcgtcattcattgtgcgggatcggttaattcagtaaatccggcatttatcagaaagactgctgacgatatacaagaagtgctggaaccgaaagtcgccgggcttcaaacattaagcagccttttcacaaatgatcctttgcgattttttgtcttattttcttcagtatctgcagtcgttccggcgctcgcggcgggacagtcggattatgcgatggcaaatgcgtacatggattatttcgcggcggctcatcagcatcaatatccgttagtcagcattcaatggccgaactggaaggaaaccggattgggtgaagtccgcacagaaagcctgaagcagaccgggctgctgggactgtccaacgcggaaggacttgacttgctggaccgcattttatctgaaaacatccgcccgtccgttctgccggctatcattgatcggagcgaatggcatcctgacaggctgctctgtcaagagaaaaaacaaaaagagactgaagccgtaataaccggcaaagccgctgaaaaacattcagacagagaactgcttcttgatacgaagacgtggcttgtgaaactgttttccgatgagctgaaaatcgctccggaagaactggaaacagacgaaccgtttcaggattacggggttgattcgatcattttagcgcagcttttacagcaaatgaatcaagcgctcaaagaggacttagatccgtctgttttatatgaacatccgacgattgacgcgtttgccgaatggcttgtatccaatggacagccgctgcttgcagaaagggaggaaccggatacagaaatcttggttccggctgccgtacaaacggttcatactgaacaaaagcgtgatgccgtaactgaagacattgccgtcgtcggaatgagctgccgttttccgggagcggaatcgttagaacaatattgggacttgctgagaagcggcaggtccgcaatcggctccgtcccggcggaacgtttcggatatgcaaatcaatacgttgcgggattgattgacaatatggatcactttgattccgaattctttttcattccggaaaacgacgcgaaagcgatggaccctcaggcattggcggtgttggaagagagcttgaaactttggtgccatgccggctattcccgtgaagaaatcaagggtatagaggccggtgtgtatatcggaggccgaagccagcatcagcctgatcctgaaattcttgcgaatacccgcaacccgatcgttgcgggcgggcagaattatttagcggcaaacgtttcgcaattttttgatctgcgcgggccgagcatcgtgcttgatacggcttgttcatcggctttgacaggaatgaacatggccgttcaggcactgagaagcggcgatatcaaagcggcggtagttggcggcgtcagcctgctgaacacggatgcggcgcaccggatgttccaagagcgcgggctgttaaacgaaaagccggcatttcacgtatttgacaagcgttcaggcggagttgtcctcggtgaaggcgtcggcatggtgctgttaaaaacagtaagccaggcgcaaaaagacggcgacacgatacacgcggtcattaaggctgccgccatgaacaatgacggcagaacggcaggcccgtcagctcctaatatgcaggcgcaaaaagatgtcatgcaaagcgcgcttttcaaaagcggcaaaaagccggaagacatcagttatatcgaagcaaacggatcgggttccgctgtaactgatttgcttgagctgaaggcgattcaagccgtataccgctctggccagcatgttccgctcggcatcgggtcaattaagccgaacatcgggcatccgctttgcgcagaaggcatcgcaagcttcattaaagtcgtattaatgctgaagcataaacaaacggttccgttcctctcaggagatgaaccgatgccgcattttgatataacaaagacagattttcattttcataaaacagccggcgaatgggatgcagccagaccgtcagcagccatcaactgctttgctgacggcggaacgaacgcccacgtcattctggaagcttgggaagaaaaagatacccgaatgaaaagaaagccgctgccggtaccgtcattaagacgtaaggctctggtaaaagaacctgacgtactagcggagaaacaagagcttcctgcgaaaaaaatgttttggaaagcatttaaatgagagaggagtgggaaggaatgatatcggaaaaaatcaatatctcactggatcatcccatcatgaagggccatatggtctttggtgaagcggtattgcccggtcttgcttatatcgatttgatttatcaggtttttcaggaacacggttatgtatacaatgagctcgaattgaagcacgttacgatttatcagcctttaaccgtcagtgaaacggctgcaatcattctgaccattcaatgttctgaaagcggagatggaaagtggctcatctccctttcggggcagaaacagcaaggtgacgagctgtcagacaatatacaatatgcgacagctgaaatggtgaaaagagacgctgtgacatttgacgaaaccattgatttcggcgatgtgaagcagtcaggcagatctctgaatcttgatgacatgtataaccgctgcctggatcatgacttggttcattccggcatgatgaaatcgaacggacgattgtacgaaacagaagacggcatgctggctgagctttcttgcaaagaaggaaaaggtttctttttattccatccggcccttttggatgcaagcggaatcggggcgagcggtttgctttctgaaacaaaccggcttcatctgcctttatattatgaatcgttcacggccgccgacaggctgcaatcagagtgcacggcgcgtatcgccgcttccggcgtccgtcaggaaaaagaactcacaacgatgacaatcgagtacttcagccgatccggacaaaaggtggcggagcttaaaaacattacgggaaaagcggttcggctcgaagaagacgagaatacagccgaacagaatcaacaggctgcagctgagccggaaagcgcagatgaaaaagacatcattctgtttttaaagaagcttttggctgataagcttggtcagccttgggagacgctggacgttctagccggctactatgagcttgggctggattcatcttctttgcttgagattgtacaagatatcagtaaaaagacgggtgcggacctcgcgcccactcttctctttgaatatacaaatataaaagaactcgcagcctttttgaaaaagaccgtttcatttgaatgtgcggaggaacgtgcagccgaaccatcacggtctgaagctgaagcaccggacgcgccagaacaccataaaaacaccggagacatcgcgattatcggaatggccggccgctatccgaaagcgaaaagtgtggcggagttttgggaaaacctgaaggcaggcacggattgcatcactgaagttccgaagtcccgatgggattggaaaacatatcaaaacgtaacgtccccgtctggaaaaacagtatcaaaatggggcggtttcattgacgatgccgactgcttcgatccgcagtttttcagaatctcaccgcgtgaggcggaaacgatggacccgcaggagcgcctgtttttagaaacatgctgggaaacgattgaagatgcggggtatacgcctgaaacgctggggaataaagggaagaaacagcatccgatcggcgtatttgcaggggtgatgcataaagattattccttaatcggcgcagaacaattatctgaaactgatccttttccggtttcactgaattatgcgcagatcgccaacagagtgtcctattattgtgacttccacgggccgagcatcgcggtggataccgtttgttcctcatcattgaccgccgttcatctggccattgaaagcatccgccgcggcgaatgtgaggccgcgcttgcgggaggggtgaacttgtcactgcacccggcgaaatatctcagctacggaagcgtgggcatgcactcaagtgacggccgctgccgtacattcggcgagggcggggacggctacgtttccggagaaggagtcggtgcggtattgctgaagccgctggaaaaagccgaacaagacggcgaccgcatttacgcggtcattaaaggaagcgccgttaaccacgtcgggaaagtaagcggaattaccgtcccgagtcctgctgcacaggcagaagtcattaaggcctgtctgaaaaaagcaggtatcagcccgcggaccgtcagttatgtggaagcccatggcacgggtacttcgttaggagacccgattgaaattgaaggtctgtcaaaagcgttcagccaagggacgcaggatcagcaattttgttccatcggttcggtcaaatcaaatatcggccacgccgaatcagcggcggggatcagcgggttaacaaaagcggcgctgcagcttcatcacaaaacgcttgtcaaatcactgcattctgcggaattaaatccttacctgaaatttgaggaatctcctttttacgtacagcagcagactgctccgtggaagcagccgtcagctgaagaaaacggtaaagtcacacattatccgcgcagggccgggctcagttccttcggtgcatcaggttcgaatgcccatatcattttagaagaatacattcagcctgaacaaaaagcgcctgcacctgaaaacgaggcagcaaaactcatcccgctgtcagcaagaaataaagacaggcttatggcctatgcggaaaaactagcccgatcgctttcagaaaagacggtgctgtctgaactggcttatactcttcagacaggacgtgaagcaatggaggagagagctgtcttccttgtaaatgacatccgcgacttgaagcaaaagctgaatgattttgtaaaaggaaacgaaaacatccccggtctttggcgcggacaagctgagctgccggagatgagcgaagccgctcccgagacggacgatccgaaacggcttgccgcgttatgggcagaagggaaaataatagattggaataagctgtacgcttcgggtaaaccgtgtaaaacaagcgttccgacttatccgtttgcaaaagaacgctattggattccgcagcagaaaaaacaggagaaaagcccgtcaggcacagtactgcatccgcttttacaagaaaatacgtcagatttttcgcagcagcgcttcacgtctctttttacgggagatgaatttttcctatctgaccatgtcgtcaacggacaaaaaatactgccgggcgtcgcctatttggaaatggccgcagaggcggtcaaaaaggctgctgccgtgacggaaaaagaccatagcgtcacactcagacaggttgtttgggtccggccggtcatgattgaagacaaaccgaaagaaatacatattgccctttacccagaagataatggtgagattacctatgaaatatacagtgaggcagaaaacggcgaagatccggttattcacagccagggtgaagcggtcattaccgaaccgggcagcgcccagatagatatcgagtctgtcaaagcttcctgcaatttaggaatcgtgactgcggaagaatgttacggtgcttttgagaagatcggtctggattacggagaggcatacaggggtgtacagcaattgctgatcggagaagaccgattattggcccatatttcactcccagctgccgtacatgacaaaaaagaacagtatacgcttcacccgagtatgatggacgccgcttttcacgcctccgtcggtctgcttgtccgctcgatacatgatgatgcagacagccacgagctttcattgccgtttgcgatgcaggaagtggaagtgttcggaccgtgtcctgaacatatctggtcatatatccggtacacagaaggcagcaaagcagatgacaacgtaagaaaatttgacattgatatgtgcgatgatgccggtacagtttatgtccggatgaaaggcgcgtctgtccgggcgcttgacacaagccaaacagcgggcggagaaacctcggaggcgcctgtcggccatacgatgctgattccggcttgggagcctgttgcgcttgaaaaagccggggaagcagcggcggataagcagacggcagtattcggcggaacggattcgagacatacgcgcataaaagaacacctgccgaacgcttcctattatccgatcagaggtacggatgatatcacatctcttacgcacatgcttgaaggacaaaaagaactggagcaaatcatttggcttgctccgcaggctgagagtgcggacagcctcaaagaacaggaaagaaacgttcttcatgtgttcaagctgatgaaagcattacttgccttagggtacggagaaaaacgtttggactggtgcatcgtcacagcaaatgcccagccgttttcggatcaagacagcgccgacccgtcacaagccgcagtcagcgga

SEQ ID NO: 23 - DNA, 9960 bp, unidentified
  source 1..9960: mol_type = genomic DNA
cttgccggaacaatggcgaaagaatatccgcattggcatatccgcatgattgacacagaaacggacggcagctggccgatcgaagaggcgctcagtctttctgccgggaaaaacggtcaaatttgggcgtaccgcaatcagcagctgtttgagcggcagctgataccgcttgacaatatgccgaaaaacggcacgctgtaccaaacaggcggggtttatgccgtgatcggaggcgcggggtatcttggcgaggcatggagtgaatacatgatccgccgctacaaagcacgcatcatttggatcggaagaagcacactgaatgattccatacaggcgaaactcgaccggctcagcgcattcggcccggcgcctgaatatgtttccgccgatgccgccgacagtgcttccttagagcgggcttatcagttcataaaagagcgccacacaaaaatgaacggaatcatccactcggcaatggatctgtctgagcaaagcttaaataatatgaagacagaagagttcaaatcggtgctgcgggcaaaagccgatgtaagtgtgtccatggcggaggtatttggacgggagccgcttgactttgcgctcttcttctcctcgcttgtggcatttatccataacgtgaaacaaagccattatgccgcgggatgcgctttcagtgacgcatttgcgaaagaacttgatcagacatggaattgtccggtcaaagtgatgaattggggctattggggcaacagcgaagcggctgaagacggcgattttattcagctgatggatcagatcggattgggattgatcgaaccggacgctgcgatggaggcgcttgaaacccttatgacgctgccggttgatcaggcggctttcattcatacgacaaaaccggtcgcagtcgaagggatgagtgaaagagaaaagatttccgtttatccgcctcagccgcttgtcttttcacaacagccgaaggtcgctccgcccggcgtacgtcttgttagagaggctgaacatcaggagctcgatgattttctgtatcagctgttgatcgcccagcttaaagaagcagacttgctgacgccgcaaagccttaaagcgtatgagcccgaacacgaatactttaaaaaatggctcaatcaaacgatcgctttccttctggaacatggctatctggaagggaatgcggattcatataaactgccggcgcgagccgaaaacacaaccgcctcgctatggcaaaaatgggagcaggcaaaagaggaatggctcaacaacgccgatacaaaagcgatgactgttttagccgagacgatgcttaaagcactgccggacattctgcgtgaagacgtgccggcgacagacatcatgttcccgcactcttcaatggaacttgttgaaggaatttataagcacaatcaagtcgcggattactttaacagcgccctgtcagatacattaacggcttatgtgaaagaaaacatcagcaaacattcaaaagccgagatccgcatactggaaatcggagcgggcacaggcggcacaagcgcaggcatatttgaaaagcttaggccgtatcaaaaccatataaaagaatactgttacaccgatgtatcgaaagcttttctcgtgcatgcggagacggaatacggcaaacggaatccttatctgacgtataagctttttaacgcagaagcgccgcttgccggccaggacatagagccgggcgcatacgacatcgtcgttgcggccaatgtgctccacgcgacgaaagacatccgccagacgctgagaaacgccaaagccgttttaaaagacaacggtttgctgctcttaaatgaaatggcgggaaacagcctgttcccgcatattactttcggactgcttgaaggctggtggctgcatgaagattctgcccttcgtattccggggtgcccgggcttgtatccggaaagctggaagcgcgtgttagaaagtgaaggctggcattctgtcttcttcccggcagaagcggcacatgaattaagccatcagattatagcggcaaaaagcaacggcattgttcgccaaaaaacgcttaaagcaggggccggaaaacggaccgctccgccaaaagcagcacatgcgccggaacttcaggcgcaaacaacggtgcaaaaaggcgacacgtctttaacagaaaaaagcaccgagtacttgaaaacgttaatcggagaaacattaaaaattccgccggctcaaattgatccgaaagcgccgcttgaaaaatacggaattgattctatcgtcgtcgtacagctgacaaacgcgttacgaaatgtattggaccaagtcagcagcactttattttttgaatatcagacgattgaggcgctcgtcgctcacttaatcaaaacgaaaaaagacgcgatgatcaaactgacgggacaaacgcctgttcaggaagaacggaaggcatcaccagaaccgcaggctgtacagccggctaagccgcttcgcactgactggcggcggaggaaaaaacgggcagccaaagagaccgtccggcaaagttcggttcgcgacgtggccatcatcggtgtttcaggacgctacccgcaggcaaaaacagcggctgagctttgggctaatattaaaaaaggaaaaaactgcatagaagaaatcccgcgggaacgctggaactggcaggattactacgatgaagagaaaggtaaagaaggctctatttatacgaaatggggcggcttcattgaggatatggacaagtttgatccgctgttctttcagatttctcctttggaagcggagcgaatggacccgcaggagcgattatttttagaaacggcgtatgcgagtatcgaggatgccggctatacgcctgacaacctgtgccagagcagaaaaatcggtgtttttgccggcgcgatgaacaaaaactacccgacaggctacggctattggtccattgccaacagggtatcttacctttttgatttccaagggccgagcattgccgttgatacagcctgctcctcttctttgacggccattcatctggcgcttgagagcatatacagcggttcaagcgaatgtgcgattgccgggggcgtaaacatagtgaccgacccaatccattatatgaatctgtcagtcatgaacatgctttccgcaggcgacacgtgcaaatcgttcggagaccgcgcggatggttttgtcgacggagaaggggtaggagcggtcgtattaaagccgttggaccgagcgattgccgacaacgatcacatttacggtgtgattaaaggaagcaccattaacagcggaggcaaaacgaacggatacaccgttccgaatccgaatgcgcaggccgccttaataaaagaagctttcgaaagggcaaacgtcccggcgcgcaccgtcagttatattgaagcccacggtacggggaccgcattaggagatccgattgaaatcgccggattaacgaaagcatttgaagaggatacaacagacaaacagttttgcgccatcggctccgcaaaatcaaatatcggccattgtgaaagcgccgcgggcattgcgggtctgacaaaaatattattccagctcaaagaccgacaaattgcgccgtccctgcacgcggatcagctgaatccgaatattgatttcgcgtatacgccgtttgccgttcagcaggagctcggtgagtggaaacgtcccgtcatcggcggcaaagagctaccgagaagggcggggttaagcagttttggcgcgggaggagcaaacgcccacctcgtcatagaagaatatacggcagacaaagaacctgatgagacgccggcagatcagcccgccatcatcgtcctgtccgcgaaaaacagcgaacggctcactgaaaaagcaaaacagctcagagacgccatccgtgaaaaaagatacagcggcagcgaccttatgagcatagcctttactcttcagacgggccgggtgccgatggaagagcgtcttggttttatcgtccattcacttcatgaacttgaggaaaaactgaatggatttaccgccggacaaaacagcgccggactgcaccgtgacggcacggaagaaaacaaacggatgcttgccgtattttcggcggatgatgatctggaaacaatcatagacgcttggatcagcaaacggaaatattctaagctgcttgagctttgggtaaaaggattggacatcgattgggcgaaattatacggagaccgtcctccggcccgcatcagcctgcctgcttatccgtttgcgaaagaacgcttctggcatcagcagcctgtacaaagcaaggccgcgcccgttaaggcgccggctgatgaaagtgatccggcggaacagtttgaagtgatggcttttagagaagttcttaaagaacaggctctgccggctgccggagaaaagcaagtcaaaacggtgatttctttcttatctgatcagaagaagcagcaggaagccgtctctgtgatcaaatcgtatgacagtggcattggcgttatttttatcgctcagggagaccggtatgaggctctgtccgccggccgctgccaagtggtgcgggaagatccgcaatcatatgtgaacgcttttgcggatattcaaaaagagtacggtgaagcttctgccattttgtatttatgggcgcttgaagataaaagctgcatcgaagattattcatgtatcgtccatattctcaaagcaatggctgagaccggtctgcagccggaaaggctgttattggcgggggaatatgcatccggtctggaaaagtgctacttggaatcatggatcggctttgagcgttcgctcggcattgttctcccgaaaacaagtgttaccggccacttcagaaaagccggtgcggccataacggactggatgagaaccatttgggacgagctgaaagctgaaacagaccataccgttttcttggaagagggaaaacgatatgtctatcacaccgaaccaaccggactaacgaaagaaaacggcaggatcagaaccggcggaacatatctcattacaggcggattaggagggctcggctacttatttgcccgccatctcgcccggaactaccaggcaaatgtcattctcacggggcggtcgccgatcaatgaggaaaagaaagaaaaaatgaaagacctggaaaatctcggcgcgggtgttctttacgccgaagccgatgtttgtgatccgatcggaatgggcgactgcatcaagcgcgcgaaagaacgattcggagccattcacggcgtcattcatgcggcgggcatcgaaagcagaacatccgtctttgaaaatgaaatcgggaatttccgcagtacgcttgaaccgaaaataaacggaagcctgcttcttgacgagtggctgaaaaacgaaacgccggatttcatctgctacttttcatccacatcggctgttttaggagatttcggctgctgtgattatgcggtcgcgaacaggttccaaatggcgtatgcccactataaaaacgaacaatcagaaagcggaaacgcttttgtcatcaactggccggtctggaaaaacggcggaatggccgtcggcagtgaagagacgacgggcatgtacctgaaatcaagcggccagcgcctgttagaaacagatgaaggcattgagctttttgagcgcatcatttctcaagacaacacccagtgccttgtcattgccggcgaaagaagccgtgccgaacgatttttaggcatacggcgggacgattcatcaaaaccggatgctcttttggaaaccgtgagccgccgcatcccggaagaaagcacggaaaaacggctcgaagcggatttgaaagagctgatacacagtctgctgaaaatatccaaagataaactcgtcctgcataaaaactgggcggaattcggatttgattccatctatttagccaaatttgccgctcttttgacgagtcattacggcattgaagtcactccggcgttattttacagctatgccacgctcggagacgtcatcagctattacctgacagaacataaagaaacgatcgaaacgttttaccgaacggaagaaaccgaaacagaagccgccgctccggataacaaggagtatacggatcaggagatcatcgccatgatgaaacaggtctctgaggggacattagattttaaaagagtgcaagacatcatagaagggagtaagacctatgaatcataaagaacttctcgatgcctacagatcaggaacgctcaccatcgctgaagtagaacaaaagcttcaggctttcaagcggacaactgcaaaacgtccgctttctgaaggccagaaaggtttgtggatgcttcagaaaatgtttccggagatgagcgcatataacattccgttatgctttcgttttcaaaagctggatatcgagacattcaaaaacgcccttttgtccgtgcagcgccagcaccctattttgaccagtgtgataaaagaggaaaacggccttccgtatcagatagaagatacggatcggcattttccgattgaagaaatggatctttctgaattaagcgggcgtgacgctctttcgtttattaaagaaaaagcaaaagaaccgttctcgcttgaaaccgggccgctcctgcgtgttcaagtattccggcgggcgaagcgggagccgattgtgttaatcaccattcaccatatcatttttgacggggtctctcttatgacgtttatgtcggcgctgctggacgcttatgtaaaagcaagtgaaggcaaagaacctgtcatgcttcagcaggccgcagattatcatgactttgtgaaatgggaaaaagagctgctggatagcgaagcgggaaaaacgcatctctcttattggaaaaagcagctggaaggaccgctgccggtgcttgagcttccgtcggattatccgagatcatcggttcaaacttgcaaagggcaggcgtatcagcgcttgcttcctgatgaactgaaccggcagattaaagtttgcgcccaaaaacataatgtcaatgaatccgttgtgtatctcgctattttcaaaggattgctctatcaatatacgaatcaggaagatgtcatcgtcggcatgccgacgatggggcgttctgaggctcaatttgagtctgtcatcggatactttattaatatgatcgcggtcagaagcagggagacgggcgccaaatcttttacggcgtttttgaaggagctgcagctgacactggcagaagggctggaccatgccgcgtatccgtttcccgcgcttgtcaaagaattgaagacggaacgctcgcctgcggtgtcgcctgtttttcaaaccgcgtttttctatcaaaacttcttccggacagaaggagcggaaaagctggcggaactatatcaatcactcggcattgaaatgatagaagacattcatcaggaaggcgaatttgagcttgcgcttgaagtctataaaaaagaaacgggaaccgtcctgcaatttctgtacaacccggatgtctacagcccttctttcatcgaacgaatggcggagcattatatgaaactggcagaaaaggtgctttctgatccggagatcgcttttgaagcctgttccggcctgctcaatcaaaaacgggatgaaagccgttcatcccatcagccgaatgagcgctgcattcatgaagtgtttgcggaaaaagccggacgccaccctgacagagaagccgtcacattcggtgaaaacagtcttacatatcgcgaattagatgaacgaagcacggcgctcgctgtgtacctgcaagaaaagggcgtcacaccggaatgcccggtcggcatttgtacggagcgttcttttgagatggtcatcggcatcctcgccattttaaaagcgggaggcgcgtatgtaccgctggacccggcttttccaaacgaacggatggcccacgtgttgactgacagcggcgtgtcaatcgtattaacgcaggaaaagacgcgcggcatgattgaacagttggccatcggcggtgtaagcacggtggaaattgaaaacagcggacgttctgcgagcggggcaaaactgacgccgcgttcaaaaccgcatcatctcgcttatattctatatacgtccggaagcacgggaaaaccgaaaggcgtcatggttgagcacagggccatcatgaatacgctgcagtttttggaagctgagtatccggtcgcacaagaggatgcctatttgttaaaaacgaattacgtatttgacgtttcgatttctgaattattcggctggtttattggaaacggacggcttgtcattttgccgcctggtgcagaaaaaaatccgcagctttgcatggaatatatccaaacccaccaggtcacgcatctgaattttgcgcccgccgtgtttaatgtatttctggaaacggtaaagcggcatacggcatttacagaagacggccctgtgaaatatgtgatggtggccggtgaggcgtttccgaaagatttagttaaaaagtcagtttccatcttcaaacatgccagaattgaaaacatatacggcccgacggaaacatccatttatgccgcttactacagctgcggccaaaaggaaatcacaagcaggaatacgccgatcgggaaaccgattcacaatacgcggctttatatcgtcgatgaaaagctcagccccgtccctgacgggttcgccggggagctttgcgtggccggcgccggtctggccagaggctattacaatcagcctgagctgacggcggcatcatttattgataatccgtttagtgaaggagaaaaactttacaaaacaggtgatttggccagatggctgcctgacggaaatatagaatatgccggccggattgacagtcaggtgaaaattcgcggcttccgcatcgaattgggagcgattgaaacgaaattgagcgaacatccggacatccttgaccaggcggttgtggttcaggaaaagaacggacataaaaagctgattgcttattatacggcccgctccggtcatacggcagacgaaaaagcgctccgcaaccatcttttatcgagtctgccggattatatggcgcctgcccattttatccgcctggatgcactgccgttaacaccgagcggaaaagtgaaccgcaaagacctaaaacagcgcaaggtaatcgttcaccggtctgaaaagacgggcagactgccgttatcagaagcggaagcgaaagtgaccgcaatatgggaagacctgctgaatacggcgggcatcaaaatcgaagacggctttttcgatgtgggaggcgactcgcttctggctgttgcggcggcagaacgcattaaacaggagctggaatgcgattttaccgtcaccgatttattcgaacactctaccattcaggcgatcagccgatttattgttgaaacaaaacaggcggctgaatcagtggcgcccggacaagatgagagagaaagagaacaaacaccgcaaaacggggtgccgtcttactttgatgacagtgtggcgatagtggggatctcctgccagttccccggcgcgaaaaatcatcatgagttttggaagcagttaagagaaggaaaggaaagcgtccgcttctactcggaagaagaacttcgggaagccggcgtgcctgaagatttgatcgaaaatcccgactatgtgccgacattatcgacgatagaaggaaaagatctctttgaccctgagttctttcacatttctccgaaagacgcagaatttatggatcctcagcttcggcttctgctgcttcattcttggaaagccgtcgaggacgcgggatatgtatcgaaagagattccgaagacaagcgtttatatgtcggcaagcaataattcgtatcggtcgcttttgccggaaaaaacgacagaaggccatgaatctcccgacgg

SEQ ID NO: 24 - DNA, 9960 bp, unidentified
  source 1..9960: mol_type = genomic DNA
ctatgtgtcatgggtgttagcgcagagcggcacgattccgacgatggtttcccataagctcggattaaaagggccaagctatttcgttcattccaattgctcatcatcattagtcggtctttattcagcctatagaagcataacgagcggagaatcggaatacgcgctcgtcggcggagcaactctgcatgcagcgaccggcatcggctatgtgcaccaaaacggattgaatttttcaagcgatgggcatgtcaaagcatttgatgcttcagccgacggtatggcgggcggagaaggagcggccgtcattctgctgaaaaaggcgtcacaagccgttcaggacggagatcacatatacgccatgctcagaggcatcggtctgaacaatgacggagcggataaagtcggtttttacgcaccgagtgtaaaaggtcagactgacgtcatccagcatgttttagacagcacgaatattcacccggagacgatttcctatatcgaagcgcacggcacaggaacgacattgggtgatccgattgaaatgtccgcgctgcagcaagtgtataaacgttacacggaccgggagcagtattgcgggatcggctccgtaaaaacgaatatcggacatttggatacagcagcaggtctggccgggtgcatcaaagtcgccatgagtttatatcatcgggagctggcgcctaccattaattacacgtcaccgaatccgaatatcaaattcagcggttcgccgttttatgtggcggataagagaaaaccgctgccggagagagagacgccgcaccgcgcagcgttaagctcattcggactcggaggaacaaacgcacacgccatttttgaacagtatgaaaacaaaacgatcagcggatcaaatgacggacagccgccttatatcgttccgctgtcagcgagaaataagcagcgtcttactgcctatgcatcctgtttatccggctttctggatgaagccgaaaacgatgtttccttgcatgatctggcatatacgtatcagaccggacgtgaagcgatggaagagagagccgtttttatcagtcatgatcgtcacgatttgaagcgtcagctgcaggattttatcaacggcaatgaccaaaacatcttacggggcgaaaaagtgagaagcagagaagtgtcagctcaggagatggaagagcttgcggcatgtcagacacgggatgaaaagctgaaagcactcgcggcgctctgggttgaaggagcgcgtgtcgattggggcttattgtatccggattccgcgccgcaaagaatcagtgcgccgacctacccgttcgcagaggagcgcttctggccggaagaaacggtgaagcaaacggataccaagcttttgcatccgctggtacatcagaatacatccgtcttgcaggaacagcggttcagttcggaattcacgggaaaagagtattttattgccgagcatatcattaaaggaatggcgatcgtgccggctgccgttacccttgagatggcacgggccgcggcggagaaaagcatcggcggggcaacagaggaagctctgggcctgcgcttaaagaatattgtatgggtccgtccggtgatagccgatcaggaggctgtccgtgtccatatcggactttatgaagaagaggacggacagattcaatacagaatgtacgggggtaacgattctatcggtgaagaagctcctctttataatcagggcatcgctgaaatcatccggaaggaaccggaggaagcggcggatattgagcagctgaagcgccggtgcacacagggaacaatcgaatctcaagcgttttacaaaggaatgatcggtgctgattacgggccgggttacaaaggggtgcagcttgtatacaaaggcgaagaccggctgcttgctaaattgaagcttcctgaatccgtctctcatacaaaagctgattatatccttcatccgagtatgatggacggtgcgcttcaggccgccgaatatttgcaaaacgtgacgagagcgctgcttgcagaaagcggggaaccattcaaagccgcattgccgtttgctttagaggagctggacgttctgaatccgtgcgctgatgatatgtgggtgcacgtcacattcagtgcgaacaataaagccggcgatcctatccaaaaagccgatattgatttatatgacaataacggcgggctttgtgtgcggatgagaggtttctcaacacggatcatggaagaaaacgcgggtgcaggcgatcctgtcagccgtacggacacattgctcgctgaaccggtctggaaagagcagccggaaccggccgatcacgagccttcgtatgaagagcatattattttcctttgtgagtacgatgaagccgtaaaacaggccgtggaagcaaagcttgaacatgcaagcgtcattatcttggacgatcgtcccgcatctattgcagagcggtatcattcttatgcgggccagctgtttgaccatatcaaaaccataatgaaaggcaagccgaagggcagcgttctttttcaggctgttacgcggtcagcaggcttacagcaggtgttctcaggcattaccggcctcttcaaaaccgcatgccttgaacattccaagctgatttgtcaaatgatcgaagccgaggagaaagaaagtgctgaaggcatagcggaaaaagcgtccgaaaaccgtatgcatgccgaaagcgtccatgtgaaatatgaaaacggcaagcgctttgccgctgcttgggaagagatggatatttctcaagcggagccggacattccgtggaaagacgaaggcgtttatctcattacaggaggtgcgggaggtttaggtctgatctttgcaaaagagatcgcaaaccgtacaaaagacgccgtcatcatactaaccggccggtccgctctcagagaaaagcagaaagacatgctggaaacagtacgtgctgccggcgcttctgttttctatgagcagaccgatgtcacggacgaagcagaagtataccgattgatccgcaatatccgcaaacggcatggcaggctggacgggattgttcacagtgccggcattataaaagacaactacatggtgaagaagacggcagaggaataccaacacgttctcgctccgaaagtgaagggccttgtgtatcttgatgaagcgtcacaacatgagaaattagatgtctttattgtcttttcatccttatcaggtgtgcttggaagcgtcggccaggctgattacgcgtctgcaaatgtctttatggagatgtatgccgagtaccgccgttctttacaagccgcaggacaaagatacggaaagacgctttctgtcagctggccgttatggaaagaaggcggaatgcaggcaggaaagcagacggaagatatgctgatgcaggaggcgggaatcgcaccgctcggcacagatgccggcatcaaggctttatatcaaggcttgatgagcgggacagcccggatgacggttgttgaaggagatgtgaaacacatgaaagcaaaactgttccgcaaagagaatggtcatccggaagcagagaccggcgtacgtcatgacattgatgctgacagcctgcttgaaaaagtgaagcatctgttaaaacagcagacggcctcactgttaaaagtaaacattgacaaaattgatcctcatgaagaaatgacaaaatacgggctggattctatctcgatgactgagtttacgaaccagttaaacaaaacgtaccggcttacgttaacgccgacgattttctttgatcatccgacaatccatgaatttgccgttcatttaatcagtgaatacgaagaggaatttgccggacggtttgcggtaaatacaaaaacggttcaatcggcaagaccggtaaaagagactcaaatcacgcgcccggccgttaagcgacgccgcactctgcctgagacacttccgcagacagtgcagcggggccaagggccggaaccgatcgcaatcgtaggcatcagcgggattttcccgatggcagaagacattgaagctttctggaagaacctgaaagaaggcaaagactgcatgaccgagattccgaaagaccgctgggactggcgggactattacggggatcccgcaaaagaagccaataaaacaaacgtcaaccaaggcgggtttattgacggaattgctgaatttgatccgttatttttcgggatttcaccccgggaagcggagcaaatggaccctcagcagcgtctcttgctgacgtacgcgtggaaagcgatcgaagatgccggatatgcgtcaaagagtctctcaggcacaaagacaggtgtgtttatcgggaccggaaataccggctacggttctcttttagccaatgccgatgcggcgattgaagggtcatccgcggcgaatacaagtccgtctgtcggaccgaaccgtatcagctatacgctcaatctgcatgggccgagcgagccgattgatactgcctgctcaagctcgctcgttgccatacaccatgccgtttcttcaattgaagaaggaacgtgcgacatggctttggcgggaggaatcaatacgattgttcttccggatgtgtatatcagctttgataaagcgggcgcgctgagtaaagaaggaagatgcaaaacgttctctgatcaggctgacggttttgcccatggtgaaggagcagggctctttttcctcaaaaagctgaaagccgctgaggcggatggggaccatatttacggcgtcattaaaggaagcgcggtgaatcacggcggccgcgcggcatctttaacaacgcctaacccgaaacaacaggcggaagtcattaaagccgcatacaaaaaagcgggcattgatccgaaaacagtctcttatatcgaagcgcacggcaccggaacagaactgggagatcccgttgaagttaacggcctgaaacaagcgttcagctcgtttgcagacggaaaagagaaggcggccggctattgcggcttagggtcggtcaaaacgaatatcggccatttatcccttgccgcgggagcagcgggcattattaaaattttgctgcagatgaagcatcggacactagtaaaaagtctccactgtgagacggtaaatccgtacattcagcttgaagacagcccgttttatctggtgcgggagacaatggaatggaagacgcagacggacgaacgcggaaacgaacagcctcgccgcgcgggcatcagctcattcggcatcggaggcgtaaatgcccacgtcgtcatcgaagaatacattccagaagacagagaggaagcagatgattctcatatcgacccatgtcttttcgttctgtccgccaaaaatgaaaaacgcctgaaagagcaggcaaaacagctgaaacgtgcgcttactgaaaaacggtacgatcaatcctccctcgttgctatggcatataccctccaaatcggaagggacgccatggaagaacgcctggcggttattgccggatctgcagaagaactgcaggaaaaactgtcacagtttatcactggagataagcacgcggacttgtacagaggaagaatagacaaaggcacgctgcaaatgctgacggaagatgaagaaattcaagaagccgttgaaaaatggatggcacgcggcaaatacgcgaagctgcttgaattatgggtcaaagggctgaacgtcgattgggcgaagctgtatggagaccggccgcctaaacgcatgagcctgccgtcgtatccttttgcaaaagaccgctattgggcgtcaaaaagcaccggaaaacacgaaaaaacaagcactgtatctgcgggaaaacaagacgcgcttcatccgcttctgcacagaaatacttcaaaccttgcagaacagcggttcagttcaatctatacaggaaaagaattttaccttgcggaccatgttgtaaaaggcgtaccgattttaccgggagtggcacatcttgaaatggcgcgcgcagcaatggaacaggcatctgaagttccggccggacaaggaagcgtcaagcttaagaatacggtttgggtgcggccgatcaccattgaagacgagccggttcatgtcaacatccgtctcattcctgaaacggcaaatgacgttcaattcgacatttacagcggccgggaaacagatcagccgtccgtttacagccaaggggcagtatcagtccgccctgacgccgaggcaccggtcattgatctccaaaagctgagatcacaatccgccgagacaccgttttctgcagatgaagtgtacgatatgtaccgcaaaatcggttttgattacggcccggctttccggggtgtcaaagcgatttatacaggtgatgatttcgtattgggcagactgtcacttgatgcggcttgtgcggacacggtagaggagtatgtcatgcatccgggcttaatggactctgcattgcaggcatcaagcatattaaccggaacaaatgatgaaaaactgatgcttccgtttgcggttcaggagcttgaagtattcagcgcatgcgctgccgagatgtgggtttacgccagactttctgaagaccacaaagccggccagattgaaaaacgcgatcttgatatttgtgatgaaaacggcaaggtttgcgtaaggctgaaaggtttgtcattcagaacggctgagaaagaacctgagaaaaaagaaaaagctccggaaacgctcatgcttgagccggtatggacccgaagcgatgaaaccgccgttcaaacggaaccggtttttgcccggcatatcgtgttgctttgtgacacagacgccggattgaaacaagccgttgaagccgcgggagctgaatgtattgaaatgacctcccggcacgggaaagcggcggcgcgcttccgtgattatacaggacggatattggagacgctgcaaaaactgctgcaggaagagacggatggagacgtccttatccagacggttctctctaaaacagataagcatcagctgctctcaggcatttcagctttgttaaaaacagccggcctggaacataaaaaactgaaaacccagctgattgaaatagaaccggcggaaaacggcagaatcacagacatcatcagagaaaataaaacacgggctgatgacagccatatcaaatatgaaaacggcatccgatacagcgcagatgtcaaagaaacggccgtgtccggcggcaaaatacgcattccgtggaacgacggaggcatatatctcattaccggaggagccgggggcttaggctttattttcgcgaaagaaatcgcccgcaaagtcaaagagccgacactggttttgacaggccgttccgcattaagcgagaatcagcggatgcagttgcaatcgcttgaatcactcggagcaaaagcggaatacaagcaggctgacgtaacaaacgctcaaactgcggcagacctggtcaaaacaattgagacggaatatggcggccttcacggcatcattcatagcgcgggtgtgatgaaagatcagtatatcgcaaagaaaacaaccgaagagttttacagtgtattggctcctaaaacggacggatttgtcaaccttgatgaagcaaccgagcatttagcgctggactttttcatcgttttctcatccatttcgggtatcaccgggaatgcgggacaggcggattatgcggcggcaaacgcatttatggacagctatgcggcataccgcaacgcgctggttaccgcaatgtaccgccacggtcacacattgtcaatcaattggccgctttggaaaaacggcggaatgcaagtcaatgaagaaatggcgaagctcaccgaaaaaagaaccggcgtaacgccgatgcgcactgagaccggaatagaagctctttacaaagcatggagcacgggaaaaacaagcgtgctcgtcatggaaggaaaccgcaccgtaatgcgggaaaagctgcttcaaaagactcctgcagccgcttttgcaccggttaagaaaacagatgaaacgcctgcgccgaaagacctgacggcaaaacttcaggaattgctggcaaaagaggtttccgggcttctgaaaataaacattgaagaaatcgatatcgaattggagtttaatcaatacggatttgattccattacgctgacggaattcgccaacaagctgaatgacacttatcagcttgatctgacaccgaccgtattttttgaatatgcgacgattcaagccttggcgcatcatttagcagaagactatcaaacacaatttgccggcagcacacagccgaaagaagaaaaacaaagcggtgtaacagatgatggcggcatcagactggcagggaacaaacgatttgcaaaaactgccgtacagcctgttcagactgcggctgaacataaaccggaaccgatcgctattgtcggcatgagcggagtgtttccgaaggccgcaaatattgatgagtattggcgcaacttagaagaaggaaaagactgtataacagaggtgccggcggatcgctgggactggcgtgagtattacggtgacccgcttaatgaagccaacaaaacaaatgtaaagtggggcggatttattgacggagttgcggattttgatccgctgttcttcggaatttcaccgctggaagccgaacagatggatccccagcagcgcttgttaatgatgtatgcgtggaaggcgatagaagatgcgggatattccgctaaaagcctttccggcacgaaaacaggcctgtatatcggcacggggaataccggatacggctcactgttttccgacctggatatcggcggtgcgtcagccgcaaatatgagtccgtcagcggggccgaaccgcgtcagctatatgcttaatctgcacgggccgagtgaaccgatcgacacggcttgttccagctcgctggtcgcgattcatcacgccgtatgcgccattgaaaacgggaactgcgatatggcgatcgccggcggcgtgaataccgtcgtcacaccgcaaggccacatcgcttatgacaaagccggcgctctgagcaaagaagggaaatgtaaaacattctccgacagggcggacggatttgccgtcagtgaaggtgccggaattctgtttttgaaaaagctgtcagaagcggaaaaagcgggcgatcatatctacggcgtcattaaaggcagcgcggtcaatcacggcggacgtgcgaactccttgacgacgccgaatccgaaagcccaggcggaagtcgtcagaacggcttatgacaaagcaggcattgatccgagaacagtcacttatattgaagcgcacggaaccggaacggagcttggcgatccggtcgaaatcaacggcctgaaatccgcctttcaaaagctgtacgaaaaaacaggcgatcccgccgtttacgcgagtcactgcggtctgggatcggccaaaaccaatatcggccacctgtcactggcagccggtgtcgcgggtgtgatcaaagtgctgatgcagatgaagcataaaacacttgcaaaaagcctccacagcgaggtcatcaacccgtatatcaagctgaaagacagtccgttttacatcgtacaggaaaaaagagaatggacggcattgaaagacgaaaacggcaatcttctgccgcgccgtgcgggaatcagttctttcggcatcggcggcgtaaacgcccatgtcgtcattgaagaatatatcccgaaggaagaaagaaaaacagctgttttcacacaccctcaaatctttgtgctgtcggccaaagaagaagagcgtctgcaggaacaggcgcagcagctggcgcaagcttcttttacggatgcggatcttgccgatgttgcttacacgcttcagcaggggcgtgatgccatggaagaaagagcggccattatcgcttcctcggctgaagaactgcgggacaa

SEQ ID NO: 25 - DNA, 9960 bp, unidentified
  source 1..9960: mol_type = genomic DNA
gctaaagagctgtgcgaaaggacgttatgatatagcgggcatttataaaggaaaagcgggtaaaacctccctgaaatccattctggcaggcgcaaacggggaagcggatatcgaagagctgataagccgccgagagaatgacaagcttgcggaattatgggtgcagggcgcggatatcaaatgggacggcctatattctgatatcaagcctcaacggatgagtcttcctgcttacccgtttgcgaaagagcggtactggatcacggacaatcggccgaaagcagagaaaccgcaagctgtgccgacgtcgcgggtgctgcacccgctgcttcacgaaaatgtatcagatttaacagagcagcggtaccgatccgaatttacgggacaagagacgattttcaccgatcatcttattaagggcaagccggttatgccgggcgttgcctacttggaaatggctagggcggcggttgctcaggcactcaaagggcaaaagcaacgtccttctgtcacccgcctgaaaaacatcgtttgggttcggccgatcgaagcgggaaaccgcccgattgaagcgcaaatcagcctgaacccgagggaaaacggtgaaatcgtgtatgacatttacacggattcacaaacagaagcaagcggcaggcagatacactgtcagggggctgccggcattctcagcgccggggaggctccgcaccgtgatataggcgcactgaaagctcaatgcggcctcagtatactgacttctgaacattgttacggcttattcagcaaaatcggcattgactacggtcccggacaacaaggaattcagcagattcatatcggaagcaatcaagcattagctgagctccggctgccttccttcctcgaagaaacggcagatcaatacgttcttcacccgtgtatgcttgattcagctttgcaagcgacaatcggactgaagctgcacgcagatgataaccagctgtcactgccgttcgcccttgaagaactggacattttcagtccgtgcacgacaagcatgtgggcgtatgtcagctcccggggaacggatgaaaaaatacagcgtatggacattgacttatgcgatgagaccggccgggtctgtgtcagaattaaagggatttcttccagaatgatggaaggccccgcgccggaaacatcattaaatggagcaaaccttctggctccggtgtgggatctcgcggaattccgtgatcaaagcccggcatataagacggaagaagttgtctttatcgcgggggatgaggaaagcgcggcctttcaattgcatcatccttctgcgaaacggcttcatatcaacccggaggatgaaatagaggctgttgcggctgaactgaaagcaacagggtctttcgggcatatcgtatggatcgctccttttcacagcgctgatgacatggtaaaagctcaggatgcgggcgtcatccagatgtacagagtgattaaggcaatgcttgctttaggatatggaggaaagcgaatcagcttgactgcagtcacaataaaaacacagtccgtacagccggcggacattattgatccggctcatgcaggcattcacggtttggtcggctcgctcgcaaaagagtatccgaactggcaaacaaggctgattgatgccggatgtccggaagatgtttcaaaggcggagctgttctcagcgccgtttgatgagcaaggcaatacatgggcgtaccggaacaatcactggtacaaacagcgccttattcctgtacagcagccggtgtcaagccggcccgcctaccaaaaaggcggcgtttatgtcgtcatcggcggtgcggggggcatcggcgaggcatggagcgaatatgccgtgaaaacatatcaggcgcgagtcgtttggatcggcagacgaaaacataacagcgccatccaggaaaaaatcgacagactcagccaattcggcccggcgccgatttatattgaagctgacgcggcaaatccgaatgagctcacacgggcgtatgaaacgatcaaacgcacgcacccgcaaattaacggaatcatccattctgcaattgttttagaggatcagagcctggcaaatatgccggaaagccgtttgaaaagcgtgctcgctgcgaaagtcaatgtcagcgtgaatatggcccgcgtgttccaaaatgagccgctggactttgctttgttcttctcatctgttcaatcatttgcacgagcggccggacaaagcaattatgctgccggatgcagtttcaaagatgcctttgccgctcatttggctcaagttcggccgtattcggcggcagtgatgaattggagctactggggcagtgtcggaatcgtatcatctgccgactatcaagaacggatgaaacaggcgggagtcggatcaattgaagcgccggaagcaatggcggcgcttgaacgattgctcggcggcccgttgaatcagttcgtcatgatgaaacaggcggaagagccggaggaaaaaccggaagaaatgatagaagtataccctgaacatcacggctcggcgatgcaaaaactgcgaagctatcagcctaaagtaacaagacatgtacaacaattgagttaaggaggggacggctttgcaaaacactgaagatcttctttacaaactattggcaggccagctgaaatctgtcggattttttacggaaaacgaaacgcagcctgtcatgcaggggttttacggcaggtggctggaagaaagcatctccatcttggtgcaaaaaggctttttgaaacagtcaggcagtacgtatgtgttaacaggtccgatagagcgtgcggacgttctgtggcaagaatgggatgaaaagaaagctcctttactcaaggacagcagcaaacaggcgatgatcaccttagtggagaccgccctcagagctttgccggatattttatccggcagagtttctgccactgatgttctgtttcctgattcctcaatggaattggtagaaggaatctataaaaacaatgaaacggcagactactttaatgaggtgctcgctgacacattgacggcttacattgaagaacgcctgaaagaagacccgaaagcggaaatccggatattagagatcggagcggggacgggcgggacgagcgccgccgttttcaaaagattaaaagagtacaaacatcatgtgaaagaatattgctacaccgatctgtcaaaggcctttttcatgcatgctgaaaaagaatacggtcccgacaatccgtatttgacttataagcggtttaatgtggaggaacccccggctttgcagggtatagaggcgggcgcatatgacgcggtgattgcggcaaatgtgcttcacgcgacaaaaaatattcggcggacacttcgcaatgccaaagcggtgcttaaaaagaatgggattttgctattaaatgagatcagcagccattcggtttattctcatctgacattcggtcttttggaaggctggtggctgtatgaagacgccaaactgcgcattcccggatgcccggggctgtatcctgaagcgtggcagtctgcgctggaaagcgagggatatcggtacgttacatttatggcggaatcgtcacatcacctcggccagcagatcgtggcggcagaaagcgacggtgttatcaggcaaaagcagccggacgcgcaggcggttgaagaaactgcggcttccaaagagccggctgatttcacagaaccgtctttctcagtcacagaccggaccgtgcaattcataaaagaaacgttagccaagtccataaagcttgctcccgagcgcattcagccggaaaccagctttgaaaaatacggcatcgactccattttgcaggtgaattttatccgcgaactggaaacattgacgggcgagctgccgaaaacggtcttatttgaacacacgaatacgaaagaacttgccgagtatttgctggaaacttacccggataagctgcaacaggcattcggagccgaacaagcgcctcctgaaacaaagcatccggaacctgcaatgaatgaaccggtgccgcttgtcacggaaacgcggcggtttatcactgaacaaaaagagccaaaagcgccaaagccggaaacagaaaccgtcaccgttaatcgttctgatcgtgacgacattgccattattggcatcagcggccgctatcccgagtctgaaaccctcgatgagctgtgggagcatttaaaagccggagacagctgtattacgaaagctccggaaaacagatggaaatccggtcttctgaaaacaatggcaaaagaaacacgaaaagaagaacgcaaaacacggtacggcggatttctgaagcatattgacgcttttgaccatcacttgtttgacatccgggaagaccatgtgatggagatgacgcctgagctccgtctaagcttagaaaccgtatgggaaacatttgaaaatggcggttactcgttagaaagagtaaacgaatggcaggaatcagacagcggcatcggtgtatttatgggcagtatgtacaatcaatatttctggaatatcccgtcattggaaaaagctgcgctcagctctaacggaggggattggcatatcgccaaccgcatttcccactttttcaatctaacgggcccgagcatgggcgttacgaccgcatgctccagctcactgagcgccattcatctcgcctgtgaaagcctgaagcttaacagctgttccatggcgattgccggaggggtcaatctcacgcttgaaccgtctaaatatgatgcgctcgaacgggcgaatttgcttgaacagggaagtgaaagcaaaagcttcggaaccggcacgggcctgatgccgggagaaggagtcggggcggtgctcttaaagccgctctccaaagcgcttgccgacaaagaccatatttacggggtcattaaaagcagcgcgttgtgccatagcggcggaaggcagatgtacacggctccggatccgaaacagcaggcgaagctcatggcggcttccatcgacaaggccggaatcaacccagaaacaatcagttatgttgaatcagcggcaaacggttctgtattgggagatccgattgaggtcattgccctgacaaatgcgttcgcgcaatatactgataaaaaacgtttttgcgcgcttggatccgtcaagtcaaatctcggccatttagaggcggcttccggcatgtcccagctcgcgaaagtgctgctgcaaatggagcgggaaacattagtcccgacgatcaacgcaaaaccgcaaaatccgaatatcaaccttgaacaaacggctttttacctgcaggaaaaaaccgagtattgggagcgtatgagggatgccgaaacgggggacatcataccgcggagaagcatgattaattccttcggggcaggcggggcatatgcaaatctcatcgtagaagaatatatcagcccgaattccgatcaggcccgtcacggcgatgaggaatatattttccctgtgtcagcgaaaacaaaatggagttttgaagcctatcttgacagactggcggcctttttagaaaagacgccatctcttcatccggccgccatcgcatccgcgctgcaaaaaagaacgcatcattcggagtatcgggcggcatttacggctgcttccgttcaggaactgctgaataagctgtccgcttaccgcatggatcagcggaatcttaatttcactgaatctgatattggccggcgtgcgcggcaatgggaaaaaggagagaatatcgcgtttgatcagttgcagcaagagcacgccgttcatctgccggcctacgcttttgaccacgggacttcttttcaattcggcgaatcagaaaacgctgccggcgttcaggcggtatacagtgatgacgatccatacgtgaaagggcatcaattcagcggagaaccggtactcgtcggcgctacatataacagtctggcagtcgatcatttttgtgatctttttcctgaagcggaaggaggacaaattaaaaaactgaattacgtcagccccattaccgtaaaaaaaggcgatcgcatcgagctgaaaacagaatcgaaacaaaacggggatggatttgaactgagcataatgcaccgcagccgcacgtctcctgattggagcatagcggcgtatgccgagtgcagcccgtatccgtttcaaaagcagcacgcggatctggcggccattcagcgctcttaccgggaagtgggccggatagatgagttatacggagacggaacgggaaccgtcgtgtggggagaagaatttaaaacgatcacccgcctgtacaaaggctctgatgccgttttagcgaaagtgaacctgaacaatagcgactcacaacaagcgcatcattatgccgtaaatccgttcatcgccaacagtgcttacttggcctcgctttctttattggatcaaacggaggcggaaagctatctgccgttcggcattaattccgtcaccttcagcagccctgaagatcttaccgattgctggctgcacatcaagctggccaaacaaacgggggaaatgattctgattaacgcagatgtcatcagcagccgatcggaagtggtgatgcgttttgaaggctatgccctgaaacggttccgcccgaaaaccgaacagatgaatcagccggcccgaacggctgtgaagaatgcgggagcaccgatttctgccggcagtctgacagatgatatccgcagctacgtccttggcaagctcgccaagatgatagatgaacccgggccatctcttcgttctgacgcgaatatcatggaattgggacttgattcagcgagcctgatcgggctgacaaaagagatcggccaagaggcggacatcgatgtgaatccaacattgtttttcgagtatccgaccgcggaggagctgacccgccacttcgcgtcagcatatgagagcaagttttctcaactgcttgccgatcataagcattcagaagaggccagggtcacggaaacggaagagcgcactttaccgaagaaacagcatacgccttgtgataacgccgatgtcaagcaaggaatcgcgattatcggtatgtcagctcagtttccgcaatccccggatatccagtctttttgggagcacatcgtaaacggggatcactgtattaccgaaattccggcggaccgctgggactggcgccgttacgccggtgatgagaatgacacgagtctcagatggggcggttttattgacggtgtcggcgaatttgacccgctctttttcggaatttcccctaaagaggcatcacaaatggggccggagcaatttctgcttttgatgcatacatggaaggcaatggaggatgcgggcctgacaaataaggcattatcaagcagaccgacaggcgttttcgttgccgcgggcaacagcgatccgaacaatggaacagccattccgtccatcattccgaacagaatatcatatgctttgaatttacaaggaccgagtgaatattacgaggctgcctgcacgtcaaccctggtggcattgcacagggcggttcaatcaatccgtcataacgaatgtgagcaggctgtcgtgggcgccgccaatattctgcagtcgccgaagggctttatcggttttgactcaatgggatatttaagcaaaaacggccgggcgaaatcgttccaaaaagatgccgatggctttgtcagaagcgaaggggcgggcgttatcatcatcaaaccggttgaggcggcaattgaagacggcgatcatattcacatggtcattaaaggaacaggagtgtctcacggcggtaaaggcatgtcgcttcacgcaccgaatccggcgggaatgaaggccgcgatgaaaaaagcgtacgaagacaccgatgttgatccgcaaaccgtcacttatattgaagcgcacggcattgcctccgaaatggcagatgcgctggagtttaatgcaatcaaagcgggttacggcgagtctgcaaatcaagaggaaagcgcgccttgttatatcagcaccgtgaaaccttgcatcgggcacggcgaactcgcttccggactggccgcccttataaaagtggcgatggccatgaagcatcataccatcccgggcattcccggatttacagctgcaaatgagcaaatggccattcagaaaagccgcttccgtttcaccgaagacaatcaggaatggacacagctcaccgaccatacgggcagaccgatcccgcgccgggccgcaattaacagctatggcttcggcggcatgaatgcgcatgtcgttttggaacagtacagcccgagccataaagacaaagaatctgtgcatgaaccgcagcttatcgtgctttctgctaagagcgaagggcgtcttaaggcggccgcaagtcagctggctgattttgtgcaaactggagatactccgtcattgcgggatattgcttatacgcttcagaccggccgtgaggcgatgaactgccgcctggcactcgttgcccgcaatcaagatgaattgctggaaaaactgaaagagtacagagcattttccgaagaaagcaatggaatgaaaagcttctttacaggcagtcagcagactggatcaggagatatcggcgttttactggaagggactctcggtacagtcgtcacggagacgctgttggctgaaaacaatctcgaaaaacttgcattttgctgggtgaaaggtgcggatataccgtgggacagactccaccaaggcaaatctgttcaaagagtgccgcttccgacatatccttttgaaaaccacaagtattggaacggcggtcagccagaagacatcagccttgcaccggtttcacagaatcctcagatccgctgcgggagcgcagctgacattgtggctgacattctggggatgaaggcagcggaaatggatcaaaataagccgctgactgaatacggcttcgattcaatctcctctattcagctgttgaaaaaacttgaggacggagacagccgcatcagtctggaggctcttcagaaatgcagcactcttcaggagatcagcatgctgttaaaaacgaaccaatcgcaggaggcgggccgaaattttccagaactcgtcaagctgaatgatagagaaaaaggccgtcctgttttttggtttcacggcggaacgggaggcgtggaagcataccagccattcgccaaaaagagtcagcgtcctttttacggcatccaggcaagaggtttgacagggaaagagccgctgcaaggaatcgaagagacggccgcttcttataagagaatcatccaagcggtacagccgaaaggcccttatgatctcggcgggtattccttgggcggaatgcttgcatatgaaaccgcgcgcctcttgcaggaggaaggtcatacggtaaaaagcgccgtcatgatcgacactccgtacagtgaaaaatggaaagaaagaaaaccatctgttaagtcagtcatgctgcaaacgatcaatacgatgcttacagcatatcttaagcctgaaaaccttgaagaggcattaatcagcagagaagacattaccgtccaggctgaggaagaagaagtattgaaagaattgatcggccttgccaaaaagcgcggtctgccaaaatccgaagaagcaatccggatgcagctggagcaaatgatgaagacgcagctcgcttacggaatggaagagtatgacatgaagccgctgcctcatccggaagcgctgcaatgctactacttccgtaataaaagcggtttgtttctcggagatcttgaacgttatctgacggcagaggaagaacaaattccgttagaccatgatgcgtattggaaagaatgggaacgccagatgaaacagtttcacctgctggacgttcactcttccaatcatgtcaaaatgctgtcggaac

SEQ ID NO: 26 - DNA, 9960 bp, unidentified
  source 1..9960: mol_type = genomic DNA
cgaagccgcaaaacgccattaaggcattctgcgaaaaactgtacgcaaacaaaggaacgataagcgcaaatttccttaaatctttcagaaaaaagctggacgaaacaaatgagttggtaaaacggtaatagagaacggccggagcaatccggccgctttttttatctatatagaactaaatttactatgtactaaattttcattgttgtattgatttctgtttataagacattaaaaaaacccccgataaacagggggtgatgttaaaatctgacaggaaatgtttcaagccctctcaaaaacacattttttctccatgttacatcctcgggcgcgcctttcagctctatatccggaaaccgttttaaaagcgcagtaaaggcaatggatgcttccatccgggcgagcggagccccgagacaaaaatgaatgccgaatccaaaagagatatgacggttcggtgttctcgctatatttaatgtatcaggatcatcaaaatattccgggtcccgattggctgaaccgatgccgataaatatcatatcgccttttttaatagaaacatctttataagtgaaatcttcaatcgcccatcgatttgccatcatgatgacgggagaggtataacgcaaaagctcttcgaccgccgtctggatattttccggctgccgctttatcttctccatctggtctttgtgcagaagaagcgccagggtgccggagccgagaaggttaaccgttgtttcaagcccggcaacgactaacaaaaacagcatggaatacagctctgattcactcagcttttggccatcctcttcagcgtaaataagcttgctcgtcaagtcttcacccggatgttttcttttttcttcgatcagtgttttcatatattgtttaaattctttgagagcctggttggtcgcatcttgtccgctttctgacgtgtcgaccattgcgtttgtccacacttgaaatttggcgcggtcctctttcggaattccgagcagctcggaaatgacgataaacggaagcggtgcggcgaaatctctcatgatatcagggtctgcttttgtttccatttcatccagcagctcttctgtgatctgctctatcccggtgcgaagctgctcaatcatccggggagtgaacccctgatgcaccaaagatcgaagtcgtgaatgatccggggggtcttttgccagcatatgctctgagacaaaatcgatatcttcactgacattcagcttcgcgatcagctccttcggcatgacccgttttacatctctcgtaattctgccgtcttttaaaaacgccatacaatcatcataacgcgtgatgagccaagccggaaaggtgcctccgaaacgctgcagttcgaatttatgtatggggtcctgctctctgaatctgcttaaaaccgcaaaagggttttcgtgaaactcgggtgagtgtgggtgaaacattgtttttttcatctggatttccccgccttcatgtttttgaaacgacataacaaacagccggtgcctgccgaccggacggcattcctccttttatctgccgaagacactgatcacaacggctcataagtatgtctaaaatatataaggaaacagaagccggttcaatagacaaatcgtaaagccgccattttcgttgattatcaacaaaacagcaaactttttttgccatttcatcatgtgtgttaaatccggctaatatacatatttacgcttcatatcaacttcttaaggaggatgtgaatgaatgcgtttatgaaatgggcggcaagtctgcttttggtgatctctcttcagttcggccttacgggcgccggtatccattcggacagtgctgctcgtgccgcatcccgatacgatcaggtgctgtatttcccgctgtcaaaatatccggagacgggaaatcatataaaagacgccatttcggcaggtcattctgagatttgtacaattgatcggggcggagcggagaatagaaggaaagaatcattgaaaggaattccgacaaagcccgggtttgaccgtgatgaatggcccatggcagtctgcacagaaggcggggcgggggctgatgtcagatatgtaaccccgtcggataaccgcggggccggttcatgggttggaaatcaaatgagcggatattccgacggcacgagagtattatttatcgttcagtagcttctccggttcgccggagttttttttatgctctgaaaaagcggcacgccggctggacatatgaaatggcaaagagtaaacttatatgttggaatcagcatgagaaaggattggatcacattgacagtgactacataccgaaatattgcacttttacttctttgtttaacagcgggaattgtggatgtgatcggttatttaagcctgggacatatctttaccgccaatatgacggggaacatcgttctgttcggcctggcgatcggcaactcgtggcaccttgccgtactcaaatcgttaacctcgttaatcggatttattgccggcgttattgccgcggccctgcttgtgggcaataaaaagaaatcattctggcctcctgcggtaactgccgctttagctattgaaggagctgttttgctgctgtttgctttgttctcctgcttttttgcagatgccatttctgtttatgtgctgattatgctgctcagcttcgcaatgggaatgcagacgacagccgcgagaaaacttggtgtagcggggatttccacgaccgtattaacgggaacgctcgctaatttttttgaagatctcaccgcaaggttttacaaagcagacaagcggaagcagtttacgagagatgctgtattgcgcgcgcttgccttggttctgtattgctttggggcggtcataggcgcttttgccgaaccggattataagtttgccgtgatctggcttccgattatcattattttaggcattatgctttgcgcattcacgatgttccacggatattctgaagaaaaaaaccaataaaaaaacgccgctgattcagggcgttttttctttttattgctctttccgggtcccgattgtataaacggcccgatctgtatgtacctttaatccgtcttgctccttttggatggtgatgtcgccgagcagagggatttcatacgcatcttggggaagcgggacatttccttcctccgtataggttgtgccgtgcccttttaagacgagttcttcgtccgctacatagtcatcgggatggtttgtccggcggacgagaacatcatcaaggtgaagcatgatctggtccatatcagaaatctcgtttcggctgtctgtcagctctccttttctgatatcaagcgtttgtccgatctgtttttgcagccattcagacaattcggtgtttgtaagtgccattgtgattcctccttttatctacaccatacttattccctatttttttagaaatacactgccttgcattgatctctttttctgacagccggttctgaagcattccgagttccttcattataaatgtttcatctgttcacctgaaaaacggagtcatctgcaggtttagacgaccccgttctcatgataaaaaggttactctttcggcacggctttttcagcgtttactgcgcccgctccgtaaatgttcggatcttcatttgtccattgatccggactttccttgatcagtgttttcacttcgtcaggcgtaagctgcgggttctgctggaggattaaggcggcaatgcccgcacagatcggagtggccatggacgtgcccgacagactgaaatacaacgaccctacacgatttgatttttgtaatttgtcaaggtatgaacgcggagaacggagagatacgatatcgaccccgggcgcaagaatgtcaggcttctcttttccgtacaccgtcgggccgcggcttgagaaagaagcgaccgtatcgtcctcattgccggccgtatcattgtcatcataggcgcctaccgtgatgaccttctcgctgacaccggggcttgaaatcgtttgagcttccggaccggagttgcccgcggcgacgcagacgacgattccttcattccatgcctcttcaacggccttgacgagcgggtcgtccgtttctttatcatatctgagcgcatctccgcccagtgacatgctgatgattttgatcggattctttgtgtgttctttattatattgtatgcaccattccacaccttcaatgatatcagccaaggttcctgatcctgatttgtttaaaaccttgactccgatcagatcagcttccggcgccggcccctgatatttaccggaagatgaagcgccgctgcttgcgatatcaccggcgcaatgggtgccgtgtccgttgtcatcatacggttctgtcttttggttgacaaagtccgcaaatccgataactctgccttctaaatcgggatgctgatagattccggtatcaataacggcgacggttacgccttttcctgttaatacggtgccgttacgggtcacttcttttgcatgagacgattcagctgccgtatcaaggagcgcttttacttcacggtttaaatagattttacggatatcccggcattgcgaaagaagcatgtgaagggcggatggagtgacttcggcgctgcagcaattgattgtttgaaactgcttttttatggtgcagcgtttttcttttgataagacctgtcctgtgctgtgaaaaccggattcatggcagccctcttcaaattcaatgatgactgacattttctttctgtttttcacctttttttcgaaaaacttgtgcaaaaagcaaggtgtccatttgaaaggtttatataactgtaatacgttttctctgagcggccagtccagcttatgagcatttgaccgcaccatttgtaccattgagtaaccaaacatatatacttttcctcccattctggcgattctctctataccatatgaacaggccgtatgttcggtcagtttgtttgtccttcaggagcgctggttacttgcggctttttcaggagaagaaatcgagttgggatggaacatttgttcgtttataatgaagccggcggaaggaggtatatacatgaacataatattgagacattccttggaaaaacagcttccgattgatattatatatatatgaaaaaagacggctccatttcaaaacgaaccattatgataaaaaaaggggatgagcgggctgtaacggcattttgctgtacaaaaaggcagatgagaacttttttgacgggctccattctgtcctgtgcatttgcttcgggaggcggatattcatttttaaaagcgtattcttgatgccgtccgtttccggccgtgaatctcccgcccctgaaaattcgggcgttcctccctttttttttgccctctctcaaccttttttcactgatgcccggcatttgagtgcttgaaccgtgacaatccattgatttttcgaaaccgggaagtttttcactgatgataatgttaatagaatgttaagataagacctattgacaaaaatccatataatgaacatgtcatttttcagaaaggagcaaagaaggagagctgatgaaaagaattttacttaatgtcatcattatcgtagcaatggtcttactaataagatgtgctcattatgcgtcagaccctgatccctcgaattcttctgatacatacacagattttaaagatttggctgaacatgaagaccccgatgattatcatatatcgtacaataagcgaaaggggagccccgtattgatcatgtcgccgcacggagggagaattgagggcggggtgagcgagatcgtccgctctttcagagatgattattcaacctatctgtttgagggattaaagacgcatgataaccagactcttcatattacgagcaccaggttcgatgagccttcagcagtcgaaagcattaagcagcaccattatgttatcgcagttcacggttataaaggcgacgagaaaaatactcttgtcggaggaagcgaccggaagcgggcgaaaaaattggtgagagctctcgaacgaaacggattttctgcagaattggcaacctcaaaaaccggtctggccggggttgacaccgaaaatatcaataaccaggcgcaaaccgggctgagcatacagcttgagatcagcagagaacagcgggaagcgttttttgataattttgatcaccgcgaaagggaattcacaaaaacggaggaattttaccggtttgtccgtacgataaaaagagtgatcaatcaagagtattcgtaagcttaaaaaaagacaaacgagtgaaagatgagtaaactgaaaaaaaggaggtttgtcccttggcaaaacatcacttttatttaaaagcaaattggcccggaaatcgcaatgacgtcggcgatataaaaagcggccggctggaaacggctgtgtccattcctaaggaaatggacggacccggagaaggcacaaatcctgatgaaatgctgcttggagcggccgctacctgttatattataacgcttgcggcgatgatggaaagaagcggcctggataaagaagatctacatatggaatctgaaggaattgtagacgtcacgaacggtgtgttcacgtaccagaaaatcattcaccgcccgtttatattattaaagccggatgcagccgaaacggactttatgctcgccaggaagttagcgcaaaaagcggagtcctcatgtatgatttcacgagcggtccaaggaaacgtggagatggagcttgaggttgaagtaaaagctgcccgtgcttgacgagttcaaacggtcagctccgggccgtttgattttttttagctttttgagacttggaacggttcagcaggatgacgcccgtgatgaccaaaacaagcccgatcatcattttaactgtcatggcctccttaaaaacaaaaaagccgacaattgccgttaaagcggtgccgacacctgaccatgtcgcgtatgcggcggataaatcaaggcttttcagtgagaagctcaaaaacataaaagaggctccgattccggcaatgacgccgagagcgggccacaggtgcgtaaaaccgtccgacagcttcagcatcgtgctcccgaacacttctgatacaatggctaatgccaagcaaagtaatcctttcattagcatgcctctcctttctaccaattcatcaaaatgacaccggccaataagaaaaagagtccgatgacggcctgcttgtccagtttttcatgaaaccatttcgctccgataatggatgcgagcaccgttccggctccgctccatgttgcataggataagctgagcggaatgagattcagtgtcagtgaaagcatataaaaggccaatgaatagcctgccactacaagcgcgctcggcaaaaaccgcgtaaatccgcgggagaccttcagcatggccgcgccgattgactctgataaaatagcaattgtgaggaagatataacccagaatcatatcaaatccccctctcatcataatccttatgccttttgtttaaggcagtaaaataaaataatatccgaactttgcccgccaagtcaagacattaggcctatacaaaaaggcccctgaaaggagccctttttgattgtcttagtagtaatacggatagtaagggtagccgtaacctccgtacaaatacggattgaacaaagcgcttccgacaagccccccgacaagccctccgacaaacggtccgccgtaaaaaccaaacggccgtccgaacccgccaaaccaagggcggccaaaccaaggccggccaaaaccaaaacccggtcgcccgaagccgaatccgaatccgggacgcccgaacccgaagcccggccgtccgaacccaaatccgggccgtccaaactgtctcatatcactatctgcttcgtatgtgtattcttcaatcataattaaggtacctccaagaataaatttgagatgataacagagtattcagcccaccaatttttgattgggcgtttatcaaaaaaataaaggagtgagcttttgtgggcctgtatcagtccgattggtcaaaaaaacctccgcacacacacgcataccgggctgttacggatgtagtgcaggggcattatcatttgattgaaggcttttcgcagccggagaacggcagcaatacagataagcatacacatttctatacgggcatcacctcgtttgaaaacggccattttcataggtattacggcatttccggccctgccatcccgagggttgacggaacacattaccatgagctttcagagaccacctttgccgcctacaatgagccgattcagatccagcacggcggcgtcgtttacgatccgcgggaggaaggacggaaaacgcaccgccatacgctcaaaggaaaaacgagagaggttgtcggaaacgagccgatcgggtggtagcggacgtttagcgctcaggaaaaagggcaaaatggataggtgcctgaagaaatgaatgctataattagggggagatttataaaagagagtgatacatattgaaaaataaaaaacagcctgtcgtcatactcgtcggaccgacggcagtcggaaaaacaaaactaagcatcggactcgcgaaaatgctgaatgcggaaatcatcagcggagattccatgcaggtatataaagggatggacatcggaacggccaaagtcaccgaagaagagacagaaggcgtgccgcatcacctgattgatattttggagccgtccgacactttttcaacggcggattatcagaaaatggtaaggggaaaaatcaccgagattgccgatagaggaaagctgccgatgattgtcggggggacgggtctgtacattcagtccgtgctgtatgactatacgttcacggaaaactccggcgaccctgtattccgggagcgcatggagaaactggcggctgaagaaggggcggaagcgcttcatgccaagcttgcgacagccgacccggaagcggcggcagccattcatcaaaacaatacgcgaagagtcatccgcgcgctcgaaatcttgcatgtatcgggaaaaacaatgtcagagcatcttaaaacccaaaaacgggagctgctttacaatgcggtccttatcggtcttacaatggacagagaagtgctgtacggccggattaacgagcgcgttgacctgatgctggaaaccggccttcttgacgaagtaaagcggctgtatgatatgaatatacgggacagccaatcgattcaggcgatcggctataaagagctgtatgaatatcttgatggaaaagtgccgctccctcaggctgtggaacaattaaaacaaaattctaggcgatatgcgaaacgccagctgacctggtttcgcaataaaatgcccgtttcatggacagatatgacgccgcctgttgatattgagcagaaaaaaaaggaaattttcgagtgcatagcaggaaaactggaaatttaatcgaaacagtattgtattgagaaccaaggaggacgaaacatgaaaccgattaatattcaagatcagtttttgaatcaaatccgaaaagacaatacgttcgtcacggtgtttttactcaacggcttccagcttcgcggccaggtaaaaggctttgacaactttaccgttttgctcgaaacggaggggaagcagcagcttatttataaacatgccatctccacgttcgccccgcaaaaaaacgtccagcttgagcttgaatagactttttgaaaaagaccatgaacaatggtctttttctatgcgcatctgtatattgcaggctgtatatcggaaacataaaagggggttcggaaaatcgtgttcgaaagtgaaaatgaactgagaagaatcaggattacgctcgtttggatcgctgtttttttgctgtttggagcttgcagcaatcatg

SEQ ID NO: 27 - DNA, 1135 bp, unidentified
  source 1..1135: mol_type = genomic DNA
agacggtggtgacagatgatgacagcggctcaacggatgccgaaccgcagacagaaggcatggtgcagctggaacataatcgtttcggcatcgttgaggacggttacttaaaaatataccgttatgatgaaaaaacgaataaaatcgaactgaaaaaagaagcggcgctggatgagcttgaataaatttcacaagcgttcgaatttacaaagcgggccgcacaagctacactaagtactgacattgatcatgcagagggtgagacgattgaaacacaaggtgataatcaaccattgggaagaaacttgtgaagatgattcctgctatgaatatggaaccagcatcatcgttgacggcaaagaactgatcagagaggcttccatcacaagcgctctgaaagccgtactgaaagaaatcggtgctgatgtggagattgaagaaaccgttgaaagcgaaaaaaaatgtgattcttacaaaaaataagaataaaagtctagactactaagcagttttttttcatgttatgatagacatcggcttacacatgacaccttgagtaggatgagagatgattctcgttctgctctcttttttgggcgttaatcgggccagagccaaagtccgctctttcaaaaatatgaattttttataaatcgatctcaatatgttgtgtttttctgagaggttagtatactatatatagtgttaagagattcattatttttgataggtcgtgataaggatggttcaaatcatatttgattcaaagacagggaatgttcagcgttttgtgaataaaacagactttcagctgatccgcaaggtggacgaaacggaccacgtgggcactccgtttgttttggtcacctacacgacaaacttcggtcaggtaccggcatcaacacaatcatttctcgaaaaatacgcccatctcttattaggagtcgctgcgagcggcaacaaagtatggggcgataactttgcaaaaagcgccgataccatttcaagacaatatcaggtgccgatcttgcacaaatttgaactcagcggcacatctaaagacgttgaattgtttactcaggaggtagaaagagttgtcacaaaatcaagtgccaaaatggatccagttaaataa

SEQ ID NO: 28 - DNA, 27 bp, unidentified
  source 1..27: mol_type = other DNA
tttgtcgagatgtattcgcagatcgcc

SEQ ID NO: 29 - DNA, 27 bp, unidentified
  source 1..27: mol_type = other DNA
tttgaattcgccgatttcagttcgtac

SEQ ID NO: 30 - DNA, 29 bp, unidentified
  source 1..29: mol_type = other DNA
tttgtcgagaccgagaagagagtcgaaac

SEQ ID NO: 31 - DNA, 29 bp, unidentified
  source 1..29: mol_type = other DNA
tttgaattcaagccgtatacaaaggcctg

SEQ ID NO: 32 - DNA, 28 bp, unidentified
  source 1..28: mol_type = other DNA
tttgtcgagtgcagaaagcagaatgccc

SEQ ID NO: 33 - DNA, 29 bp, unidentified
  source 1..29: mol_type = other DNA
tttgaattccacgcatgagaagcgttgaa

SEQ ID NO: 34 - DNA, 29 bp, unidentified
  source 1..29: mol_type = other DNA
tttgtcgagattccgacagcgtgaaacag

SEQ ID NO: 35 - DNA, 29 bp, unidentified
  source 1..29: mol_type = other DNA
tttgaattcgagaaagcagaagctcttcg

(19) United States
(12) Patent Application Publication
(10) Pub. No.: US 2025/0151732 A1
(43) Pub. Date: May 15, 2025

(54) MICROBIAL INOCULANT CONTAINING BACILLUS VELEZENSIS XY40-1, PREPARATION METHOD, AND APPLICATION THEREOF

(71) Applicants: Hunan Vegetable Research Institute, Changsha (CN); Hunan Xiangyan Seed Industry Co., Ltd., Changsha (CN); Hunan Suncas Agricultural Development Co., Ltd., Changde (CN); Hunan Longke Wisdom Agricultural Technology Co., Ltd., Changsha (CN)

(72) Inventors: Xin Li, Changsha (CN); Yu Tao, Changsha (CN); Rongyun Liu, Changsha (CN); Jianhua Zhang, Changde (CN); Xingxi Li, Changsha (CN); Chi Zhou, Changsha (CN); Wu Miao, Changsha (CN); Le Liu, Changde (CN); Xuefeng Li, Changsha (CN)

(21) Appl. No.: 18/443,310

(22) Filed: Feb. 16, 2024

(30) Foreign Application Priority Data
Nov. 13, 2023 (CN) 202311504993.1

(51) Int. Cl. A01N 63/22; A01P 1/00; C12N 1/20; C12R 1/07

(52) U.S. Cl. CPC A01N 63/22; A01P 1/00; C12N 1/205; C12R 2001/07

(57) ABSTRACT

A microbial inoculant containing Bacillus velezensis XY40-1, a preparation method and an application thereof are disclosed, which creatively improves the fermentation medium formula for seed liquid, bacterial solution, and other fermentation processes, greatly shortens the fermentation cycle, significantly reduces fermentation costs, enriches the content and types of microbial metabolites, and enhances the functions of series products such as Bacillus velezensis inoculant in terms of biocontrol and growth promotion. The biological pesticide suspension agent provided by the present disclosure can effectively prevent and control pepper phytophthora blight. Microbial seed coating agents can significantly improve the germination rate of seeds of pepper, eggplant, and cabbage, and have strong application prospects.

### CROSS-REFERENCE TO RELATED APPLICATIONS

This application claims priority of Chinese Patent Application No. 202311504993.1, filed on Nov. 13, 2023, the entire contents of which are incorporated herein by reference.

### DEPOSIT MATERIALS

Bacillus velezensis XY40-1, is deposited in “China Center for Type Culture Collection” which has address as “Wuhan University Wuhan 430072 China” in March 29 2022, with a CCTCC designation number CCTCC M 2022342. The viability of the culture was tested on Apr. 5 2022, and the deposited culture was viable.

### INCORPORATES BY REFERENCE OF SEQUENCE LISTING XML

A XML file is incorporates by reference. The file name is 18443310UPDATED.xml; the creation date is May 22 2024, and the size of the file is 278020 bytes.

### TECHNICAL FIELD

The present disclosure relates to the field of microorganisms and plant disease prevention and control technology, and specifically relates to a microbial inoculant containing Bacillus velezensis XY40-1, a preparation method and an application thereof.

### BACKGROUND

Bacillus velezensis, as a new species of Bacillus, is widely distributed in nature and has strong enzyme production ability, abundant secondary metabolites, strong stress resistance, and high antibacterial activity. Research has shown that Bacillus velezensis can effectively inhibit various plant pathogens, such as Phytophthora capsici, Fusarium solani, Fusarium oxysporum, and Rhizoctonia solani, as well as several pathogenic fungi on leafy vegetables, including Rhizoctonia solani, Phoma, Botrytis cinerea, and Sclerotinia sclerotiorum effectively preventing and controlling various plant diseases.

Microbial control exerts inhibitory effects on agricultural pests and diseases by extracting and modifying organisms such as bacteria, fungi, and viruses. Chemical control poses problems such as overuse and environmental risks. Prolonged use of chemical agents can lead to the development of antibiotic resistance in pathogens and increase agricultural production costs. The use of microorganisms as biological pesticide has the characteristics of being green, efficient, and continuously controlling pests and diseases.

In agriculture, seeds are an indispensable means of production and the foundation of agricultural production. At present, pests and diseases affect the germination rate and quality of seeds, and the traditional use of pesticides to control pests and diseases has brought unbearable pressure to the ecological environment. The use of seed coating technology can not only improve the germination rate of seeds, but also enhance their ability to resist pests and diseases without affecting the ecological environment. It can also save costs and improve economic benefits. However, the negative impact of chemical seed coating agents on crop production is becoming increasingly significant, and there is an urgent need to develop green, low toxicity, and green biological seed coating agents. The microbial based biological seed coating agents have become a development trend. At present, the development of biological seed coating agents in China is only limited to Bacillus subtilis biological seed coating agents, and there are relatively few other microbial based biological seed coating agents, especially the research and development of Bacillus velezensis biological seed coating agents is relatively lagging behind.

### SUMMARY

The objective of the present disclosure is to provide a microbial inoculant containing Bacillus velezensis XY40-1, a preparation method and an application thereof, aiming to reduce the utilization of chemical pesticides and seek for new directions of Bacillus velezensis in microbial control.

The present disclosure is realized as follows:

In the previous studies, the inventor screened a strain of Bacillus velezensis XY40-1, which has good antagonistic effects against Geotrichum candidum, Curvularia, and coconut gray spot disease. The strain was deposited on Mar. 29, 2022 at the China Center for Type Culture Collection, located at Wuhan University in Wuhan, China, with the deposit number CCTCC NO: M 2022342.

A method for preparing a microbial inoculant containing Bacillus velezensis XY40-1 is provided by the present disclosure, including the following steps:
1) strain activation: inoculating Bacillus velezensis XY40-1 with the deposit number of CCTCC NO: M 2022342 onto LB solid culture medium, and incubating at 28-30° C. for 1-2 days to obtain activated strains;
2) fermented seed cultivation: inoculating the activated strains into LB liquid culture medium, then shaking in a shaker at 160-200 rpm for 7-10 hours under temperature of 36-39° C. to obtain seed liquid;
3) fermentation: inoculating the seed liquid with an inoculation amount of 0.5%-1.5% into the fermentation medium, and then shaking in a shaker at 160-200 rpm for 10-18 hours under temperature of 36-39° C. to obtain the microbial inoculant.

Further, in step 1), the formula of the LB solid culture medium is: 10 g/L of tryptone, 5 g/L of yeast extract, 10 g/L of sodium chloride, 15 mmol/L of agar, with a pH of 7.2-7.3.

Further, in step 2), the formula of the LB liquid culture medium is: 20 g/L of peptone, 10 g/L of sodium chloride, 10 g/L of yeast extract, 50 mmol/L of zinc sulfate, 1000 nmol/L of PQQ (pyrroloquinoline quinone), with a pH of 7.2-7.3.

Further, in step 3), the formula of the fermentation medium is: 20 g/L of soybean meal, 14 g/L of glucose, 10 g/L of magnesium sulfate, 50 mmol/L of zinc sulfate, 20 g/L of peptone, 10 g/L of yeast extract, 10 g/L of sodium chloride, 10 g/L of potassium dihydrogen phosphate, 1000 nmol/L of PQQ, and the pH of a fermentation tank is 7.2-7.3.

A microbial inoculant containing Bacillus velezensis XY40-1 is prepared by the preparation method mentioned above.

The microbial inoculant is an unconcentrated original bacterial solution.

Further, in the microbial inoculant, the effective viable count of Bacillus velezensis XY40-1 is between 8-8.5 billion CFU/ml.

Further, the microbial inoculant further includes active secondary metabolites produced by fermentation of Bacillus velezensis XY40-1, including locillomycin, bacillaene, butirosin, and macrolactin H.

Further, a biosynthetic gene cluster of the locillomycin is a class III lanthipeptide gene cluster, with a nucleotide sequence consisting of a full-length sequence sequentially connected by sequences of SEQ ID NO: 1, SEQ ID NO: 2 and SEQ ID NO: 3, and the class III lanthipeptide gene cluster is derived from the 193714-215381 base sequence of Bacillus velezensis XY40-1.

Further, a biosynthetic gene cluster of butirosin is a PKS-like gene cluster, with a nucleotide sequence consisting of a full-length sequence sequentially connected by sequences of SEQ ID NO: 4, SEQ ID NO: 5, SEQ ID NO: 6 and SEQ ID NO: 7, and the PKS-like gene cluster is derived from the 936194-972547 base sequence of Bacillus velezensis XY40-1.

Further, a biosynthetic gene cluster of the Macrolactin H is a trans-AT PKS gene cluster, with a nucleotide sequence consisting of a full-length sequence sequentially connected by sequences of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16, and the transAT-PKS gene cluster is derived from the 1423622-1511453 base sequence of Bacillus velezensis XY40-1.

Further, a biosynthetic gene cluster of bacillaene is a NRPS-TransAT-PKS hetero-gene cluster, with a nucleotide sequence consisting of a full-length sequence sequentially connected by sequences of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 and SEQ ID NO: 27, and the NRPS-TransAT-PKS hetero-gene cluster is derived from the 1730988-1831722 base sequence of Bacillus velezensis XY40-1.

The preparation method of a microbial inoculant containing Bacillus velezensis XY40-1 provided by the present disclosure creatively improves the fermentation medium formula of seed liquid, bacterial liquid, and other fermentation stages compared to the fermentation method disclosed in the international application (patent number: Ser. No. 18/163,866) “Bacillus venlezensis, Fermentation Method, Fermentation Product and its Application” previously applied by the inventor, this greatly shortens the fermentation cycle, significantly reduces fermentation costs, enriches the content and types of microbial metabolites, and improves the functions of series products such as Bacillus velezensis in terms of biocontrol and growth promotion, so it has more advantages in product application.

The present disclosure further provides an application of a microbial inoculant containing Bacillus velezensis XY40-1 in inhibiting growth of pathogenic bacteria or in preparing growth inhibitors for pathogenic bacteria.

Further, the pathogenic microbes are at least one of Phytophthora capsici Leonian, Sclerotium rolfsii, Colletotrichum capsici, Alternaria alternata, Fusarium oxysporum f.sp. cucumerinum, Pyrenochaeta lycopersici, and Ralstonia solanacearum.

The present disclosure further provides an application of a microbial inoculant containing Bacillus velezensis XY40-1 in a preparation of a pesticide for prevention and control of at least one of Phytophthora blight of pepper (Phytophthora capsici Leonian), southern blight pathogens of pepper (Sclerotium rolfsii), pepper Anthracnose (Colletotrichum capsici), tobacco brown spot (Alternaria alternata), cucumber fusarium wilt (Fusarium oxysporum f.sp. cucumerinum), tomato root rot (Pyrenochaeta lycopersici), and pepper bacterial wilt (Ralstonia solanacearum).

The present disclosure further provides a biological pesticide suspension agent, including a microbial inoculant containing Bacillus velezensis XY40-1.

Further, the biological pesticide suspension agent further includes components of the following mass concentrations: 3.5%-3.8% of wetting dispersant, 6%-8% of thickening agent, 0.16%-0.2% of preservative, 4%-5% of antifreeze, 0.5%-0.6% of ultraviolet protectant, 0.15-0.25% of penetrant, 6.6-7% of synergist, 0.1%-0.5% of defoamer, and components of the following molar concentrations: 900-1100 nmol/L of PQQ.

Preferably, the biological pesticide suspension agent further includes components of the following mass concentrations: 3.8% of wetting dispersant, 8% of thickening agent, 0.16% of preservative, 4% of antifreeze, 0.5% of ultraviolet protectant, 0.2 of penetrant, 6.8% of synergist, 0.2% of defoamer, and components of the following molar concentrations: 1100 nmol/L of PQQ.

It should be noted that in biological pesticide suspension agents, the main function of PQQ is to promote microbial fermentation and maintain activity.

Furthermore, the wetting dispersant is tween+sodium lignosulfonate, wherein the mass ratio of the tween to the sodium lignosulfonate is 1:(0.75-0.9); the thickening agent is a 2% xanthan gum mother liquor; the preservative is casson preservative; the antifreeze is ethylene glycol; the ultraviolet protectant is skimmed milk powder; the penetrant is orange peel essential oil; the synergist is lactose peptide+butanol ester, wherein the mass ratio of the lactose peptide to the butanol ester is 5:95; and the defoamer is dimethyl silicone oil.

Furthermore, the main components of lactose peptides are organic acids, free amino acids, potassium, phosphorus, etc. The concentration of organic acids is 200-250 g/L, the concentration of free amino acids is 100-150 g/L, and the molecular weight is less than 300. The potassium content is 50-80 g/L, and the phosphorus content is 10-15 g/L, which is a natural mixture extracted from corn fermentation.

Furthermore, the main components of organic acids in lactose peptides are propionic acid, lactic acid, citric acid, succinic acid, formic acid, acetic acid, and malonic acid. The main components of the free amino acids are tryptophan, cysteine, methionine, aspartic acid, threonine, serine, glutamic acid, glycine, alanine, valine, leucine, isoleucine, tyrosine, phenylalanine, lysine, histidine, arginine, and proline.

It should be noted that the present disclosure has found that lactose peptides have significant effects on promoting plant growth and inhibiting diseases, but are sensitive to concentration differences. Lower or higher concentrations are not conducive to plant growth, and only the concentration range in the present disclosure has good effects or significant differences in promoting plant growth, improving quality, and inhibiting diseases.

The biological pesticide suspension agent of the present disclosure is diluted 100-200 times before spraying when applied to the prevention and control of Phytophthora capsici.

The present disclosure further provides a microbial seed coating agent containing a microbial inoculant containing Bacillus velezensis XY40-1.

Further, the microbial seed coating agent further includes the following mass concentration components: 3.5%-3.8% of wetting dispersant, 6%-8% of thickening agent, 0.16%-0.2% of preservative, 4%-5% of antifreeze, 0.5%-0.6% of ultraviolet protectant, 0.1%-0.5% of defoamer, 4%-10% of film-forming agent, 0.2%-1% of warning color, 0.05%-0.15% of synergist, and components of the following molar concentrations: 40-60 nmol/L of PQQ.

Preferably, the microbial seed coating agent further includes the following mass concentration components: 3.8% of wetting dispersant, 8% of thickening agent, 0.16% of preservative, 4% of antifreeze, 0.5% of ultraviolet protectant, 0.2% of defoamer, 6% of film-forming agent, 0.65% of warning color, 0.1% of synergist, and components of the following molar concentrations: 50 nmol/L of PQQ.

Furthermore, the wetting dispersant is tween+sodium lignosulfonate, wherein the mass ratio of the tween to the sodium lignosulfonate is 1:(0.75-0.9); the thickening agent is a 2% xanthan gum mother liquor; the preservative is casson preservative; the antifreeze is ethylene glycol; the ultraviolet protectant is skimmed milk powder; the defoamer is dimethyl silicone oil; the film-forming agent is polyvinyl pyrrolidone; the warning color is alkaline fuchsin; and the synergist is gibberellin.

It should be noted that in microbial seed coating agents, PQQ mainly promotes seed germination.

An application of the microbial seed coating agent in seeds of pepper, eggplant, and cabbage.

Preferably, the weight ratio of the microbial seed coating agent to the seed is 1:(25-30).

An application of a microbial inoculant containing Bacillus velezensis XY40-1 in a preparation of preservatives.

The advantageous effects of the present disclosure:

The control effect of Bacillus velezensis XY40-1 on Sclerotium rolfsii, Colletotrichum gloeosporioides, and Alternaria alternata can reach over 80%, with rates of 85.78%, 81.70%, and 80.72%, respectively, so that it can be used in the field of microbial control technology. The biological pesticide suspension agent provided by the present disclosure can effectively prevent and control pepper phytophthora blight. Microbial seed coating agents can significantly improve the germination rate of seeds of capsicum, eggplant, and cabbage, and have strong application prospects.

### BRIEF DESCRIPTION OF THE DRAWINGS

FIG. 1 shows the colony morphology of Bacillus velezensis XY40-1 on LB solid culture medium;

FIG. 2 shows the antagonistic effects of Bacillus velezensis XY40-1 on pepper anthracnose, tobacco brown spot, pepper southern blight, tomato root rot disease, cucumber fusarium wilt, pepper phytophthora blight, and pepper bacterial wilt disease;

FIG. 3 shows the pepper seeds coated with the microbial seed coating agent of the present disclosure;

FIG. 4 shows the germination results of pepper seeds after being coated with the microbial seed coating agents of the present disclosure;

FIG. 5 shows eggplant seeds coated with microbial seed coating agents of the present disclosure;

FIG. 6 shows the germination results of eggplant seeds after being coated with the microbial seed coating agents of the present disclosure;

FIG. 7 shows the Chinese cabbage seeds coated with the microbial seed coating agents of the present disclosure; and

FIG. 8 shows the germination results of Chinese cabbage seeds after being coated with the microbial seed coating agents of the present disclosure.

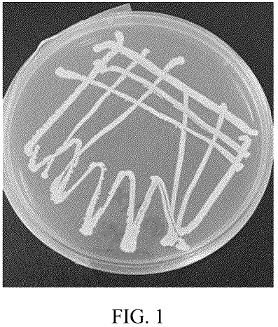

### DETAILED DESCRIPTION OF THE EMBODIMENTS

In order to clarify the objective, technical solution, and advantages of the present disclosure, the following will provide a clear and complete description of the technical solutions in the embodiments of the present disclosure, in conjunction with the embodiments of the present disclosure. Obviously, the described embodiments are a part of the embodiments of the present disclosure, not all of them. Based on the embodiments in the disclosure, all other embodiments obtained by ordinary technicians in this field without creative labor fall within the scope of protection of the present disclosure.

The Bacillus velezensis XY40-1 involved in the present disclosure was deposited on Mar. 29, 2022 at the China Center for Type Culture Collection, located at Wuhan University, Wuhan City, China, with the deposit number of CCTCC NO: M 2022342.

The bacterial culture medium used in the following embodiments is as follows:

LB solid culture medium (g/L): 10 g/L of tryptone, 5 g/L of yeast extract, 10 g/L of sodium chloride, 15 g/L of agar, dissolved in water, finally diluted to 1000 mL, pH 7.2, sterilized under high pressure (121° C., 30 minutes).

LB liquid culture medium (g/L): 20 g/L of peptone, 10 g/L of sodium chloride, 10 g/L of yeast extract, 50 mmol/L of zinc sulfate, 1000 nmol/L of PQQ, finally diluted to 1000 mL, pH 7.2, sterilized under high pressure (121° C., 30 minutes).

Fermentation medium (g/L): 20 g/L of soybean meal, 14 g/L of glucose, 10 g/L of magnesium sulfate, 50 mmol/L of zinc sulfate, 20 g/L of peptone, 10 g/L of yeast extract, 10 g/L of sodium chloride, 10 g/L of potassium dihydrogen phosphate, 1000 nmol/L of PQQ, finally diluted to 1000 mL, pH 7.2, sterilized under high pressure (121° C., 30 minutes).

### Embodiment 1: Antagonistic Experiment of Bacillus velezensis XY40-1 Against Seven Plant Pathogens

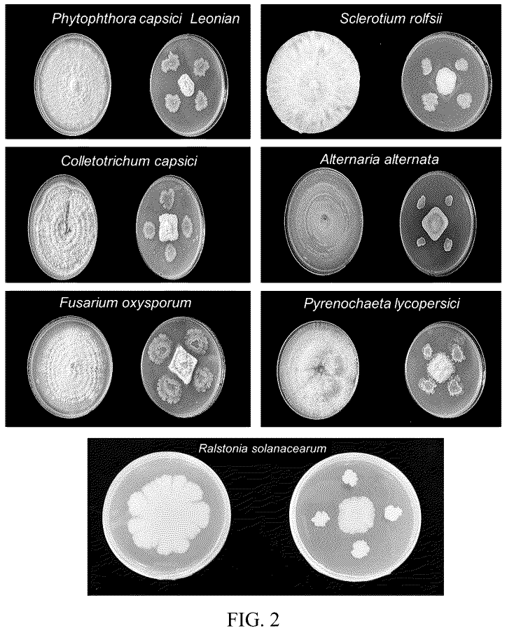

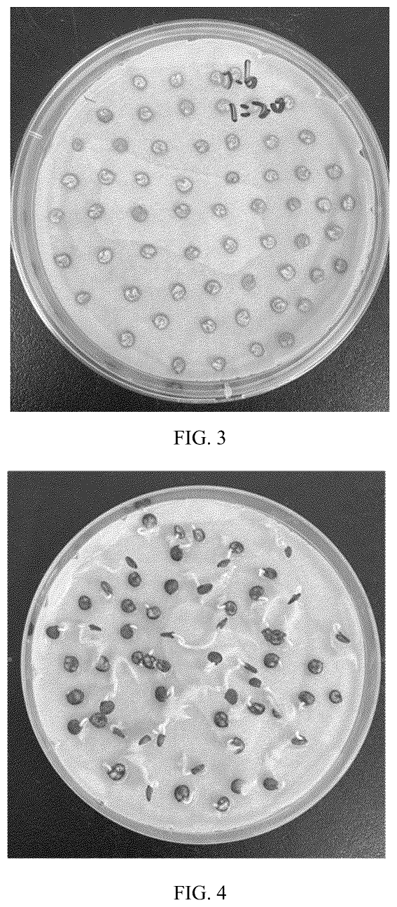

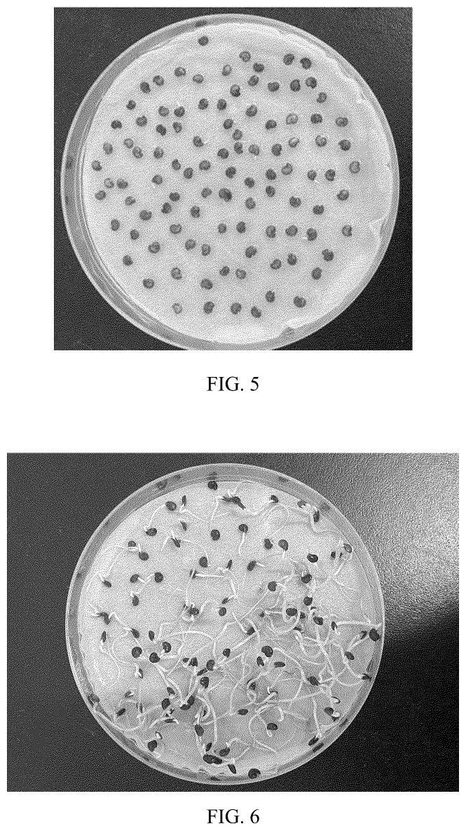

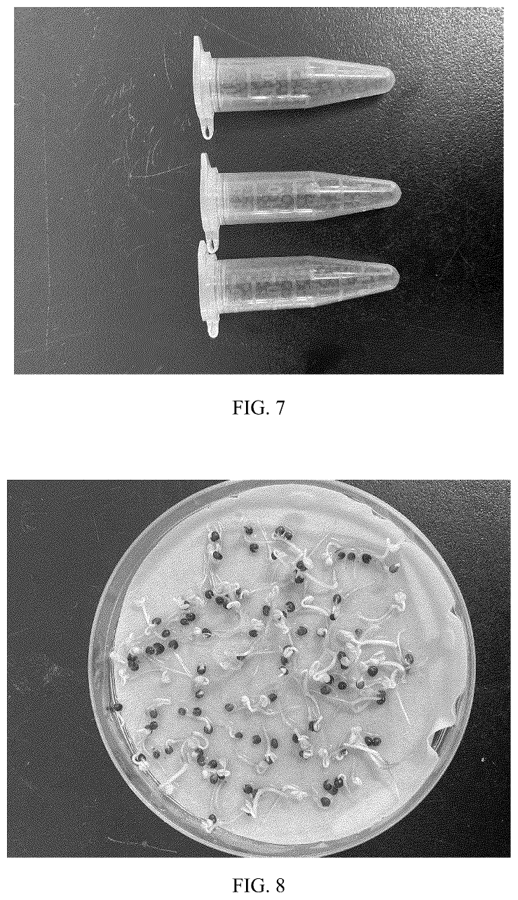

Using plate confrontation method: it refers to inoculating phytophthora, Sclerotium rolfsii, colletotrichum gloeosporioides, Alternaria alternata, Fusarium oxysporum, Pyrenochaeta lycopersici and Ralstonia solanacearum in the center of the PDA plate, and then connecting the strain Bacillus velezensis XY40-1 to a position 2 cm away from the center of the culture medium using a cross over method. After inoculation with pathogenic bacteria, the control group was not inoculated with the strain Bacillus velezensis XY40-1. The incubation was carried out at a constant temperature of 28° C. After the pathogenic bacteria in the control group were fully grown on the agar plate, the width of the antibacterial zone was measured. Each group was treated and repeated for 6 times. Antibacterial inhibition rate (%)=(control colony diameter−treatment colony diameter)/control colony diameter*100%. The results are shown in FIG. 2 and Table 1.

TABLE 1
Inhibition effect of Bacillus velezensis
XY40-1 on 7 plant pathogens

| | CK pathogen | Processed pathogen | Antibacterial |
|---|---|---|---|
| Pathogen name | diameter (cm) | diameter (cm) | efficiency (%) |
| Phytophthora | 8.1 | 1.83 ± 0.15 | 77.47 |
| capsici | | | |
| Sclerotium | 8.3 | 1.18 ± 0.79 | 85.78 |
| rolfsii | | | |
| Colletotrichum | 8.2 | 1.5 ± 0.24 | 81.70 |
| capsici | | | |
| Alternaria | 8.3 | 1.6 ± 0.08 | 80.72 |
| alternata | | | |
| Fusarium | 8.3 | 2.15 ± 0.40 | 74.10 |
| oxysporum | | | |
| Pyrenochaeta | 8.2 | 1.95 ± 0.32 | 76.22 |
| lycopersici | | | |
| Ralstonia | 5.9 | 2.32 ± 0.15 | 60.68 |
| solanacearum | | | |

The experimental results from FIG. 2 and Table 1 indicate that Bacillus velezensis XY40-1 can effectively inhibit the growth of phytophthora, Sclerotium rolfsii, colletotrichum gloeosporioides, Alternaria alternata, Fusarium oxysporum, Pyrenochaeta lycopersici and Ralstonia solanacearum. Especially, the control effect on Sclerotium rolfsii, colletotrichum gloeosporioides, and Alternaria alternata can reach over 80%, with rates of 85.78%, 81.70%, and 80.72%, respectively, and can be used in the field of microbial control technology.

### Embodiment 2: Preparation of Microbial Inoculant Containing Bacillus velezensis XY40-1

The following steps are shown as below:
1) Strain activation: inoculating Bacillus velezensis XY40-1 with the deposit number of CCTCC NO: M 2022342 onto LB solid culture medium, and incubating at 30° C. for 1 days to obtain activated strains;
2) Fermented seed cultivation: inoculating the activated strains into LB liquid culture medium, then shaking in a shaker at 160 rpm for 8 hours under temperature of 36° C. to obtain seed liquid;
3) Fermentation: inoculating the Bacillus velezensis XY40-1 seed liquid with an inoculation amount of 1% into the fermentation medium of the fermentation tank, and then shaking in a shaker at 190 rpm for 12 hours under temperature of 36° C. to obtain the microbial inoculant containing Bacillus velezensis XY40-1.

Test the microbial inoculant prepared in this embodiment, the results shows that, in the microbial inoculant which is an unconcentrated original bacterial solution, the effective viable count of Bacillus velezensis XY40-1 is between 8-8.5 billion CFU/ml.

Using ultra performance liquid chromatography-mass spectrometry for detection (chromatography column: Acquity UPLC HSS T3 1.8 um 2.1*100 mm), the detection and analysis showed that, the microbial inoculant further includes metabolites produced by fermentation, including locillomycin, bacillaene, butirosin, and macrolactin H.

### Embodiment 3: Silencing Expression and Validation of Metabolic Product Synthesis Gene Clusters

Based on the antiSMASH software, bioinformatics analysis was conducted on the biosynthetic gene clusters of locillomycin, bacillaene, butirosin, and macrolactin H, which are derived from Bacillus velezensis XY40-1. It was found that there were synthetic gene clusters for each metabolite mentioned above, and each metabolite had one synthetic gene cluster. The biosynthetic gene cluster of the locillomycin is a class III lanthipeptide gene cluster, with a nucleotide sequence consisting of a full-length sequence sequentially connected by sequences of SEQ ID NO: 1, SEQ ID NO: 2, and SEQ ID NO: 3. The biosynthetic gene cluster of butirosin is a PKS-like gene cluster, with a nucleotide sequence consisting of a full-length sequence sequentially connected by sequences of SEQ ID NO: 4, SEQ ID NO: 5, SEQ ID NO: 6, and SEQ ID NO: 7. The biosynthetic gene cluster of the Macrolactin H is a trans-AT PKS gene cluster, with a nucleotide sequence consisting of a full-length sequence sequentially connected by sequences of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16. The biosynthetic gene cluster of bacillaene is a NRPS-TransAT-PKS hetero-gene cluster, with a nucleotide sequence consisting of a full-length sequence sequentially connected by sequences of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

By conducting silencing expression on the synthetic gene clusters of the above metabolites, their antagonism against 7 major pathogenic microorganisms was verified after fermentation.

Using Bacillus velezensis XY40-1 as a template, primers were designed for the upstream and downstream flanking homologous arm regions of the target genes required for the locillomycin, butirosin, macrolactin H, and bacillaene (see Table 2). The gene segments were amplified and subjected to double enzyme digestion (EcoRI/XhoI) with the resistance vector PTK. The two digested products were mixed with T4 ligase, and connected at a constant temperature of 16° C. for 3 hours. The ligation products were then transformed into the competent cells of Escherichia coli DH5a, then coated on a plate containing 100 mg/L ampicillin resistance and incubated overnight at 36° C. to screen for recombinants, which were preliminarily verified to be correct by enzymatic digestion. A single colony of Bacillus velezensis XY40-1 is selected and inoculated into 5 mL of antibiotic free LB liquid culture medium, shaking and culturing at 36° C., 160 rpm/min for 8 hours. Taking 1 mL of Bacillus velezensis XY40-1 bacterial solution, the bacterial solution is added to 24 mL of new antibiotic free LB liquid culture medium, shaking and culturing on a shaker at 36° C. and 180 rpm/min. Taking 1 mL of culture medium at 5 time intervals of 2 h, 2.5 h, 3 h, 3.5 h, and 4 h, and add the taken culture medium to 1 mL of antibiotic free LB liquid culture medium, shaking and culturing at 36° C. and 200 rpm/min for 1 hour. After 1 hour of bacterial culture at each time period, 50 μL of constructed plasmids were added, then after shaking at 36° C. and 180 rpm/min for 0.5 hours, 20 mL of bacterial solution was taken and evenly coated on a dual antibody plate containing erythromycin (1 ug/mL) and kanamycin (5 ug/mL), incubating overnight at 36° C. Performing biocontrol confrontations on the silencing expression of the biosynthetic gene clusters of the aforementioned metabolites in Bacillus velezensis XY40-1 (as shown in Table 3) to verify their biocontrol differences.

TABLE 2
Primers required for metabolite synthesis

| | Primer | | Sequence |
|---|---|---|---|
| Metabolites | name | Sequence (5′-3′) | number |
| Locillomycin | LKM-F | TTTGTCGAGATGTATTCGCAGATCGCC | SEQ IDNO: 28 |
| | LKM-R | TTTGAATTCGCCGATTTCAGTTCGTAC | SEQ IDNO: 29 |
| Butirosin | DX-F | TTTGTCGAGACCGAGAAGAGAGTCGAAAC | SEQ IDNO: 30 |
| | DX-R | TTTGAATTCAAGCCGTATACAAAGGCCTG | SEQ IDNO: 31 |
| Macrolactin H | DH-F | TTTGTCGAGTGCAGAAAGCAGAATGCCC | SEQ IDNO: 32 |
| | DH-R | TTTGAATTCCACGCATGAGAAGCGTTGAA | SEQ IDNO: 33 |
| Bacillaene | BAC-F | TTTGTCGAGATTCCGACAGCGTGAAACAG | SEQ IDNO: 34 |
| | BAC-R | TTTGAATTCGAGAAAGCAGAAGCTCTTCG | SEQ IDNO: 35 |

TABLE 3
Inhibition effect of Bacillus velezensis XY40-1 on pathogenic bacteria
after silencing expression of various metabolite synthesis gene clusters

| | | Antibacterial | Antibacterial | Antibacterial | Antibacterial |
|---|---|---|---|---|---|
| | | efficiency (%) | efficiency (%) | efficiency (%) | efficiency (%) |
| | Antibacterial | after silencing | after silencing | after silencing | after silencing |
| | efficiency (%) | expression of | expression of | expression of | expression of |
| | of untreated | the synthetic | the synthetic | the synthetic | the synthetic |
| Pathogen | Bacillus | gene cluster of | gene cluster | gene cluster of | gene cluster of |
| name | velezensis | locillomycin | of butirosin | macrolactin H | bacillaene |
| Phytophthora | 77.47 | 68.92 | 66.31 | 55.37 | 71.52 |
| capsici | | | | | |
| Sclerotium | 85.78 | 82.81 | 80.54 | 80.42 | 73.82 |
| rolfsii | | | | | |
| Colletotrichum | 81.70 | 76.52 | 72.05 | 80.82 | 69.27 |
| capsici | | | | | |
| Alternaria | 80.72 | 73.88 | 73.15 | 71.67 | 67.12 |
| alternata | | | | | |
| Fusarium | 74.10 | 70.36 | 71.53 | 63.73 | 69.32 |
| oxysporum | | | | | |
| Pyrenochaeta | 76.22 | 73.89 | 71.54 | 72.36 | 71.11 |
| lycopersici | | | | | |
| Ralstonia | 60.68 | 58.32 | 55.15 | 58.28 | 52.52 |
| solanacearum | | | | | |

### Embodiment 4: Preparation of Biological Pesticide Suspension Agent

In the microbial inoculant containing Bacillus velezensis XY40-1 prepared in Embodiment 2, Tween-80, sodium lignosulfonate, 2% xanthan gum, ethylene glycol, casson preservative, skimmed milk powder, orange peel essential oil, lactose peptide, butanol ester, and dimethyl silicone oil are added. Wherein, the amount of Tween-80 is 2% of the mass of the microbial inoculant, the amount of sodium lignosulfonate is 1.8% of the mass of the microbial inoculant, the amount of 2% xanthan gum is 8% of the mass of microbial inoculant, the amount of ethylene glycol is 4% of the mass of microbial inoculant, the amount of casson preservative is 0.16% of the mass of microbial inoculant, the amount of skimmed milk powder is 0.5% of the mass of microbial inoculant, the amount of orange peel essential oil is 0.2% of the mass of microbial inoculant, the amount of lactose peptide is 0.34% of the mass of microbial inoculant, the amount of butanol ester is 6.46% of the mass of microbial inoculant, and the amount of dimethyl silicone oil is 0.2% of the mass of microbial inoculant, mixing thoroughly, and finally adding PQQ, then mixing uniformly to obtain a biological pesticide suspension. The concentration of PQQ in biological pesticide suspension is 1000 nmol/L. Lactose peptide can be purchased through commercial channels (Zhucheng HAOTIAN Pharm Co., Ltd.), and its main components are organic acids, free amino acids, potassium, phosphorus, etc. The concentration of organic acids is 200-250 g/L, the concentration of free amino acids is 100-150 g/L, the molecular weight is less than 300, the potassium content is 50-80 g/L, and the phosphorus content is 10-15 g/L, which is a natural mixture extracted from corn fermentation.

### Embodiment 5: Prevention and Control of Pepper Phytophthora Blight with Biological Pesticide Suspension Agent

Conducting Verification on the field efficacy of the biological pesticide suspension agent prepared in Embodiment 4 on the biocontrol of pepper phytophthora blight. The biological pesticide suspension agent is diluted with water 150 times, and irrigated the roots of the pepper variety Xingshu 215. Each treatment covers an area of 330 square meters, are applied twice before and at the beginning of the disease. The control group is treated with water and the chemical pesticide of 20% pyraoxystrobin suspension agent (diluted 2000 times, applied twice before and at the beginning of the disease). The results are shown in Table 4.

TABLE 4
Control results of Bacillus velezensis biological pesticide
suspension agent on pepper phytophthora blight

| | Biological pesticide | Chemical | No |
|---|---|---|---|
| Treatment | suspension agent | medicine | treatment |
| Incidence rate of | 57 | 55 | 81 |
| Phytophthora capsici | | | |

### Embodiment 6: Preparation of Microbial Seed Coating Agent

In the microbial inoculant containing Bacillus velezensis XY40-1 prepared in Embodiment 2, Tween-80, sodium lignosulfonate, 2% xanthan gum, ethylene glycol, casson preservative, skimmed milk powder, dimethyl silicone oil, alkaline fuchsin, gibberellin, and polyvinyl pyrrolidone are added. Wherein the amount of Tween-80 is 2% of the mass of the microbial inoculant, and the amount of sodium lignosulfonate is 1.8% of the mass of the microbial inoculant, the amount of 2% xanthan gum is 8% of the mass of microbial inoculant, the amount of ethylene glycol is 4% of the mass of microbial inoculant, the amount of casson preservative is 0.16% of the mass of microbial inoculant, the amount of skimmed milk powder is 0.5% of the mass of microbial inoculant, the amount of dimethyl silicone oil is 0.2% of the mass of microbial inoculant, the amount of alkaline fuchsin is 0.65% of the mass of microbial inoculant, the amount of gibberellin is 0.1% of the mass of microbial inoculant (dissolved in 2 milliliters of anhydrous ethanol), and the amount of polyvinyl pyrrolidone is 6% of the mass of microbial inoculant, mixing thoroughly, and finally adding 50 nmol/L PQQ, then mixing uniformly to obtain microbial seed coating agent.

### Comparative Example 1

Compared with Embodiment 6, the only difference in Comparative Example is that PQQ is not added at the end, and other conditions remain unchanged.

### Embodiment 7: Determination of the Effect of Microbial Seed Coating Agent on Crop Germination

### 1. The Effect of the Microbial Seed Coating Agents on the Germination Rate of Pepper Seeds

Taking microbial seed coating agent in Embodiment 6, mixing it with pepper seeds in a ratio of 1:25 by weight, then coating, and then drying in the shade. Taking it out and place it in a culture dish with moist filter paper for sowing. Adding 3 mL of sterilized water, and then adding 1 mL of sterilized water every day. The seed soaked in clean water CK and the microbial seed coating agent prepared in comparative example 1 are taken as control groups. Incubating at 28° C., and after 4 days, the seed germination potential is counted and the number of sprouts is counted daily. After 7 days, the germination rate of pepper seeds is counted. The experimental results are shown in Table 5.

$$Germination potential ( \% ) = ( number of germinated seeds in 4 days / total number of tested seeds ) \times 100$$

$$Germination rate ( \% ) = ( number of germinated seeds in 7 days / total number of tested seeds ) \times 100$$

TABLE 5
Effects of microbial seed coating agents
on the germination of pepper seeds

| | Germination | Germination |
|---|---|---|
| Treatment | energy (%) | rate (%) |
| CK | 20 | 66 |
| Microbial coating agent of | 35 | 78 |
| Comparative Example 1 | | |
| Microbial seed coating agent | 45 | 90 |
| of Embodiment 6 | | |

### 2. The Effect of Microbial Seed Coating Agents on the Germination Rate of Eggplant Seeds

Taking microbial seed coating agent in Embodiment 6, mixing it with eggplant seeds in a ratio of 1:30 by weight, then coating, and then drying in the shade. Taking it out and place it in a culture dish with moist filter paper for sowing. Adding 3 mL of sterilized water, and then adding 1 mL of sterilized water every day. The seed soaked in clean water CK and the microbial seed coating agent prepared in comparative example 1 are taken as control groups. Incubating at 28° C., and after 4 days, the seed germination potential is counted and the number of sprouts is counted daily. After 7 days, the germination rate of eggplant seeds is counted. The experimental results are shown in Table 6.

$$Germination potential ( \% ) = ( number of germinated seeds in 4 days / total number of tested seeds ) \times 100$$

$$Germination rate ( \% ) = ( number of germinated seeds in 7 days / total number of tested seeds ) \times 100$$

TABLE 6
Effects of microbial seed coating
agents on eggplant seed germination

| | Germination | Germination |
|---|---|---|
| Treatment | energy (%) | rate (%) |
| CK | 1 | 70 |
| Microbial coating agent of | 27 | 82 |
| Comparative Example 1 | | |
| Microbial seed coating agent | 34 | 98 |
| of Embodiment 6 | | |

### 3. The Effect of Microbial Seed Coating Agents on the Germination Rate of Chinese Cabbage Seeds

Taking microbial seed coating agent in Embodiment 6, mixing it with Chinese cabbage seeds in a ratio of 1:27 by weight, then coating, and then drying in the shade. Taking it out and place it in a culture dish with moist filter paper for sowing. Adding 3 mL of sterilized water, and then adding 1 mL of sterilized water every day. The seed soaked in clean water CK and the microbial seed coating agent prepared in comparative example 1 are taken as control groups. Incubating at 28° C., and after 2 days, the germination rate of Chinese cabbage seeds is counted. The experimental results are shown in Table 7.

TABLE 7
Effects of microbial seed coating agents
on germination of Chinese cabbage seeds

| | Germination |
|---|---|
| Treatment | rate (%) |
| CK | 74 |
| Microbial coating agent of | 88 |
| Comparative Example 1 | |
| Microbial seed coating agent | 99 |
| of Embodiment 6 | |

The experimental results in FIGS. 4, 6, 8, and Tables 5-7 indicate that the microbial seed coating agent provided by the present disclosure can significantly improve the germination rate of seeds of pepper, eggplant, and cabbage.

1. A method for preparing a microbial inoculant containing Bacillus velezensis XY40-1, comprising the following steps:
1) strain activation: inoculating Bacillus velezensis XY40-1 with the deposit number of CCTCC NO: M 2022342 onto LB solid culture medium, and incubating at 28-30° C. for 1-2 days to obtain activated strains;
2) fermented seed cultivation: inoculating the activated strains into LB liquid culture medium, then shaking in a shaker at 160-200 rpm for 7-10 hours under temperature of 36-39° C. to obtain seed liquid;
3) fermentation: inoculating the seed liquid with an inoculation amount of 0.5%-1.5% into the fermentation medium, and then shaking in a shaker at 160-200 rpm for 10-18 hours under temperature of 36-39° C. to obtain the microbial inoculant.

2. The preparation method according to claim 1, wherein in step 2), the formula of the LB liquid culture medium is: 20 g/L of peptone, 10 g/L of sodium chloride, 10 g/L of yeast extract, 50 mmol/L of zinc sulfate, 1000 nmol/L of PQQ, with a pH of 7.2-7.3.

3. The preparation method according to claim 1, wherein in step 3), the formula of the fermentation medium is: 20 g/L of soybean meal, 14 g/L of glucose, 10 g/L of magnesium sulfate, 50 mmol/L of zinc sulfate, 20 g/L of peptone, 10 g/L of yeast extract, 10 g/L of sodium chloride, 10 g/L of potassium dihydrogen phosphate, 1000 nmol/L of PQQ, and the pH of a fermentation tank is 7.2-7.3.

4. The microbial inoculant containing according to claim 1, wherein the microbial inoculant is an unconcentrated original bacterial solution, and an effective viable count of Bacillus velezensis XY40-1 is between 8-8.5 billion CFU/ml, and the microbial inoculant is applied in inhibiting growth of pathogenic bacteria or in preparing growth inhibitors for pathogenic bacteria.

5. The microbial inoculant according to claim 4, wherein the microbial inoculant further comprises active secondary metabolites produced by fermentation of Bacillus velezensis XY40-1, comprising locillomycin, bacillaene, butirosin, and macrolactin H.

6. The microbial inoculant according to claim 5, wherein a biosynthetic gene cluster of the locillomycin is a class III lanthipeptide gene cluster, with a nucleotide sequence consisting of a full-length sequence sequentially connected by sequences of SEQ ID NO: 1, SEQ ID NO: 2, and SEQ ID NO: 3; the class III lanthipeptide gene cluster is derived from the 193714-215381 base sequence of Bacillus velezensis XY40-1; a biosynthetic gene cluster of butirosin is a PKS-like gene cluster, with a nucleotide sequence consisting of a full-length sequence sequentially connected by sequences of SEQ ID NO: 4, SEQ ID NO: 5, SEQ ID NO: 6, and SEQ ID NO: 7; the PKS-like gene cluster is derived from the 936194-972547 base sequence of Bacillus velezensis XY40-1; a biosynthetic gene cluster of the Macrolactin H is a trans-AT PKS gene cluster, with a nucleotide sequence consisting of a full-length sequence sequentially connected by sequences of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16; the transAT-PKS gene cluster is derived from the 1423622-1511453 base sequence of Bacillus velezensis XY40-1; a biosynthetic gene cluster of bacillaene is a NRPS-TransAT-PKS hetero-gene cluster, with a nucleotide sequence consisting of a full-length sequence sequentially connected by sequences of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27; and the NRPS-TransAT-PKS hetero-gene cluster is derived from the 1730988-1831722 base sequence of Bacillus velezensis XY40-1.

7. The microbial inoculant according to claim 4 wherein the pathogenic bacteria are at least one of Phytophthora capsici Leonian, Sclerotium rolfsii, Colletotrichum capsici, Alternaria alternata, Fusarium oxysporum f.sp. cucumerinum, Pyrenochaeta lycopersici, and Ralstonia solanacearum.

8. The microbial inoculant according to claim 4, wherein the microbial inoculant is applied in a preparation of a pesticide for prevention and control of at least one of Phytophthora blight of pepper (Phytophthora capsici Leonian), southern blight pathogens of pepper (Sclerotium rolfsii), pepper Anthracnose (Colletotrichum capsici), tobacco brown spot (Alternaria alternata), cucumber fusarium wilt (Fusarium oxysporum f.sp. cucumerinum), tomato root rot (Pyrenochaeta lycopersici), and pepper bacterial wilt (Ralstonia solanacearum).

9. A biological pesticide suspension agent, comprising a microbial inoculant containing Bacillus velezensis XY40-1 as claimed in claim 4.

10. The biological pesticide suspension agent according to claim 9, wherein the biological pesticide suspension agent further comprises components of the following mass concentrations: 3.5%-3.8% of wetting dispersant, 6%-8% of thickening agent, 0.16%-0.2% of preservative, 4%-5% of antifreeze, 0.5%-0.6% of ultraviolet protectant, 0.15-0.25% of penetrant, 6.6-7% of synergist, 0.1%-0.5% of defoamer, and components of the following molar concentrations: 900-1100 nmol/L of PQQ.

11. The biological pesticide suspension agent according to claim 10, wherein the wetting dispersant is tween+sodium lignosulfonate, a mass ratio of the tween to the sodium lignosulfonate is 1:(0.75-0.9); the thickening agent is a 2% xanthan gum mother liquor; the preservative is casson preservative; the antifreeze is ethylene glycol; the ultraviolet protectant is skimmed milk powder; the penetrant is orange peel essential oil; the synergist is lactose peptide+butanol ester, wherein a mass ratio of the lactose peptide to the butanol ester is 5:95; and the defoamer is dimethyl silicone oil.

12. A microbial seed coating agent containing a microbial inoculant containing Bacillus velezensis XY40-1 as claimed in claim 4.

13. The microbial seed coating agent according to claim 12, wherein the microbial seed coating agent further comprises the following mass concentration components: 3.5%-3.8% of wetting dispersant, 6%-8% of thickening agent, 0.16%-0.2% of preservative, 4%-5% of antifreeze, 0.5%-0.6% of ultraviolet protectant, 0.1%-0.5% of defoamer, 4%-10% of film-forming agent, 0.2%-1% of warning color, 0.05%-0.15% of synergist, and components of the following molar concentrations: 40-60 nmol/L of PQQ.

14. The microbial seed coating agent according to claim 13, wherein the wetting dispersant is tween+sodium lignosulfonate, wherein a mass ratio of the tween to the sodium lignosulfonate is 1:(0.75-0.9); the thickening agent is a 2% xanthan gum mother liquor; the preservative is casson preservative; the antifreeze is ethylene glycol; the ultraviolet protectant is skimmed milk powder; the defoamer is dimethyl silicone oil; the film-forming agent is polyvinyl pyrrolidone; the warning color is alkaline fuchsin; and the synergist is gibberellin.

15. The microbial seed coating agent as claimed in claim 14, wherein the microbial seed coating agent is applied in seeds of pepper, eggplant, or cabbage.

16. The microbial seed coating agent according to claim 15, wherein a weight ratio of the microbial seed coating agent to the seeds is 1:(25-30).

17. The microbial inoculant according to claim 4, wherein the microbial inoculant is used as preservatives.
